(19) United States
(12) Patent Application Publication
(10) Pub. No.: US 2024/0279221 A1
(43) Pub. Date: Aug. 22, 2024

(54) HETEROCYCLIC COMPOUNDS AND METHODS OF USE

(71) Applicant: Schrödinger, Inc., New York, NY (US)

(72) Inventors: Adam Marc Levinson, Astoria, NY (US); Evelyne Houang, Astoria, NY (US); Hideyuki Igawa, Creskill, NJ (US); Kim Louise Hirst, Saffron Walden (GB); Byungchan Kim, West New York, NJ (US); Abba Elias Leffler, Bronx, NY (US); Maria Angel Palomero-Vazquez, Cambridge (GB); Eric Therrien, Blainville, CA (US); Xianhai Huang, Warren, NJ (US); Karl Shawn Watts, Portland, OR (US); Steven Albanese, Brooklyn, NY (US); James Alexander Gordon, Suffolk (GB); Andrew Placzek, New York, NY (US)

(21) Appl. No.: 18/564,291

(22) PCT Filed: May 26, 2022
(86) PCT No.: PCT/US22/31133

§ 371 Date: Nov. 27, 2023

Related U.S. Application Data
(60) Provisional application No. 63/226,980, filed on Jul. 29, 2021.
(60) Provisional application No. 63/193,831, filed on May 27, 2021.

(51) Int. Cl. C07D 471/04; A61K 31/5025; A61K 31/5377; C07D 519/00

(52) U.S. Cl. CPC C07D 471/04; A61K 31/5025; A61K 31/5377; C07D 519/00

(57) ABSTRACT

The present application relates to compounds of Formula (I), as defined herein, and pharmaceutically acceptable salts thereof. The present application also describes pharmaceutical composition comprising a compound of Formula (I), and pharmaceutically acceptable salts thereof, and methods of using the compounds and compositions for inhibiting certain protein-protein interactions, and for treating cancer.

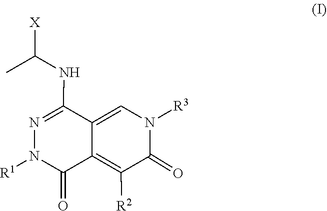

### TECHNICAL FIELD

This present application relates to heterocyclic compounds that are useful for treating proliferative disorders such as cancer.

### BACKGROUND

Cancer is characterized by aberrant cell growth and proliferation. Ras proteins are critical components of signaling networks responsible for controlling cellular proliferation, differentiation, and survival. See, e.g., Fernandes-Medarde and Santos, Genes Cancer, Vol. 2, No. 3, pp. 344-358 (2011). Ras is a GTPase that acts as a molecular switch between an active GTP-bound state and an inactive GDP-bound state—GTP-bound Ras can activate several downstream signaling pathways involved in cell cycle progression, survival, and apoptosis.

Guanine nucleotide exchange factors (GEFs), such as SOS1, are required to activate Ras by facilitating the exchange of GDP (inactive Ras) for GTP (active Ras). SOS1 is itself activated by Ras via an allosteric interaction, which strongly activates the GEF function of SOS1, thus creating a positive feedback loop between SOS1 and Ras. See, e.g., Bandaru, et al., Cold Spring Harb. Perspect Med., Vol. 9, No. 2, a031534 (2019). Mutations in Ras occur in many human cancers, but currently no drug targeting Ras proteins has been approved. See Hillig, et al., Proc. Nat. Acad. Sci., Vol. 117, No. 7, pp. 2551-2560 (2019). Thus, there remains a need for novel therapeutics to disrupt Ras signaling.

### SUMMARY

It has now been found that certain fused compounds are inhibitors of SOS1 activity, and are useful for treating various diseases and disorders, such as cancers.

Accordingly, provided herein is a compound of the Formula (I):

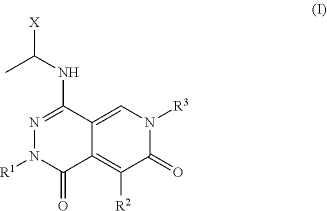

or a pharmaceutically acceptable salt thereof, wherein R¹, R², R³, X and other variables are as defined herein.

Also provided herein is a pharmaceutical composition comprising a compound of Formula (I), or a pharmaceutically acceptable salt thereof, and at least one pharmaceutically acceptable excipient.

Also provided herein is a method of inhibiting mammalian cell proliferation, in vitro or in vivo, comprising contacting a cell with an effective amount of a compound of Formula (I) or a pharmaceutically acceptable salt thereof, or a pharmaceutical composition thereof as defined herein.

Also provided herein is a method of treating cancer in a subject in need of such treatment, comprising administering to the subject an effective amount of a compound of Formula (I), or a pharmaceutically acceptable salt thereof, or a pharmaceutical composition thereof as defined herein.

Also provided herein is a method of treating a SOS1-associated cancer in a subject in need of such treatment, comprising administering to the subject an effective amount of a compound of Formula (I), or a pharmaceutically acceptable salt thereof, or a pharmaceutical composition thereof as defined herein.

Also provided herein is a method of treating a Ras pathway-associated disease or disorder in a subject, comprising administering to a subject identified or diagnosed as having a Ras pathway-associated disease or disorder an effective amount of a compound of Formula (I), or a pharmaceutically acceptable salt thereof, or a pharmaceutical composition thereof as defined herein, to the subject.

Also provided herein is a method of treating a Ras pathway-associated cancer in a subject, comprising administering to a subject identified or diagnosed as having a Ras pathway-associated cancer an effective amount of a compound of Formula (I), or a pharmaceutically acceptable salt thereof, or a pharmaceutical composition thereof as defined herein, to the subject.

Also provided herein is a method of treating a Ras-associated disease or disorder in a subject, comprising administering to a subject identified or diagnosed as having a Ras-associated disease or disorder an effective amount of a compound of Formula (I), or a pharmaceutically acceptable salt thereof, or a pharmaceutical composition thereof as defined herein, to the subject.

Also provided herein is a method of treating a Ras-associated cancer in a subject, comprising administering to a subject identified or diagnosed as having a Ras-associated cancer an effective amount of a compound of Formula (I), or a pharmaceutically acceptable salt thereof, or a pharmaceutical composition thereof as defined herein, to the subject.

Also provided herein is a method of treating a SOS1-associated cancer in a subject, comprising administering to a subject identified or diagnosed as having a SOS1-associated cancer an effective amount of a compound of Formula (I), or a pharmaceutically acceptable salt thereof, or a pharmaceutical composition thereof as defined herein, to the subject.

Also provided herein is a method for treating cancer in a subject in need thereof, comprising:
(a) determining that the cancer is associated with a dysregulation of a Ras pathway gene, a Ras pathway protein, or expression or activity or level of any of the same; and
(b) administering to the subject an effective amount of a compound of Formula (I), or a pharmaceutically acceptable salt thereof, or a pharmaceutical composition thereof as defined herein, to the subject.

Also provided herein is a method for treating cancer in a subject in need thereof, comprising administering to the subject an effective amount of a compound of Formula (I), or a pharmaceutically acceptable salt thereof, or a pharmaceutical composition thereof as defined herein to a subject determined to have a cancer is associated with a dysregulation of a Ras pathway gene, a Ras pathway protein, or expression or activity or level of any of the same.

Also provided herein is a method for treating cancer in a subject in need thereof, comprising:
(a) determining that the cancer is associated with a dysregulation of a Ras gene, a Ras protein, or expression or activity or level of any of the same; and
(b) administering to the subject an effective amount of a compound of Formula (I), or a pharmaceutically acceptable salt thereof, or a pharmaceutical composition thereof as defined herein, to the subject.

Also provided herein is a method for treating cancer in a subject in need thereof, comprising administering to the subject an effective amount of a compound of Formula (I), or a pharmaceutically acceptable salt thereof, or a pharmaceutical composition thereof as defined herein to a subject determined to have a cancer is associated with a dysregulation of a Ras gene, a Ras protein, or expression or activity or level of any of the same.

Also provided herein is a method for treating cancer in a subject in need thereof, comprising administering to the subject an effective amount of a compound of Formula (I), or a pharmaceutically acceptable salt thereof, or a pharmaceutical composition thereof as defined herein to a subject determined to have a cancer is associated with a dysregulation of a SOS1 gene, a SOS1 protein, or expression or activity or level of any of the same.

Also provided herein is a method for treating cancer in a subject in need thereof, comprising:
(a) determining that the cancer is associated with a dysregulation of a SOS1 gene, a SOS1 protein, or expression or activity or level of any of the same; and
(b) administering to the subject an effective amount of a compound of Formula (I), or a pharmaceutically acceptable salt thereof, or a pharmaceutical composition thereof as defined herein, to the subject.

Also provided herein is a method for inhibiting mammalian cell proliferation, comprising contacting the mammalian cell with a compound of Formula (I), or a pharmaceutically acceptable salt thereof.

Also provided herein is a method for inhibiting Ras pathway activity in a mammalian cell, comprising contacting the mammalian cell with a compound of Formula (I), or a pharmaceutically acceptable salt thereof.

Also provided herein is a method for inhibiting SOS1 activity in a mammalian cell, comprising contacting the mammalian cell with a compound of Formula (I), or a pharmaceutically acceptable salt thereof.

Also provided herein is a method for inhibiting Ras activity in a mammalian cell, comprising contacting the mammalian cell with a compound of Formula (I), or a pharmaceutically acceptable salt thereof.

Also provided herein is a method for inhibiting a SOS1-Ras protein-protein interaction in a mammalian cell, comprising contacting the mammalian cell with a compound of Formula (I), or a pharmaceutically acceptable salt thereof.

Also provided herein is a method for inhibiting metastasis in a subject having a particular cancer in need of such treatment, comprising administering to the subject an effective amount of a compound of Formula (I), or a pharmaceutically acceptable salt thereof, or a pharmaceutical composition comprising a compound of Formula (I), or a pharmaceutically acceptable salt thereof.

Also provided herein is a compound of Formula (I), or a pharmaceutically acceptable salt thereof, or a pharmaceutical composition thereof as defined herein for use in the treatment of cancer.

Also provided herein is a compound of Formula (I), or a pharmaceutically acceptable salt thereof, or a pharmaceutical composition thereof as defined herein for use in the treatment of a Ras pathway-associated disease or disorder.

Also provided herein is a compound of Formula (I), or a pharmaceutically acceptable salt thereof, or a pharmaceutical composition thereof as defined herein for use in the treatment of a Ras pathway-associated cancer.

Also provided herein is a compound of Formula (I), or a pharmaceutically acceptable salt thereof or a pharmaceutical composition thereof as defined herein for use in the treatment of cancer and/or inhibiting metastasis associated with a particular cancer.

Also provided herein is a compound of Formula (I), or a pharmaceutically acceptable salt thereof for use in the inhibition of a SOS1-Ras protein-protein interaction in a mammalian cell.

Also provided herein is a compound of Formula (I), or a pharmaceutically acceptable salt thereof, defined herein in the manufacture of a medicament for the inhibition of a SOS1-Ras protein-protein interaction in a mammalian cell.

Also provided herein is a compound of Formula (I), or a pharmaceutically acceptable salt thereof, as defined herein, in the manufacture of a medicament for the treatment of a Ras pathway-associated disease or disorder.

Also provided herein is a compound of Formula (I), or a pharmaceutically acceptable salt thereof, as defined herein, in the manufacture of a medicament for the treatment of a Ras pathway-associated cancer.

Also provided herein is a process for preparing a compound of Formula (I), or a pharmaceutically acceptable salt thereof.

Also provided herein is a compound of Formula (I), or a pharmaceutically acceptable salt thereof obtained by a process of preparing the compound as defined herein.

Other features and advantages of the invention will be apparent from the following detailed description and figures, and from the claims.

### DETAILED DESCRIPTION

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Definitions

The term “compound,” as used herein is meant to include all stereoisomers, geometric isomers, tautomers, and isotopically enriched variants of the structures depicted. Compounds herein identified by name or structure as one particular tautomeric form are intended to include other tautomeric forms unless otherwise specified.

The term “tautomer,” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the invention, and the naming of the compounds does not exclude any tautomer. An example of a tautomeric forms includes the following example:

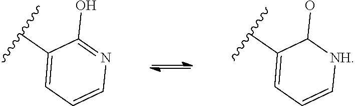 [SMILES: *c1cccnc1O ; *C1=CC=CNC1O]

It will be appreciated that certain compounds provided herein may contain one or more centers of asymmetry and may therefore be prepared and isolated in a mixture of isomers such as a racemic mixture, or in an enantiomerically pure form.

The term “halo” refers to one of the halogens, group 17 of the periodic table. In particular the term refers to fluorine, chlorine, bromine and iodine. Preferably, the term refers to fluorine or chlorine.

The term “C₁-C₆ alkyl” refers to a linear or branched saturated hydrocarbon chain containing 1, 2, 3, 4, 5 or 6 carbon atoms, for example, methyl, ethyl, n-propyl, iso-propyl, n-butyl, sec-butyl, tert-butyl, n-pentyl and n-hexyl.

The term “C₁-C₆ haloalkyl” refers to a C₁-C₆ alkyl group, as defined herein, substituted with at least one halogen atom independently chosen at each occurrence, for example fluorine, chlorine, bromine, and iodine. The halogen atom(s) may be present at any position on the alkyl group. For example, C₁-C₆ haloalkyl may refer to chloromethyl, fluoromethyl, trifluoromethyl, chloroethyl e.g., 1-chloroethyl and 2-chloroethyl, trichloroethyl e.g., 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl.

The term “C₁-C₆ alkoxy” refers to a C₁-C₆ alkyl group, as defined herein, which is attached to a molecule via oxygen. This includes moieties where the alkyl part may be linear or branched, such as methoxy, ethoxy, n-propoxy, iso-propoxy, n-butoxy, sec-butoxy, tert-butoxy, n-pentoxy and n-hexoxy.

The term “C₁-C₆ haloalkoxy” refers to a C₁-C₆ alkoxy group, as defined herein, substituted with at least one halogen atom independently chosen at each occurrence, for example fluorine, chlorine, bromine, and iodine. The halogen atom may be present at any position on the alkyl group. For example, C₁-C₆ haloalkoxy may refer to fluoromethoxy (e.g., 1-fluoromethoxy, 1,1-difluoromethoxy, and 1,1,1-trifluoromethoxy), fluoroethoxy (e.g., 2-fluoroethoxy, 1,2,2-trifluoroethoxy, and 2,2,2-trifluoroethoxy), or chloroethoxy (e.g., 1-chloroethoxy and 2-chloroethoxy, 1,2,2-trichloroethoxy and 2,2,2-trichloroethoxy).

The term “C₁-C₆ hydroxyalkyl” refers to a C₁-C₆ alkyl group, as defined herein, substituted with one or more hydroxyl radical(s). The hydroxyl radical(s) may be present at any position on the hydrocarbon chain. For example, C₁-C₆ hydroxyalkyl may refer to hydroxymethyl, hydroxyethyl (e.g., 1-hydroxyethyl or 2-hydroxyethyl), and 2-hydroxyisopropyl.

The term “C₁-C₆ alkoxyalkyl” refers to a C₁-C₆ alkyl group which is substituted with one or more C₁-C₆ alkoxy groups, as defined herein, wherein the alkoxy group(s) are attached to the alkyl group via an oxygen. This includes moieties where the alkyl part of the C₁-C₆ alkyl or the C₁-C₆ alkoxy may be independently linear or branched, such as methoxyethyl, ethoxyethyl, or 1,3 dimethoxypropyl.

As used herein, the term “cyano” refers to a —CN radical.

As used herein, the term “nitro” refers to a —NO₂ radical.

As used herein, the term “hydroxyl” refers to an —OH radical.

As used herein, the term “amino” refers to a —NH₂ radical.

As used herein, the term “heteroaryl” refers to a 5-10 membered mono- or bicyclic group wherein at least one ring in the system is aromatic; and wherein one or more carbon atoms in at least one ring in the system is/are replaced with an heteroatom independently selected from N, O, and S. Non-limiting examples of heteroaryl groups include furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine.

As used herein, the term “cycloalkyl” refers to a saturated or partially unsaturated mono- or bicyclic carbon group having 3 to 10 carbon atoms, such as C₃-C₁₀ cycloalkyl groups and C₃-C₆ cycloalkyl groups. Bicyclic cycloalkyl groups include fused, spiro, and bridged ring systems. Non-limiting examples of cycloalkyl groups include phenyl, 2,3-dihydro-1H-indene, cyclopropyl, cyclohexyl, spiro[2.3]hexyl, and bicyclo[1.1.1]pentyl.

The term “heterocyclyl” refers to a saturated or partially unsaturated hydrocarbon monocyclic or bicyclic ring system, having 3 to 10 ring atoms, that is not aromatic, having at least one heteroatom within the ring selected from N, O and S. Bicyclic heterocyclyl groups include fused, spiro, and bridged ring systems. The heterocyclyl group may be denoted as, for example, a “5 to 10 membered heterocyclyl group,” which is a ring system containing 5, 6, 7, 8, 9 or 10 atoms at least one being a heteroatom. Heterocyclyl groups can, for example, have 1, 2, 3, or more, heteroatoms. In some embodiments, a heterocyclyl group has one or two independently selected heteroatoms. A heterocycle may further contain one or more carbonyl or thiocarbonyl functionalities, so as to make the definition include oxo-systems and thio-systems such as lactams, lactones, cyclic imides, cyclic thioimides and cyclic carbamates. The heterocyclyl group may be bonded to the rest of the molecule through any carbon atom or through a heteroatom such as nitrogen. Exemplary heterocyclyl groups include, but are not limited to 1,3-dioxolane, 1,4-dioxolane, maleimide, succinimide, dioxopiperazine, hydantoin, imidazoline, imidazolidine, isoxazoline, isoxazolidine, oxazoline, oxazolidine, oxazolidinone, thiazoline, thiazolidine, morpholine, oxirane, piperidine N-oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, azetidine, oxetane, and 2-azaspiro[3.3]heptanyl.

As used herein, when a ring is described as being “partially unsaturated”, it means said ring has one or more additional degrees of unsaturation (in addition to the degree of unsaturation attributed to the ring itself, e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

As used herein, the term “oxo” refers to an “═O” group attached to a carbon atom.

As used herein, the symbol [symbol] depicts the point of attachment of an atom or moiety to the indicated atom or group in the remainder of the molecule.

The compounds of Formula (I) (e.g., Formula (Ia) or (Ib)) include pharmaceutically acceptable salts thereof. In addition, the compounds of Formula (I) also include other salts of such compounds which are not necessarily pharmaceutically acceptable salts, and which may be useful as intermediates for preparing and/or purifying compounds of Formula (I) and/or for separating enantiomers of compounds of Formula (I).

It will further be appreciated that the compounds of Formula (I) or their salts may be isolated in the form of solvates, and accordingly that any such solvate is included within the scope of the present invention. For example, compounds of Formula (I) and salts thereof can exist in unsolvated as well as solvated forms with pharmaceutically acceptable solvents such as water, ethanol, and the like.

In some embodiments, the compounds of Formula (I) include the compounds of Examples 1-182 and stereoisomers and pharmaceutically acceptable salts and solvates thereof. In some embodiments, the compounds of Examples 1-182 are in the free base form. In some embodiments, the compounds of Examples 1-182 are in the form of a pharmaceutically acceptable salt.

The term “pharmaceutically acceptable salt” refers to a formulation of a compound that does not cause significant irritation to an organism to which it is administered and does not abrogate the biological activity and properties of the compound. In some embodiments, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some embodiments, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salts not specifically limited as far as it can be used in medicaments. Examples of a salt that the compounds described herein with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, for example addition salts with mineral acids such as hydrochloric acid, hydrobromic acid, hydroiodic acid, sulfuric acid, nitric acid, and phosphoric acid: organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

Protecting groups can be a temporary substituent which protects a potentially reactive functional group from undesired chemical transformations. The choice of the particular protecting group employed is well within the skill of one of ordinary skill in the art. A number of considerations can determine the choice of protecting group including, but not limited to, the functional group being protected, other functionality present in the molecule, reaction conditions at each step of the synthetic sequence, other protecting groups present in the molecule, functional group tolerance to conditions required to remove the protecting group, and reaction conditions for the thermal decomposition of the compounds provided herein. The field of protecting group chemistry has been reviewed (Greene, T. W.; Wuts, P. G. M. Protective Groups in Organic Synthesis, 2.sup.nd ed.; Wiley: New York, 1991).

A nitrogen protecting group can be any temporary substituent which protects an amine moiety from undesired chemical transformations. Examples of moieties formed when such protecting groups are bonded to an amine include, but are not limited to allylamine, benzylamines (e.g., benzylamine, p-methoxybenzylamine, 2,4-dimethoxybenzylamine, and tritylamine), acetylamide, trichloroacetammide, trifluoroacetamide, pent-4-enamide, phthalimides, carbamates (e.g., methyl carbamate, t-butyl carbamate, benzyl carbamate, allyl carbamates, 2,2,2-trichloroethyl carbamate, and 9-fluorenylmethyl carbamate), imines, and sulfonamides (e.g., benzene sulfonamide, p-toluenesulfonamide, and p-nitrobenzenesulfonamide).

An oxygen protecting group can be any temporary substituent which protects a hydroxyl moiety from undesired chemical transformations. Examples of moieties formed when such protecting groups are bonded to a hydroxyl include, but are not limited to esters (e.g., acetyl, t-butyl carbonyl, and benzoyl), benzyl (e.g., benzyl, p-methoxybenzyl, and 2,4-dimethoxybenzyl, and trityl), carbonates (e.g., methyl carbonate, allyl carbonate, 2,2,2-trichloroethyl carbonate and benzyl carbonate) ketals, and acetals, and ethers.

Compounds provided herein may also contain unnatural proportions of atomic isotopes at one or more of the atoms that constitute such compounds. That is, an atom, in particular when mentioned in relation to a compound according to Formula (I), comprises all isotopes and isotopic mixtures of that atom, either naturally occurring or synthetically produced, either with natural abundance or in an isotopically enriched form. For example, when hydrogen is mentioned, it is understood to refer to ¹H, ²H, ³H or mixtures thereof; when carbon is mentioned, it is understood to refer to ¹¹C, ¹²C, ¹³C, ¹⁴C or mixtures thereof; when nitrogen is mentioned, it is understood to refer to ¹³N, ¹⁴N, ¹⁵N or mixtures thereof; when oxygen is mentioned, it is understood to refer to ¹⁴O, ¹⁵O, ¹⁶O, ¹⁷O, ¹⁸O or mixtures thereof; and when fluoro is mentioned, it is understood to refer to ¹⁸F, ¹⁹F or mixtures thereof; unless expressly noted otherwise. For example, in deuteroalkyl and deuteroalkoxy groups, where one or more hydrogen atoms are specifically replaced with deuterium (²H). As some of the aforementioned isotopes are radioactive, the compounds provided herein therefore also comprise compounds with one or more isotopes of one or more atoms, and mixtures thereof, including radioactive compounds, wherein one or more non-radioactive atoms has been replaced by one of its radioactive enriched isotopes. Radiolabeled compounds are useful as therapeutic agents, e.g., cancer therapeutic agents, research reagents, e.g., assay reagents, and diagnostic agents, e.g., in vivo imaging agents. All isotopic variations of the compounds provided herein, whether radioactive or not, are intended to be encompassed within the scope of the present invention.

The ability of test compounds to act as inhibitors of the SOS1-Ras (e.g., KRas (e.g., KRas G12C)) interaction may be demonstrated by the biological assays described herein. IC₅₀ values for inhibiting the SOS1-Ras interaction are shown in Table A and K_{D} values for binding to SOS1 are shown in Table B.

In some embodiments, the compounds provided herein exhibit brain and/or central nervous system (CNS) penetrance. Such compounds are capable of crossing the blood brain barrier and inhibiting SOS1 activity in the brain and/or other CNS structures. In some embodiments, the compounds provided herein are capable of crossing the blood brain barrier in an effective amount. For example, treatment of a subject with cancer (e.g., a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof) such as a Ras pathway-associated brain or CNS cancer) can include administration (e.g., oral administration) of the compound to the subject. In some such embodiments, the compounds provided herein are useful for treating a primary brain tumor or metastatic brain tumor. For example, a Ras pathway-associated primary brain tumor or metastatic brain tumor.

Compounds of Formula (I), or a pharmaceutically acceptable salt thereof, are useful for treating diseases and disorders which can be treated with a SOS1 inhibitor, such as a Ras pathway-associated disease or disorder (e.g., a SOS1-associated disease or disorder, a Ras-associated disease or disorder (e.g., a KRas-associated disease or disorder, a HRas-associated disease or disorder, and/or a NRas-associated disease or disorder), an EGFR-associated disease or disorder, an ErbB2-associated disease or disorder, an ErbB3-associated disease or disorder, an ErbB4-associated disease or disorder, a NF1-associated disease or disorder, a PDGFR-A-associated disease or disorder, a PDGFR-B-associated disease or disorder, a FGFR1-associated disease or disorder, FGFR2-associated disease or disorder, FGFR3-associated disease or disorder, a IGF1 R-associated disease or disorder, a INSR-associated disease or disorder, a ALK-associated disease or disorder, a ROS-associated disease or disorder, a TrkA-associated disease or disorder, a TrkB-associated disease or disorder, a TrkC-associated disease or disorder, a RET-associated disease or disorder, a c-MET-associated disease or disorder, a VEGFR1-associated disease or disorder, a VEGFR2-associated disease or disorder, a VEGFR3-associated disease or disorder, an AXL-associated disease or disorder, a SHP2-associated disease or disorder, a RAF-associated disease or disorder (e.g., a BRAF-associated disease or disorder), a PI3K-associated disease or disorder, an AKT-associated disease or disorder, an mTOR-associated disease or disorder, a MEK-associated disease or disorder, an ERK-associated disease or disorder, or a combination thereof), including hematological cancers, solid tumors, Neurofibromatosis type 1 (NF1), Noonan Syndrome (NS), LEOPARD syndrome, Capillary Malformation-Arteriovenous Malformation Syndrome (CM-AVM), Costello Syndrome (CS), Cardio-Facio-Cutaneous Syndrome (CFC), Legius Syndrome, and Hereditary gingival fibromatosis.

Compounds of Formula (I), or a pharmaceutically acceptable salt thereof, are useful for treating diseases and disorders which can be treated with a SOS1 inhibitor, such as a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof), including hematological cancers and solid tumors.

In some embodiments, SOS1 inhibitors can exhibit a dissociation constant (K_{D}) with a SOS1 protein of less than about 1000 nM, less than about 500 nM, less than about 200 nM, less than about 100 nM, less than about 50 nM, less than about 25 nM, less than about 10 nM, or less than about 1 nM as measured in an assay as described herein. In some embodiments, a SOS1 inhibitors can exhibit a dissociation constant (K_{D}) with a SOS1 protein of less than about 25 nM, less than about 10 nM, less than about 5 nM, or less than about 1 nM as measured in an assay as provided herein. In some embodiments, SOS1 inhibitors can exhibit inhibition activity (IC₅₀) of a SOS1-KRas (e.g., the G12C mutant of KRas) protein-protein interaction of less than about 1000 nM, less than about 500 nM, less than about 200 nM, less than about 100 nM, less than about 50 nM, less than about 25 nM, less than about 10 nM, or less than about 1 nM as measured in an assay as described herein. In some embodiments, SOS1 inhibitors can exhibit inhibition activity (IC₅₀) of a SOS1-KRas (e.g., the G12C mutant of KRas) protein-protein interaction of less than about 25 nM, less than about 10 nM, less than about 5 nM, or less than about 1 nM as measured in an assay as provided herein.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the term “subject” refers to any animal, including mammals such as mice, rats, other rodents, rabbits, dogs, cats, swine, cattle, sheep, horses, primates, and humans. In some embodiments, the subject is a human. In some embodiments, the subject has experienced and/or exhibited at least one symptom of the disease or disorder to be treated and/or prevented.

In some embodiments, the subject has been identified or diagnosed as having a cancer with a dysregulation of a Ras pathway gene (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), a Ras pathway protein (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or expression or activity, or level of any of the same (a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof)) (e.g., as determined using a regulatory agency-approved, e.g., FDA-approved, assay or kit). In some embodiments, the subject has a tumor that is positive for a dysregulation of a Ras pathway gene, a Ras pathway protein, or expression or activity, or level of any of the same (e.g., as determined using a regulatory agency-approved assay or kit). The subject can be a subject with a tumor(s) that is positive for a dysregulation of a Ras pathway gene, a Ras pathway protein, or expression or activity, or level of any of the same (e.g., identified as positive using a regulatory agency-approved, e.g., FDA-approved, assay or kit). The subject can be a subject whose tumors have a dysregulation of a Ras pathway gene, a Ras pathway protein, or expression or activity, or a level of the same (e.g., where the tumor is identified as such using a regulatory agency-approved, e.g., FDA-approved, kit or assay). In some embodiments, the subject is suspected of having a Ras pathway-associated cancer. In some embodiments, the subject has a clinical record indicating that the subject has a tumor that has a dysregulation of a Ras pathway gene, a Ras pathway protein, or expression or activity, or level of any of the same (and optionally the clinical record indicates that the subject should be treated with any of the compositions provided herein). In some embodiments, the subject is a pediatric subject. In some embodiments, the subject has been identified or diagnosed as having a cancer that, based on histological examination, is determined to be associated with a dysregulation of a Ras pathway gene, a Ras pathway protein, or expression or activity, or level of any of the same (a Ras pathway-associated cancer).

In some embodiments, the subject has been identified or diagnosed as having a cancer with a dysregulation of a Ras gene, a Ras protein, or expression or activity, or level of any of the same (a Ras-associated cancer) (e.g., as determined using a regulatory agency-approved, e.g., FDA-approved, assay or kit). In some embodiments, the subject has a tumor that is positive for a dysregulation of a Ras gene, a Ras protein, or expression or activity, or level of any of the same (e.g., as determined using a regulatory agency-approved assay or kit). The subject can be a subject with a tumor(s) that is positive for a dysregulation of a Ras gene, a Ras protein, or expression or activity, or level of any of the same (e.g., identified as positive using a regulatory agency-approved, e.g., FDA-approved, assay or kit). The subject can be a subject whose tumors have a dysregulation of a Ras gene, a Ras protein, or expression or activity, or a level of the same (e.g., where the tumor is identified as such using a regulatory agency-approved, e.g., FDA-approved, kit or assay). In some embodiments, the subject is suspected of having a Ras-associated cancer. In some embodiments, the subject has a clinical record indicating that the subject has a tumor that has a dysregulation of a Ras gene, a Ras protein, or expression or activity, or level of any of the same (and optionally the clinical record indicates that the subject should be treated with any of the compositions provided herein). In some embodiments, the subject is a pediatric subject. In some embodiments, the subject has been identified or diagnosed as having a cancer that, based on histological examination, is determined to be associated with a dysregulation of a Ras gene, a Ras protein, or expression or activity, or level of any of the same (a Ras-associated cancer).

In some embodiments, the subject has been identified or diagnosed as having a cancer with a dysregulation of a KRas gene, a KRas protein, or expression or activity, or level of any of the same (a KRas-associated cancer) (e.g., as determined using a regulatory agency-approved, e.g., FDA-approved, assay or kit). In some embodiments, the subject has a tumor that is positive for a dysregulation of a KRas gene, a KRas protein, or expression or activity, or level of any of the same (e.g., as determined using a regulatory agency-approved assay or kit). The subject can be a subject with a tumor(s) that is positive for a dysregulation of a KRas gene, a KRas protein, or expression or activity, or level of any of the same (e.g., identified as positive using a regulatory agency-approved, e.g., FDA-approved, assay or kit). The subject can be a subject whose tumors have a dysregulation of a KRas gene, a KRas protein, or expression or activity, or a level of the same (e.g., where the tumor is identified as such using a regulatory agency-approved, e.g., FDA-approved, kit or assay). In some embodiments, the subject is suspected of having a KRas-associated cancer. In some embodiments, the subject has a clinical record indicating that the subject has a tumor that has a dysregulation of a KRas gene, a KRas protein, or expression or activity, or level of any of the same (and optionally the clinical record indicates that the subject should be treated with any of the compositions provided herein). In some embodiments, the subject is a pediatric subject. In some embodiments, the subject has been identified or diagnosed as having a cancer that, based on histological examination, is determined to be associated with a dysregulation of a KRas gene, a KRas protein, or expression or activity, or level of any of the same (a KRas-associated cancer).

In some embodiments, the subject has been identified or diagnosed as having a cancer with a dysregulation of a HRas gene, a HRas protein, or expression or activity, or level of any of the same (a HRas-associated cancer) (e.g., as determined using a regulatory agency-approved, e.g., FDA-approved, assay or kit). In some embodiments, the subject has a tumor that is positive for a dysregulation of a HRas gene, a HRas protein, or expression or activity, or level of any of the same (e.g., as determined using a regulatory agency-approved assay or kit). The subject can be a subject with a tumor(s) that is positive for a dysregulation of a HRas gene, a HRas protein, or expression or activity, or level of any of the same (e.g., identified as positive using a regulatory agency-approved, e.g., FDA-approved, assay or kit). The subject can be a subject whose tumors have a dysregulation of a HRas gene, a HRas protein, or expression or activity, or a level of the same (e.g., where the tumor is identified as such using a regulatory agency-approved, e.g., FDA-approved, kit or assay). In some embodiments, the subject is suspected of having a HRas-associated cancer. In some embodiments, the subject has a clinical record indicating that the subject has a tumor that has a dysregulation of a HRas gene, a HRas protein, or expression or activity, or level of any of the same (and optionally the clinical record indicates that the subject should be treated with any of the compositions provided herein). In some embodiments, the subject is a pediatric subject. In some embodiments, the subject has been identified or diagnosed as having a cancer that, based on histological examination, is determined to be associated with a dysregulation of a HRas gene, a HRas protein, or expression or activity, or level of any of the same (a HRas-associated cancer).

In some embodiments, the subject has been identified or diagnosed as having a cancer with a dysregulation of a NRas gene, a NRas protein, or expression or activity, or level of any of the same (a NRas-associated cancer) (e.g., as determined using a regulatory agency-approved, e.g., FDA-approved, assay or kit). In some embodiments, the subject has a tumor that is positive for a dysregulation of a NRas gene, a NRas protein, or expression or activity, or level of any of the same (e.g., as determined using a regulatory agency-approved assay or kit). The subject can be a subject with a tumor(s) that is positive for a dysregulation of a NRas gene, a NRas protein, or expression or activity, or level of any of the same (e.g., identified as positive using a regulatory agency-approved, e.g., FDA-approved, assay or kit). The subject can be a subject whose tumors have a dysregulation of a NRas gene, a NRas protein, or expression or activity, or a level of the same (e.g., where the tumor is identified as such using a regulatory agency-approved, e.g., FDA-approved, kit or assay). In some embodiments, the subject is suspected of having a NRas-associated cancer. In some embodiments, the subject has a clinical record indicating that the subject has a tumor that has a dysregulation of a NRas gene, a NRas protein, or expression or activity, or level of any of the same (and optionally the clinical record indicates that the subject should be treated with any of the compositions provided herein). In some embodiments, the subject is a pediatric subject. In some embodiments, the subject has been identified or diagnosed as having a cancer that, based on histological examination, is determined to be associated with a dysregulation of a NRas gene, a NRas protein, or expression or activity, or level of any of the same (a NRas-associated cancer).

In some embodiments, the subject has been identified or diagnosed as having a cancer with a dysregulation of a SOS1 gene, a SOS1 protein, or expression or activity, or level of any of the same (a SOS1-associated cancer) (e.g., as determined using a regulatory agency-approved, e.g., FDA-approved, assay or kit). In some embodiments, the subject has a tumor that is positive for a dysregulation of a SOS1 gene, a SOS1 protein, or expression or activity, or level of any of the same (e.g., as determined using a regulatory agency-approved assay or kit). The subject can be a subject with a tumor(s) that is positive for a dysregulation of a SOS1 gene, a SOS1 protein, or expression or activity, or level of any of the same (e.g., identified as positive using a regulatory agency-approved, e.g., FDA-approved, assay or kit). The subject can be a subject whose tumors have a dysregulation of a SOS1 gene, a SOS1 protein, or expression or activity, or a level of the same (e.g., where the tumor is identified as such using a regulatory agency-approved, e.g., FDA-approved, kit or assay). In some embodiments, the subject is suspected of having a SOS1-associated cancer. In some embodiments, the subject has a clinical record indicating that the subject has a tumor that has a dysregulation of a SOS1 gene, a SOS1 protein, or expression or activity, or level of any of the same (and optionally the clinical record indicates that the subject should be treated with any of the compositions provided herein). In some embodiments, the subject is a pediatric subject. In some embodiments, the subject has been identified or diagnosed as having a cancer that, based on histological examination, is determined to be associated with a dysregulation of a SOS1 gene, a SOS1 protein, or expression or activity, or level of any of the same (a SOS1-associated cancer).

The term “pediatric subject” as used herein refers to a subject under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First LR. Pediatric Medicine, 2nd Ed. Baltimore: Williams & Wilkins; 1994. In some embodiments, a pediatric subject is from birth through the first 28 days of life, from 29 days of age to less than two years of age, from two years of age to less than 12 years of age, or 12 years of age through 21 years of age (up to, but not including, the twenty-second birthday). In some embodiments, a pediatric subject is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

In certain embodiments, compounds of Formula (I), or a pharmaceutically acceptable salt thereof are useful for preventing diseases and disorders as defined herein (for example, autoimmune diseases, inflammatory diseases, and cancer). The term “preventing” as used herein means the prevention of the onset, recurrence or spread, in whole or in part, of the disease or condition as described herein, or a symptom thereof.

In certain embodiments, compounds of Formula (I), or a pharmaceutically acceptable salt thereof are useful for preventing diseases and disorders as defined herein (for example, Ras pathway-associated diseases or disorders (e.g., autoimmune diseases, inflammatory diseases, and cancer) as described herein. The term “preventing” as used herein means the prevention of the onset, recurrence or spread, in whole or in part, of the disease or condition as described herein, or a symptom thereof.

Aberrant cell growth and proliferation is a hallmark of cancer. One such pathway through which such aberrant cell growth can occur is through Ras family protein signaling. The human Ras proteins (e.g., KRas (V-Ki-Ras2 Kirsten Rat Sarcoma 2 Viral Oncogene Homolog), HRas (V-Ha-Ras Harvey Rat Sarcoma Viral Oncogene Homolog), and/or NRas (Neuroblastoma RAS Viral (V-Ras) Oncogene Homolog); sometimes also called KRAS, HRAS, and NRAS, or K-Ras H-Ras, and N-Ras, respectively) are membrane-bound guanosine triphosphate (GTP)/guanosine diphosphate (GDP)-binding (G) proteins that are implicated in many oncogenic signaling cascades. Each of these proteins is approximately 21 kD in size. KRas has two common isoforms known as KRas4A and KRas4B.

Mature Ras proteins are typically associated with the cellular membrane via post-translational modification, such as prenylation (e.g., farnesylation of a “CAAX box”, where C represents cysteine, A represents an aliphatic amino acid, and X is methionine, serine, leucine, or glutamine). In the inactive state, Ras proteins are bound to GDP. See, e.g., Adjei, J. Nat'l. Cancer Inst. 93.14 (2001): 1062-1074.

Activation of Ras proteins can be initiated via multiple types of cell-surface receptors including receptor tyrosine kinases (TKIs) (e.g., EGFR, ErbB2, ErbB3, ErbB4, PDGFR-A/B, FGFR1/2/3, IGF1 R, INSR, ALK, ROS, TrkA, TrkB, TrkC, RET, c-MET, VEGFR1/2/3, AXL), T-cell receptors, B-cell receptors, monocyte colony-stimulating factor receptor, G-protein coupled receptors (GPCRs), and integrin family proteins. Activation of one of these types of cell-surface receptors generally leads, directly or indirectly, to activation of one or more guanine nucleotide exchange factors (GEFs), which promote Ras proteins to release GDP, allowing GTP to bind. Non-limiting examples of GEFs include the SOS (Son of Sevenless Homolog) proteins and RASGRF1 (Ras protein specific guanine nucleotide releasing factor 1; also sometimes called Cdc25). For example, upon activation, dimerization, and auto-phosphorylation of EGFR, the receptor can bind to the SH2 domain of the adaptor protein growth-factor-receptor-bound protein 2 (GRB2), which can then bind to a SOS protein (e.g., SOS1 or SOS2, sometimes also called SOS-1 and SOS-2, respectively), thereby co-localizing the SOS protein with the Ras family protein at the cellular membrane. See, e.g., Xuehua et al., Proc. Nat. Acad. Sci. November 2017, 114 (47) E10092-E10101; Vetter and Wittinghofer, Science 294.5545 (2001): 1299-1304; Downward, Nat. Rev. Cancer 3.1 (2003): 11-22; Pierre and Coumoul, Biochem. Pharmacol. 82.9 (2011): 1049-1056.Kortum, et al. Proc. Nat. Acad. Sci. 108.30 (2011): 12407-12412; U.S. Appl. Publ. Nos. 2019/0358230 and 2019/0194192; and PCT Publication Nos. WO 2018/172250 and WO 2019/201848.

Once activated by binding GTP, the Ras proteins can bind to and activate a number of downstream effectors, including the RAF family proteins, phosphatidyl inositol 3-kinases (PI3Ks), and RAL family proteins. See, e.g., Gurung and Bhattacharjee. Oncology & Hematology Review, 2015; 11(2):147-52 (2015). For instance, signaling through the Ras-RAF-MAPK pathway has been implicated in many cancers, including, but not limited to, pancreatic cancer, thyroid cancer (e.g., papillary thyroid cancer), colon cancer, lung cancer (e.g., non-small cell lung cancer), melanoma, biliary tract cancer, small intestinal cancer, endometrial cancer, ovarian cancer, cervical cancer, prostate cancer, soft tissue cancers, peritoneal cancer, stomach cancer, liver cancer, urinary tract cancer, breast cancer, and combinations thereof. See, e.g., Kinsey, et al. Nat. Medicine 25.4 (2019): 620-627; Roberts and Der. Oncogene 26.22 (2007): 3291-3310; Santarpia, et al. Expert Opinion on Therapeutic Targets 16.1 (2012): 103-119. As another example, signaling through the Ras-PI3K/AKT/mammalian target of rapamycin (mTOR) pathway has been shown to play a role in many cancers, including, but not limited to, melanoma, ovarian cancer, cervical cancer, endometrial cancer, breast cancer, prostate cancer, brain cancer (e.g., glioblastoma), lung cancer (e.g., non-small cell lung cancer), pancreatic cancer, bladder cancer, colon cancer, head and neck cancer, leukemia, thyroid cancer, lymphoma, bowel cancer, gastric cancer, and combinations thereof. See, e.g., Chappell, et al. Oncotarget 2.3 (2011): 135; Vara, et al. Cancer Treatment Reviews 30.2 (2004): 193-204; Hennessy, et al. Nat. Rev. Drug Disc. 4.12 (2005): 988-1004; Osaki, et al. Apoptosis 9.6 (2004): 667-676; Luo, et al. Cancer Cell 4.4 (2003): 257-262.

Though Ras proteins have intrinsic GTPase activity, it is typically not physiologically relevant. Instead, hydrolysis of the bound GTP is enhanced (e.g., by up to about 5 orders of magnitude) by the binding of a GTPase-activating protein (GAP), such as neurofibromatosis type 1 (NF1) or p120^{GAP}. See, e.g., Adjei, Journal of the National Cancer Institute 93.14 (2001): 1062-1074; Downward, Nature Reviews Cancer 3.1 (2003): 11-22; Scheffzek, et al. Science 277.5324 (1997): 333-339.

Activating mutations (especially, e.g., at residues G12, G13, and/or Q61) in Ras family proteins are estimated to be present in up to about 30% of all human cancers. Commonly, activating mutations in Ras family proteins render the Ras protein insensitive to the activity of GAPs. See, e.g., Santarpia, et al. Expert Opinion on Therapeutic Targets 16.1 (2012): 103-119. Exemplary, non-limiting examples of Ras mutations are presented in Tables 1 (KRas mutations), 2 (HRas mutations), and 3 (NRas mutations).

The term “Ras pathway-associated disease or disorder” as used herein refers to diseases or disorders associated with or having a dysregulation of a gene in a Ras pathway, a protein in a Ras pathway, or the expression or activity or level of any (e.g., one or more) of the same (e.g., any of the types of dysregulation of a gene in a Ras pathway, a protein in a Ras pathway, or the expression or activity or level of any of the same, as described herein). Non-limiting examples of a Ras pathway-associated diseases or disorders include, for example, Neurofibromatosis type 1 (NF1), Noonan Syndrome (NS), LEOPARD syndrome, Capillary Malformation-Arteriovenous Malformation Syndrome (CM-AVM), Costello Syndrome (CS), Cardio-Facio-Cutaneous Syndrome (CFC), Legius Syndrome, Hereditary gingival fibromatosis, and cancers.

In some embodiments, a Ras pathway-associated disease or disorder is a Ras pathway-associated cancer, such as a KRas-associated cancer, a HRas-associated cancer, a NRas-associated cancer, a SOS1-associated cancer, an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof.

The term “Ras pathway-associated cancer” as used herein refers to cancers associated with or having a dysregulation of a gene in a Ras pathway, a protein in a Ras pathway, or the expression or activity or level of any (e.g., one or more) of the same (e.g., any of the types of dysregulation of a gene in a Ras pathway, a protein in a Ras pathway, or the expression or activity or level of any of the same, as described herein). Non-limiting examples of a Ras pathway-associated cancer are described herein. In some embodiments, a Ras pathway-associated cancer can be a KRas-associated cancer, a HRas-associated cancer, a NRas-associated cancer, a SOS1-associated cancer, an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof.

The term “Ras-associated cancer” as used herein refers to cancers associated with or having a dysregulation of a Ras gene, a Ras protein, or the expression or activity or level of any (e.g., one or more) of the same (e.g., any of the types of dysregulation of a Ras gene, a Ras protein, or the expression or activity or level of any of the same, as described herein). Non-limiting examples of a Ras-associated cancer are described herein. In some embodiments, a Ras-associated cancer can be a KRas-associated cancer, a HRas-associated cancer, a NRas-associated cancer, or a combination thereof.

The phrase “dysregulation of a Ras gene, a Ras protein, or the expression or activity or level of any of the same” refers to a genetic mutation (e.g., a Ras (e.g., KRas, NRas, or HRas) gene translocation that results in the expression of a fusion protein, a mutation in a Ras gene that results in the expression of a Ras protein that includes a deletion of at least one amino acid as compared to a wild type Ras protein, a mutation in a Ras gene that results in the expression of a Ras protein with one or more point mutations as compared to a wild type Ras protein, a mutation in a Ras gene that results in the expression of a Ras protein with at least one inserted amino acid as compared to a wild type Ras protein, a gene duplication that results in an increased level of Ras protein in a cell, or a mutation in a regulatory sequence (e.g., a promoter and/or enhancer) that results in an increased level of Ras protein in a cell), an alternative spliced version of a Ras mRNA (e.g., that results in a Ras protein having a deletion of at least one amino acid in the Ras protein as compared to the wild type Ras protein or that results in a Ras protein having an insertion of at least one amino acid in the Ras protein as compared to the wild type Ras protein), or increased expression (e.g., increased levels) of a wild type Ras protein in a mammalian cell due to aberrant cell signaling and/or dysregulated autocrine/paracrine signaling (e.g., as compared to a control non-cancerous cell). As another example, a dysregulation of a Ras gene, a Ras protein, or expression or activity, or level of any of the same, can be a mutation in a Ras gene that encodes a Ras protein that is constitutively active or has increased activity as compared to a protein encoded by a Ras gene that does not include the mutation. In some embodiments of any of the methods described herein, a dysregulation of a Ras gene, a Ras protein, or the expression or activity or level of any of the same can be selected from the group consisting of a G12 mutation, a G13 mutation, a Q61 mutation, and a combination thereof.

Table 1 lists some non-limiting exemplary KRas mutations. Table 1A lists a non-limiting exemplary KRas fusion. In some embodiments of any of the methods described herein, a dysregulation of a KRas gene, a KRas protein, or the expression or activity or level of any of the same can be selected from the group consisting of a G12 mutation (e.g., G12I, G12A, G12C, G12D, G12E, G12F, G12L, G12N, G12R, G12S, G12T, G12V, G12W, or G12Y), a G13 mutation (e.g., G13A, G13C, G13D, G13E, G13F, G13I, G13M, G13N, G13P, G13R, G13S, G13V, or G13Y), a Q61 mutation (e.g., Q61D, Q61E, Q61H, Q61K, Q61L, Q61P, Q61R), and a combination thereof.

Table 2 lists some non-limiting exemplary HRas mutations. In some embodiments of any of the methods described herein, a dysregulation of a HRas gene, a HRas protein, or the expression or activity or level of any of the same can be selected from the group consisting of a G12 mutation (e.g., G12A, G12C, G12D, G12R, G12S, G12V), a G13 mutation (e.g., G13A, G13C, G13D, G13R, G13S, G13V), a Q61 mutation (e.g., Q61H, Q61K, Q61L, Q61P, Q61R, Q61*), and a combination thereof.

Table 3 lists some non-limiting exemplary HRas mutations. In some embodiments of any of the methods described herein, a dysregulation of a HRas gene, a HRas protein, or the expression or activity or level of any of the same can be selected from the group consisting of a G12 mutation (e.g., G12A, G12C, G12D, G12R, G12S, G12V, G12W, G12N), a G13 mutation (e.g., G13A, G13C, G10D, G13R, G13S, G V), a Q61 mutation (e.g., Q61E, Q61H, Q61K, Q61L, Q61P, Q61R, Q61E, Q61N), and a combination thereof.

TABLE 1
KRAS Protein Amino Acid Substitutions/Insertions/Deletions^{A}

| Amino Acid | Non-Limiting | Non-Limiting Exemplary |
|---|---|---|
| Position(s) | Exemplary Mutations | RAS-Associated Cancers |
| 5 | K5E², K5N², K5R¹ | |
| 6 | L6F¹ | |
| 7 | V7E¹, V7M¹ | |
| 8 | V8A¹, V8V^{1†} | |
| 9 | V9I¹, V9V^{1†} | |
| 10 | G10E¹, G10G^{1†}, G10R¹ | |
| 11 | A11A^{1†}, A11P¹, A11T¹, | |
| | A11V² | |
| 12 | G12I¹, G12A², G12C², | Bile duct carcinoma¹¹, |
| | G12D², G12E¹, G12F², | gall bladder carcinoma¹¹, |
| | G12G*^{1†}, G12L², G12N², | colorectal cancer¹¹, |
| | G12R², G12S², G12T¹, | haematopoietic neoplasm¹¹, |
| | G12V², G12W¹, G12Y¹ | lymphoid neoplasm¹¹, |
| | | lung adenocarcinoma¹¹, |
| | | NSCLC¹¹, pancreatic |
| | | ductal carcinoma¹¹, |
| | | prostate cancer¹¹, |
| | | melanoma¹¹, gastric |
| | | adenocarcinoma¹¹ |
| 13 | G13A², G13C¹, G13D², | colorectal cancer¹¹, |
| | G13E¹, G13F¹, G13G^{1†}, | haematopoietic neoplasm¹¹, |
| | G13I¹, G13M¹, G13N², | lymphoid neoplasm¹¹, |
| | G13P¹, G13R², G13S², | gastric adenocarcinoma¹¹ |
| | G13V², G13Y¹ | |
| 14 | V14A¹, V14G¹, V14I², | |
| | V14L¹ | |
| 15 | G15D², G15G^{1†}, G15S¹, | |
| | G15W⁹ | |
| 16 | K16R¹ | |
| 17 | S17G², S17N¹ | |
| 18 | A18D², A18T¹, A18V¹ | |
| 19 | L19F² | |
| 20 | T20A¹ | |
| 20 | T20A¹, T20M¹, T20R², | |
| | T20S¹, T20T^{1†} | |
| 21 | I21R¹ | |
| 22 | Q22*¹, Q22K¹, Q22Q^{1†}, | |
| | Q22R² | |
| 23 | L23I¹, L23R² | |
| 24 | I24F¹, I24N², I24V² | |
| 27 | H27H^{1†}, H27L², H27N² | |
| 28 | F28S¹ | |
| 30 | D30E¹ | |
| 31 | E31K¹, E31Q¹, E37G⁵, | |
| | E31Q¹ | |
| 33 | D33E¹ | |
| 34 | P34L¹, P34S² | |
| 35 | T35A¹, T35I², T35S⁵, | |
| | T35T^{1†} | |
| 36 | I36L¹, I36M¹ | |
| 37 | E37G⁵, E37K¹ | |
| 40 | Y40C⁵ | |
| 45 | V45V^{1†} | |
| 49 | E49*¹, E49K¹ | |
| 51 | C51C^{1†} | |
| 52 | L52F¹ | |
| 57 | D57N¹ | |
| 58 | T58I², T58T^{1†} | |
| 59 | A59A^{1†}, A59D⁴, A59del¹, | |
| | A59E², A59G², A59S¹, | |
| | A59T¹ | |
| 60 | G60A¹, G60D², G60E⁸, | |
| | G60G^{1†}, G60R¹, G60V¹, | |
| | G61H² | |
| 61 | Q61D¹, Q61E², Q61H², | Lung adenocarcinoma¹¹, |
| | Q61K², Q61L², Q61P², | NSCLC¹¹, colorectal |
| | Q61R¹ | cancer¹¹ |
| 62 | E62D², E62G¹, E62K¹ | |
| 63 | E63del¹, E62E^{1†}, E63K² | |
| 64 | Y64D¹, Y64H¹, Y64N¹ | |
| 65 | S65I¹ | |
| 67 | M67L² | |
| 66 | A66T¹⁰, A66V¹⁰, | |
| 68 | R68G¹, R68M¹, R68S¹ | |
| 69 | D69G¹ | |
| 70 | Q70P¹ | |
| 71 | Y71C¹ | |
| 72 | M72I², M72T¹, M72V¹ | |
| 73 | R73M¹ | |
| 74 | T74P², T74T^{1†} | |
| 75 | G75E¹⁰ | |
| 76 | E76G⁶, E76K⁶, E76Q⁶ | |
| 77 | G77A¹ | |
| 80 | C80S¹, C80Y¹ | |
| 86 | N86K¹ | |
| 88 | K88*¹, K88K^{1†} | |
| 91 | E91K¹ | |
| 92 | D92G¹, D92Y¹ | |
| 95 | H95L¹ | |
| 97 | R97I¹ | |
| 98 | E98*¹ | |
| 102 | R102fs*2¹ | |
| 110 | P110S¹ | |
| 117 | K117E¹, K117N², K117R¹ | |
| 118 | C118S¹ | |
| 120 | L120V⁷ | |
| 121 | P121H¹, P121S¹ | |
| 127 | T127I¹ | |
| 130 | A130V¹ | |
| 134 | A134T¹ | |
| 134 | S136N¹ | |
| 135 | R135T¹ | |
| 138 | G138E¹, G138R¹ | |
| 140 | P140S¹ | |
| 145 | S145T⁷ | |
| 146 | A146A^{1†}, A146G¹, A146P², | |
| | A146T², A146V² | |
| 147 | A147T³, K147N¹ | |
| 153 | D153N¹, D153V¹ | |
| 154 | D154delD¹ | |
| 156 | F156L² | |
| 161 | R161*¹ | |
| 164 | R164L¹, R164Q¹, R164R^{1†} | |
| 173 | D173D¹ | |
| 183 | T183_K184delTK¹ | |
| 185 | C185R¹, C185S¹ | |
| 188 | M188L¹ | |
| Insertions and Deletions | | |
| | A11_G12insGA¹ | |
| | V14_G15insG² | |
| | A66_M67insEEYSA¹ | |
| | D69fs*4¹ | |
| | E3fs*3¹ | |
| | E62 S65 > D² | |
| | (c.186 194del9) | |
| | G10 AllinsG² | |
| | (c.30 31insGGA) | |
| | G12 G13insA² | |
| | (c.37 37insGCG) | |
| | G12 G13insG² | |
| | (c.36 37insGGT) | |
| | G12fs*3¹ | |
| | G12V9F¹ | |
| | G13 V14 > DI¹ | |
| | G13 V14insG² | |
| | (c.39 40insGGC) | |
| | G60fs*27¹ | |
| | K16_S17insW¹ | |
| | L19_T20 > FA¹ | |
| | M72_R73ins15¹ | |
| | S65_A66ins15¹ | |
| | T183_K184delTK¹ | |
| | T58_A59insVA¹ | |
| | D154delD¹ | |
| | V9 G10insG² | |
| | (c.27 28insGTA) | |
| | V9 G10insV¹ | |

^{A}The KRAS mutations shown may be activating mutations and/or confer increased resistance of KRAS to a KRAS modulator (e.g., a KRAS inhibitor), e.g., as compared to a wild type KRAS.
^{†}Indicates a synonymous mutation which may affect KRAS protein expression. See, e.g., Waters et al.. PLOS One 2016; 11(9). doi: 10.1371/journal.pone.0163272.
¹U.S. Pat. No. 9,810,690
²U.S. Publication No. 2014/0199405
³P.C.T. Publication No. WO 2012/016050
⁴U.S. Pat. No. 10,238,650
⁵P.C.T. Publication No. WO 2009/052467
⁶U.S. Publication No. 2013/0317037
⁷P.C.T. Publication No. WO 2020/012068
⁸U.S. Publication No. 2017/0130271
⁹U.S. Publication No. 2017/0051356
¹⁰Abe et al. Biochemical and Biophysical Research Communications. 2020:522(3): P.360-696.
¹¹Prior et al. Cancer Res. 2012 May 15; 72(10): 2457-2467.

TABLE 1A
Exemplary KRAS Fusion Proteins and Cancers

| Non-limiting Exemplary | Non-limiting Exemplary KRAS- |
|---|---|
| KRAS Fusions | associated Cancer(s) |
| UBE2L3-KRAS¹ | Metastatic Prostate Cancer |

¹Wang et al. Cancer Discovery. 2011; 9(1): 35-43. doi: 10.1158-2159-8274.CD-10-0022.

TABLE 2
HRAS Protein Amino Acid Substitutions/Insertions/Deletions^{A}

| | | Non-Limiting |
|---|---|---|
| | | Exemplary |
| Amino Acid | Non-Limiting | HRAS-Associated |
| Position(s) | Exemplary Mutations | Cancers |
| 12 | G12A, G12C, G12D, G12R, | Salivary gland |
| | G12S, G12V¹ | adenocarcinoma, prostate |
| | | cancer, melanoma, gastric |
| | | adenocarcinoma¹, |
| | | Epithelial-Myoepithelial |
| | | Carcinoma³ |
| 13 | G13A, G13C, G13D, G13R, | |
| | G13S, G13V¹ | |
| 18 | A18V² | Oral squamous cell |
| | | carcinoma² |
| 61 | Q61H, Q61K, Q61L, Q61P, | Epithelial-Myoepithelial |
| | Q61R, Q61E, Q61*^{1, 3, 4} | Carcinoma³ |

^{A}The HRAS mutations shown may be activating mutations and/or confer increased resistance of HRAS to a HRAS modulator (e.g., a HRAS inhibitor), e.g., as compared to a wild type HRAS.
¹Prior et al. Cancer Res. 2012 May 15; 72(10): 2457-2467.
²Koumaki, Dimitra, et al. Oncology Reports 27 (2012): 1555-1560.
³Urano, Makoto, et al. The American journal of surgical pathology 43.7 (2019): 984-994.
⁴U.S. Pat. No. 10,722,484

TABLE 3
NRAS Protein Amino Acid Substitutions/Insertions/Deletions^{A}

| | | Non-Limiting |
|---|---|---|
| | | Exemplary |
| Amino Acid | Non-Limiting | NRAS-Associated |
| Position(s) | Exemplary Mutations | Cancers |
| 12 | G12A, G12C, G12D, G12R, G12S, | Myeloid leukemia², |
| | G12V, G12W, G12N^{1,2,4} | colorectal cancer⁴ |
| 13 | G13A, G13C, G13D, G13R, G13S, | Myeloid leukemia² |
| | G13V^{1,2} | |
| 15 | G15W⁶ | Colorectal cancer⁶ |
| 60 | G60E^{2,6} | Myeloid leukemia² |
| 61 | Q61E, Q61H, Q61K, Q61L, Q61P, | Myeloid leukemia², |
| | Q61R, Q61E, Q61N^{1,2,4,5} | colorectal cancer^{4,5} |
| 117 | K117N⁴ | Colorectal cancer⁴ |
| 146 | A146T, A146P, A146V^{3,4} | Colorectal cancer⁴ |

^{A}The NRAS mutations shown may be activating mutations and/or confer increased resistance of NRAS to a NRAS modulator (e.g., a NRAS inhibitor), e.g., as compared to a wild type NRAS.
¹Prior et al. Cancer Res. 2012 May 15; 72(10): 2457-2467.
²Tyner, Jeffrey W., et al. Blood, The Journal of the American Society of Hematology 113.8 (2009): 1749-1755.
³U.S. Pat. No. 10,668,063
⁴Payandeh, et al. American Journal of Cancer Prevention 3.1 (2015): 19-22.
⁵Villahermosa, et al. Journal of Clinical Oncology 2014 32:15_suppl, e22159-e22159
⁶Shen, et al. PloS One 8.12 (2013): e81628.

However, the Ras proteins have often been considered to be “undruggable”, and no direct Ras inhibitor has been approved by the United States Food and Drug Administration. Accordingly, other targets in Ras signaling pathways have been targeted in order to curb aberrant signaling through these pathways, including targets both upstream and downstream of the Ras family proteins. See, e.g., Cox, et al. Nat. Rev. Drug Disc. 13.11 (2014): 828-851; Khan, et al. Biochimica et Biophysica Acta (BBA)—Molecular Cell Research 1867.2 (2020): 118570; Kessler, et al. Proc. Nat. Acad. Sci. 116.32 (2019): 15823-15829; Dang, et al. Nat. Rev. Cancer 17.8 (2017): 502; Baker and Der, Nature 497.7451 (2013): 577-578.

Guanine nucleotide exchange factors, which promote the exchange of GDP for GTP bound by Ras family proteins, can be suitable targets to reduce signaling through Ras pathways. Inhibition of a GEF may promote the inactive (GDP bound) state of Ras family proteins and therefore decreased signaling through the pathway. See, e.g., Evelyn, et al. Chemistry & Biology 21.12 (2014): 1618-1628; Hillig, et al. Proc. Nat. Acad Sci. 116.7 (2019): 2551-2560; Patgiri, et al. Nat. Chem. Bio. 7.9 (2011): 585-587: Maurer, et al. Proc. Nat. Acad Sci. 109.14 (2012): 5299-5304; Winter, et al. J. Med Chem. 58.5 (2015): 2265-2274.

SOS1 has a central “catalytic” core (SOS^{cat}) of about 500 residues, which is sufficient for Ras-activating activity. SOS1 has a primary (sometimes also called the “catalytic” site) Ras binding site (e.g., including a Cdc25 homology domain) that can bind to and distort the nucleotide binding site of a Ras protein, thereby promoting the release of the bound nucleotide (e.g., GDP), allowing another nucleotide (e.g., GTP). SOS1 can bind two Ras molecules in a ternary complex, wherein binding of a Ras GTP complex to a second (sometimes also called the “allosteric” site) site on SOS1, further activating the catalytic activity of SOS1 in a positive feedback-type mechanism. See, e.g., Margarit, et al. Cell 112.5 (2003): 685-695; Freedman, et al. Proc. Nat. Acad Sci. 103.45 (2006): 16692-16697. Further, it has been shown that small-molecule binders of SOS1 can modulate its GEF activity. See, e.g., Burns, et al. Proc. Nat. Acad Sci. 111.9 (2014): 3401-3406. In some cases, small-molecule binders of SOS1 can negatively modulate its GEF activity with Ras proteins; such molecules can also be called herein “SOS1 inhibitors” and referred to as inhibiting “SOS1 activity.” Some SOS1 inhibitors have been shown to bind proximal to the primary Ras binding site, for example, causing a movement in the sidechain of Tyr884 and reducing favorable stacking interactions with Arg73 of KRas. Further, antiproliferative activity of some such SOS1 inhibitors has been demonstrated. See, e.g., Hillig, et al., Proc. Nat. Acad. Sci. 116.7 (2019): 2551-2560; U.S. Patent Appl. Publ. Nos. 2019/0358230 and 2019/0194192; and PCT Publication Nos WO 2018/172250 and WO 2019/201848.

The term “SOS1-associated cancer” as used herein refers to cancers associated with or having a dysregulation of a SOS1 gene, a SOS1-GEF (also called herein SOS1 protein), or the expression or activity or level of any (e.g., one or more) of the same (e.g., any of the types of dysregulation of a SOS1 gene, a SOS1 protein, or the expression or activity or level of any of the same, as described herein). Non-limiting examples of a SOS1-associated cancer are described herein.

The phrase “dysregulation of a SOS1 gene, a SOS1 protein, or the expression or activity or level of any of the same” refers to a genetic mutation (e.g., a SOS1 gene translocation that results in the expression of a fusion protein, a mutation in a SOS1 gene that results in the expression of a SOS1 protein that includes a deletion of at least one amino acid as compared to a wild type SOS1 protein, a mutation in a SOS1 gene that results in the expression of a SOS1 protein with one or more point mutations as compared to a wild type SOS1 protein, a mutation in a SOS1 gene that results in the expression of a SOS1 protein with at least one inserted amino acid as compared to a wild type SOS1 protein, a gene duplication that results in an increased level of SOS1 protein in a cell, or a mutation in a regulatory sequence (e.g., a promoter and/or enhancer) that results in an increased level of SOS1 protein in a cell), an alternative spliced version of a SOS1 mRNA (e.g., that results in a SOS1 protein having a deletion of at least one amino acid in the SOS1 protein as compared to the wild type SOS1 protein or that results in a SOS1 protein having an insertion of at least one amino acid in the SOS1 protein as compared to the wild type SOS1 protein), or increased expression (e.g., increased levels) of a wild type SOS1 protein in a mammalian cell due to aberrant cell signaling and/or dysregulated autocrine/paracrine signaling (e.g., as compared to a control non-cancerous cell). As another example, a dysregulation of a SOS1 gene, a SOS1 protein, or expression or activity, or level of any of the same, can be a mutation in a SOS1 gene that encodes a SOS1 protein that is constitutively active or has increased activity as compared to a protein encoded by a SOS1 gene that does not include the mutation. Non-limiting examples of SOS1 protein point mutations/insertions/deletions are described in Table 4. Table 4A lists a non-limiting exemplary SOS1 fusion.

TABLE 4
SOS1 Protein Amino Acid Substitutions/Insertions/Deletions^{A}

| Amino Acid | Non-Limiting | Non-Limiting Exemplary |
|---|---|---|
| Position(s) | Exemplary Mutations | SOS1-Associated Cancers |
| 65 | R65R^{1†} | Colon cancer¹ |
| 102 | P102R³ | Rhabdomyoscarcoma³ |
| 248 | R248H² | T-ALL² |
| 233 | N233Y⁴ | Lung adenocarcinoma⁴ |
| 265 | D265N⁴ | Lung adenocarcinoma⁴ |
| 316 | S316L¹ | Colon cancer¹ |
| 327 | I327T⁴ | Lung adenocarcinoma⁴ |
| 340 | P340S¹ | Colon cancer¹ |
| 378 | T378A³ | Sertoli cell tumor³ |
| 410 | Q410Q^{2†} | Lung adenocarcinoma² |
| 414 | G414G^{1†} | Colon cancer¹ |
| 477 | Q477*¹ | Colon cancer¹ |
| 478 | P478L⁴ | Lung adenocarcinoma⁴ |
| 494 | F494L⁵ | AML⁵ |
| 535 | N535S⁴ | Lung adenocarcinoma⁴ |
| 552 | R552G³ | Granular cell tumors of the |
| | | skin³ |
| 604 | G604V⁴ | Lung adenocarcinoma⁴ |
| 610 | I610T⁵ | AML⁵ |
| 684 | P684S¹ | Colon cancer¹ |
| 688 | R688Q² | Pancreatic cancer² |
| 733 | I733V, I733F⁴ | Lung adenocarcinoma⁴ |
| 806 | G806R¹ | Colon cancer¹ |
| 861 | V861I¹ | Colon cancer¹ |
| 888 | H888Q² | Lung adenocarcinoma² |
| 938 | L938F⁴ | Lung adenocarcinoma⁴ |
| 1019 | R1019Q¹ | Colon cancer¹ |
| 1051 | R1051G⁴ | Lung adenocarcinoma⁴ |
| 1236 | P1236P^{1†} | Colon cancer¹ |
| Insertions, Deletions, and UTRs | | |
| | SOS1-116G > T¹ | Colon cancer¹ |
| | IVS8 + 5G > C¹ | Colon cancer¹ |

^{A}The SOS1 mutations shown may be activating mutations and/or confer increased resistance of SOSI to a SOS1 modulator (e.g., a SOS1 inhibitor), e.g., as compared to a wild type SOS1.
^{†}Indicates a synonymous mutation, which may or may not affect SOS1 protein expression or other aspects of SOS1 regulation or function.
¹U.S. Patent Application Publication No. 2010/0227778
²Swanson, et al. Genes, Chromosomes and Cancer 47.3 (2008): 253-259. doi: 10.1002/gcc.20527
³Denayer, et al. Genes, Chromosomes and Cancer 49.3 (2010): 242-252. doi: 10.1002/gcc.20735
⁴Cai, et al. Mol. Cancer Res, 17.4 (2019): 1002-1012. doi: 10.1158/1541-7786.MCR-18-0316
⁵Tanizaki, et al. International Journal of Hematology 88.4 (2008): 460-462.

TABLE 4A
Exemplary SOS1 Fusion Proteins and Cancers

| Non-limiting Exemplary | Non-limiting Exemplary SOS1- |
|---|---|
| SOS1 Fusion Partners | associated Cancer(s) |
| ADCY3¹ | Lung adenocarcinoma¹ |

¹P.C.T. Publication No. WO 2013/113942

The term “wild type” describes a nucleic acid (e.g., a SOS1 gene or mRNA) or protein (e.g., a SOS1 protein) that is found in a subject that does not have a disease or disorder associated with that nucleic acid or protein (e.g., a SOS1-related disease or disorder), e.g., a cancer associated with that nucleic acid or protein (and optionally also does not have an increased risk of developing a disease or disorder associated with that nucleic acid or protein and/or is not suspected of having a disease or disorder associated with that nucleic acid or protein), or is found in a cell or tissue from a subject that does not have a disease associated with that nucleic acid or protein, e.g., a cancer associated with that nucleic acid or protein (and optionally also does not have an increased risk of developing a disease or disorder associated with that nucleic acid or protein and/or is not suspected of having a disease or disorder associated with that nucleic acid or protein).

The term “regulatory agency” refers to a country's agency for the approval of the medical use of pharmaceutical agents with the country. For example, a non-limiting example of a regulatory agency is the U.S. Food and Drug Administration (FDA).

### Compounds

Provided herein are compounds of Formula (I):

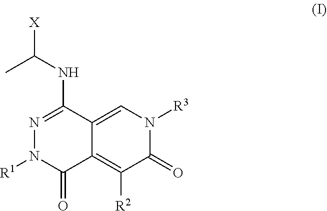

or a pharmaceutically acceptable salt thereof, wherein:
X is phenyl, naphthyl, 3-10 membered cycloalkyl, 3-10 membered heterocyclyl, or 5-10 membered heteroaryl, each of which is optionally substituted by 1-3 independently selected R^{A};
R¹ is C₁-C₆ alkyl, —(C₁-C₆ alkyl)-NR^{B}R^{C}, C₁-C₆ hydroxyalkyl, or 5-6 membered heteroaryl;
R² is hydrogen, halogen, cyano, hydroxyl, —NR^{D}R^{E}, C₁-C₆ alkyl, C₁-C₆ alkoxy optionally substituted with 3-10 membered cycloalkyl or heterocyclyl, C(O)C₁-C₆alkyl, C₁-C₆ hydroxycycloalkyl optionally substituted with 1-3 fluoro, C₁-C₆ hydroxyheterocyclyl optionally substituted with 1-3 fluoro, or 3-10 membered heterocyclyl optionally substituted with amino;
R³ is
(A) C₃-C₈ cycloalkyl optionally substituted with hydroxyl, cyano, C₁-C₆ alkyl or 1-3 fluoro, C₃-C₆ cycloalkyl, 5-10 membered heteroaryl optionally substituted with 1-3 fluoro, 3-10 membered —CH₂-heteroyclyl substituted with 1-3 fluoro, C₁-C₆ haloalkyl, C₁-C₆ hydroxyalkyl —C(O)NR^{F}R^{G} or C₁-C₆ alkyl, or
(B) 3-10 membered heterocyclyl optionally independently substituted with one or more halogen, C₁-C₆ alkyl, C₁-C₆ haloalkyl, —C(O)C₁-C₆alkyl, —C(O)C₁-C₆ cycloalkyl optionally substituted by halogen, C₁-C₆ alkyl or C₁-C₆ alkoxy, or —C(O)NR^{F}R^{G};
each R^{A} is independently selected from one or more halogen, cyano, nitro, —(C₁-C₆ alkyl)ₚ-NR^{H}R^{I}; C₁-C₆ alkyl, C₁-C₆ alkoxy, C₁-C₆ haloalkoxy, C₁-C₆ hydroxyalkyl optionally substituted with 1-3 fluoro, C₁-C₆ alkoxyalkyl optionally independently substituted with one or more 1-3 fluoro or C₁-C₆ alkyl, C₁-C₆ haloalkyl optionally substituted with 3-10 membered heteroyclyl, —CH2C(O)NR^{F}R^{G} optionally substituted with 1-3 fluoro, 5-6 membered —CH₂-heteroaryl substituted with 1-3 fluoro, and 3-10 membered cycloalkyl optionally substituted with —(C₁-C₆ alkyl)ₚ-NR^{J}R^{K};
each R^{B}, R^{C}, R^{D}, R^{E}, R^{F}, R^{G}, R^{H}, R^{I}, R^{J}, R^{K} are independently selected from hydrogen and C₁-C₆ alkyl, or R^{B} and R^{C} together with the atom to which they are bonded can form a 5-10 membered heterocyclyl optionally substituted with one or more of halogen, C₁-C₆ alkyl, and C₁-C₆ alkoxy; and
p is 0 or 1.

In one embodiment are compounds of Formula (I):

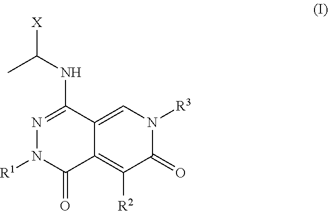

or a pharmaceutically acceptable salt thereof, wherein:
X is phenyl, naphthyl, 3-10 membered cycloalkyl, or 5-10 membered heteroaryl, each of which is optionally substituted by 1-3 independently selected R^{A};
R¹ is C₁-C₆ alkyl, —(C₁-C₆ alkyl)-NR^{B}R^{C}, C₁-C₆ hydroxyalkyl, or 5-6 membered heteroaryl;
R² is hydrogen, halogen, cyano, hydroxyl, —NR^{D}R^{E}, C₁-C₆ alkyl, C₁-C₆ alkoxy optionally substituted with 3-10 membered cycloalkyl, C(O)C₁-C₆alkyl, or 3-10 membered heterocyclyl optionally substituted with amino;
R³ is
(A) C₃-C₆ cycloalkyl optionally substituted with hydroxyl, cyano, 5-10 membered heteroaryl, C₁-C₆ haloalkyl, —C(O)NR^{F}R^{G} or C₁-C₆ alkyl, or
(B) 3-10 membered heterocyclyl optionally substituted with halogen, C₁-C₆ alkyl, —C(O)C₁-C₆alkyl, or —C(O)NR^{F}R^{G};
each R^{A} is independently selected from halogen, cyano, nitro, —(C₁-C₆ alkyl)ₚ-NR^{H}R^{L}; C₁-C₆ alkyl, C₁-C₆ alkoxy, C₁-C₆ haloalkoxy, C₁-C₆ hydroxyalkyl optionally substituted with 1-3 fluoro, C₁-C₆ alkoxyalkyl optionally substituted with 1-3 fluoro, C₁-C₆ haloalkyl optionally substituted with 3-10 membered heteroyclyl, and 3-10 membered cycloalkyl optionally substituted with —(C₁-C₆ alkyl)ₚ-NR^{J}R^{K};
each R^{B}, R^{C}, R^{D}, R^{E}, R^{F}, R^{G}, R^{H}, R^{I}, R^{J}, R^{K} are independently selected from hydrogen and C₁-C₆ alkyl, or R^{B} and R^{C} together with the atom to which they are bonded can form a 5-10 membered heterocyclyl optionally substituted with one or more of halogen, C₁-C₆ alkyl, and C₁-C₆ alkoxy; and
p is 0 or 1.

In some embodiments, R¹ is C₁-C₆ alkyl, such as methyl, ethyl, n-propyl, isopropyl, n-butyl, sec-butyl, iso-butyl, t-butyl, pentyl (straight chain or branched), or hexyl (straight chain or branched). In some embodiments, R¹ is methyl or ethyl. In some embodiments, R¹ is methyl.

In some embodiments, R¹ is —(C₁-C₆ alkyl)-NR^{B}R^{C}, such as methylamino, ethyamino, dimethylamino(ethyl), and the like.

In some embodiments, each R^{B} and R^{C} are both hydrogen. In some embodiments, each R^{B} and R^{C} are independently C₁-C₆ alkyl. In some embodiments, one of R^{B} and R^{C} is hydrogen and the other of R^{B} and R^{C} is C₁-C₆ alkyl.

In some embodiments, R¹ is 5-6 membered heteroaryl. In some embodiments, R¹ is 5 membered heteroaryl, for example, furan, thiophene, pyrrole, imidazole, pyrazole, oxazole, thiazole, or triazole. In some embodiments, R¹ is 5 membered heteroaryl, for example pyridine, pyridazine, pyrimidine, pyrazine. In some embodiments, R¹ is oxazole.

In some embodiments, R¹ is C₁-C₆ hydroxyalkyl. In some embodiments R¹ is ethoxy.

In some embodiments, R² is halogen. In some embodiments, R² is fluoro. In some embodiments, R² is chloro. In some embodiments, R² is bromo.

In some embodiments, R² is hydrogen.

In some embodiments, R² is cyano.

In some embodiments, R² is hydroxyl.

In some embodiments, R² is —NR^{D}R^{E}.

In some embodiments, R² is C₁-C₆ alkoxy optionally substituted with 3-10 membered cycloalkyl. In some embodiments R² is methoxy. In some embodiments, R² is cyclopropylmethoxy. In some embodiments, R² is C(O)C₁-C₆alkyl. In some embodiments, R² is acetyl. In some embodiments, R² is 3-10 membered heteroyclyl optionally substituted with amino.

In some embodiments, each R^{D} and R^{E} are both hydrogen (e.g., R² is amino). In some embodiments, each R^{D} and R^{E} are independently C₁-C₆ alkyl. In some embodiments, one of R^{D} and R^{E} is hydrogen and the other of R^{D} and R^{E} is C₁-C₆ alkyl.

In some embodiments, R² is C₁-C₆ alkyl, such as methyl, ethyl, n-propyl, isopropyl, n-butyl, sec-butyl, iso-butyl, t-butyl, pentyl (straight chain or branched), or hexyl (straight chain or branched). In some embodiments, R² is methyl.

In some embodiments, R² is C₁-C₆ alkoxy, such as methoxy, ethoxy, n-propoxy, isopropoxy, n-butoxy, sec-butoxy, iso-butoxy, t-butoxy, pentyl (straight chain or branched), or hexyl (straight chain or branched). In some embodiments, R² is methoxy.

In some embodiments, R³ is C₃-C₆ cycloalkyl optionally substituted with hydroxyl, cyano, 5-10 membered heteroaryl, C₁-C₆ haloalkyl, —C(O)NR^{F}R^{G}, or C₁-C₆ alkyl. In some embodiments, R³ is C₃-C₆ cycloalkyl substituted with hydroxyl, cyano, 5-10 membered heteroaryl, C₁-C₆ haloalkyl, —C(O)NR^{F}R^{G}, or C₁-C₆ alkyl. In some embodiments, R³ is C₃-C₆ cycloalkyl substituted with C₁-C₆ alkyl. In some embodiments, R³ is C₃-C₆ cycloalkyl substituted with methyl. In some embodiments, R³ is C₃-C₆ cycloalkyl substituted with hydroxyl. In some embodiments, R³ is an unsubstituted C₃-C₆ cycloalkyl. In some embodiments, R³ is cyclopropyl, cyclobutyl, or bicyclopentyl. In some embodiments, R³ is cyclopropyl. In some embodiments, R³ is cyclobutyl. In some embodiments, R³ is bicyclopentyl.

In some embodiments, R³ is a C₃-C₆ cycloalkyl optionally substituted with cyano. In some embodiments, R³ is a C₃-C₆ cycloalkyl optionally substituted with 5-10 membered heteroaryl. In some embodiments, R³ is a C₃-C₆ cycloalkyl optionally substituted with C₁-C₆ haloalkyl. In some embodiments, R³ is a C₃-C₆ cycloalkyl optionally substituted with —C(O)NR^{F}R^{G}.

In some embodiments, R³ is selected from:

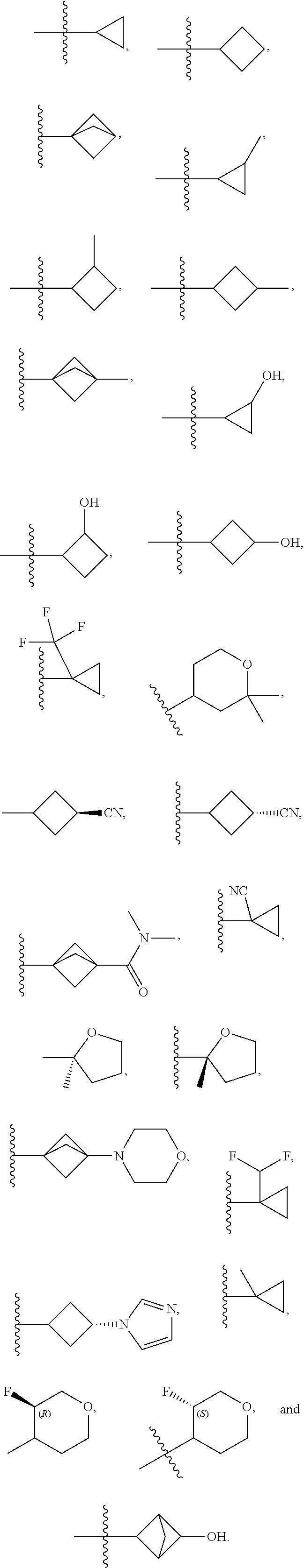

In some embodiments, R³ is a 3-10 membered heterocyclyl optionally substituted with halogen, C₁-C₆ alkyl, C₁-C₆ haloalkyl, —C(O)C₁-C₆ alkyl, or —C(O)NR^{F}R^{G}. In some embodiments, R³ is a 3-10 membered heterocyclyl substituted with halogen, C₁-C₆ alkyl, —C(O)C₁-C₆ alkyl, or —C(O)NR^{F}R^{G}. In some embodiments, R³ is a 3-10 membered heterocyclyl substituted with C₁-C₆ alkyl. In some embodiments R³ is a a 3-6 membered heterocyclyl optionally substituted with C₁-C₆ haloalkyl, or —C(O)C₁-C₆ cycloalkyl optionally substituted by halogen, C₁-C₆ alkyl or C₁-C₆ alkoxy.

In some embodiments, R³ is a 6-10 membered heterocyclyl substituted with C₁-C₆ alkyl. In some embodiments, R³ is a 6-10 membered heterocyclyl substituted with C₁-C₆ haloalkyl. In some embodiments, R³ is a 3-10 membered heterocyclyl substituted with halogen. In some embodiments, R³ is a 6-10 membered heterocyclyl substituted with methyl. In some embodiments, R³ is a 3-10 membered heterocyclyl substituted with —C(O)C₁-C₆ alkyl. In some embodiments, R³ is a 6-10 membered heterocyclyl substituted with —C(O)C₁-C₆ alkyl. In some embodiments, R³ is a 6-10 membered heterocyclyl substituted with acetyl. In some embodiments R³ is a a 3-6 membered heterocyclyl optionally substituted with —C(O)C₁-C₆ cycloalkyl optionally substituted by halogen, C₁-C₆ alkyl or C₁-C₆ alkoxy. In some embodiments, R³ is a 3-10 membered heterocyclyl substituted with —C(O)NR^{F}R^{G}. In some embodiments, R³ is a 6-10 membered heterocyclyl substituted with —C(O)NR^{F}R^{G}.

In some embodiments, each R^{F} and R^{G} are both hydrogen. In some embodiments, each R^{F} and R^{G} are independently C₁-C₆ alkyl. In some embodiments, one of R^{F} and R^{G} is hydrogen and the other of R^{F} and R^{G} is C₁-C₆ alkyl.

In some embodiments, R³ is 2-oxaspiro[3.3]heptane, piperidine, or tetrahydropyran, optionally substituted with C₁-C₆ alkyl, —C(O)C₁-C₆ alkyl, or —C(O)NR^{F}R^{G}, as described herein. In some embodiments, R³ is 2-oxaspiro[3.3]heptane, piperidine, or tetrahydropyran, substituted with C₁-C₆ alkyl, —C(O)C₁-C₆ alkyl, or —C(O)NR^{F}R^{G}, as described herein. In some embodiments, R³ is an unsubstituted 2-oxaspiro[3.3]heptane, piperidine, or tetrahydropyran. In some embodiments, R³ is tetrahydropyran.

In some embodiments, R³ is selected from:

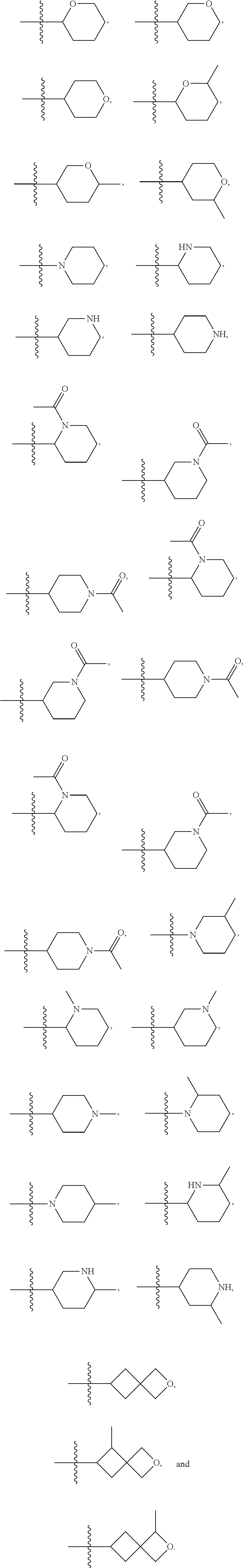

In some embodiments, R³ is

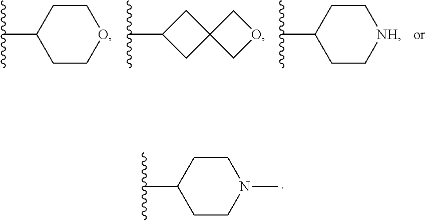

In some embodiments, R³ is

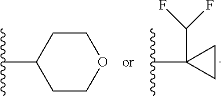 [SMILES: *C1CCOCC1 ; *C1(C(F)F)CC1]

In some embodiments, X is phenyl optionally substituted by 1-3 independently selected R^{A}. The R^{A} groups can be at any of the five available positions in the phenyl ring.

In some embodiments, X is phenyl substituted by 1 R^{A}. The one R^{A} group can be in the ortho, meta, or para position, relative to the bond connecting X to the remainder of the molecule. In some embodiments, X is phenyl substituted by 2 independently selected R^{A}. The two independently selected R^{A} groups can be in the ortho, meta, or para position relative to one another. In some embodiments, X is phenyl substituted by 3 independently selected R^{A}. The three independently selected R^{A} groups can be located at any combination of the five available positions on the phenyl ring. In some embodiments, X is an unsubstituted phenyl.

In some embodiments, X is selected from:

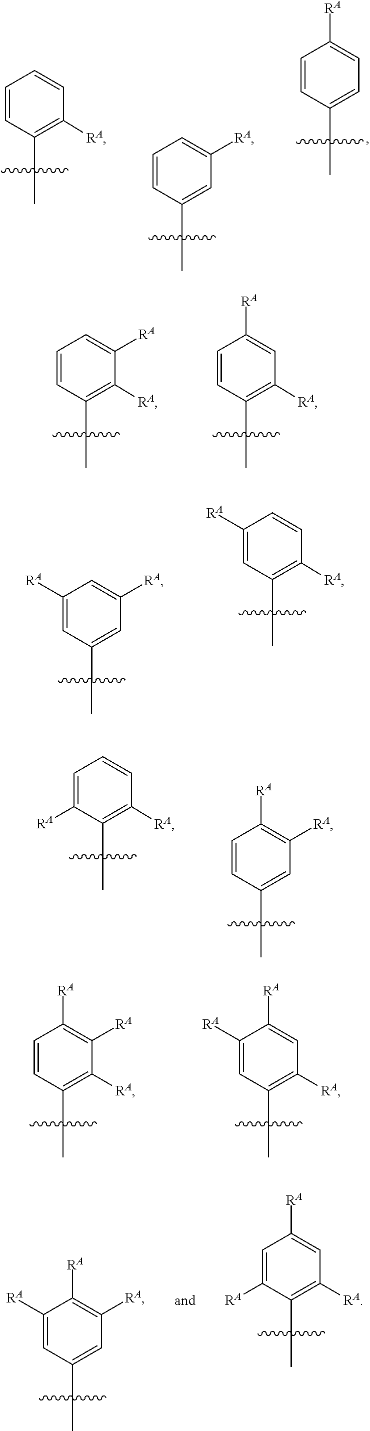

In some embodiments, X is selected from:

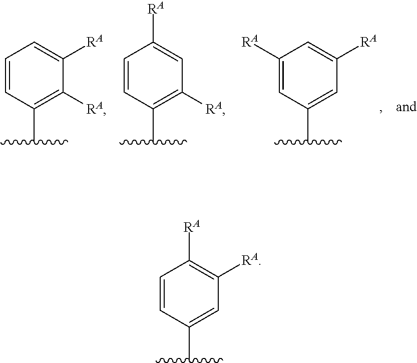

In some embodiments, X is

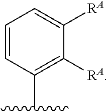

In some embodiments, X is

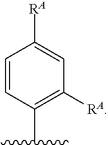

In some embodiments, X is

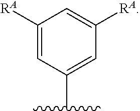

In some embodiments, X is

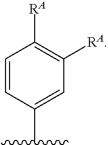

In some embodiments X is

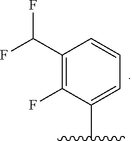 [SMILES: *c1cccc(C(F)F)c1F]

In some embodiments, X is naphthyl optionally substituted by 1-3 independently selected R^{A}. In some embodiments, X is naphthyl substituted by 1 R^{A}. In some embodiments, X is naphthyl substituted by 2 independently selected R^{A}. In some embodiments, X is naphthyl substituted by 3 independently selected R^{A}. The R^{A} groups can be on either a single ring (such as the ring having the bond to the rest of the compound, or the ring not having the bond to the rest of the compound) or on both rings. In some embodiments, the naphthyl is attached to the —CH(CH₃)NH— moiety at a position of the naphthyl that is bonded to a fusion carbon.

In some embodiments, X is unsubstituted naphthyl. In some embodiments, X is

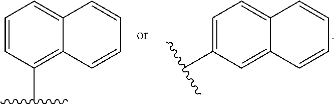 [SMILES: *c1cccc2ccccc12 ; *c1ccc2ccccc2c1]

In some embodiments, X is a 3-10 membered cycloalkyl optionally substituted by 1-3 independently selected R^{A}. In some embodiments, X is a 3-10 membered cycloalkyl optionally substituted by 1 selected R^{A}. In some embodiments, X is a 3-10 membered cycloalkyl substituted by 2 independently selected R^{A}. In some embodiments, X is a 3-10 membered cycloalkyl substituted by 3 independently selected R^{A}. In some embodiments, X is a 3, 4, 5, 6, 9, or 10 membered cycloalkyl.

In some embodiments, X is

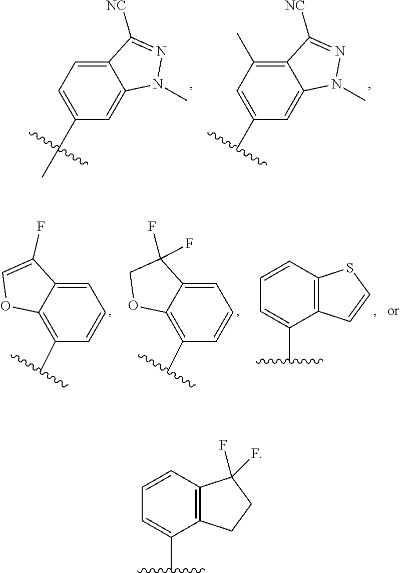

In some embodiments, X is a 5-10 membered heteroaryl or 3-10 membered heterocyclyl optionally substituted by 1-3 independently selected R^{A}. In some embodiments, X is a 5-10 membered heteroaryl optionally substituted by 1 selected R^{A}. In some embodiments, X is a 5-10 membered heteroaryl substituted by 2 independently selected R^{A}. In some embodiments, X is a 5-10 membered heteroaryl substituted by 3 independently selected R^{A}. In some embodiments, X is a 6, 9, or 10 membered heteroaryl.

In some embodiments, X is pyridine, pyrazine, pyrimidine, indole, indazole, benzimidazole, thiophene, or pyrrolopyrimidine. In some embodiments, X is pyridine.

In some embodiments, each R^{A} is independently selected from halogen, cyano, nitro, —(C₁-C₃ alkyl)ₚ-NR^{H}R^{I}; C₁-C₃ alkyl, C₁-C₃ alkoxy, C₁-C₃ haloalkyl optionally substituted with 3-10 membered heteroyclyl, C₁-C₃ haloalkoxy, C₁-C₃ hydroxyalkyl optionally substituted with 1-3 fluoro, 3-10 membered cycloalkyl optionally substituted with —(C₁-C₆ alkyl)ₚ-NR^{J}R^{K}, and C₁-C₃ alkoxyalkyl optionally substituted with 1-3 fluoro.

In some embodiments, each R^{A} is independently selected from cyano, —(C₁-C₃ alkyl)ₚ-NR^{H}R^{I}; C₁-C₃ alkyl, C₁-C₃ alkoxy, C₁-C₃ haloalkyl optionally substituted with 3-10 membered heteroyclyl, C₁-C₃ haloalkoxy, C₁-C₃ hydroxyalkyl optionally substituted with 1-3 fluoro, and C₁-C₃ alkoxyalkyl optionally substituted with 1-3 fluoro.

In some embodiments, each R^{A} is independently selected from cyano, —(C₁-C₃ alkyl)ₚ-NR^{H}R^{I}; C₁-C₃ alkyl, C₁-C₃ alkoxy, C₁-C₃ haloalkyl optionally substituted with 3-10 membered heteroyclyl, C₁-C₃ haloalkoxy, C₁-C₃ hydroxyalkyl substituted with 1-3 fluoro, and C₁-C₃ alkoxyalkyl substituted with 1-3 fluoro.

In some embodiments, each R^{A} is independently selected from cyano, —(C₁-C₃ alkyl)ₚ-NR^{H}R^{I}; C₁-C₃ alkyl, C₁-C₃ alkoxy, C₁-C₃ haloalkyl optionally substituted with 3-10 membered heteroyclyl, C₁-C₃ haloalkoxy, C₁-C₃ hydroxyalkyl, and C₁-C₃ alkoxyalkyl.

In some embodiments, p is 0. In some embodiments, p is 1.

In some embodiments, each R^{H} and R^{I} are both hydrogen.

In some embodiments, each R^{H} and R^{I} are independently C₁-C₆ alkyl. In some embodiments, one of R^{H} and R^{I} is hydrogen and the other of R^{H} and R^{I} is C₁-C₆ alkyl.

In some embodiments, R^{A} can be amino, where R^{A} is —(C₁-C₃ alkyl)ₚ-NR^{H}R^{I}, p is 0, and R^{D} and R^{E} are both hydrogen.

In some embodiments, each R^{A} is independently selected from cyano, C₁-C₃ alkyl, and C₁-C₃ haloalkyl optionally substituted with 3-10 membered heteroyclyl.

In some embodiments, each R^{A} is independently selected from cyano, methyl, methoxy, C₁-C₃ haloalkyl optionally substituted with 3-10 membered heteroyclyl, C₁-C₃ haloalkoxy, C₁-C₃ hydroxyalkyl, 3-10 membered cycloalkyl optionally substituted with —(C₁-C₆ alkyl)ₚ-NR^{J}R^{K}, 5-6 membered —CH₂-heteroaryl substituted with 1-3 fluoro, and C₁-C₃ alkoxyalkyl.

In some embodiments, each R^{A} is independently selected from fluoro, chloro, cyano, nitro, —(C₁-C₃ alkyl)-NH₂, —NH₂ (amino), —(C₁-C₃ alkyl)-NHMe, —NHMe, —(C₁-C₃ alkyl)-N(Me)₂, —N(Me)₂, methyl, methoxy, trifluoromethyl, 2,2,2-trifluoroethyl, trifluoromethoxy, 1-hydroxyethyl, 2-hydroxyethyl, 1-hydroxyethyl, 2-hydroxyethyl, 2,2,2-trifluorethan-1-ol, 1,1-difluoroethyl-2-ol, and methoxyethyl.

In some embodiments, each R^{A} is independently selected from cyano, —(C₁-C₃ alkyl)-NH₂, —NH₂, —(C₁-C₃ alkyl)-N(Me)₂, —N(Me)₂, methyl, methoxy, trifluoromethyl, 2,2,2-trifluoroethyl, trifluoromethoxy, 1-hydroxyethyl, 2-hydroxyethyl, 1-hydroxyethyl, 2-hydroxyethyl, 2,2,2-trifluorethan-1-ol, 1,1-difluoroethyl-2-ol, and methoxyethyl.

In some embodiments, each R^{A} is independently selected from cyano, amino, methyl, methoxy, C₁-C₃ haloalkyl optionally substituted with 3-10 membered heteroyclyl, C₁-C₃ haloalkoxy, C₁-C₃ hydroxyalkyl, 3-10 membered cycloalkyl optionally substituted with —(C₁-C₆ alkyl)ₚ-NR^{J}R^{K}, and C₁-C₃ alkoxyalkyl.

In some embodiments, each R^{A} is independently selected from cyano, methyl, fluoro, methoxy, difluoromethyl, trifluoromethyl, trifluoromethoxy, and hydroxyethyl.

In some embodiments, each R^{A} is independently selected from cyano, methyl, difluoromethyl, and trifluoromethyl.

In some embodiments, each R^{A} is independently selected from cyano and methyl.

In some embodiments, each R^{A} is independently selected from amino and methyl.

In some embodiments, each R^{A} is independently selected from methyl and trifluoromethyl.

In some embodiments, at least one R^{A} is methyl. In some embodiments, at least one R^{A} is difluoromethyl. In some embodiments, at least one R^{A} is trifluoromethyl. In some embodiments, at least one R^{A} is amino. In some embodiments, one R^{A} is methyl. In some embodiments, one R^{A} is trifluoromethyl. In some embodiments, one R^{A} is amino.

In some embodiments, one R^{A} is amino and one R^{A} is trifluoromethyl. In some embodiments, one R^{A} is methyl and one R^{A} is trifluoromethyl. In some embodiments, one R^{A} is fluoro and one R^{A} is difluromethyl.

In some embodiments, R^{B} and R^{C} together with the atom to which they are bonded can form a 5-10 membered heterocyclyl optionally substituted with one or more of halogen, C₁-C₆ alkyl, and C₁-C₆ alkoxy. In some embodiments, R^{B} and R^{C} together with the atom to which they are bonded form a 5-10 membered heterocyclyl optionally substituted with one or more of halogen. In some embodiments, R^{B} and R^{C} together with the atom to which they are bonded form a 5-10 membered heterocyclyl optionally substituted with C₁-C₆ alkyl. In some embodiments, R^{B} and R^{C} together with the atom to which they are bonded form a 5-10 membered heterocyclyl optionally substituted with C₁-C₆ alkoxy.

In some embodiments, X is

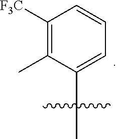 [SMILES: *c1cccc(C(F)(F)F)c1C]

In some embodiments, X is

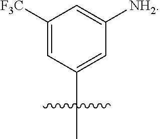 [SMILES: *c1cc(N)cc(C(F)(F)F)c1]

In some embodiments, X is

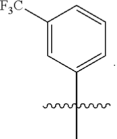 [SMILES: *c1cccc(C(F)(F)F)c1]

In some embodiments, X is

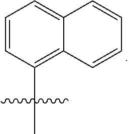 [SMILES: *c1cccc2ccccc12]

In some embodiments, X is an unsubstituted phenyl, unsubstituted naphthyl, or unsubstituted 5-10 membered heteroaryl.

In some embodiments, the compound is a compound of Formula (Ia):

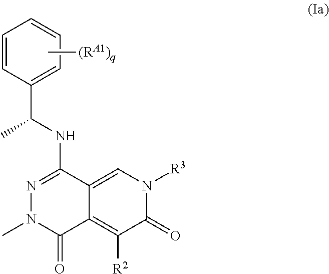

or a pharmaceutically acceptable salt thereof, wherein:
R² is hydrogen, methyl, methoxy, amino, or halogen;
R³ is C₃-C₆ cycloalkyl optionally substituted with hydroxyl or C₁-C₆ alkyl, 3-10 membered heterocyclyl optionally substituted with C₁-C₆ alkyl, —C(O)C₁-C₆ alkyl, or —C(O)NR^{F}R^{G};
each R^{A1} is independently selected from halogen, cyano, nitro, —(C₁-C₆ alkyl)ₚ-N^{H}R^{L}; C₁-C₆ alkyl, C₁-C₆ alkoxy, C₁-C₆ haloalkyl, C₁-C₆ haloalkoxy, C₁-C₆ hydroxyalkyl optionally substituted with 1-3 fluoro, and C₁-C₆ alkoxyalkyl optionally substituted with 1-3 fluoro; or two adjacent R^{A1}, when present, together with the atoms to which they are attached join to form a 5-6 membered heteroaryl or a phenyl, wherein the 5-6 membered heteroaryl and phenyl are optionally substituted by 1-3 independently selected R^{A2};
each R^{A2} is independently selected from halogen, cyano, nitro, —(C₁-C₆ alkyl)ₒ-NR^{J}R^{K}; C₁-C₆ alkyl, C₁-C₆ alkoxy, C₁-C₆ haloalkyl, C₁-C₆ haloalkoxy, C₁-C₆ hydroxyalkyl optionally substituted with 1-3 fluoro, and C₁-C₆ alkoxyalkyl optionally substituted with 1-3 fluoro;
each R^{F}, R^{G}, R^{H}, R^{I}, R^{J}, and R^{K} is independently selected from hydrogen and C₁-C₆ alkyl;
o is 0 or 1;
p is 0 or 1;
q is 0, 1, or 2.

In some embodiments, R² is hydrogen. In some embodiments, R² is methyl. In some embodiments, R² is methoxy. In some embodiments, R² is amiNo. In some embodiments, R² is halogen, such as fluoro, chloro, or bromo. In some embodiments, R² is chloro. In some embodiments, R² is bromo.

In some embodiments, R³, R^{B}, and R^{C}, are as described for Formula (I).

In some embodiments, the compound is a compound of Formula (Ib):

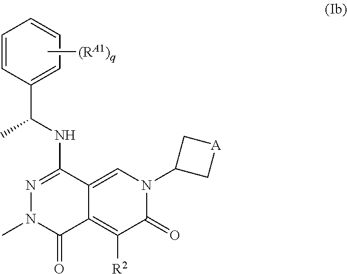

or a pharmaceutically acceptable salt thereof, wherein:
A is a bond, —C(R^{3A})(R^{3B})—, or —CH₂OCH₂—;
R² is hydrogen, methyl, methoxy, amino, or halogen;
each R^{A1} is independently selected from halogen, cyano, nitro, —(C₁-C₆ alkyl)ₚ-NR^{H}R^{I}; C₁-C₆ alkyl, C₁-C₆ alkoxy, C₁-C₆ haloalkyl, C₁-C₆ haloalkoxy, C₁-C₆ hydroxyalkyl optionally substituted with 1-3 fluoro, and C₁-C₆ alkoxyalkyl optionally substituted with 1-3 fluoro; or
two adjacent R^{A1}, when present, together with the atoms to which they are attached join to form a 5-6 membered heteroaryl or a phenyl, wherein the 5-6 membered heteroaryl and phenyl are optionally substituted by 1-3 independently selected R^{A2};
each R^{A2} is independently selected from halogen, cyano, nitro, —(C₁-C₆ alkyl)ₒ-NR^{J}R^{K}; C₁-C₆ alkyl, C₁-C₆ alkoxy, C₁-C₆ haloalkyl, C₁-C₆ haloalkoxy, C₁-C₆ hydroxyalkyl optionally substituted with 1-3 fluoro, and C₁-C₆ alkoxyalkyl optionally substituted with 1-3 fluoro;
R^{3A} and R^{3B} are independently selected from H and hydroxyl;
R^{3C} is H, C₁-C₆ alkyl, or —C(O)C₁-C₆ alkyl;
each R^{H}, R^{I}, R^{J}, and R^{K} are independently selected from hydrogen and C₁-C₆ alkyl; and
o is 0 or 1;
p is 0 or 1;
q is 0, 1, or 2.

In some embodiments, R² is hydrogen. In some embodiments, R² is methyl. In some embodiments, R² is methoxy. In some embodiments, R² is amino. In some embodiments, R² is halogen, such as fluoro, chloro, or bromo. In some embodiments, R² is chloro. In some embodiments, R² is bromo.

In some embodiments, A is a bond.

In some embodiments, A is —C(R^{3A})(R^{3B})—. In some embodiments, R^{3A} and R^{3B} are independently selected from hydrogen and hydroxyl. In some embodiments, R^{3A} and R^{3B} are both hydrogen. In some embodiments, one of R^{3A} and R^{3B} is hydrogen and the other of R^{3A} and R^{3B} is hydroxyl.

In some embodiments, A is —CH₂OCH₂—.

In some embodiments, q is 1. In some embodiments, q is 2.

In some embodiments,

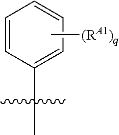

is selected from:

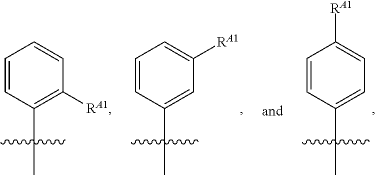

In some embodiments,

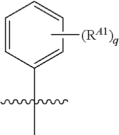

is selected from:

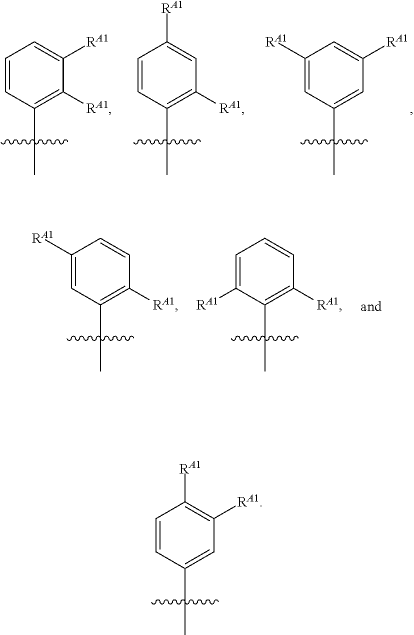

In some embodiments,

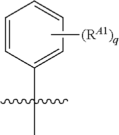

is selected from:

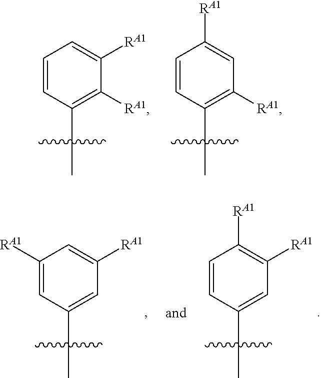

In some embodiments, is

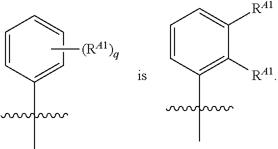

In some embodiments, is

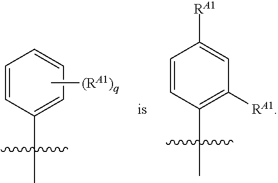

In some embodiments, is

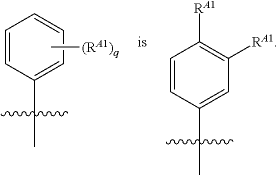

In some embodiments, is

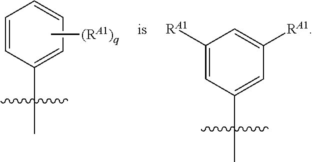

In some embodiments, each R^{A1} is independently selected from halogen, cyano, nitro, —(C₁-C₃ alkyl)ₚ-NR^{H}R^{L}; C₁-C₃ alkyl, C₁-C₃ alkoxy, C₁-C₃ haloalkyl, C₁-C₃ haloalkoxy, C₁-C₃ hydroxyalkyl optionally substituted with 1-3 fluoro, and C₁-C₃ alkoxyalkyl optionally substituted with 1-3 fluoro.

In some embodiments, each R^{A1} is independently selected from cyano, —(C₁-C₃ alkyl)ₚ-NR^{H}R^{I}; C₁-C₃ alkyl, C₁-C₃ alkoxy, C₁-C₃ haloalkyl, C₁-C₃ haloalkoxy, C₁-C₃ hydroxyalkyl optionally substituted with 1-3 fluoro, and C₁-C₃ alkoxyalkyl optionally substituted with 1-3 fluoro.

In some embodiments, each R^{A1} is independently selected from cyano, —(C₁-C₃ alkyl)ₚ-NR^{H}R^{I}; C₁-C₃ alkyl, C₁-C₃ alkoxy, C₁-C₃ haloalkyl, C₁-C₃ haloalkoxy, C₁-C₃ hydroxyalkyl substituted with 1-3 fluoro, and C₁-C₃ alkoxyalkyl substituted with 1-3 fluoro.

In some embodiments, each R^{A1} is independently selected from cyano, —(C₁-C₃ alkyl)ₚ-NR^{H}R^{I}; C₁-C₃ alkyl, C₁-C₃ alkoxy, C₁-C₃ haloalkyl, C₁-C₃ haloalkoxy, C₁-C₃ hydroxyalkyl, and C₁-C₃ alkoxyalkyl.

In some embodiments, p is 0. In some embodiments, p is 1.

In some embodiments, each R^{H} and R^{I} are both hydrogen. In some embodiments, each R^{H} and R^{I} are independently C₁-C₆ alkyl. In some embodiments, one of R^{H} and R^{I} is hydrogen and the other of R^{H} and R^{I} is C₁-C₆ alkyl.

In some embodiments, each R^{A1} is independently selected from cyano, amino, methyl, methoxy, C₁-C₃ haloalkyl, C₁-C₃ haloalkoxy, C₁-C₃ hydroxyalkyl, and C₁-C₃ alkoxyalkyl.

In some embodiments, each R^{A1} is independently selected from cyano, amino, methyl, methoxy, C₁-C₃ haloalkyl, C₁-C₃ haloalkoxy, C₁-C₃ hydroxyalkyl, and C₁-C₃ alkoxyalkyl.

In some embodiments, each R^{A1} is independently selected from cyano, amino, methyl, methoxy, trifluoromethyl, trifluoromethoxy, and hydroxyethyl.

In some embodiments, each R^{A1} is independently selected from cyano, amino, methyl, and trifluoromethyl.

In some embodiments, each R^{A1} is independently selected from cyano and methyl.

In some embodiments, each R^{A1} is independently selected from amino and methyl.

In some embodiments, each R^{A1} is independently selected from methyl and trifluoromethyl.

In some embodiments, at least one R^{A1} is methyl. In some embodiments, at least one R^{A1} is trifluoromethyl. In some embodiments, at least one R^{A1} is amiNo. In some embodiments, one R^{A1} is methyl and one R^{A1} is trifluoromethyl.

In some embodiments, two adjacent R^{A1}, when present, together with the atoms to which they are attached join to form a 5-6 membered heteroaryl or a phenyl, wherein the 5-6 membered heteroaryl and phenyl are optionally substituted by 1-3 independently selected R^{A2}.

In some embodiments, two adjacent R^{A1}, when present, together with the atoms to which they are attached join to form a 5-6 membered heteroaryl, wherein the 5-6 membered heteroaryl is optionally substituted by 1-3 independently selected R^{A2}.

In some embodiments, the 5-6 membered heteroaryl is substituted by 2 independently selected R^{A2}.

In some embodiments, the 5-6 membered heteroaryl is pyrazolyl. In some embodiments, one R^{A2} is attached to the N1 position of the pyrazolyl and the other R^{A2} is attached to the C₃ position of the pyrazolyl.

In some embodiments, two adjacent R^{A1}, when present, together with the atoms to which they are attached join to form a phenyl, wherein the phenyl is optionally substituted by 1-3 independently selected R^{A2}.

In some embodiments, the phenyl is unsubstituted (e.g., when q is 0).

In some embodiments, each R^{A2} is independently selected from halogen, cyano, nitro, —(C₁-C₃ alkyl)ₒ-NR^{J}R^{K}; C₁-C₃ alkyl, C₁-C₃ alkoxy, C₁-C₃ haloalkyl, C₁-C₃ haloalkoxy, C₁-C₃ hydroxyalkyl optionally substituted with 1-3 fluoro, and C₁-C₃ alkoxyalkyl optionally substituted with 1-3 fluoro.

In some embodiments, each R^{A2} is independently selected from cyano, —(C₁-C₃ alkyl)ₒ-NR^{J}R^{K}; C₁-C₃ alkyl, C₁-C₃ alkoxy, C₁-C₃ haloalkyl, C₁-C₃ haloalkoxy, C₁-C₃ hydroxyalkyl optionally substituted with 1-3 fluoro, and C₁-C₃ alkoxyalkyl optionally substituted with 1-3 fluoro.

In some embodiments, each R^{A2} is independently selected from cyano, —(C₁-C₃ alkyl)ₒ-NR^{J}R^{K}; C₁-C₃ alkyl, C₁-C₃ alkoxy, C₁-C₃ haloalkyl, C₁-C₃ haloalkoxy, C₁-C₃ hydroxyalkyl substituted with 1-3 fluoro, and C₁-C₃ alkoxyalkyl substituted with 1-3 fluoro.

In some embodiments, each R^{A2} is independently selected from cyano, —(C₁-C₃ alkyl)ₒ-NR^{J}R^{K}; C₁-C₃ alkyl, C₁-C₃ alkoxy, C₁-C₃ haloalkyl, C₁-C₃ haloalkoxy, C₁-C₃ hydroxyalkyl, and C₁-C₃ alkoxyalkyl.

In some embodiments, o is 0. In some embodiments, o is 1.

In some embodiments, each R^{J} and R^{K} are both hydrogen. In some embodiments, each R^{J} and R^{K} are independently C₁-C₆ alkyl. In some embodiments, one of R^{J} and R^{K} is hydrogen and the other of R^{J} and R^{K} is C₁-C₆ alkyl.

In some embodiments, each R^{A2} is independently selected from cyano, amino, methyl, methoxy, C₁-C₃ haloalkyl, C₁-C₃ haloalkoxy, C₁-C₃ hydroxyalkyl, and C₁-C₃ alkoxyalkyl.

In some embodiments, each R^{A2} is independently selected from cyano, amino, methyl, methoxy, C₁-C₃ haloalkyl, C₁-C₃ haloalkoxy, C₁-C₃ hydroxyalkyl, and C₁-C₃ alkoxyalkyl.

In some embodiments, each R^{A2} is independently selected from cyano, amino, methyl, methoxy, trifluoromethyl, trifluoromethoxy, and hydroxyethyl. In some embodiments, each R^{A2} is independently selected from cyano, amino, methyl, and trifluoromethyl. In some embodiments, each R^{A2} is independently selected from cyano and methyl. In some embodiments, each R^{A} is independently selected from amino and methyl. In some embodiments, each R^{A2} is independently selected from methyl and trifluoromethyl.

In some embodiments, at least one R^{A2} is methyl. In some embodiments, at least one R^{A2} is trifluoromethyl. In some embodiments, at least one R^{A} is amiNo. In some embodiments, one R^{A2} is methyl and one R^{A2} is trifluoromethyl.

In some embodiments, A is a bond and

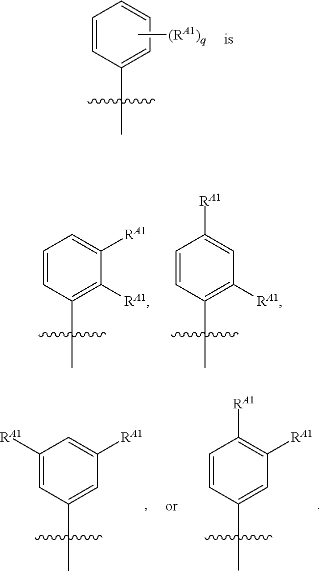

In some embodiments, A is a bond and

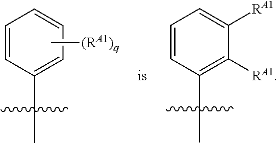

In some embodiments, A is —CH₂— and

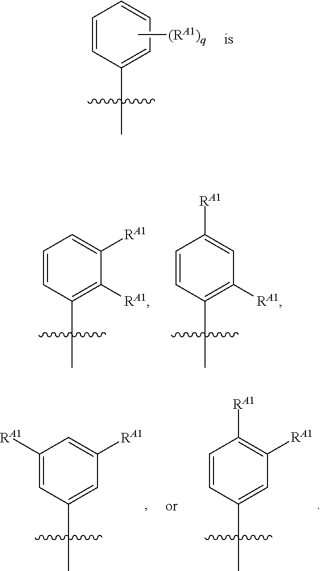

In some embodiments, A is —CH₂— and

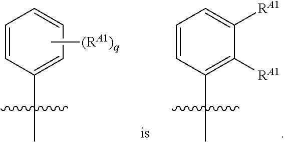

In some embodiments, A is —CH₂—O—CH₂— and

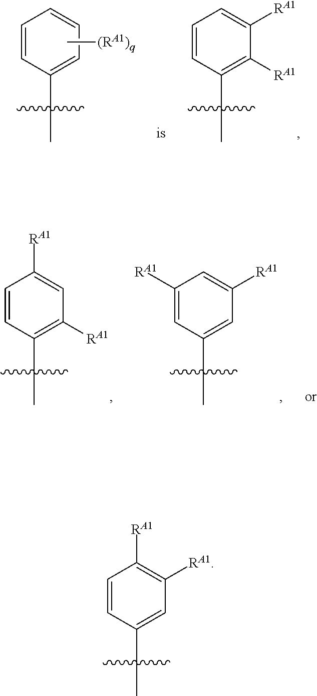

In some embodiments, A is —CH₂—O—CH₂— and

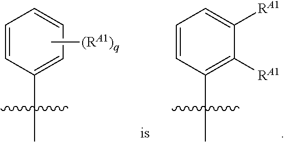

In some embodiments, A is a bond, —CH₂—, or —CH₂—O—CH₂—; and

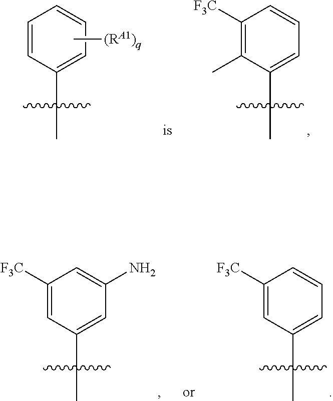

### Methods of Treatment

Provided herein is a method of treating cancer (e.g., a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof)) in a subject in need of such treatment, the method comprising administering to the subject an effective amount of a compound of Formula (I), or a pharmaceutically acceptable salt thereof or a pharmaceutical composition thereof. In some embodiments, a cancer is a Ras pathway-associated cancer. In some embodiments, a cancer is a Ras-associated cancer. In some embodiments, a cancer is a KRas-associated cancer. In some embodiments, a cancer is a HRas-associated cancer. In some embodiments, a cancer is a NRas-associated cancer. In some embodiments, a cancer is a SOS1-associated cancer.

For example, provided herein are methods for treating a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof) in a subject in need of such treatment, the method comprising a) detecting a dysregulation of a Ras pathway gene (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), a Ras pathway protein (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g. BRAF), PI3K, AKT, mTOR, MEK, ERK, or a combination thereof), or the expression or activity or level of any of the same in a sample from the subject; and b) administering a effective amount of a compound of Formula (I), or a pharmaceutically acceptable salt thereof. In some embodiments, the dysregulation of a Ras pathway gene, a Ras pathway protein, or the expression or activity or level of any of the same includes one or more fusion proteins.

For example, provided herein are methods for treating a Ras-associated cancer in a subject in need of such treatment, the method comprising a) detecting a dysregulation of a Ras gene, a Ras protein, or the expression or activity or level of any of the same in a sample from the subject; and b) administering a effective amount of a compound of Formula (I), or a pharmaceutically acceptable salt thereof. In some embodiments, the dysregulation of a Ras gene, a Ras protein, or the expression or activity or level of any of the same includes one or more fusion proteins.

For example, provided herein are methods for treating a KRas-associated cancer in a subject in need of such treatment, the method comprising a) detecting a dysregulation of a KRas gene, a KRas protein, or the expression or activity or level of any of the same in a sample from the subject; and b) administering a effective amount of a compound of Formula (I), or a pharmaceutically acceptable salt thereof. In some embodiments, the dysregulation of a KRas gene, a KRas protein, or the expression or activity or level of any of the same includes one or more fusion proteins.

For example, provided herein are methods for treating a HRas-associated cancer in a subject in need of such treatment, the method comprising a) detecting a dysregulation of a HRas gene, a HRas protein, or the expression or activity or level of any of the same in a sample from the subject; and b) administering a effective amount of a compound of Formula (I), or a pharmaceutically acceptable salt thereof. In some embodiments, the dysregulation of a HRas gene, a HRas protein, or the expression or activity or level of any of the same includes one or more fusion proteins.

For example, provided herein are methods for treating a NRas-associated cancer in a subject in need of such treatment, the method comprising a) detecting a dysregulation of a NRas gene, a NRas protein, or the expression or activity or level of any of the same in a sample from the subject; and b) administering a effective amount of a compound of Formula (I), or a pharmaceutically acceptable salt thereof. In some embodiments, the dysregulation of a NRas gene, a NRas protein, or the expression or activity or level of any of the same includes one or more fusion proteins.

For example, provided herein are methods for treating a SOS1-associated cancer in a subject in need of such treatment, the method comprising a) detecting a dysregulation of a SOS1 gene, a SOS1 protein, or the expression or activity or level of any of the same in a sample from the subject; and b) administering a effective amount of a compound of Formula (I), or a pharmaceutically acceptable salt thereof. In some embodiments, the dysregulation of a SOS1 gene, a SOS1 protein, or the expression or activity or level of any of the same includes one or more fusion proteins.

Also provided are methods for treating cancer in a subject in need thereof, the method comprising: (a) detecting a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof) in the subject; and (b) administering to the subject an effective amount of a compound of Formula (I), or a pharmaceutically acceptable salt thereof, or a pharmaceutical composition thereof. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a Ras pathway-associated cancer through the use of a regulatory agency-approved, e.g., FDA-approved test or assay for identifying dysregulation of a Ras pathway gene (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), a Ras pathway protein (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: (a) detecting a Ras-associated cancer in the subject; and (b) administering to the subject an effective amount of a compound of Formula (I), or a pharmaceutically acceptable salt thereof, or a pharmaceutical composition thereof. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a Ras-associated cancer through the use of a regulatory agency-approved, e.g., FDA-approved test or assay for identifying dysregulation of a Ras gene, a Ras protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: (a) detecting a KRas-associated cancer in the subject; and (b) administering to the subject an effective amount of a compound of Formula (I), or a pharmaceutically acceptable salt thereof, or a pharmaceutical composition thereof. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a KRas-associated cancer through the use of a regulatory agency-approved, e.g., FDA-approved test or assay for identifying dysregulation of a KRas gene, a KRas protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: (a) detecting a HRas-associated cancer in the subject; and (b) administering to the subject an effective amount of a compound of Formula (I), or a pharmaceutically acceptable salt thereof, or a pharmaceutical composition thereof. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a HRas-associated cancer through the use of a regulatory agency-approved, e.g., FDA-approved test or assay for identifying dysregulation of a HRas gene, a HRas protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: (a) detecting a NRas-associated cancer in the subject; and (b) administering to the subject an effective amount of a compound of Formula (I), or a pharmaceutically acceptable salt thereof, or a pharmaceutical composition thereof. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a NRas-associated cancer through the use of a regulatory agency-approved, e.g., FDA-approved test or assay for identifying dysregulation of a NRas gene, a NRas protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: (a) detecting a SOS1-associated cancer in the subject; and (b) administering to the subject an effective amount of a compound of Formula (I), or a pharmaceutically acceptable salt thereof, or a pharmaceutical composition thereof. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a SOS1-associated cancer through the use of a regulatory agency-approved, e.g., FDA-approved test or assay for identifying dysregulation of a SOS1 gene, a SOS1 protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: administering an effective amount of a compound of Formula (I), or a pharmaceutically acceptable salt thereof, or a pharmaceutical composition thereof to a subject determined to have a cancer associated with a dysregulation of a Ras pathway gene (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), a Ras pathway protein (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or expression or activity or level of any of the same. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof) through the use of a regulatory agency-approved, e.g., FDA-approved test or assay for identifying dysregulation of a Ras pathway gene, a Ras pathway protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: administering an effective amount of a compound of Formula (I), or a pharmaceutically acceptable salt thereof, or a pharmaceutical composition thereof to a subject determined to have a cancer associated with a dysregulation of a Ras gene, a Ras protein, or expression or activity or level of any of the same. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a Ras-associated cancer through the use of a regulatory agency-approved, e.g., FDA-approved test or assay for identifying dysregulation of a Ras gene, a Ras protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: administering an effective amount of a compound of Formula (I), or a pharmaceutically acceptable salt thereof, or a pharmaceutical composition thereof to a subject determined to have a cancer associated with a dysregulation of a KRas gene, a KRas protein, or expression or activity or level of any of the same. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a KRas-associated cancer through the use of a regulatory agency-approved, e.g., FDA-approved test or assay for identifying dysregulation of a KRas gene, a KRas protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: administering an effective amount of a compound of Formula (I), or a pharmaceutically acceptable salt thereof, or a pharmaceutical composition thereof to a subject determined to have a cancer associated with a dysregulation of a HRas gene, a HRas protein, or expression or activity or level of any of the same. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a HRas-associated cancer through the use of a regulatory agency-approved, e.g., FDA-approved test or assay for identifying dysregulation of a HRas gene, a HRas protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: administering an effective amount of a compound of Formula (I), or a pharmaceutically acceptable salt thereof, or a pharmaceutical composition thereof to a subject determined to have a cancer associated with a dysregulation of a NRas gene, a NRas protein, or expression or activity or level of any of the same. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a NRas-associated cancer through the use of a regulatory agency-approved, e.g., FDA-approved test or assay for identifying dysregulation of a NRas gene, a NRas protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: administering an effective amount of a compound of Formula (I), or a pharmaceutically acceptable salt thereof, or a pharmaceutical composition thereof to a subject determined to have a cancer associated with a dysregulation of a SOS1 gene, a SOS1 protein, or expression or activity or level of any of the same. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a SOS1-associated cancer through the use of a regulatory agency-approved, e.g., FDA-approved test or assay for identifying dysregulation of a SOS1 gene, a SOS1 protein, or expression or activity or level of any of the same, in a subject or a biopsy sample from the subject or by performing any of the non-limiting examples of assays described herein. In some embodiments, the test or assay is provided as a kit.

Also provided are methods of treating a subject that include performing an assay on a sample obtained from the subject to determine whether the subject has a dysregulation of a Ras pathway gene (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), a Ras pathway protein (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or expression or activity or level of any of the same, and administering (e.g., specifically or selectively administering) an effective amount of a compound of Formula (I), or a pharmaceutically acceptable salt thereof, or a pharmaceutical composition thereof, to the subject determined to have a dysregulation of a Ras pathway gene, a Ras pathway protein, or expression or activity or level of any of the same. Some embodiments of these methods further include administering to the subject another anticancer agent (e.g., a small molecule or immunotherapy). In some embodiments of these methods, the subject was previously treated with another anticancer treatment, e.g., at least partial resection of a tumor or radiation therapy. In some embodiments, the subject is a subject suspected of having a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof), a subject presenting with one or more symptoms of a Ras pathway-associated cancer, or a subject having an elevated risk of developing a Ras pathway-associated cancer. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art.

Also provided are methods of treating a subject that include performing an assay on a sample obtained from the subject to determine whether the subject has a dysregulation of a Ras gene, a Ras protein, or expression or activity or level of any of the same, and administering (e.g., specifically or selectively administering) an effective amount of a compound of Formula (I), or a pharmaceutically acceptable salt thereof, or a pharmaceutical composition thereof, to the subject determined to have a dysregulation of a Ras gene, a Ras protein, or expression or activity or level of any of the same. Some embodiments of these methods further include administering to the subject another anticancer agent (e.g., a small molecule or immunotherapy). In some embodiments of these methods, the subject was previously treated with another anticancer treatment, e.g., at least partial resection of a tumor or radiation therapy. In some embodiments, the subject is a subject suspected of having a Ras-associated cancer, a subject presenting with one or more symptoms of a Ras-associated cancer, or a subject having an elevated risk of developing a Ras-associated cancer. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art.

Also provided are methods of treating a subject that include performing an assay on a sample obtained from the subject to determine whether the subject has a dysregulation of a KRas gene, a KRas protein, or expression or activity or level of any of the same, and administering (e.g., specifically or selectively administering) an effective amount of a compound of Formula (I), or a pharmaceutically acceptable salt thereof, or a pharmaceutical composition thereof, to the subject determined to have a dysregulation of a KRas gene, a KRas protein, or expression or activity or level of any of the same. Some embodiments of these methods further include administering to the subject another anticancer agent (e.g., a small molecule or immunotherapy). In some embodiments of these methods, the subject was previously treated with another anticancer treatment, e.g., at least partial resection of a tumor or radiation therapy. In some embodiments, the subject is a subject suspected of having a KRas-associated cancer, a subject presenting with one or more symptoms of a KRas-associated cancer, or a subject having an elevated risk of developing a KRas-associated cancer. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art.

Also provided are methods of treating a subject that include performing an assay on a sample obtained from the subject to determine whether the subject has a dysregulation of a HRas gene, a HRas protein, or expression or activity or level of any of the same, and administering (e.g., specifically or selectively administering) an effective amount of a compound of Formula (I), or a pharmaceutically acceptable salt thereof, or a pharmaceutical composition thereof, to the subject determined to have a dysregulation of a HRas gene, a HRas protein, or expression or activity or level of any of the same. Some embodiments of these methods further include administering to the subject another anticancer agent (e.g., a small molecule or immunotherapy). In some embodiments of these methods, the subject was previously treated with another anticancer treatment, e.g., at least partial resection of a tumor or radiation therapy. In some embodiments, the subject is a subject suspected of having a HRas-associated cancer, a subject presenting with one or more symptoms of an HRas-associated cancer, or a subject having an elevated risk of developing a HRas-associated cancer. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art.

Also provided are methods of treating a subject that include performing an assay on a sample obtained from the subject to determine whether the subject has a dysregulation of a NRas gene, a NRas protein, or expression or activity or level of any of the same, and administering (e.g., specifically or selectively administering) an effective amount of a compound of Formula (I), or a pharmaceutically acceptable salt thereof, or a pharmaceutical composition thereof, to the subject determined to have a dysregulation of a NRas gene, a NRas protein, or expression or activity or level of any of the same. Some embodiments of these methods further include administering to the subject another anticancer agent (e.g., a small molecule or immunotherapy). In some embodiments of these methods, the subject was previously treated with another anticancer treatment, e.g., at least partial resection of a tumor or radiation therapy. In some embodiments, the subject is a subject suspected of having a NRas-associated cancer, a subject presenting with one or more symptoms of an NRas-associated cancer, or a subject having an elevated risk of developing a NRas-associated cancer. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art.

Also provided are methods of treating a subject that include performing an assay on a sample obtained from the subject to determine whether the subject has a dysregulation of a SOS1 gene, a SOS1 protein, or expression or activity or level of any of the same, and administering (e.g., specifically or selectively administering) an effective amount of a compound of Formula (I), or a pharmaceutically acceptable salt thereof, or a pharmaceutical composition thereof, to the subject determined to have a dysregulation of a SOS1 gene, a SOS1 protein, or expression or activity or level of any of the same. Some embodiments of these methods further include administering to the subject another anticancer agent (e.g., a small molecule or immunotherapy). In some embodiments of these methods, the subject was previously treated with another anticancer treatment, e.g., at least partial resection of a tumor or radiation therapy. In some embodiments, the subject is a subject suspected of having a SOS1-associated cancer, a subject presenting with one or more symptoms of a SOS1-associated cancer, or a subject having an elevated risk of developing a SOS1-associated cancer. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art.

Also provided is a compound of Formula (I), or a pharmaceutically acceptable salt thereof, or a pharmaceutical composition thereof, for use in treating a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof) in a subject identified or diagnosed as having a Ras pathway-associated cancer through a step of performing an assay (e.g., an in vitro assay) on a sample obtained from the subject to determine whether the subject has a dysregulation of a Ras pathway protein (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), a Ras pathway protein (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or expression or activity or level of any of the same, where the presence of a dysregulation of a Ras pathway gene, a Ras pathway protein, or expression or activity or level of any of the same, identifies that the subject has a Ras pathway-associated cancer. Also provided is the use of a compound of Formula (I), or a pharmaceutically acceptable salt thereof, for the manufacture of a medicament for treating a Ras pathway-associated cancer in a subject identified or diagnosed as having a Ras pathway-associated cancer through a step of performing an assay on a sample obtained from the subject to determine whether the subject has a dysregulation of a Ras pathway gene, a Ras pathway protein, or expression or activity or level of any of the same where the presence of dysregulation of a Ras pathway gene, a Ras pathway protein, or expression or activity or level of any of the same, identifies that the subject has a Ras pathway-associated cancer. Some embodiments of any of the methods or uses described herein further include recording in the subject's clinical record (e.g., a computer readable medium) that the subject is determined to have a dysregulation of a Ras pathway gene, a Ras pathway protein, or expression or activity or level of any of the same, through the performance of the assay, should be administered a compound of Formula (I), or a pharmaceutically acceptable salt thereof, or a pharmaceutical composition thereof. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy.

Also provided is a compound of Formula (I), or a pharmaceutically acceptable salt thereof, or a pharmaceutical composition thereof, for use in treating a Ras-associated cancer in a subject identified or diagnosed as having a Ras-associated cancer through a step of performing an assay (e.g., an in vitro assay) on a sample obtained from the subject to determine whether the subject has a dysregulation of a Ras gene, a Ras protein, or expression or activity or level of any of the same, where the presence of a dysregulation of a Ras gene, a Ras protein, or expression or activity or level of any of the same, identifies that the subject has a Ras-associated cancer. Also provided is the use of a compound of Formula (I), or a pharmaceutically acceptable salt thereof, for the manufacture of a medicament for treating a Ras-associated cancer in a subject identified or diagnosed as having a Ras-associated cancer through a step of performing an assay on a sample obtained from the subject to determine whether the subject has a dysregulation of a Ras gene, a Ras protein, or expression or activity or level of any of the same where the presence of dysregulation of a Ras gene, a Ras protein, or expression or activity or level of any of the same, identifies that the subject has a Ras-associated cancer. Some embodiments of any of the methods or uses described herein further include recording in the subject's clinical record (e.g., a computer readable medium) that the subject is determined to have a dysregulation of a Ras gene, a Ras protein, or expression or activity or level of any of the same, through the performance of the assay, should be administered a compound of Formula (I), or a pharmaceutically acceptable salt thereof, or a pharmaceutical composition thereof. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy.

Also provided is a compound of Formula (I), or a pharmaceutically acceptable salt thereof, or a pharmaceutical composition thereof, for use in treating a KRas-associated cancer in a subject identified or diagnosed as having a KRas-associated cancer through a step of performing an assay (e.g., an in vitro assay) on a sample obtained from the subject to determine whether the subject has a dysregulation of a KRas gene, a KRas protein, or expression or activity or level of any of the same, where the presence of a dysregulation of a KRas gene, a KRas protein, or expression or activity or level of any of the same, identifies that the subject has a KRas-associated cancer. Also provided is the use of a compound of Formula (I), or a pharmaceutically acceptable salt thereof, for the manufacture of a medicament for treating a KRas-associated cancer in a subject identified or diagnosed as having a KRas-associated cancer through a step of performing an assay on a sample obtained from the subject to determine whether the subject has a dysregulation of a KRas gene, a KRas protein, or expression or activity or level of any of the same where the presence of dysregulation of a KRas gene, a KRas protein, or expression or activity or level of any of the same, identifies that the subject has a KRas-associated cancer. Some embodiments of any of the methods or uses described herein further include recording in the subject's clinical record (e.g., a computer readable medium) that the subject is determined to have a dysregulation of a KRas gene, a KRas protein, or expression or activity or level of any of the same, through the performance of the assay, should be administered a compound of Formula (I), or a pharmaceutically acceptable salt thereof, or a pharmaceutical composition thereof. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy.

Also provided is a compound of Formula (I), or a pharmaceutically acceptable salt thereof, or a pharmaceutical composition thereof, for use in treating a HRas-associated cancer in a subject identified or diagnosed as having a HRas-associated cancer through a step of performing an assay (e.g., an in vitro assay) on a sample obtained from the subject to determine whether the subject has a dysregulation of a HRas gene, a HRas protein, or expression or activity or level of any of the same, where the presence of a dysregulation of a HRas gene, a HRas protein, or expression or activity or level of any of the same, identifies that the subject has a HRas-associated cancer. Also provided is the use of a compound of Formula (I), or a pharmaceutically acceptable salt thereof, for the manufacture of a medicament for treating a HRas-associated cancer in a subject identified or diagnosed as having a HRas-associated cancer through a step of performing an assay on a sample obtained from the subject to determine whether the subject has a dysregulation of a HRas gene, a HRas protein, or expression or activity or level of any of the same where the presence of dysregulation of a HRas gene, a HRas protein, or expression or activity or level of any of the same, identifies that the subject has a HRas-associated cancer. Some embodiments of any of the methods or uses described herein further include recording in the subject's clinical record (e.g., a computer readable medium) that the subject is determined to have a dysregulation of a HRas gene, a HRas protein, or expression or activity or level of any of the same, through the performance of the assay, should be administered a compound of Formula (I), or a pharmaceutically acceptable salt thereof, or a pharmaceutical composition thereof. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy.

Also provided is a compound of Formula (I), or a pharmaceutically acceptable salt thereof, or a pharmaceutical composition thereof, for use in treating a NRas-associated cancer in a subject identified or diagnosed as having a NRas-associated cancer through a step of performing an assay (e.g., an in vitro assay) on a sample obtained from the subject to determine whether the subject has a dysregulation of a NRas gene, a NRas protein, or expression or activity or level of any of the same, where the presence of a dysregulation of a NRas gene, a NRas protein, or expression or activity or level of any of the same, identifies that the subject has a NRas-associated cancer. Also provided is the use of a compound of Formula (I), or a pharmaceutically acceptable salt thereof, for the manufacture of a medicament for treating a NRas-associated cancer in a subject identified or diagnosed as having a NRas-associated cancer through a step of performing an assay on a sample obtained from the subject to determine whether the subject has a dysregulation of a NRas gene, a NRas protein, or expression or activity or level of any of the same where the presence of dysregulation of a NRas gene, a NRas protein, or expression or activity or level of any of the same, identifies that the subject has a NRas-associated cancer. Some embodiments of any of the methods or uses described herein further include recording in the subject's clinical record (e.g., a computer readable medium) that the subject is determined to have a dysregulation of a NRas gene, a NRas protein, or expression or activity or level of any of the same, through the performance of the assay, should be administered a compound of Formula (I), or a pharmaceutically acceptable salt thereof, or a pharmaceutical composition thereof. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy.

Also provided is a compound of Formula (I), or a pharmaceutically acceptable salt thereof, or a pharmaceutical composition thereof, for use in treating a SOS1-associated cancer in a subject identified or diagnosed as having a SOS1-associated cancer through a step of performing an assay (e.g., an in vitro assay) on a sample obtained from the subject to determine whether the subject has a dysregulation of a SOS1 gene, a SOS1 protein, or expression or activity or level of any of the same, where the presence of a dysregulation of a SOS1 gene, a SOS1 protein, or expression or activity or level of any of the same, identifies that the subject has a SOS1-associated cancer. Also provided is the use of a compound of Formula (I), or a pharmaceutically acceptable salt thereof, for the manufacture of a medicament for treating a SOS1-associated cancer in a subject identified or diagnosed as having a SOS1-associated cancer through a step of performing an assay on a sample obtained from the subject to determine whether the subject has a dysregulation of a SOS1 gene, a SOS1 protein, or expression or activity or level of any of the same where the presence of dysregulation of a SOS1 gene, a SOS1 protein, or expression or activity or level of any of the same, identifies that the subject has a SOS1-associated cancer. Some embodiments of any of the methods or uses described herein further include recording in the subject's clinical record (e.g., a computer readable medium) that the subject is determined to have a dysregulation of a SOS1 gene, a SOS1 protein, or expression or activity or level of any of the same, through the performance of the assay, should be administered a compound of Formula (I), or a pharmaceutically acceptable salt thereof, or a pharmaceutical composition thereof. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy.

In some embodiments of any of the methods or uses described herein, the subject has been identified or diagnosed as having a cancer with a dysregulation of a Ras pathway gene, a Ras pathway protein, or expression or activity or level of any of the same. In some embodiments of any of the methods or uses described herein, the subject has a tumor that is positive for a dysregulation of a Ras pathway gene (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), a Ras pathway protein (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or expression or activity or level of any of the same. In some embodiments of any of the methods or uses described herein, the subject can be a subject with a tumor(s) that is positive for a dysregulation of a Ras pathway gene, a Ras pathway protein, or expression or activity or level of any of the same. In some embodiments of any of the methods or uses described herein, the subject can be a subject whose tumors have a dysregulation of a Ras pathway gene, a Ras pathway protein, or expression or activity or level of any of the same. In some embodiments of any of the methods or uses described herein, the subject is suspected of having a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof). In some embodiments, provided herein are methods for treating a Ras pathway-associated cancer in a subject in need of such treatment, the method comprising a) detecting a dysregulation of a Ras pathway gene, a Ras pathway protein, or the expression or activity or level of any of the same in a sample from the subject; and b) administering an effective amount of a compound of Formula (I), or a pharmaceutically acceptable salt thereof. In some embodiments, the dysregulation of a Ras pathway gene, a Ras pathway protein, or the expression or activity or level of any of the same includes one or more Ras pathway protein point mutations/insertions/deletions.

In some embodiments of any of the methods or uses described herein, the subject has been identified or diagnosed as having a cancer with a dysregulation of a Ras gene, a Ras protein, or expression or activity or level of any of the same. In some embodiments of any of the methods or uses described herein, the subject has a tumor that is positive for a dysregulation of a Ras gene, a Ras protein, or expression or activity or level of any of the same. In some embodiments of any of the methods or uses described herein, the subject can be a subject with a tumor(s) that is positive for a dysregulation of a Ras gene, a Ras protein, or expression or activity or level of any of the same. In some embodiments of any of the methods or uses described herein, the subject can be a subject whose tumors have a dysregulation of a Ras gene, a Ras protein, or expression or activity or level of any of the same. In some embodiments of any of the methods or uses described herein, the subject is suspected of having a Ras-associated cancer. In some embodiments, provided herein are methods for treating a Ras-associated cancer in a subject in need of such treatment, the method comprising a) detecting a dysregulation of a Ras gene, a Ras protein, or the expression or activity or level of any of the same in a sample from the subject; and b) administering an effective amount of a compound of Formula (I), or a pharmaceutically acceptable salt thereof. In some embodiments, the dysregulation of a Ras gene, a Ras protein, or the expression or activity or level of any of the same includes one or more Ras protein point mutations/insertions/deletions.

In some embodiments of any of the methods or uses described herein, the subject has been identified or diagnosed as having a cancer with a dysregulation of a KRas gene, a KRas protein, or expression or activity or level of any of the same. In some embodiments of any of the methods or uses described herein, the subject has a tumor that is positive for a dysregulation of a KRas gene, a KRas protein, or expression or activity or level of any of the same. In some embodiments of any of the methods or uses described herein, the subject can be a subject with a tumor(s) that is positive for a dysregulation of a KRas gene, a KRas protein, or expression or activity or level of any of the same. In some embodiments of any of the methods or uses described herein, the subject can be a subject whose tumors have a dysregulation of a KRas gene, a KRas protein, or expression or activity or level of any of the same. In some embodiments of any of the methods or uses described herein, the subject is suspected of having a KRas-associated cancer. In some embodiments, provided herein are methods for treating a KRas-associated cancer in a subject in need of such treatment, the method comprising a) detecting a dysregulation of a KRas gene, a KRas protein, or the expression or activity or level of any of the same in a sample from the subject; and b) administering an effective amount of a compound of Formula (I), or a pharmaceutically acceptable salt thereof. In some embodiments, the dysregulation of a KRas gene, a KRas protein, or the expression or activity or level of any of the same includes one or more KRas protein point mutations/insertions/deletions. Non-limiting examples of KRas protein point mutations/insertions/deletions are described in Table 1.

In some embodiments of any of the methods or uses described herein, the subject has been identified or diagnosed as having a cancer with a dysregulation of a HRas gene, a HRas protein, or expression or activity or level of any of the same. In some embodiments of any of the methods or uses described herein, the subject has a tumor that is positive for a dysregulation of a HRas gene, a HRas protein, or expression or activity or level of any of the same. In some embodiments of any of the methods or uses described herein, the subject can be a subject with a tumor(s) that is positive for a dysregulation of a HRas gene, a HRas protein, or expression or activity or level of any of the same. In some embodiments of any of the methods or uses described herein, the subject can be a subject whose tumors have a dysregulation of a HRas gene, a HRas protein, or expression or activity or level of any of the same. In some embodiments of any of the methods or uses described herein, the subject is suspected of having a HRas-associated cancer. In some embodiments, provided herein are methods for treating a HRas-associated cancer in a subject in need of such treatment, the method comprising a) detecting a dysregulation of a HRas gene, a HRas protein, or the expression or activity or level of any of the same in a sample from the subject; and b) administering an effective amount of a compound of Formula (I), or a pharmaceutically acceptable salt thereof. In some embodiments, the dysregulation of a HRas gene, a HRas protein, or the expression or activity or level of any of the same includes one or more HRas protein point mutations/insertions/deletions. Non-limiting examples of HRas protein point mutations/insertions/deletions are described in Table 2.

In some embodiments of any of the methods or uses described herein, the subject has been identified or diagnosed as having a cancer with a dysregulation of a NRas gene, a NRas protein, or expression or activity or level of any of the same. In some embodiments of any of the methods or uses described herein, the subject has a tumor that is positive for a dysregulation of a NRas gene, a NRas protein, or expression or activity or level of any of the same. In some embodiments of any of the methods or uses described herein, the subject can be a subject with a tumor(s) that is positive for a dysregulation of a NRas gene, a NRas protein, or expression or activity or level of any of the same. In some embodiments of any of the methods or uses described herein, the subject can be a subject whose tumors have a dysregulation of a NRas gene, a NRas protein, or expression or activity or level of any of the same. In some embodiments of any of the methods or uses described herein, the subject is suspected of having a NRas-associated cancer. In some embodiments, provided herein are methods for treating a NRas-associated cancer in a subject in need of such treatment, the method comprising a) detecting a dysregulation of a NRas gene, a NRas protein, or the expression or activity or level of any of the same in a sample from the subject; and b) administering an effective amount of a compound of Formula (I), or a pharmaceutically acceptable salt thereof. In some embodiments, the dysregulation of a NRas gene, a NRas protein, or the expression or activity or level of any of the same includes one or more NRas protein point mutations/insertions/deletions. Non-limiting examples of NRas protein point mutations/insertions/deletions are described in Table 3.

In some embodiments of any of the methods or uses described herein, the subject has been identified or diagnosed as having a cancer with a dysregulation of a SOS1 gene, a SOS1 protein, or expression or activity or level of any of the same. In some embodiments of any of the methods or uses described herein, the subject has a tumor that is positive for a dysregulation of a SOS1 gene, a SOS1 protein, or expression or activity or level of any of the same. In some embodiments of any of the methods or uses described herein, the subject can be a subject with a tumor(s) that is positive for a dysregulation of a SOS1 gene, a SOS1 protein, or expression or activity or level of any of the same. In some embodiments of any of the methods or uses described herein, the subject can be a subject whose tumors have a dysregulation of a SOS1 gene, a SOS1 protein, or expression or activity or level of any of the same. In some embodiments of any of the methods or uses described herein, the subject is suspected of having a SOS1-associated cancer. In some embodiments, provided herein are methods for treating a SOS1-associated cancer in a subject in need of such treatment, the method comprising a) detecting a dysregulation of a SOS1 gene, a SOS1 protein, or the expression or activity or level of any of the same in a sample from the subject; and b) administering an effective amount of a compound of Formula (I), or a pharmaceutically acceptable salt thereof. In some embodiments, the dysregulation of a SOS1 gene, a SOS1 protein, or the expression or activity or level of any of the same includes one or more SOS1 protein point mutations/insertions/deletions. Non-limiting examples of SOS1 protein point mutations/insertions/deletions are described in Table 4.

In some embodiments, the cancer with a dysregulation of a Ras pathway gene (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), a Ras pathway protein (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or expression or activity or level of any of the same is determined using a regulatory agency-approved, e.g., FDA-approved, assay or kit. In some embodiments, the tumor with a dysregulation of a Ras pathway gene, a Ras pathway protein, or expression or activity or level of any of the same is determined using a regulatory agency-approved, e.g., FDA-approved, assay or kit.

In some embodiments, the cancer with a dysregulation of a Ras gene, a Ras protein, or expression or activity or level of any of the same is determined using a regulatory agency-approved, e.g., FDA-approved, assay or kit. In some embodiments, the tumor with a dysregulation of a Ras gene, a Ras protein, or expression or activity or level of any of the same is determined using a regulatory agency-approved, e.g., FDA-approved, assay or kit.

In some embodiments, the cancer with a dysregulation of a KRas gene, a KRas protein, or expression or activity or level of any of the same is determined using a regulatory agency-approved, e.g., FDA-approved, assay or kit. In some embodiments, the tumor with a dysregulation of a KRas gene, a KRas protein, or expression or activity or level of any of the same is determined using a regulatory agency-approved, e.g., FDA-approved, assay or kit.

In some embodiments, the cancer with a dysregulation of a HRas gene, a HRas protein, or expression or activity or level of any of the same is determined using a regulatory agency-approved, e.g., FDA-approved, assay or kit. In some embodiments, the tumor with a dysregulation of a HRas gene, a HRas protein, or expression or activity or level of any of the same is determined using a regulatory agency-approved, e.g., FDA-approved, assay or kit.

In some embodiments, the cancer with a dysregulation of a NRas gene, a NRas protein, or expression or activity or level of any of the same is determined using a regulatory agency-approved, e.g., FDA-approved, assay or kit. In some embodiments, the tumor with a dysregulation of a NRas gene, a NRas protein, or expression or activity or level of any of the same is determined using a regulatory agency-approved, e.g., FDA-approved, assay or kit.

In some embodiments, the cancer with a dysregulation of a SOS1 gene, a SOS1 protein, or expression or activity or level of any of the same is determined using a regulatory agency-approved, e.g., FDA-approved, assay or kit. In some embodiments, the tumor with a dysregulation of a SOS1 gene, a SOS1 protein, or expression or activity or level of any of the same is determined using a regulatory agency-approved, e.g., FDA-approved, assay or kit.

In some embodiments of any of the methods or uses described herein, the subject has a clinical record indicating that the subject has a tumor that has a dysregulation of a Ras pathway gene (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), a Ras pathway protein (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or expression or activity or level of any of the same. Also provided are methods of treating a subject that include administering an effective amount of a compound of Formula (I), or a pharmaceutically acceptable salt thereof, to a subject having a clinical record that indicates that the subject has a dysregulation of a Ras pathway gene, a Ras pathway protein, or expression or activity or level of any of the same.

In some embodiments of any of the methods or uses described herein, the subject has a clinical record indicating that the subject has a tumor that has a dysregulation of a Ras gene, a Ras protein, or expression or activity or level of any of the same. Also provided are methods of treating a subject that include administering an effective amount of a compound of Formula (I), or a pharmaceutically acceptable salt thereof, to a subject having a clinical record that indicates that the subject has a dysregulation of a Ras gene, a Ras protein, or expression or activity or level of any of the same.

In some embodiments of any of the methods or uses described herein, the subject has a clinical record indicating that the subject has a tumor that has a dysregulation of a KRas gene, a KRas protein, or expression or activity or level of any of the same. Also provided are methods of treating a subject that include administering an effective amount of a compound of Formula (I), or a pharmaceutically acceptable salt thereof, to a subject having a clinical record that indicates that the subject has a dysregulation of a KRas gene, a KRas protein, or expression or activity or level of any of the same.

In some embodiments of any of the methods or uses described herein, the subject has a clinical record indicating that the subject has a tumor that has a dysregulation of a HRas gene, a HRas protein, or expression or activity or level of any of the same. Also provided are methods of treating a subject that include administering an effective amount of a compound of Formula (I), or a pharmaceutically acceptable salt thereof, to a subject having a clinical record that indicates that the subject has a dysregulation of a HRas gene, a HRas protein, or expression or activity or level of any of the same.

In some embodiments of any of the methods or uses described herein, the subject has a clinical record indicating that the subject has a tumor that has a dysregulation of a NRas gene, a NRas protein, or expression or activity or level of any of the same. Also provided are methods of treating a subject that include administering an effective amount of a compound of Formula (I), or a pharmaceutically acceptable salt thereof, to a subject having a clinical record that indicates that the subject has a dysregulation of a NRas gene, a NRas protein, or expression or activity or level of any of the same.

In some embodiments of any of the methods or uses described herein, the subject has a clinical record indicating that the subject has a tumor that has a dysregulation of a SOS1 gene, a SOS1 protein, or expression or activity or level of any of the same. Also provided are methods of treating a subject that include administering an effective amount of a compound of Formula (I), or a pharmaceutically acceptable salt thereof, to a subject having a clinical record that indicates that the subject has a dysregulation of a SOS1 gene, a SOS1 protein, or expression or activity or level of any of the same.

In some embodiments, the methods provided herein include performing an assay on a sample obtained from the subject to determine whether the subject has a dysregulation of a Ras pathway gene (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), a Ras pathway protein (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or expression or level of any of the same. In some such embodiments, the method also includes administering to a subject determined to have a dysregulation of a Ras pathway gene, a Ras pathway protein, or expression or activity, or level of any of the same an effective amount of a compound of Formula (I), or a pharmaceutically acceptable salt thereof. In some embodiments, the method includes determining that a subject has a dysregulation of a Ras pathway gene, a Ras pathway protein, or expression or level of any of the same via an assay performed on a sample obtained from the subject. In such embodiments, the method also includes administering to a subject an effective amount of a compound of Formula (I), or a pharmaceutically acceptable salt thereof.

In some embodiments, the methods provided herein include performing an assay on a sample obtained from the subject to determine whether the subject has a dysregulation of a Ras gene, a Ras protein, or expression or level of any of the same. In some such embodiments, the method also includes administering to a subject determined to have a dysregulation of a Ras gene, a Ras protein, or expression or activity, or level of any of the same an effective amount of a compound of Formula (I), or a pharmaceutically acceptable salt thereof. In some embodiments, the method includes determining that a subject has a dysregulation of a Ras gene, a Ras protein, or expression or level of any of the same via an assay performed on a sample obtained from the subject. In such embodiments, the method also includes administering to a subject an effective amount of a compound of Formula (I), or a pharmaceutically acceptable salt thereof.

In some embodiments, the methods provided herein include performing an assay on a sample obtained from the subject to determine whether the subject has a dysregulation of a KRas gene, a KRas protein, or expression or level of any of the same. In some such embodiments, the method also includes administering to a subject determined to have a dysregulation of a KRas gene, a KRas protein, or expression or activity, or level of any of the same an effective amount of a compound of Formula (I), or a pharmaceutically acceptable salt thereof. In some embodiments, the method includes determining that a subject has a dysregulation of a KRas gene, a KRas protein, or expression or level of any of the same via an assay performed on a sample obtained from the subject. In such embodiments, the method also includes administering to a subject an effective amount of a compound of Formula (I), or a pharmaceutically acceptable salt thereof.

In some embodiments, the methods provided herein include performing an assay on a sample obtained from the subject to determine whether the subject has a dysregulation of a HRas gene, a HRas protein, or expression or level of any of the same. In some such embodiments, the method also includes administering to a subject determined to have a dysregulation of a HRas gene, a HRas protein, or expression or activity, or level of any of the same an effective amount of a compound of Formula (I), or a pharmaceutically acceptable salt thereof. In some embodiments, the method includes determining that a subject has a dysregulation of a HRas gene, a HRas protein, or expression or level of any of the same via an assay performed on a sample obtained from the subject. In such embodiments, the method also includes administering to a subject an effective amount of a compound of Formula (I), or a pharmaceutically acceptable salt thereof.

In some embodiments, the methods provided herein include performing an assay on a sample obtained from the subject to determine whether the subject has a dysregulation of a NRas gene, a NRas protein, or expression or level of any of the same. In some such embodiments, the method also includes administering to a subject determined to have a dysregulation of a NRas gene, a NRas protein, or expression or activity, or level of any of the same an effective amount of a compound of Formula (I), or a pharmaceutically acceptable salt thereof. In some embodiments, the method includes determining that a subject has a dysregulation of a NRas gene, a NRas protein, or expression or level of any of the same via an assay performed on a sample obtained from the subject. In such embodiments, the method also includes administering to a subject an effective amount of a compound of Formula (I), or a pharmaceutically acceptable salt thereof.

In some embodiments, the methods provided herein include performing an assay on a sample obtained from the subject to determine whether the subject has a dysregulation of a SOS1 gene, a SOS1 protein, or expression or level of any of the same. In some such embodiments, the method also includes administering to a subject determined to have a dysregulation of a SOS1 gene, a SOS1 protein, or expression or activity, or level of any of the same an effective amount of a compound of Formula (I), or a pharmaceutically acceptable salt thereof. In some embodiments, the method includes determining that a subject has a dysregulation of a SOS1 gene, a SOS1 protein, or expression or level of any of the same via an assay performed on a sample obtained from the subject. In such embodiments, the method also includes administering to a subject an effective amount of a compound of Formula (I), or a pharmaceutically acceptable salt thereof.

In some embodiments of any of the methods or uses described herein, the cancer (e.g., Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof)) is a hematological cancer. Examples of hematological cancers (e.g., hematological cancers that are Ras pathway-associated cancers) include, for example, leukemias (e.g., acute myeloid leukemia, acute lymphoblastic leukemia, chronic lymphocytic leukemia, chronic myelogenous leukemia, specify juvenile myelomonocytic leukemia (JMML), and hairy cell leukemia) and lymphomas (e.g., non-Hodgkin's lymphoma, Hodgkin's disease cutaneous T-cell lymphoma, and Burkitt lymphoma).

In some embodiments of any of the methods or uses described herein, the cancer (e.g., Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof)) is a solid tumor. Examples of solid tumors (e.g., solid tumors that are Ras pathway-associated cancers) include, for example, thyroid cancer (e.g., papillary thyroid carcinoma, medullary thyroid carcinoma), lung cancer (e.g., non-small cell lung cancer, small-cell lung carcinoma, bronchial adenoma, and pleuropulmonary blastoma), pancreatic cancer, pancreatic ductal carcinoma, biliary tract cancer, breast cancer (e.g., invasive ductal carcinoma, invasive lobular carcinoma, ductal carcinoma in situ, and lobular carcinoma in situ), stomach cancer, small intestinal cancer, colon cancer, colorectal cancer, peritoneal cancer, ovarian cancer, uterine cancer, liver cancer, endometrial cancer, prostate cancer (including benign prostatic hyperplasia), testicular cancer, bladder cancer, urinary tract cancer, cervical cancer, head and neck cancer, brain cancer (e.g., glioblastoma, brain stem and hypophtalmic glioma, cerebellar and cerebral astrocytoma, medulloblastoma, and ependymoma), squamous cell carcinoma, and melanoma.

In some embodiments, the subject is a human.

Compounds of Formula (I) and pharmaceutically acceptable salts and solvates thereof are also useful for treating a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof).

Accordingly, also provided herein is a method for treating a subject diagnosed with or identified as having a Ras pathway-associated cancer, e.g., any of the exemplary Ras pathway-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-85, or a pharmaceutically acceptable salt thereof.

Accordingly, also provided herein is a method for treating a subject diagnosed with or identified as having a Ras pathway-associated cancer, e.g., any of the exemplary Ras pathway-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-182, or a pharmaceutically acceptable salt thereof.

Compounds of Formula (I) and pharmaceutically acceptable salts and solvates thereof are also useful for treating a Ras-associated cancer.

Accordingly, also provided herein is a method for treating a subject diagnosed with or identified as having a Ras-associated cancer, e.g., any of the exemplary Ras-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-182, or a pharmaceutically acceptable salt thereof.

Accordingly, also provided herein is a method for treating a subject diagnosed with or identified as having a Ras-associated cancer, e.g., any of the exemplary Ras-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-182, or a pharmaceutically acceptable salt thereof.

Compounds of Formula (I) and pharmaceutically acceptable salts and solvates thereof are also useful for treating a KRas-associated cancer.

Accordingly, also provided herein is a method for treating a subject diagnosed with or identified as having a KRas-associated cancer, e.g., any of the exemplary KRas-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-182, or a pharmaceutically acceptable salt thereof.

Accordingly, also provided herein is a method for treating a subject diagnosed with or identified as having a KRas-associated cancer, e.g., any of the exemplary KRas-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-182, or a pharmaceutically acceptable salt thereof.

Compounds of Formula (I) and pharmaceutically acceptable salts and solvates thereof are also useful for treating a HRas-associated cancer.

Accordingly, also provided herein is a method for treating a subject diagnosed with or identified as having a HRas-associated cancer, e.g., any of the exemplary HRas-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-182, or a pharmaceutically acceptable salt thereof.

Accordingly, also provided herein is a method for treating a subject diagnosed with or identified as having a HRas-associated cancer, e.g., any of the exemplary HRas-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-182, or a pharmaceutically acceptable salt thereof.

Compounds of Formula (I) and pharmaceutically acceptable salts and solvates thereof are also useful for treating a NRas-associated cancer.

Accordingly, also provided herein is a method for treating a subject diagnosed with or identified as having a NRas-associated cancer, e.g., any of the exemplary NRas-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-182, or a pharmaceutically acceptable salt thereof.

Accordingly, also provided herein is a method for treating a subject diagnosed with or identified as having a NRas-associated cancer, e.g., any of the exemplary NRas-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-182, or a pharmaceutically acceptable salt thereof.

Compounds of Formula (I) and pharmaceutically acceptable salts and solvates thereof are also useful for treating a SOS1-associated cancer.

Accordingly, also provided herein is a method for treating a subject diagnosed with or identified as having a SOS1-associated cancer, e.g., any of the exemplary SOS1-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-182, or a pharmaceutically acceptable salt thereof.

Dysregulation of a Ras pathway protein (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), a Ras pathway gene (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or the expression or activity or level of any (e.g., one or more) of the same can contribute to tumorigenesis. For example, a fusion protein can have increased activity as compared to a wild type Ras pathway protein (e.g., for SOS1, increased Ras activation through more advantageous binding and/or increased GEF activity), increased expression (e.g., increased levels) of a wild type Ras pathway protein in a mammalian cell can occur due to aberrant cell signaling and/or dysregulated autocrine/paracrine signaling (e.g., as compared to a control non-cancerous cell), Ras pathway mRNA splice variants may also result in dysregulation of Ras pathway.

In some embodiments, the compounds provided herein exhibit brain and/or central nervous system (CNS) penetrance. Such compounds are capable of crossing the blood brain barrier and inhibiting Ras pathway (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof)) activity in the brain and/or other CNS structures. In some embodiments, the compounds provided herein are capable of crossing the blood brain barrier in an effective amount. For example, treatment of a subject with cancer (e.g., a Ras pathway-associated cancer such as a Ras pathway-associated brain or CNS cancer) can include administration (e.g., oral administration) of the compound to the subject. In some such embodiments, the compounds provided herein are useful for treating a primary brain tumor or metastatic brain tumor. For example, the compounds can be used in the treatment of one or more of gliomas such as glioblastoma (also known as glioblastoma multiforme), astrocytomas, oligodendrogliomas, ependymomas, and mixed gliomas, meningiomas, medulloblastomas, gangliogliomas, schwannomas (neurilemmomas), and craniopharyngiomas (see, for example, the tumors listed in Louis, D. N. et al. Acta Neuropathol 131(6), 803-820 (June 2016)). In some embodiments, the brain tumor is a primary brain tumor. In some embodiments, the subject has previously been treated with another anticancer agent, e.g., another Ras pathway inhibitor (e.g., a compound that is not a compound of General Formula (I), or an inhibitor of another Ras pathway gene or protein (e.g., Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or a combination thereof). In some embodiments, the brain tumor is a metastatic brain tumor. In some embodiments, the subject has previously been treated with another anticancer agent, e.g., another Ras pathway inhibitor (e.g., a compound that is not a compound of Formula (I), or an inhibitor of another Ras pathway gene or protein (e.g., Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or a combination thereof).

The ability of the compounds described herein, to cross the BBB can be demonstrated by assays known in the art. Such assays include BBB models such as the transwell system, the hollow fiber (dynamic in vitro BBB) model, other microfluidic BBB systems, the BBB spheroid platform, and other cell aggregate-based BBB models. See, e.g., Cho et al. Nat Commun. 2017; 8: 15623; Bagchi, et al. Drug Des Devel Ther. 2019; 13: 3591-3605; Gastfriend, et al. Curr Opin Biomed Eng. 2018 March; 5: 6-12; and Wang et al. Biotechnol Bioeng. 2017 January; 114(1): 184-194. In some embodiments, the compounds described herein, are fluorescently labeled, and the fluorescent label can be detected using microscopy (e.g., confocal microscopy). In some such embodiments, the ability of the compound to penetrate the surface barrier of the model can be represented by the fluorescence intensity at a given depth below the surface. In some assays, such as a calcein-AM-based assay, the fluorescent label is non-fluorescent until it permeates live cells and is hydrolyzed by intracellular esterases to produce a fluorescent compound that is retained in the cell and can be quantified with a spectrophotometer. Non-limiting examples of fluorescent labels that can be used in the assays described herein include Cy5, rhodamine, infrared IRDye® CW-800 (LICOR #929-71012), far-red IRDye® 650 (LICOR #929-70020), sodium fluorescein (Na—F), lucifer yellow (LY), 5′carboxyfluorescein, and calcein-acetoxymethylester (calcein-AM). In some embodiments, the BBB model (e.g., the tissue or cell aggregate) can be sectioned, and a compound described herein can be detected in one or more sections using mass spectrometry (e.g., MALDI-MSI analyses). In some embodiments, the ability of a compound described herein to cross the BBB through a transcellular transport system, such as receptor-mediated transport (RMT), carrier-mediated transport (CMT), or active efflux transport (AET), can be demonstrated by assays known in the art. See, e.g., Wang, et al. Drug Deliv. 2019; 26(1): 551-565. In some embodiments, assays to determine if compounds can be effluxed by the P-glycoprotein (Pgp) include monolayer efflux assays in which movement of compounds through Pgp is quantified by measuring movement of digoxin, a model Pgp substrate (see, e.g., Doan et al. 2002. J Pharmacol Exp Ther. 303(3):1029-1037). Alternative in vivo assays to identify compounds that pass through the blood-brain barriers include phage-based systems (see, e.g., Peng et al. 2019. ChemRxiv. Preprint doi.org/10.26434/chemrxiv.8242871.v1). In some embodiments, binding of the compounds described herein to brain tissue is quantified. For example, a brain tissue binding assay can be performed using equilibrium dialysis, and the fraction of a compound described herein unbound to brain tissue can be detected using LC-MS/MS (Cyprotex: Brain Tissue Binding Assay www.cyprotex.com/admepk/protein binding/brain-tissue-binding/).

In some embodiments, the subject has been identified or diagnosed as having a cancer with a dysregulation of a Ras pathway gene (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), a Ras pathway protein (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or expression or activity, or level of any of the same (a Ras pathway-associated cancer) (e.g., as determined using a regulatory agency-approved, e.g., FDA-approved, assay or kit). In some embodiments, the subject has a tumor that is positive for a dysregulation of a Ras pathway gene, a Ras pathway protein, or expression or activity, or level of any of the same (e.g., as determined using a regulatory agency-approved assay or kit). The subject can be a subject with a tumor(s) that is positive for a dysregulation of a Ras pathway gene, a Ras pathway protein, or expression or activity, or level of any of the same (e.g., identified as positive using a regulatory agency-approved, e.g., FDA-approved, assay or kit). The subject can be a subject whose tumors have a dysregulation of a Ras pathway gene, a Ras pathway protein, or expression or activity, or a level of the same (e.g., where the tumor is identified as such using a regulatory agency-approved, e.g., FDA-approved, kit or assay). In some embodiments, the subject is suspected of having a Ras pathway-associated cancer. In some embodiments, the subject has a clinical record indicating that the subject has a tumor that has a dysregulation of a Ras pathway gene, a Ras pathway protein, or expression or activity, or level of any of the same (and optionally the clinical record indicates that the subject should be treated with any of the compositions provided herein).

In some embodiments, the subject has been identified or diagnosed as having a cancer with a dysregulation of a Ras gene, a Ras protein, or expression or activity, or level of any of the same (a Ras-associated cancer) (e.g., as determined using a regulatory agency-approved, e.g., FDA-approved, assay or kit). In some embodiments, the subject has a tumor that is positive for a dysregulation of a Ras gene, a Ras protein, or expression or activity, or level of any of the same (e.g., as determined using a regulatory agency-approved assay or kit). The subject can be a subject with a tumor(s) that is positive for a dysregulation of a Ras gene, a Ras protein, or expression or activity, or level of any of the same (e.g., identified as positive using a regulatory agency-approved, e.g., FDA-approved, assay or kit). The subject can be a subject whose tumors have a dysregulation of a Ras gene, a Ras protein, or expression or activity, or a level of the same (e.g., where the tumor is identified as such using a regulatory agency-approved, e.g., FDA-approved, kit or assay). In some embodiments, the subject is suspected of having a Ras-associated cancer. In some embodiments, the subject has a clinical record indicating that the subject has a tumor that has a dysregulation of a Ras gene, a Ras protein, or expression or activity, or level of any of the same (and optionally the clinical record indicates that the subject should be treated with any of the compositions provided herein).

In some embodiments, the subject has been identified or diagnosed as having a cancer with a dysregulation of a KRas gene, a KRas protein, or expression or activity, or level of any of the same (a KRas-associated cancer) (e.g., as determined using a regulatory agency-approved, e.g., FDA-approved, assay or kit). In some embodiments, the subject has a tumor that is positive for a dysregulation of a KRas gene, a KRas protein, or expression or activity, or level of any of the same (e.g., as determined using a regulatory agency-approved assay or kit). For example, the subject has a tumor that is positive for a mutation as described in Table 1. The subject can be a subject with a tumor(s) that is positive for a dysregulation of a KRas gene, a KRas protein, or expression or activity, or level of any of the same (e.g., identified as positive using a regulatory agency-approved, e.g., FDA-approved, assay or kit). The subject can be a subject whose tumors have a dysregulation of a KRas gene, a KRas protein, or expression or activity, or a level of the same (e.g., where the tumor is identified as such using a regulatory agency-approved, e.g., FDA-approved, kit or assay). In some embodiments, the subject is suspected of having a KRas-associated cancer. In some embodiments, the subject has a clinical record indicating that the subject has a tumor that has a dysregulation of a KRas gene, a KRas protein, or expression or activity, or level of any of the same (and optionally the clinical record indicates that the subject should be treated with any of the compositions provided herein).

In some embodiments, the subject has been identified or diagnosed as having a cancer with a dysregulation of a HRas gene, a HRas protein, or expression or activity, or level of any of the same (a HRas-associated cancer) (e.g., as determined using a regulatory agency-approved, e.g., FDA-approved, assay or kit). In some embodiments, the subject has a tumor that is positive for a dysregulation of a HRas gene, a HRas protein, or expression or activity, or level of any of the same (e.g., as determined using a regulatory agency-approved assay or kit). For example, the subject has a tumor that is positive for a mutation as described in Table 2. The subject can be a subject with a tumor(s) that is positive for a dysregulation of a HRas gene, a HRas protein, or expression or activity, or level of any of the same (e.g., identified as positive using a regulatory agency-approved, e.g., FDA-approved, assay or kit). The subject can be a subject whose tumors have a dysregulation of a HRas gene, a HRas protein, or expression or activity, or a level of the same (e.g., where the tumor is identified as such using a regulatory agency-approved, e.g., FDA-approved, kit or assay). In some embodiments, the subject is suspected of having a HRas-associated cancer. In some embodiments, the subject has a clinical record indicating that the subject has a tumor that has a dysregulation of a HRas gene, a HRas protein, or expression or activity, or level of any of the same (and optionally the clinical record indicates that the subject should be treated with any of the compositions provided herein).

In some embodiments, the subject has been identified or diagnosed as having a cancer with a dysregulation of a NRas gene, a NRas protein, or expression or activity, or level of any of the same (a NRas-associated cancer) (e.g., as determined using a regulatory agency-approved, e.g., FDA-approved, assay or kit). In some embodiments, the subject has a tumor that is positive for a dysregulation of a NRas gene, a NRas protein, or expression or activity, or level of any of the same (e.g., as determined using a regulatory agency-approved assay or kit). For example, the subject has a tumor that is positive for a mutation as described in Table 3. The subject can be a subject with a tumor(s) that is positive for a dysregulation of a NRas gene, a NRas protein, or expression or activity, or level of any of the same (e.g., identified as positive using a regulatory agency-approved, e.g., FDA-approved, assay or kit). The subject can be a subject whose tumors have a dysregulation of a NRas gene, a NRas protein, or expression or activity, or a level of the same (e.g., where the tumor is identified as such using a regulatory agency-approved, e.g., FDA-approved, kit or assay). In some embodiments, the subject is suspected of having a NRas-associated cancer. In some embodiments, the subject has a clinical record indicating that the subject has a tumor that has a dysregulation of a NRas gene, a NRas protein, or expression or activity, or level of any of the same (and optionally the clinical record indicates that the subject should be treated with any of the compositions provided herein).

In some embodiments, the subject has been identified or diagnosed as having a cancer with a dysregulation of a SOS1 gene, a SOS1 protein, or expression or activity, or level of any of the same (a SOS1-associated cancer) (e.g., as determined using a regulatory agency-approved, e.g., FDA-approved, assay or kit). In some embodiments, the subject has a tumor that is positive for a dysregulation of a SOS1 gene, a SOS1 protein, or expression or activity, or level of any of the same (e.g., as determined using a regulatory agency-approved assay or kit). For example, the subject has a tumor that is positive for a mutation as described in Table 4. The subject can be a subject with a tumor(s) that is positive for a dysregulation of a SOS1 gene, a SOS1 protein, or expression or activity, or level of any of the same (e.g., identified as positive using a regulatory agency-approved, e.g., FDA-approved, assay or kit). The subject can be a subject whose tumors have a dysregulation of a SOS1 gene, a SOS1 protein, or expression or activity, or a level of the same (e.g., where the tumor is identified as such using a regulatory agency-approved, e.g., FDA-approved, kit or assay). In some embodiments, the subject is suspected of having a SOS1-associated cancer. In some embodiments, the subject has a clinical record indicating that the subject has a tumor that has a dysregulation of a SOS1 gene, a SOS1 protein, or expression or activity, or level of any of the same (and optionally the clinical record indicates that the subject should be treated with any of the compositions provided herein).

In some embodiments of any of the methods or uses described herein, an assay used to determine whether the subject has a dysregulation of a Ras pathway gene (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or a Ras pathway protein (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or expression or activity or level of any of the same, using a sample from a subject can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof. Assays can utilize other detection methods known in the art for detecting dysregulation of a Ras pathway gene, a Ras pathway protein, or expression or activity or levels of any of the same. In some embodiments, the sample is a biological sample or a biopsy sample (e.g., a paraffin-embedded biopsy sample) from the subject. In some embodiments, the subject is a subject suspected of having a Ras pathway-associated cancer, a subject having one or more symptoms of a Ras pathway-associated cancer, and/or a subject that has an increased risk of developing a Ras pathway-associated cancer).

In some embodiments, dysregulation of a Ras pathway gene (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), a Ras pathway protein (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or the expression or activity or level of any of the same can be identified using a liquid biopsy (variously referred to as a fluid biopsy or fluid phase biopsy). See, e.g., Karachialiou et al., “Real-time liquid biopsies become a reality in cancer treatment”, Ann. Transl. Med., 3(3):36, 2016. Liquid biopsy methods can be used to detect total tumor burden and/or the dysregulation of a Ras pathway gene, a Ras pathway protein, or the expression or activity or level of any of the same. Liquid biopsies can be performed on biological samples obtained relatively easily from a subject (e.g., via a simple blood draw) and are generally less invasive than traditional methods used to detect tumor burden and/or dysregulation of a Ras pathway gene, a Ras pathway protein, or the expression or activity or level of any of the same. In some embodiments, liquid biopsies can be used to detect the presence of dysregulation of a Ras pathway gene, a Ras pathway protein, or the expression or activity or level of any of the same at an earlier stage than traditional methods. In some embodiments, the biological sample to be used in a liquid biopsy can include, blood, plasma, urine, cerebrospinal fluid, saliva, sputum, broncho-alveolar lavage, bile, lymphatic fluid, cyst fluid, stool, ascites, and combinations thereof. In some embodiments, a liquid biopsy can be used to detect circulating tumor cells (CTCs). In some embodiments, a liquid biopsy can be used to detect cell-free DNA. In some embodiments, cell-free DNA detected using a liquid biopsy is circulating tumor DNA (ctDNA) that is derived from tumor cells. Analysis of ctDNA (e.g., using sensitive detection techniques such as, without limitation, next-generation sequencing (NGS), traditional PCR, digital PCR, or microarray analysis) can be used to identify dysregulation of a Ras pathway gene, a Ras pathway protein, or the expression or activity or level of any of the same.

In some embodiments, a liquid biopsy can be used to detect circulating tumor cells (CTCs). In some embodiments, a liquid biopsy can be used to detect cell-free DNA. In some embodiments, cell-free DNA detected using a liquid biopsy is circulating tumor DNA (ctDNA) that is derived from tumor cells. Analysis of ctDNA (e.g., using sensitive detection techniques such as, without limitation, next-generation sequencing (NGS), traditional PCR, digital PCR, or microarray analysis) can be used to identify dysregulation of a Ras pathway gene (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), a Ras pathway protein (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or the expression or activity or level of any of the same.

In some embodiments, ctDNA derived from a single gene can be detected using a liquid biopsy. In some embodiments, ctDNA derived from a plurality of genes (e.g., 2, 3, 4, 5, 6, 7, 8, 9, 10, 15, 20, 25, 30, 35, 40, 45, 50, 55, 60, 65, 70, 75, 80, 85, 90, 95, 100 or more, or any number of genes in between these numbers) can be detected using a liquid biopsy. In some embodiments, ctDNA derived from a plurality of genes can be detected using any of a variety of commercially-available testing panels (e.g., commercially-available testing panels designed to detect dysregulation of a Ras pathway gene (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), a Ras pathway protein (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or the expression or activity or level of any of the same). Liquid biopsies can be used to detect dysregulation of a Ras pathway gene, a Ras pathway protein, or the expression or activity or level of any of the same including, without limitation, point mutations or single nucleotide variants (SNVs), copy number variants (CNVs), genetic fusions (e.g., translocations or rearrangements), insertions, deletions, or any combination thereof. In some embodiments, a liquid biopsy can be used to detect a germline mutation. In some embodiments, a liquid biopsy can be used to detect a somatic mutation. In some embodiments, a liquid biopsy can be used to detect a primary genetic mutation (e.g., a primary mutation or a primary fusion that is associated with initial development of a disease, e.g., cancer). In some embodiments, a dysregulation of a Ras pathway gene, a Ras pathway protein, or the expression or activity or level of any of the same identified using a liquid biopsy is also present in a cancer cell that is present in the subject (e.g., in a tumor). In some embodiments, any of the types of dysregulation of a Ras pathway gene, a Ras pathway protein, or the expression or activity or level of any of the same described herein can be detected using a liquid biopsy. In some embodiments, a genetic mutation identified via a liquid biopsy can be used to identify the subject as a candidate for a particular treatment. For example, detection of dysregulation of a Ras pathway gene, a Ras pathway protein, or the expression or activity or level of any of the same in the subject can indicate that the subject will be responsive to a treatment that includes administration of a compound of Formula (I), or a pharmaceutically acceptable salt thereof.

Some embodiments of these methods can further include administering to the subject at least one dose of the compound of Formula (I), or a pharmaceutically acceptable salt thereof, between the first and second time points. For example, a reduction (e.g., a 1% to about a 99% reduction, a 1% to about a 95% reduction, a 1% to about a 90% reduction, a 1% to about a 85% reduction, a 1% to about a 80% reduction, a 1% to about a 75% reduction, a 1% reduction to about a 70% reduction, a 1% reduction to about a 65% reduction, a 1% reduction to about a 60% reduction, a 1% reduction to about a 55% reduction, a 1% reduction to about a 50% reduction, a 1% reduction to about a 45% reduction, a 1% reduction to about a 40% reduction, a 1% reduction to about a 35% reduction, a 1% reduction to about a 30% reduction, a 1% reduction to about a 25% reduction, a 1% reduction to about a 20% reduction, a 1% reduction to about a 15% reduction, a 1% reduction to about a 10% reduction, a 1% to about a 5% reduction, about a 5% to about a 99% reduction, about a 10% to about a 99% reduction, about a 15% to about a 99% reduction, about a 20% to about a 99% reduction, about a 25% to about a 99% reduction, about a 30% to about a 99% reduction, about a 35% to about a 99% reduction, about a 40% to about a 99% reduction, about a 45% to about a 99% reduction, about a 50% to about a 99% reduction, about a 55% to about a 99% reduction, about a 60% to about a 99% reduction, about a 65% to about a 99% reduction, about a 70% to about a 99% reduction, about a 75% to about a 95% reduction, about a 80% to about a 99% reduction, about a 90% reduction to about a 99% reduction, about a 95% to about a 99% reduction, about a 5% to about a 10% reduction, about a 5% to about a 25% reduction, about a 10% to about a 30% reduction, about a 20% to about a 40% reduction, about a 25% to about a 50% reduction, about a 35% to about a 55% reduction, about a 40% to about a 60% reduction, about a 50% reduction to about a 75% reduction, about a 60% reduction to about 80% reduction, or about a 65% to about a 85% reduction) in the allele frequency (AF) of the dysregulation of a Ras pathway gene (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof) in the cfDNA obtained from the subject at the second time point as compared to the allele frequency (AF) of the dysregulation of a Ras pathway gene in the cfDNA obtained from the subject at the first time point indicates that the compound of Formula (I), or a pharmaceutically acceptable salt thereof, was effective in the subject. In some embodiments, the AF is reduced such that the level is below the detection limit of the instrument. Alternatively, an increase in the allele frequency (AF) of the dysregulation of a Ras pathway gene in the cfDNA obtained from the subject at the second time point as compared to the allele frequency (AF) of the dysregulation of a Ras pathway gene in the cfDNA obtained from the subject at the first time point indicates that the compound of Formula (I), or a pharmaceutically acceptable salt thereof, was not effective in the subject. Some embodiments of these methods can further include, administering additional doses of a compound of Formula (I), or a pharmaceutically acceptable salt thereof, to a subject in which a compound of Formula (I), or a pharmaceutically acceptable salt thereof, was determined to be effective. Some embodiments of these methods can further include, administering a different treatment (e.g., a treatment that does not include the administration of a compound of Formula (I), or a pharmaceutically acceptable salt thereof, as a monotherapy) to a subject in which a compound of Formula (I), or a pharmaceutically acceptable salt thereof, was determined not to be effective.

In some examples of these methods, the time difference between the first and second time points can be about 1 day to about 1 year, about 1 day to about 11 months, about 1 day to about 10 months, about 1 day to about 9 months, about 1 day to about 8 months, about 1 day to about 7 months, about 1 day to about 6 months, about 1 day to about 5 months, about 1 day to about 4 months, about 1 day to about 3 months, about 1 day to about 10 weeks, about 1 day to about 2 months, about 1 day to about 6 weeks, about 1 day to about 1 month, about 1 day to about 25 days, about 1 day to about 20 days, about 1 day to about 15 days, about 1 day to about 10 days, about 1 day to about 5 days, about 2 days to about 1 year, about 5 days to about 1 year, about 10 days to about 1 year, about 15 days to about 1 year, about 20 days to about 1 year, about 25 days to about 1 year, about 1 month to about 1 year, about 6 weeks to about 1 year, about 2 months to about 1 year, about 3 months to about 1 year, about 4 months to about 1 year, about 5 months to about 1 year, about 6 months to about 1 year, about 7 months to about 1 year, about 8 months to about 1 year, about 9 months to about 1 year, about 10 months to about 1 year, about 11 months to about 1 year, about 1 day to about 7 days, about 1 day to about 14 days, about 5 days to about 10 days, about 5 day to about 20 days, about 10 days to about 20 days, about 15 days to about 1 month, about 15 days to about 2 months, about 1 week to about 1 month, about 2 weeks to about 1 month, about 1 month to about 3 months, about 3 months to about 6 months, about 4 months to about 6 months, about 5 months to about 8 months, or about 7 months to about 9 months. In some embodiments of these methods, the subject can be previously identified as having a cancer having a dysregulated Ras pathway gene (e.g., any of the examples of a dysregulated Ras pathway gene described herein). In some embodiments of these methods, a subject can have been previously diagnosed as having any of the types of cancer described herein. In some embodiments of these methods, the subject can have one or more metastases (e.g., one or more brain metastases).

In some of the above embodiments, the cfDNA comprises ctDNA such as Ras pathway-associated ((e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof)-associated) ctDNA. For example, the cfDNA is ctDNA such as Ras pathway-associated ctDNA. In some embodiments, at least some portion of cfDNA is determined to be Ras pathway-associated ctDNA, for example, a sequenced and/or quantified amount of the total cfDNA is determined to have a Ras pathway fusion and/or overexpression of Ras pathway.

### Combinations

In the field of medical oncology it is normal practice to use a combination of different forms of treatment to treat each subject with cancer. In medical oncology the other component(s) of such conjoint treatment or therapy in addition to compositions provided herein may be, for example, surgery, radiotherapy, and chemotherapeutic agents, such as other Ras pathway inhibitors (e.g., inhibitors of SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof, kinase inhibitors, signal transduction inhibitors, and/or monoclonal antibodies. For example, a surgery may be open surgery or minimally invasive surgery. Compounds of Formula (I), or a pharmaceutically acceptable salt thereof therefore may also be useful as adjuvants to cancer treatment, that is, they can be used in combination with one or more additional therapies or therapeutic agents, for example, a chemotherapeutic agent that works by the same or by a different mechanism of action. In some embodiments, a compound of Formula (I), or a pharmaceutically acceptable salt thereof, can be used prior to administration of an additional therapeutic agent or additional therapy. For example, a subject in need thereof can be administered one or more doses of a compound of Formula (I), or a pharmaceutically acceptable salt thereof for a period of time and then undergo at least partial resection of the tumor. In some embodiments, the treatment with one or more doses of a compound of Formula (I), or a pharmaceutically acceptable salt thereof reduces the size of the tumor (e.g., the tumor burden) prior to the at least partial resection of the tumor. In some embodiments, a subject in need thereof can be administered one or more doses of a compound of Formula (I), or a pharmaceutically acceptable salt thereof for a period of time and under one or more rounds of radiation therapy. In some embodiments, the treatment with one or more doses of a compound of Formula (I), or a pharmaceutically acceptable salt thereof reduces the size of the tumor (e.g., the tumor burden) prior to the one or more rounds of radiation therapy.

A “Ras pathway targeted therapeutic agent” as used herein includes any compound exhibiting inactivation activity (e.g., active site (e.g., competitive) inhibition, allosteric inhibition, inhibition of dimerization, inhibition of expression, inhibition of protein-protein interaction, and induction of degradation) of any protein in a Ras pathway. Non-limiting examples of a protein in a Ras pathway include any one of the proteins in the Ras-RAF-MAPK pathway or PI3K/AKT pathway such as Ras (e.g., KRas, HRas, and/or NRas), EGFR, ErbB2, ErbB3, ErbB4, NF1, PDGFR-A, PDGFR-B, FGFR1, FGFR2, FGFR3, IGF1R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof. In some embodiments, a Ras pathway targeted therapeutic agent can be selective for a protein in a Ras pathway. For example, the Ras pathway targeted therapeutic agent can be selective for a Ras protein (e.g., KRas, HRas, and/or NRas, or mutated forms of any thereof); such an agent can also be called a “Ras modulator”). In some embodiments, a Ras modulator is a covalent inhibitor. In some embodiments, a Ras pathway targeted therapeutic agent can be selective for a particular Ras protein (e.g., KRas, HRas, or NRas), or a mutated form thereof (e.g., a G12 mutant, a G13 mutant, or a Q61 mutant). Non-limiting examples of KRas-targeted therapeutic agents (e.g., KRas inhibitors (such as KRas G12C inhibitors)) include AMG 510, ARS-3248, ARS1620, SML-8-73-1, SML-10-70-1, VSA9, AA12, MRTX-849, MRTX849, LY3499446, JNJ-74699157, ARS853, AZD4785, and JNJ-74699157.

Compounds of Formula (I), or pharmaceutically acceptable salts or thereof, can be used in combination with one or more additional therapies or therapeutic agents, for example, a chemotherapeutic agent that works by the same or by a different mechanism of action. In some embodiments, a compound of Formula (I), or a pharmaceutically acceptable salt thereof, can be used prior to administration of an additional therapeutic agent or additional therapy. For example, a subject in need thereof can be administered one or more doses of a compound of Formula (I), or a pharmaceutically acceptable salt thereof, for a period of time and then undergo at least partial resection of the tumor. In some embodiments, the treatment with one or more doses of a compound of Formula (I), or a pharmaceutically acceptable salt thereof, reduces the size of the tumor (e.g., the tumor burden) prior to the at least partial resection of the tumor. In some embodiments, a subject in need thereof can be administered one or more doses of a compound of Formula (I), or a pharmaceutically acceptable salt thereof, for a period of time and under one or more rounds of radiation therapy. In some embodiments, the treatment with one or more doses of a compound of Formula (I), or a pharmaceutically acceptable salt thereof, reduces the size of the tumor (e.g., the tumor burden) prior to the one or more rounds of radiation therapy.

In some embodiments, the one or more additional therapies or therapeutic agents are independently selected from: EGFR inhibitors (e.g., afatinib, erlotinib, gefitinib, lapatinib, cetuximab, panitumumab, osimertinib, and olmutinib), ErbB2/Her2 inhibitors (e.g., afatinib, lapatinib, trastuzumab, and pertuzumab), ALK inhibitors (e.g., crizotinib, alectinib, entrectinib, brigatinib), ROS1 inhibitors (e.g., crizotinib, entrectinib, lorlatinib, ceritinib, and merestinib), MEK inhibitors (e.g., trametinib, cobimetinib, binimetinib, selumetinib, refametinib), RAS (KRas, HRas, and/or NRas) inhibitors (e.g., MRTX849, LY3499446, JNJ-74699157, AMG 510, and AZD4785), Bcr-Abl inhibitors (e.g., imatinib, dasatinib, nilotinib), FGFR1, 2, or 3 inhibitors (e.g., nintedanib), MET inhibitors (e.g., capmatinib), AXL inhibitors (e.g., sitravatinib), RET inhibitors (e.g., sunitinib and selpercatinib), ERK inhibitors (e.g., ulixertinib), Shp2 inhibitors (e.g., RLY-1971, RMC-4630, TNO155, and JAB-3068), Bcl-2 inhibitors (e.g., ABT-263, obatoclax, ABT-737, and navitoclax), mTOR inhibitors (e.g., everolimus and tacrolimus), Trk inhibitors (e.g., larotrectinib and entrectinib), checkpoint inhibitors (e.g., ipilimumab, nivolumab, pembrolizumab, atezolizumab, avelumab, durvalumab, and pidilizumab) or other immunotherapies (e.g., monoclonal antibodies), PARP inhibitors (e.g., olaparib), PI3K inhibitors (e.g., buparlisib), BET inhibitors (e.g., GSK1210151A), Raf inhibitors (e.g., encorafenib), MCL-1 inhibitors (e.g., AZD5991), AKT inhibitors (e.g., miltefosine), PDK1 inhibitors (e.g., GSK 2334470), and other chemotherapeutic agents such as taxanes (e.g., paclitaxel and docetaxel), platinum-based agents (e.g., cisplatin and carboplatin), cytoxic agents (e.g., 5-fluorouracil, capecitabine, floxuridine, cytarabine, and gemcitabine), farnesyltransferase inhibitors, topoisomerase inhibitors (e.g., topotecan and irinotecan), DNA synthesis inhibitors (e.g., capecitabine (Xeloda®) and gemcitabine hydrochloride (Gemzar®)), alkylating agents (e.g., temozolomide (Temodar® and Temodal®), dactinomycin (also known as actinomycin-D, Cosmegen®), carmustine (BiCNU®), bendamustine (Treanda®), and lomustine (CeeNU®)), and cytotoxic agents (e.g., vincristine, cytarabine, and pemetrexed).

Epidermal growth factor receptor (EGFR) inhibitors such as osimertinib (AZD9291, merelectinib, TAGRISSO®), erlotinib (TARCEVA®), gefitinib (IRESSA®), cetuximab (ERBITUX®), necitumumab (PORTRAZZA®, IMC-11F8), neratinib (HKI-272, NERLYNX®), lapatinib (TYKERB®), panitumumab (ABX-EGF, VECTIBIX®), vandetanib (CAPRELSA®), rociletinib (CO-1686), olmutinib (OLITA®, HM61713, BI-1482694), naquotinib (ASP8273), nazartinib (EGF816, NVS-816), PF-06747775, icotinib (BPI-2009H), afatinib (BIBW 2992, GILOTRIF®), dacomitinib (PF-00299804, PF-804, PF-299, PF-299804), avitinib (AC0010), AC0010MA EAI045, matuzumab (EMD-7200), nimotuzumab (h-R3, BIOMAb EGFR®), zalutumab, MDX447, depatuxizumab (humanized mAb 806, ABT-806), depatuxizumab mafodotin (ABT-414), ABT-806, mAb 806, canertinib (CI-1033), shikonin, shikonin derivatives (e.g., deoxyshikonin, isobutyrylshikonin, acetylshikonin, 0,0-dimethylacrylshikonin and acetylalkannin), poziotinib (NOV120101, HM781-36B), AV-412, ibrutinib, WZ4002, brigatinib (AP26113, ALUNBRIG®), pelitinib (EKB-569), tarloxotinib (TH-4000, PR610), BPI-15086, Hemay022, ZN-e4, tesevatinib (KD019, XL647), YH25448, epitinib (HMPL-813), CK-101, MM-151, AZD3759, ZD6474, PF-06459988, varlintinib (ASLAN001, ARRY-334543), AP32788, HLX07, D-0316, AEE788, HS-10296, avitinib, GW572016, pyrotinib (SHR1258), SCT200, CPGJ602, Sym004, MAb-425, Modotuximab (TAB-H49), futuximab (992 DS), zalutumumab, KL-140, R05083945, IMGN289, JNJ-61186372, LY3164530, Sym013, AMG 595, BDTX-189, avatinib, Disruptin, CL-387785, EGFRBi-Armed Autologous T Cells, and EGFR CAR-T Therapy. In some embodiments, the EGFR-targeted therapeutic agent is selected from osimertinib, gefitinib, erlotinib, afatinib, lapatinib, neratinib, AZD-9291, CL-387785, CO-1686, or WZ4002.

Human Epidermal Growth Factor Receptor 2 (HER2 receptor) (also known as Neu, ErbB-2, CD340, or p185) inhibitors such as trastuzumab (e.g., TRAZIMERA™ HERCEPTIN®), pertuzumab (e.g., PERJETA®), trastuzumab emtansine (T-DM1 or ado-trastuzumab emtansine, e.g., KADCYLA®), lapatinib, KU004, neratinib (e.g., NERLYNX®), dacomitinib (e.g., VIZIMPRO®), afatinib (GILOTRIF®), tucatinib (e.g., TUKYSA™), erlotinib (e.g., TARCEVA®), pyrotinib, poziotinib, CP-724714, CUDC-101, sapitinib (AZD8931), tanespimycin (17-AAG), IPI-504, PF299, pelitinib, S-22261 1, and AEE-788.

In some embodiments, the FGFR inhibitor is selected from infigratinib, AZD4547, erdafitinib (JNJ-42756493), nintedanib dovitinib, ponatinib, and TAS120.

In some embodiments, the ALK inhibitor is selected from alectinib, crizotinib (XALKORI®), ceritinib, AP26113, ASP3026, TSR-011, PF-06463922, X-396, and CEP-37440.

In some embodiments, the ROS1 inhibitor is selected from crizotinib (XALKORI®), ceritinib, lorlatinib, brigatinib, cabozantinib, and repotrectinib.

In some embodiments, the mTOR inhibitor is selecte from everolimus, tacrolimus rapamycin, perifosine, and temsirolimus.

In some embodiments, the Trk inhibitor is selected from larotrectinib, lestaurtinib, and entrectinib.

In some embodiments, the RET inhibitors is selected from sunitinib (Sutent®), selpercatinib (RETEVMO®), vandetanib (Caprelsa®), motesanib (AMG706), sorafenib, regorafenib, and danusertib.

In some embodiments, the MET inhibitor is selected from capmatinib, tepotinib, savolitinib, crizotinib, cabozantinib, tivantinib, bozitinib, merestinib, glesatinib, sitravatinib, onartuzumab, and emibetuzumab.

In some embodiments, the AXL inhibitor is selected from sitravatinib, bemcentinib, dubermatinib, DS-1205, SLC-391, INCB081776, ONO-7475, and BA3011.

In some embodiments, the Shp2 inhibitor is selected from TNO155, BBP-398, JAB-3068, RMC-4360, and RLY-1971.

In some embodiments, the RAF inhibitor is a BRAF inhibitor, such as vemurafenib (ZELBORAF®), dabrafenib (TAFINLAR®), encorafenib (BRAFTOVI®), BMS-908662, sorafenib, LGX818, PLX3603, RAF265, RO5185426, GSK2118436, ARQ 736, GDC-0879, PLX-4720, AZ304, PLX-8394, HM95573, RO5126766, and LXH254.

In some embodiments, the PI3K inhibitor is selected from buparlisib (BKM120), alpelisib (BYL719), WX-037, copanlisib (ALIQOPA®, BAY80-6946), dactolisib (NVP-BEZ235, BEZ-235), taselisib (GDC-0032, RG7604), sonolisib (PX-866), CUDC-907, PQR309, ZSTK474, SF1126, AZD8835, GDC-0077, ASN003, pictilisib (GDC-0941), pilaralisib (XL147, SAR245408), gedatolisib (PF-05212384, PKI-587), serabelisib (TAK-117, MLN1117, INK 1117), BGT-226 (NVP-BGT226), PF-04691502, apitolisib (GDC-0980), omipalisib (GSK2126458, GSK458), voxtalisib (XL756, SAR245409), AMG 511, CH5132799, GSK1059615, GDC-0084 (RG7666), VS-5584 (SB2343), PKI-402, wortmannin, LY294002, PI-103, rigosertib, XL-765, LY2023414, SAR260301, KIN-193 (AZD-6428), GS-9820, AMG319, and GSK2636771.

In some embodiments, the AKT inhibitor is selected from miltefosine (IMPADIVO®), wortmannin, NL-71-101, H-89, GSK690693, CCT128930, AZD5363, ipatasertib (GDC-0068, RG7440), A-674563, A-443654, AT7867, AT13148, uprosertib, afuresertib, DC120, MK-2206, edelfosine, miltefosine, perifosine, erucylphophocholine, erufosine, SR13668, OSU-A9, PH-316, PHT-427, PIT-1, DM-PIT-1, triciribine, API-1, ARQ092, BAY 1125976, 3-oxo-tirucallic acid, lactoquinomycin, GSK2141795, ONC201, tricirbine, A674563, and AT7867.

In some embodiments, the MEK inhibitor is selected from trametinib (MEKINIST®), cobimetinib (COTELLIC®), binimetinib (MEKTOVI®), selumetinib (AZD6244), PD0325901, MSC1936369B, SHR7390, TAK-733, R05126766, CS3006, WX-554, PD98059, CI1040 (PD184352), and hypothemycin.

In some embodiments, the ERK inhibitor is selected from FRI-20 (ON-01060), VTX-11e, 25-OH-D3-3-BE (B3CD, bromoacetoxycalcidiol), FR-180204, AEZ-131 (AEZS-131), AEZS-136, AZ-13767370, BL-EI-001, LY-3214996, LTT-462, KO-947, MK-8353 (SCH900353), SCH772984, ulixertinib (BVD-523), CC-90003, GDC-0994 (RG-7482), ASN007, FR148083, 5-7-Oxozeaenol, 5-iodotubercidin, GDC0994, and ONC201.

In some embodiments, the PARP inhibitors include olaparib (LYNPARZA®), talazoparib, rucaparib, niraparib, veliparib, BGB-290 (pamiparib), CEP 9722, E7016, iniparib, IMP4297, NOV1401, 2X-121, ABT-767, RBN-2397, BMN 673, KU-0059436 (AZD2281), BSI-201, PF-01367338, INO-1001, and JPI-289.

In some embodiments, the RAS inhibitor is MRTX849, LY3499446, JNJ-74699157, AMG 510, ARS3248, ARS853, ARS1620, AZD4785, JNJ-74699157, SML-8-73-1, SML-10-70-1, VSA9, AA12, and MRTX-849.

In some embodiments, the PDK-1 inhibitor is selected from GSK 2334470, JX06, SNS-510, and AR-12.

In some embodiments, the BET inhibitor is selected from GSK1210151A, GSK525762, OTX-015, TEN-010, CPI-203, CPI-0610, olinone, RVX-208, ABBV-744, LY294002, AZD5153, MT-1, and MS645.

In some embodiments, the MCL-1 inhibitor is AZD5991.

In some embodiments, the Bcl-2 protein family inhibitor is selected from ABT-263, Tetrocarcin A, Antimycin, Gossypol ((−)BL-193), obatoclax, HA14-1, oblimersen (Genasense®); (−)-Gossypol acetic acid (AT-101); ABT-737, and navitoclax.

In some embodiments, the Bcr/Abl kinase inhibitor is selected from imatinib (Gleevec®), inilotinib, nilotinib (Tasigna®), dasatinib (BMS-345825), bosutinib (SKI-606), ponatinib (AP24534), bafetinib (INNO406), danusertib (PHA-739358), AT9283, saracatinib (AZD0530), and PF-03814735.

In some embodiments, the checkpoint inhibitor is selected from ipilimumab (YERVOY®), pembrolizumab (KEYTRUDA®), nivolumab (OPDIVO®), cemiplimab (LIBTAYO®), atezolizumab (TECENTRIQ®), avelumab (BAVENCIO®), durvalumab (IMNFINZI®), PIP701 (LAG525), CPI-444, MBG453, enoblituzumab, JNJ-61610588, and indoximod. See, e.g., Marin-Acevedo, et. al., J Hematol Oncol. 11: 39 (2018).

In some embodiments, the other immunotherapy is an antibody therapy (e.g., a monoclonal antibody). In some embodiments, the antibody therapy is selected from bevacizumab (Mvasti™, Avastin®), trastuzumab (Herceptin®), rituximab (MabThera™, Rituxan®), edrecolomab (Panorex), daratumuab (Darzalex®), olaratumab (Lartruvo™), ofatumumab (Arzerra®), alemtuzumab (Campath®), cetuximab (Erbitux®), oregovomab, dinutiximab (Unituxin®), obinutuzumab (Gazyva®), tremelimumab (CP-675,206), ramucirumab (Cyramza®), ublituximab (TG-1101), panitumumab (Vectibix®), elotuzumab (Empliciti™), necitumumab (Portrazza™), cirmtuzumab (UC-961), ibritumomab (Zevalin®), isatuximab (SAR650984), nimotuzumab, fresolimumab (GC1008), lirilumab (INN), mogamulizumab (Poteligeo®), ficlatuzumab (AV-299), denosumab (Xgeva®), ganitumab, urelumab, pidilizumab, and amatuximab.

In some embodiments, the other chemotherapeutic agents are selected from an anthracycline, an alkylating agent, a taxane, a platinum-based agent, eribulin (HALAVEN™), a farnesyl transferase inhibitor, a topoisomerase inhibitor, a DNA synthesis inhibitor, and cytotoxic agents.

In some embodiments, the taxane is selected from paclitaxel, docetaxel, cabazitaxel, abraxane, and taxotere.

In some embodiments, the anthracycline is selected from daunorubicin, doxorubicin, epirubicin, idarubicin, and combinations thereof.

In some embodiments, the platinum-based agent is selected from carboplatin, cisplatin, oxaliplatin, nedplatin, triplatin tetranitrate, phenanthriplatin, picoplatin, and satraplatin.

In some embodiments, the farnesyl transferase inhibitor is selected from lonafarnib, tipifarnib, BMS-214662, L778123, L744832, and FTI-277.

In some embodiments, the topoisomerase inhibitor is a topoisomerase I inhibitor (e.g., irinotecan (Camptosar®), topotecan (Hycamtin®), and 7-Ethyl-10-hydroxycampothecin (SN38)) or a topoisomerase II inhibitor (e.g., etoposide (Toposar®, VePesid®, and Etopophos®), teniposide (VM-26, Vumon®), and tafluposide.

In some embodiments, the DNA synthesis inhibitor is selected from capecitabine (Xeloda®), gemcitabine hydrochloride (Gemzar®), nelarabine (Arranon® and Atriance®), and sapacitabine.

In some embodiments, the alkylating agent is selected from temozolomide (Temodar® and Temodal®), dactinomycin (also known as actinomycin-D, Cosmegen®), melphalan (Alkeran®), altretamine (Hexalen®), carmustine (BiCNU®), bendamustine (Treanda®), busulfan (Busulfex® and Myleran®), lomustine (CeeNU®), chlorambucil (Leukeran®), cyclophosphamide (Cytoxan® and Neosar®), dacarbazine (DTIC-Dome®), altretamine (Hexalen®), ifosfamide (Ifex®), prednumustine, procarbazine (Matulane®), mechlorethamine (Mustargen®), streptozocin (Zanosar®), and thiotepa (Thioplex®).

In some embodiments, the cytotoxic agent is selected from bleomycin, cytarabine, dacarbazine, methotrexate, mitomycin C, pemetrexed, and vincristine.

Also provided herein is (i) a pharmaceutical combination for treating a cancer in a subject in need thereof, which comprises (a) a compound of Formula (I), or a pharmaceutically acceptable salt thereof, (b) at least one additional therapeutic agent (e.g., any of the exemplary additional therapeutic agents described herein or known in the art), and (c) optionally at least one pharmaceutically acceptable carrier for simultaneous, separate or sequential use for the treatment of cancer, wherein the amounts of the compound of Formula (I), or pharmaceutically acceptable salt thereof, and of the additional therapeutic agent are together effective in treating the cancer; (ii) a pharmaceutical composition comprising such a combination; (iii) the use of such a combination for the preparation of a medicament for the treatment of cancer; and (iv) a commercial package or product comprising such a combination as a combined preparation for simultaneous, separate or sequential use; and to a method of treatment of cancer in a subject in need thereof. In some embodiments, the cancer is a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof).

The term “pharmaceutical combination”, as used herein, refers to a pharmaceutical therapy resulting from the mixing or combining of more than one active ingredient and includes both fixed and non-fixed combinations of the active ingredients. The term “fixed combination” means that a compound of Formula (I), or a pharmaceutically acceptable salt thereof, and at least one additional therapeutic agent (e.g., a chemotherapeutic agent), are both administered to a subject simultaneously in the form of a single composition or dosage. The term “non-fixed combination” means that a compound of Formula (I), or a pharmaceutically acceptable salt thereof, and at least one additional therapeutic agent (e.g., chemotherapeutic agent) are formulated as separate compositions or dosages such that they may be administered to a subject in need thereof simultaneously, concurrently or sequentially with variable intervening time limits, wherein such administration provides effective levels of the two or more compounds in the body of the subject. These also apply to cocktail therapies, e.g., the administration of three or more active ingredients.

Accordingly, also provided herein is a method of treating a cancer, comprising administering to a subject in need thereof a pharmaceutical combination for treating cancer which comprises (a) a compound of Formula (I), or pharmaceutically acceptable salt thereof, and (b) an additional therapeutic agent, wherein the compound of Formula (I) and the additional therapeutic agent are administered simultaneously, separately or sequentially, wherein the amounts of the compound of Formula (I), or pharmaceutically acceptable salt thereof, and the additional therapeutic agent are together effective in treating the cancer. In some embodiments, the compound of Formula (I), or pharmaceutically acceptable salt thereof, and the additional therapeutic agent are administered simultaneously as separate dosages. In some embodiments, the compound of Formula (I), or pharmaceutically acceptable salt thereof, and the additional therapeutic agent are administered as separate dosages sequentially in any order, in jointly effective amounts, e.g., in daily or intermittently dosages. In some embodiments, the compound of Formula (I), or pharmaceutically acceptable salt thereof, and the additional therapeutic agent are administered simultaneously as a combined dosage. In some embodiments, the cancer is a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof).

Accordingly, also provided herein are methods for inhibiting, preventing, aiding in the prevention, or decreasing the symptoms of metastasis of a cancer in a subject in need thereof, the method comprising administering to the subject an effective amount of a compound of Formula (I), or a pharmaceutically acceptable salt thereof or a pharmaceutical composition thereof. Such methods can be used in the treatment of one or more of the cancers described herein. See, e.g., US Publication No. 2013/0029925; International Publication No. WO 2014/083567; and U.S. Pat. No. 8,568,998. See also, e.g., Hezam K et al., Rev Neurosci 2018 Jan. 26; 29:93-98; Gao L, et al., Pancreas 2015 January; 44:134-143; Ding K et al., J Biol Chem 2014 Jun. 6; 289:16057-71; and Amit M et al., Oncogene 2017 Jun. 8; 36:3232-3239. In some embodiments, the cancer is a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof). In some embodiments, the compound of Formula (I), or a pharmaceutically acceptable salt thereof is used in combination with an additional therapy or another therapeutic agent, such as those described herein.

The term “metastasis” is an art known term and means the formation of an additional tumor (e.g., a solid tumor) at a site distant from a primary tumor in a subject, where the additional tumor includes the same or similar cancer cells as the primary tumor.

Also provided are methods of decreasing the risk of developing a metastasis or an additional metastasis in a subject having a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof) that include: selecting, identifying, or diagnosing a subject as having a Ras pathway-associated cancer, and administering an effective amount of a compound of Formula (I), or a pharmaceutically acceptable salt thereof to the subject selected, identified, or diagnosed as having a Ras pathway-associated cancer. Also provided are methods of decreasing the risk of developing a metastasis or an additional metastasis in a subject having a Ras pathway-associated cancer that includes administering an effective amount of a compound of Formula (I), or a pharmaceutically acceptable salt thereof to a subject having a Ras pathway-associated cancer. The decrease in the risk of developing a metastasis or an additional metastasis in a subject having a Ras pathway-associated cancer can be compared to the risk of developing a metastasis or an additional metastasis in the subject prior to treatment, or as compared to a subject or a population of subjects having a similar or the same Ras pathway-associated cancer that has received no treatment or a different treatment. In some embodiments, the additional therapeutic agent is selected from MRTX849, LY3499446, JNJ-74699157, AMG 510, ARS3248, ARS853, ARS1620, AZD4785, JNJ-74699157, SML-8-73-1, SML-10-70-1, VSA9, AA12, and MRTX-849. In some embodiments, the subject has been administered one or more doses of a compound of Formula (I), or a pharmaceutically acceptable salt thereof, prior to administration of the pharmaceutical composition.

Also provided are methods of decreasing the risk of developing a metastasis or an additional metastasis in a subject having a Ras-associated cancer that include: selecting, identifying, or diagnosing a subject as having a Ras-associated cancer, and administering an effective amount of a compound of Formula (I), or a pharmaceutically acceptable salt thereof to the subject selected, identified, or diagnosed as having a Ras-associated cancer. Also provided are methods of decreasing the risk of developing a metastasis or an additional metastasis in a subject having a Ras-associated cancer that includes administering an effective amount of a compound of Formula (I), or a pharmaceutically acceptable salt thereof to a subject having a Ras-associated cancer. The decrease in the risk of developing a metastasis or an additional metastasis in a subject having a Ras-associated cancer can be compared to the risk of developing a metastasis or an additional metastasis in the subject prior to treatment, or as compared to a subject or a population of subjects having a similar or the same Ras-associated cancer that has received no treatment or a different treatment. In some embodiments, the additional therapeutic agent is selected from MRTX849, LY3499446, JNJ-74699157, AMG 510, ARS3248, ARS853, ARS1620, AZD4785, JNJ-74699157, SML-8-73-1, SML-10-70-1, VSA9, AA12, and MRTX-849. In some embodiments, the subject has been administered one or more doses of a compound of Formula (I), or a pharmaceutically acceptable salt thereof, prior to administration of the pharmaceutical composition.

Also provided are methods of decreasing the risk of developing a metastasis or an additional metastasis in a subject having a KRas-associated cancer that include: selecting, identifying, or diagnosing a subject as having a KRas-associated cancer, and administering an effective amount of a compound of Formula (I), or a pharmaceutically acceptable salt thereof to the subject selected, identified, or diagnosed as having a KRas-associated cancer. Also provided are methods of decreasing the risk of developing a metastasis or an additional metastasis in a subject having a KRas-associated cancer that includes administering an effective amount of a compound of Formula (I), or a pharmaceutically acceptable salt thereof to a subject having a KRas-associated cancer. The decrease in the risk of developing a metastasis or an additional metastasis in a subject having a KRas-associated cancer can be compared to the risk of developing a metastasis or an additional metastasis in the subject prior to treatment, or as compared to a subject or a population of subjects having a similar or the same KRas-associated cancer that has received no treatment or a different treatment. In some embodiments, the additional therapeutic agent is selected from MRTX849, LY3499446, JNJ-74699157, AMG 510, ARS3248, ARS853, ARS1620, AZD4785, JNJ-74699157, SML-8-73-1, SML-10-70-1, VSA9, AA12, and MRTX-849. In some embodiments, the subject has been administered one or more doses of a compound of Formula (I), or a pharmaceutically acceptable salt thereof, prior to administration of the pharmaceutical composition.

Also provided are methods of decreasing the risk of developing a metastasis or an additional metastasis in a subject having a HRas-associated cancer that include: selecting, identifying, or diagnosing a subject as having a HRas-associated cancer, and administering an effective amount of a compound of Formula (I), or a pharmaceutically acceptable salt thereof to the subject selected, identified, or diagnosed as having a HRas-associated cancer. Also provided are methods of decreasing the risk of developing a metastasis or an additional metastasis in a subject having a HRas-associated cancer that includes administering an effective amount of a compound of Formula (I), or a pharmaceutically acceptable salt thereof to a subject having a HRas-associated cancer. The decrease in the risk of developing a metastasis or an additional metastasis in a subject having a HRas-associated cancer can be compared to the risk of developing a metastasis or an additional metastasis in the subject prior to treatment, or as compared to a subject or a population of subjects having a similar or the same HRas-associated cancer that has received no treatment or a different treatment. In some embodiments, the additional therapeutic agent is selected from MRTX849, LY3499446, JNJ-74699157, AMG 510, ARS3248, ARS853, ARS1620, AZD4785, JNJ-74699157, SML-8-73-1, SML-10-70-1, VSA9, AA12, and MRTX-849. In some embodiments, the subject has been administered one or more doses of a compound of Formula (I), or a pharmaceutically acceptable salt thereof, prior to administration of the pharmaceutical composition.

Also provided are methods of decreasing the risk of developing a metastasis or an additional metastasis in a subject having a NRas-associated cancer that include: selecting, identifying, or diagnosing a subject as having a NRas-associated cancer, and administering an effective amount of a compound of Formula (I), or a pharmaceutically acceptable salt thereof to the subject selected, identified, or diagnosed as having a NRas-associated cancer. Also provided are methods of decreasing the risk of developing a metastasis or an additional metastasis in a subject having a NRas-associated cancer that includes administering an effective amount of a compound of Formula (I), or a pharmaceutically acceptable salt thereof to a subject having a NRas-associated cancer. The decrease in the risk of developing a metastasis or an additional metastasis in a subject having a NRas-associated cancer can be compared to the risk of developing a metastasis or an additional metastasis in the subject prior to treatment, or as compared to a subject or a population of subjects having a similar or the same NRas-associated cancer that has received no treatment or a different treatment. In some embodiments, the additional therapeutic agent is selected from MRTX849, LY3499446, JNJ-74699157, AMG 510, ARS3248, ARS853, ARS1620, AZD4785, JNJ-74699157, SML-8-73-1, SML-10-70-1, VSA9, AA12, and MRTX-849. In some embodiments, the subject has been administered one or more doses of a compound of Formula (I), or a pharmaceutically acceptable salt thereof, prior to administration of the pharmaceutical composition.

Also provided are methods of decreasing the risk of developing a metastasis or an additional metastasis in a subject having a SOS1-associated cancer that include: selecting, identifying, or diagnosing a subject as having a SOS1-associated cancer, and administering an effective amount of a compound of Formula (I), or a pharmaceutically acceptable salt thereof to the subject selected, identified, or diagnosed as having a SOS1-associated cancer. Also provided are methods of decreasing the risk of developing a metastasis or an additional metastasis in a subject having a SOS1-associated cancer that includes administering an effective amount of a compound of Formula (I), or a pharmaceutically acceptable salt thereof to a subject having a SOS1-associated cancer. The decrease in the risk of developing a metastasis or an additional metastasis in a subject having a SOS1-associated cancer can be compared to the risk of developing a metastasis or an additional metastasis in the subject prior to treatment, or as compared to a subject or a population of subjects having a similar or the same SOS1-associated cancer that has received no treatment or a different treatment. In some embodiments, the additional therapeutic agent is selected from MRTX849, LY3499446, JNJ-74699157, AMG 510, ARS3248, ARS853, ARS1620, AZD4785, JNJ-74699157, SML-8-73-1, SML-10-70-1, VSA9, AA12, and MRTX-849. In some embodiments, the subject has been administered one or more doses of a compound of Formula (I), or a pharmaceutically acceptable salt thereof, prior to administration of the pharmaceutical composition.

The phrase “risk of developing a metastasis” means the risk that a subject having a primary tumor will develop an additional tumor (e.g., a solid tumor) at a site distant from a primary tumor in a subject over a set period of time, where the additional tumor includes the same or similar cancer cells as the primary tumor. Methods for reducing the risk of developing a metastasis in a subject having a cancer are described herein.

The phrase “risk of developing additional metastases” means the risk that a subject having a primary tumor and one or more additional tumors at sites distant from the primary tumor (where the one or more additional tumors include the same or similar cancer cells as the primary tumor) will develop one or more further tumors distant from the primary tumor, where the further tumors include the same or similar cancer cells as the primary tumor. Methods for reducing the risk of developing additional metastasis are described herein.

Treatment of a subject having a cancer with a multi-kinase inhibitor (MKI) or target-specific kinase inhibitor (e.g., a BRAF inhibitor, an EGFR inhibitor, a MEK inhibitor, an ALK inhibitor, a ROS1 inhibitor, a MET inhibitor, an aromatase inhibitor, a RAF inhibitor, a RET inhibitor, or a RAS inhibitor) can result in dysregulation of a Ras pathway gene (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), a Ras pathway protein (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or the expression or activity or level of the same in the cancer. See, e.g., Bhinge et al., Oncotarget 8:27155-27165, 2017; Chang et al., Yonsei Med. J. 58:9-18, 2017; and Lopez-Delisle et al., doi: 10.1038/s41388-017-0039-5, Oncogene 2018.

Treatment of a subject having a cancer with a SOS1 inhibitor in combination with a multi-kinase inhibitor or a target-specific kinase inhibitor (e.g., a BRAF inhibitor, an EGFR inhibitor, a MEK inhibitor, an ALK inhibitor, a ROS1 inhibitor, a MET inhibitor, an aromatase inhibitor, a RAF inhibitor, a RET inhibitor, or a RAS inhibitor) can have increased therapeutic efficacy as compared to treatment of the same subject or a similar subject with the SOS1 inhibitor as a monotherapy, or the multi-kinase inhibitor or the target-specific kinase inhibitor as a monotherapy. See, e.g., Tang et al., doi: 10.1038/modpathol.2017.109, Mod. Pathol. 2017; Andreucci et al., Oncotarget 7:80543-80553, 2017; Nelson-Taylor et al., Mol. Cancer Ther. 16:1623-1633, 2017; and Kato et al., Clin. Cancer Res. 23:1988-1997, 2017.

Provided herein are methods of treating a subject having a cancer (e.g., any of the cancers described herein) and previously administered a multi-kinase inhibitor (MKI) or a target-specific kinase inhibitor (e.g., a Ras inhibitor, a BRAF inhibitor, an EGFR inhibitor, a MEK inhibitor, an ALK inhibitor, a ROS1 inhibitor, a MET inhibitor, an aromatase inhibitor, a RAF inhibitor, a RET inhibitor, or a RAS inhibitor) (e.g., as a monotherapy) that include: administering to the subject (i) an effective dose of a compound of Formula (I), or a pharmaceutically acceptable salt thereof as a monotherapy, or (ii) an effective dose of a compound of Formula (I), or a pharmaceutically acceptable salt thereof, and an effective dose of the previously administered MKI or the previously administered target-specific kinase inhibitor.

Also provided is a method for inhibiting SOS1 activity in a mammalian cell, comprising contacting the mammalian cell with a compound of Formula (I). In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof to a subject having a mammalian cell having SOS1 activity. In some embodiments, the mammalian cell is a mammalian cancer cell. In some embodiments, the mammalian cancer cell is any cancer as described herein. In some embodiments, the mammalian cancer cell is a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof) cell.

Also provided is a method for inhibiting Ras activity in a mammalian cell, comprising contacting the mammalian cell with a compound of Formula (I). In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof to a subject having a mammalian cell having Ras activity. In some embodiments, the mammalian cell is a mammalian cancer cell. In some embodiments, the mammalian cancer cell is any cancer as described herein. In some embodiments, the mammalian cancer cell is a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof) cell.

Also provided is a method for inhibiting a SOS1-Ras (e.g., KRas, HRas, and/or NRas) protein-protein interaction in a mammalian cell, comprising contacting the mammalian cell with a compound of Formula (I). In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof to a subject having a mammalian cell having a SOS1-Ras (e.g., KRas, HRAs, and/or NRas) protein-protein interaction. In some embodiments, the mammalian cell is a mammalian cancer cell. In some embodiments, the mammalian cancer cell is any cancer as described herein. In some embodiments, the mammalian cancer cell is a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof) cell.

Also provided is a method for inhibiting Ras pathway activity in a mammalian cell, comprising contacting the mammalian cell with a compound of Formula (I). In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof to a subject having a mammalian cell having Ras pathway activity. In some embodiments, the mammalian cell is a mammalian cancer cell. In some embodiments, the mammalian cancer cell is any cancer as described herein. In some embodiments, the mammalian cancer cell is a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof) cell.

As used herein, the term “contacting” refers to the bringing together of indicated moieties in an in vitro system or an in vivo system. For example, “contacting” a SOS1 protein with a compound provided herein includes the administration of a compound provided herein to a subject, such as a human, having a SOS1 protein, as well as, for example, introducing a compound provided herein into a sample containing a mammalian cellular or purified preparation containing the SOS1 protein.

Also provided herein is a method of inhibiting mammalian cell proliferation, in vitro or in vivo, the method comprising contacting a mammalian cell with an effective amount of a compound of Formula (I), or a pharmaceutically acceptable salt thereof, or a pharmaceutical composition thereof as defined herein.

The phrase “effective amount” means an amount of compound that, when administered to a subject in need of such treatment, is sufficient to (i) treat a Ras pathway-associated disease or disorder (such as a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof)), (ii) attenuate, ameliorate, or eliminate one or more symptoms of the particular disease, condition, or disorder, or (iii) delay the onset of one or more symptoms of the particular disease, condition, or disorder described herein. The amount of a compound of Formula (I), or a pharmaceutically acceptable salt thereof that will correspond to such an amount will vary depending upon factors such as the particular compound, disease condition and its severity, the identity (e.g., weight) of the subject in need of treatment, but can nevertheless be routinely determined by one skilled in the art.

### Pharmaceutical Compositions

When employed as pharmaceuticals, compounds of Formula (I),including pharmaceutically acceptable salts thereof, can be administered in the form of pharmaceutical compositions. These compositions can be prepared in a manner well known in the pharmaceutical art, and can be administered by a variety of routes, depending upon whether local or systemic treatment is desired and upon the area to be treated. Administration can be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable.

Also provided herein are pharmaceutical compositions which contain, as the active ingredient, a compound of Formula (I) or pharmaceutically acceptable salt thereof, in combination with one or more pharmaceutically acceptable excipients. For example, a pharmaceutical composition prepared using a compound of Formula (I) or a pharmaceutically acceptable salt thereof. In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is a solid oral formulation. In some embodiments, the composition is formulated as a tablet or capsule.

Further provided herein are pharmaceutical compositions containing a compound of Formula (I) or a pharmaceutically acceptable salt thereof with a pharmaceutically acceptable carrier. Pharmaceutical compositions containing a compound of Formula (I) or a pharmaceutically acceptable salt thereof as the active ingredient can be prepared by intimately mixing the compound of Formula (I), or a pharmaceutically acceptable salt thereof with a pharmaceutical carrier according to conventional pharmaceutical compounding techniques. The carrier can take a wide variety of forms depending upon the desired route of administration (e.g., oral, parenteral). In some embodiments, the composition is a solid oral composition.

Suitable pharmaceutically acceptable carriers are well known in the art. Descriptions of some of these pharmaceutically acceptable carriers can be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In preparing the compositions in oral dosage form, any of the usual pharmaceutical media can be employed. Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Solid oral preparations can also be coated with substances such as sugars or be enteric-coated so as to modulate major site of absorption. For parenteral administration, the carrier will usually consist of sterile water and other ingredients can be added to increase solubility or preservation. Injectable suspensions or solutions can also be prepared utilizing aqueous carriers along with appropriate additives. The pharmaceutical compositions herein will contain, per dosage unit, e.g., tablet, capsule, powder, injection, teaspoonful and the like, an amount of the active ingredient necessary to deliver an effective dose as described herein.

The compositions comprising a compound of Formula (I) or a pharmaceutically acceptable salt thereof can be formulated in a unit dosage form, each dosage containing from about 5 to about 1,000 mg (1 g), more usually about 100 mg to about 500 mg, of the active ingredient. The term “unit dosage form” refers to physically discrete units suitable as unitary dosages for human subjects and other subjects, each unit containing a predetermined quantity of active material (i.e., a compound of Formula (I) or a pharmaceutically acceptable salt thereof) calculated to produce the desired therapeutic effect, in association with a suitable pharmaceutical excipient.

In some embodiments, the compositions provided herein contain from about 5 mg to about 50 mg of the active ingredient. One having ordinary skill in the art will appreciate that this embodies compounds or compositions containing about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg of the active ingredient.

In some embodiments, the compositions provided herein contain from about 50 mg to about 500 mg of the active ingredient. One having ordinary skill in the art will appreciate that this embodies compounds or compositions containing about 50 mg to about 100 mg, about 100 mg to about 150 mg, about 150 mg to about 200 mg, about 200 mg to about 250 mg, about 250 mg to about 300 mg, about 350 mg to about 400 mg, or about 450 mg to about 500 mg of the active ingredient. In some embodiments, the compositions provided herein contain about 10 mg, about 20 mg, about 80 mg, or about 160 mg of the active ingredient.

In some embodiments, the compositions provided herein contain from about 500 mg to about 1,000 mg of the active ingredient. One having ordinary skill in the art will appreciate that this embodies compounds or compositions containing about 500 mg to about 550 mg, about 550 mg to about 600 mg, about 600 mg to about 650 mg, about 650 mg to about 700 mg, about 700 mg to about 750 mg, about 750 mg to about 800 mg, about 800 mg to about 850 mg, about 850 mg to about 900 mg, about 900 mg to about 950 mg, or about 950 mg to about 1,000 mg of the active ingredient.

The daily dosage of the compound of Formula (I) or a pharmaceutically acceptable salt thereof can be varied over a wide range from 1.0 to 10,000 mg per adult human per day, or higher, or any range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 160, 200, 250 and 500 milligrams of the active ingredient for the symptomatic adjustment of the dosage to the subject to be treated. An effective amount of the drug is ordinarily supplied at a dosage level of from about 0.1 mg/kg to about 1000 mg/kg of body weight per day, or any range therein. Preferably, the range is from about 0.5 to about 500 mg/kg of body weight per day, or any range therein. More preferably, from about 1.0 to about 250 mg/kg of body weight per day, or any range therein. More preferably, from about 0.1 to about 100 mg/kg of body weight per day, or any range therein. In an example, the range can be from about 0.1 to about 50.0 mg/kg of body weight per day, or any amount or range therein. In another example, the range can be from about 0.1 to about 15.0 mg/kg of body weight per day, or any range therein. In yet another example, the range can be from about 0.5 to about 7.5 mg/kg of body weight per day, or any amount to range therein. Pharmaceutical compositions containing a compound of Formula (I) or a pharmaceutically acceptable salt thereof can be administered on a regimen of 1 to 4 times per day or in a single daily dose.

The active compound may be effective over a wide dosage range and is generally administered in a pharmaceutically effective amount. Optimal dosages to be administered can be readily determined by those skilled in the art. It will be understood, therefore, that the amount of the compound actually administered will usually be determined by a physician, and will vary according to the relevant circumstances, including the mode of administration, the actual compound administered, the strength of the preparation, the condition to be treated, and the advancement of the disease condition. In addition, factors associated with the particular subject being treated, including subject response, age, weight, diet, time of administration and severity of the subject's symptoms, will result in the need to adjust dosages.

In some embodiments, the compounds provided herein can be administered in an amount ranging from about 1 mg/kg to about 100 mg/kg. In some embodiments, the compound provided herein can be administered in an amount of about 1 mg/kg to about 20 mg/kg, about 5 mg/kg to about 50 mg/kg, about 10 mg/kg to about 40 mg/kg, about 15 mg/kg to about 45 mg/kg, about 20 mg/kg to about 60 mg/kg, or about 40 mg/kg to about 70 mg/kg. For example, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg. In some embodiments, such administration can be once-daily or twice-daily (BID) administration.

One skilled in the art will recognize that both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy subjects and/or those suffering from a given disorder, can be completed according to methods well known in the clinical and medical arts.

Provided herein are pharmaceutical kits useful, for example, in the treatment of Ras pathway-associated diseases or disorders, such as cancer, which include one or more containers containing a pharmaceutical composition comprising an effective amount of a compound provided herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### EXAMPLES

### Materials and Methods

The compounds provided herein, including salts thereof, can be prepared using known organic synthesis techniques and can be synthesized according to any of numerous possible synthetic routes.

The reactions for preparing the compounds provided herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in Protecting Group Chemistry, 1^{st} Ed., Oxford University Press, 2000; March's Advanced Organic Chemistry: Reactions, Mechanisms, and Structure, 5^{th} Ed., Wiley-Interscience Publication, 2001; and Peturssion, S. et al., “Protecting Groups in Carbohydrate Chemistry,” J. Chem. Educ., 74(11), 1297 (1997).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) (“Preparative LC-MS Purification: Improved Compound Specific Method Optimization” K. F. Blom, et al., J. Combi. Chem. 6(6), 874 (2004), normal phase silica chromatography, and supercritical fluid chromatography (SFC).

All solvents and reagents were obtained from commercial sources and used without further purification unless indicated otherwise. Anhydrous solvents were purchased and used as supplied. Reactions were monitored by thin-layer chromatography (TLC), visualizing with a UV lamp (254 nm) and KMnO₄ stain. NMR spectra were obtained on a Bruker Neo 400M spectrometer operating at 400 MHz. Chemical shifts are reported in parts per million (δ) from the tetramethysilane resonance in the indicated solvent. LC-Mass spectra were taken with Agilent 1260-6125B single quadrupole mass spectrometer using a Welch Biomate column (C18, 2.7 μm, 4.6*50 mm) or waters H-Class SQD2 system. The detection was by DAD (254 nm and 210 nm and 280 nm). Chiral HPLC was performed on the Waters acquity UPC2 system under base-containing on Daicel chiralpak AD-H (5 μm, 4.6*250 mm), Daicel chiralpak OD-H (5 μm, 4.6*250 mm), Daicel chiralpak IG-3 (3 μm, 4.6*150 mm), Chiral Technologies Europe AD-3 (3 μm, 3.0*150 mm) and Trefoil™ Technology Trefoil™ AMY1 (2.5 μm, 3.0*150 mm). The detection was by DAD (254 nm). Preparative HPLC was performed on GILSON Trilution LC system using a Welch XB-C18 column (5 um, 21.2*150 mm). Flash chromatography was carried out on Biotage Isolera Prime system using Welch WelFlash flash columns (40-63 μm). The compounds synthesized are all with purity ≥95% unless otherwise specified.

### ABBREVIATIONS

° C.=degrees Celsius
¹H NMR=proton nuclear magnetic resonance spectrum
ACN=acetonitrile
AcOH=acetic acid
BAST=Bis(2-methoxyethyl)aminosulfur trifluoride
DAST=Diethylaminosulfur trifluoride
Boc=tert-butoxycarbonyl
con.=concentrated
d=doublet
DCM=dichloromethane
DIPEA=N,N-diisopropylethylamine
DMF=N,N-dimethylformamide
DMF-DMA=dimethylformamide dimethyl acetal
DMSO=dimethylsulfoxide
Et=ethyl
EtOAc (or EA)=ethyl acetate
EtOH=ethanol
ESI=electrospray ionization
g=gram(s)
hr=hour(s)
HATU=1-[Bis(dimethylamino)methylene]-1H-1,2,3-triazolo[4,5-b]pyridinium 3-oxide
hexafluorophosphate
HPLC=high-performance liquid chromatography
IBX=2-iodoxybenzoic acid
IPA=2-propanol
LCMS=liquid chromatograph-mass spectrum
M=mass
m/z=mass-to-charge ratio
Me=methyl
MeCN=acetonitrile
MeOH=methanol
MeONa=sodium methoxide
mg=milligram(s)
mL=milliliter
mmol=millimole(s)
mol=mole(s)
MS=mass spectrum
NBS=N-bromosuccinimide
obsd.=observed
Pd(OAc)₂=palladium (II) acetate
Pd(dppf)Cl₂=(1,1′-Bis(diphenylphosphino)ferrocene)palladium(II) dichloride
Pd₂(dba)₃=Tris(dibenzylideneacetone)dipalladium(O)
PE=petroleum ether
ppm=parts per million
rt=room temperature
RuPhos=2-Dicyclohexylphosphino-2′,6′-diisopropoxybiphenyl
RuPhos-Pd-G3=(2-Dicyclohexylphosphino-2′,6′-diisopropoxy-1,1′-biphenyl)[2-(2′-amino-1,1′-biphenyl)]palladium(II) methanesulfonate
s=singlet
T3P=Propylphosphonic anhydride
t=triplet
TBAF=tetrabutylammonium fluoride
tBu=tert-butyl
TEA=triethylamine
Tf=trifluoromethanesulfonate
TFA=trifluoroacetic acid
THF=tetrahydrofuran
TLC=thin-layer chromatography
XantPhos=(9,9-Dimethyl-9H-xanthene-4,5-diyl)bis(diphenylphosphane)
XantPhos-Pd-G3=[(4,5-Bis(diphenylphosphino)-9,9-dimethylxanthene)-2-(2′-amino-1,1′-biphenyl)]palladium(II) methanesulfonate

### Intermediate 1: 6-cyclopropyl-2-methyl-2,3-dihydropyrido[3,4-d]pyridazine-1,4,7(6H)-trione

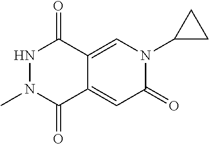 [SMILES: Cn1[nH]c(=O)c2cn(C3CC3)c(=O)cc2c1=O]

### Step A: 3-(tert-butyl) 4-methyl 1-cyclopropyl-6-oxo-1,6-dihydropyridine-3,4-dicarboxylate

To a solution cyclopropanamine (4.53 g, 79.27 mmol, 5.49 mL) in EtOH/H₂O (450 mL/4 mL) was added tert-butyl prop-2-ynoate (10 g, 79.27 mmol, 10.88 mL). The mixture was stirred at RT for 16 hrs, then dimethyl but-2-ynedioate (11.26 g, 79.27 mmol, 9.71 mL) was added and the mixture was stirred at 85° C. for 16 hrs. The mixture was cooled to RT, concentrated, and purified by flash column chromatography (0 to 25% ethyl acetate in petroleum ether) to afford 3-(tert-butyl) 4-methyl 1-cyclopropyl-6-oxo-1,6-dihydropyridine-3,4-dicarboxylate (9.12 g, 31.09 mmol, 39% yield) as a yellow liquid. MS obsd. (ESI⁺): 294.2 [(M+H)⁺].

### Step B: 5-(tert-butoxycarbonyl)-1-cyclopropyl-2-oxo-1,2-dihydropyridine-4-carboxylic acid

3-(tert-butyl) 4-methyl 1-cyclopropyl-6-oxo-1,6-dihydropyridine-3,4-dicarboxylate (3.2 g, 10.90 mmol, 1.0 eq) was dissolved in MeOH/H₂O (24 mL/6 mL). To the solution was added LiOH (393 mg, 16.4 mmol, 1.5 eq) at 0° C. The solution was then stirred at RT for 2 hrs and then concentrated. To the crude mixture was added ethyl acetate and water. The mixture was adjusted to pH=5˜6 by addition of 2M HCl, extracted with ethyl acetate (50 mL*3) and the combined organic phases were dried and concentrated to give 5-(tert-butoxycarbonyl)-1-cyclopropyl-2-oxo-1,2-dihydropyridine-4-carboxylic acid (2.8 g, 10.07 mmol, 98%) as a light yellow solid. MS obsd. (ESI⁺): 280.2 [(M+H)⁺].

### Step C: tert-butyl 4-(2-(tert-butoxycarbonyl)-1-methylhydrazine-1-carbonyl)-1-cyclopropyl-6-oxo-1,6-dihydropyridine-3-carboxylate

5-(tert-butoxycarbonyl)-1-cyclopropyl-2-oxo-1,2-dihydropyridine-4-carboxylic acid (2.9 g, 10.38 mmol) was dissolved in THE (30 mL) followed by the addition DIPEA (1.61 g, 12.46 mmol), HATU (4.74 g, 12.46 mmol, 1.2 eq), and tert-butyl 2-methylhydrazine-1-carboxylate (1.82 g, 12.46 mmol). The solution was stirred at RT for 2 hrs, then concentrated and reduced, and partitioned between ethyl acetate (150 mL) and sodium bicarbonate saturated solution (50 mL). The organic phase was washed with brine (30 mL), dried, concentrated, and purified by flash column chromatography (10-100% ethyl acetate in petroleum ether) to provide tert-butyl 4-(2-(tert-butoxycarbonyl)-1-methylhydrazine-1-carbonyl)-1-cyclopropyl-6-oxo-1,6-dihydropyridine-3-carboxylate (4.14 g, 10.16 mmol, 97% yield) as a white solid. MS obsd. (ESI⁺): 408.4 [(M+H)⁺].

### Step D: 6-cyclopropyl-2-methyl-2,3-dihydropyrido[3,4-d]pyridazine-1,4,7(6H)-trione

A mixture of tert-butyl 4-(2-(tert-butoxycarbonyl)-1-methylhydrazine-1-carbonyl)-1-cyclopropyl-6-oxo-1,6-dihydropyridine-3-carboxylate (4.01 g, 9.82 mmol, 1.0 eq) and HCl (4M in 1,4-dioxane, 90 mL) was stirred at RT for 30 min. then heated at 100° C. for 30 min. The mixture was cooled to RT, filtered, and the filter cake was washed with ethyl acetate (10 mL*3) to afford 6-cyclopropyl-2-methyl-2,3-dihydropyrido[3,4-d]pyridazine-1,4,7(6H)-trione (1.66 g, 7.12 mmol, 73% yield) as a yellow solid. MS obsd. (ESI⁺): 234.4[(M+H)⁺].

### Example 1: (R)-6-cyclopropyl-2-methyl-4-((1-(naphthalen-1-yl)ethyl)amino)-2,6-dihydropyrido [3,4-d]pyridazine-1,7-dione

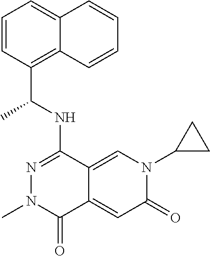 [SMILES: C[C@@H](Nc1nn(C)c(=O)c2cc(=O)n(C3CC3)cc12)c1cccc2ccccc12]

### Step A: 6-cyclopropyl-2-methyl-1,7-dioxo-1,2,6,7-tetrahydropyrido[3,4-d]pyridazin-4-yl trifluoromethanesulfonate

A mixture of 6-cyclopropyl-2-methyl-2,3-dihydropyrido[3,4-d]pyridazine-1,4,7(6H)-trione (100 mg, 0.43 mmol) and pyridine (50.87 mg, 643.16 μmol) in DCM (1.5 mL) was cooled to 0° C. under N₂. A solution of triflic anhydride (181.46 mg, 643.16 μmol) in DCM (1.5 mL) was added and the mixture was stirred at 0° C. for 1 hr. The mixture was diluted with DCM (20 mL) and quenched with water (5 mL). The organic phase was dried, concentrated, and purified by flash column chromatography (0-100% ethyl acetate in cyclohexane) to afford 6-cyclopropyl-2-methyl-1,7-dioxo-1,2,6,7-tetrahydropyrido[3,4-d]pyridazin-4-yl trifluoromethanesulfonate (102 mg, 0.28 mmol, 65% yield) as a yellow solid. ¹H NMR (400 MHz, CDCl₃) δ 8.04 (s, 1H), 7.35 (s, 1H), 3.63 (s, 3H), 3.59-3.54 (m, 1H), 1.35-1.31 (m, 2H), 1.01-0.95 (m, 2H).

### Step B: (R)-6-cyclopropyl-2-methyl-4-((1-(naphthalen-1-yl)ethyl)amino)-2,6-dihydropyrido[3,4-d]pyridazine-1,7-dione

A mixture of 6-cyclopropyl-2-methyl-1,7-dioxo-1,2,6,7-tetrahydropyrido[3,4-d]pyridazin-4-yl trifluoromethanesulfonate (30 mg, 0.082 mmol), (R)-1-(naphthalen-1-yl)ethan-1-amine (16.88 mg, 0.098 mmol), Cs₂CO₃ (53.5 mg, 0.16 mmol) and Xantphos (4.75 mg, 0.008 mmol) in 1,4-dioxane (1.0 mL) was degassed with N₂ for 5 min., then Pd₂(dba)₃ (7.5 mg, 0.0081 mmol) was added and the mixture was heated to 100° C. for 20 hrs. The reaction mixture was concentrated and the residue was taken up in DMSO and purified by reverse phase HPLC to afford (R)-6-cyclopropyl-2-methyl-4-((1-(naphthalen-1-yl)ethyl)amino)-2,6-dihydropyrido[3,4-d]pyridazine-1,7-dione (3.1 mg, 0.008 mmol, 9% yield) as a yellow solid. MS obsd. (ESI⁺): 387.3 [(M+H)⁺].

### Example 2: (R)-6-cyclopropyl-2-methyl-4-((1-(2-methyl-3-(trifluoromethyl)phenyl) ethylamino)-2,6-dihydropyrido[3,4-d]pyridazine-1,7-dione

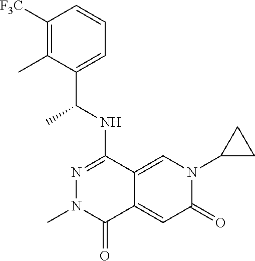 [SMILES: Cc1c([C@@H](C)Nc2nn(C)c(=O)c3cc(=O)n(C4CC4)cc23)cccc1C(F)(F)F]

### Step A: 6-cyclopropyl-2-methyl-1,7-dioxo-1,2,6,7-tetrahydropyrido[3,4-d]pyridazin-4-yl 2,4,6-triisopropylbenzenesulfonate

6-cyclopropyl-2-methyl-2,3-dihydropyrido[3,4-d]pyridazine-1,4,7(6H)-trione (400 mg, 1.7 mmol, 1.0 eq) and N,N-diisopropylethylamine (658 mg, 5.1 mmol, 3.0 eq) were dissolved in DCM (20 mL), and 2,4,6-triisopropylbenzenesulfonyl chloride (566 mg, 1.87 mmol, 1.1 eq) was added at 0° C. under N₂. The mixture was warmed to RT and stirred for 2 hrs. The solvent was concentrated and the crude mixture was washed with sodium bicarbonate saturated solution (50 mL) and extracted with DCM (150 mL). The organic phase was dried over sodium sulfate, concentrated and purified by flash column chromatography (10-20% ethyl acetate and 1% DCM in petroleum ether) to afford 6-cyclopropyl-2-methyl-1,7-dioxo-1,2,6,7-tetrahydropyrido[3,4-d]pyridazin-4-yl 2,4,6-triisopropylbenzenesulfonate (560 mg, 1.12 mmol, 66% yield) as a yellow solid. MS obsd. (ESI⁺): 500.5 [(M+H)⁺].

### Step B: (R)-6-cyclopropyl-2-methyl-4-((1-(2-methyl-3-(trifluoromethyl)phenyl)ethyl)amino)-2,6-dihydropyrido[3,4-d]pyridazine-1,7-dione

6-cyclopropyl-2-methyl-1,7-dioxo-1,2,6,7-tetrahydropyrido[3,4-d]pyridazin-4-yl 2,4,6-triisopropylbenzenesulfonate (50 mg, 0.10 mmol, 1.0 eq), (R)-1-(2-methyl-3-(trifluoromethyl)phenyl)ethan-1-amine (28 mg, 0.12 mmol, 1.2 eq), XantPhos Pd G3 (9 mg, 0.01 mmol, 0.1 eq) and Cs₂CO₃ (98 mg, 0.3 mmol, 3.0 eq) were dissolved in 1,4-dioxane (2 mL) under N₂. The mixture was stirred at 100° C. for 16 hrs. The mixture was cooled, filtered, and concentrated. The crude residue was pre-purified by preparative TLC (3% MeOH in DCM) to afford an impure product residue (20 mg, yellow solid). The product was further purified by reverse phase HPLC to afford (R)-6-cyclopropyl-2-methyl-4-((1-(2-methyl-3-(trifluoromethyl)phenyl)ethyl)amino)-2,6-dihydropyrido[3,4-d]pyridazine-1,7-dione (15 mg, 0.036 mmol, 35% yield) as a yellow solid. MS obsd. (ESI⁺): 419.2 [(M+H)⁺]. ¹H NMR (400 MHz, DMSO-d6) dppm: 8.74 (s, 1H), 7.72 (d, J=7.6 Hz, 1H), 7.52 (d, J=7.4 Hz, 1H), 7.35 (s, 1H), 7.23 (d, J=6.4 Hz, 1H), 6.81 (s, 1H), 5.33-5.07 (m, 1H), 3.61-3.45 (m, 1H), 3.24 (s, 3H), 2.53 (s, 3H), 1.46 (d, J=6.8 Hz, 3H), 1.19-1.11 (m, 2H), 1.08 (m, 2H).

### Example 3: (R)-8-bromo-6-cyclopropyl-2-methyl-4-((1-(2-methyl-3-(trifluoromethyl)phenyl)ethyl)amino)-2,6-dihydropyrido[3,4-d]pyridazine-1,7-dione

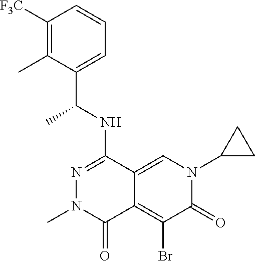 [SMILES: Cc1c([C@@H](C)Nc2nn(C)c(=O)c3c(Br)c(=O)n(C4CC4)cc23)cccc1C(F)(F)F]

To a solution of (R)-6-cyclopropyl-2-methyl-4-((1-(2-methyl-3-(trifluoromethyl)phenyl)ethyl)amino)-2,6-dihydropyrido[3,4-d]pyridazine-1,7-dione (300 mg, 0.71 mmol, 1.0 eq), and potassium acetate (70 mg, 0.71 mmol, 1.0 eq) in acetic acid (10 mL) was added bromine (138 mg, 0.86 mmol, 1.2 eq). The mixture was heated to 70° C. and stirred for 2 hrs. The mixture was washed with water (30 mL) and extracted with ethyl acetate. The organic phase was dried, concentrated, and purified by preparative-TLC (3% MeOH in DCM) to obtain an impure product (120 mg). The crude product was further purified by reverse phase HPLC to afford (R)-8-bromo-6-cyclopropyl-2-methyl-4-((1-(2-methyl-3-(trifluoromethyl)phenyl)ethyl)amino)-2,6-dihydropyrido[3,4-d]pyridazine-1,7-dione (37 mg, 0.072 mmol, 10% yield) as a yellow solid. ¹H NMR (400 MHz, DMSO) δ ppm 8.68 (s, 1H), 7.72 (d, J=7.8 Hz, 1H), 7.52 (d, J=7.6 Hz, 1H), 7.34 (t, J=7.8 Hz, 1H), 7.16 (d, J=6.2 Hz, 1H), 5.23-5.03 (m, 1H), 3.67-3.49 (m, 1H), 3.21 (s, 3H), 2.53 (s, 3H), 1.46 (d, J=6.8 Hz, 3H), 1.19-1.05 (m, 4H). MS obsd. (ESI⁺): 497.2/499.2 (⁷⁹Br/⁸¹Br) [(M+H)⁺].

### Example 4: (R)-2-methyl-4-((1-(2-methyl-3-(trifluoromethyl)phenyl)ethyl)amino)-6-(tetrahydro-2H-pyran-4-yl)-2,6-dihydropyrido[3,4-d]pyridazine-1,7-dione

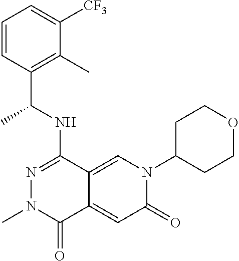 [SMILES: Cc1c([C@@H](C)Nc2nn(C)c(=O)c3cc(=O)n(C4CCOCC4)cc23)cccc1C(F)(F)F]

### Step A: 3-(tert-butyl) 4-methyl 6-oxo-1-(tetrahydro-2H-pyran-4-yl)-1,6-dihydropyridine-3,4-dicarboxylate

Tetrahydro-2H-pyran-4-amine (1 g, 9.88 mmol, 1.0 eq) was dissolved in Ethanol/H₂O (300 mL, V/V=500/1), and tert-butyl propiolate (1.2 g, 9.88 mmol, 1.0 eq) was added dropwise. The mixture was stirred at RT for 24 hrs. At this time, dimethyl but-2-ynedioate (1.4 g, 9.88 mmol, 1.0 eq) was added and the mixture was heated to reflux for 24 hrs. The mixture was cooled to RT and sodium methoxide (523 mg, 9.88 mmol, 1.0 eq) was added and stirred at RT for a further 8 hrs. The mixture was concentrated and was directly purified by flash column chromatography (10-20% ethyl acetate in petroleum ether) to afford 3-(tert-butyl) 4-methyl 6-oxo-1-(tetrahydro-2H-pyran-4-yl)-1,6-dihydropyridine-3,4-dicarboxylate (2.2 g, 6.53 mmol, 66% yield) as a light yellow solid. MS obsd. (ESI⁺): 338.3 [(M+H)⁺].

### Step B: 5-(tert-butoxycarbonyl)-2-oxo-1-(tetrahydro-2H-pyran-4-yl)-1,2-dihydropyridine-4-carboxylic acid

3-(tert-butyl) 4-methyl 6-oxo-1-(tetrahydro-2H-pyran-4-yl)-1,6-dihydropyridine-3,4-dicarboxylate (1.1 g, 3.26 mmol, 1.0 eq) was dissolved in MeOH (20 mL), and lithium hydroxide (156 mg, 6.52 mmol, 2.0 eq) was added. The mixture was stirred at RT overnight and then concentrated. The residue was partitioned between ethyl acetate (50 mL) and 1M HCl solution (5 mL). The organic phase was dried over sodium sulfate and concentrated to afford 5-(tert-butoxycarbonyl)-2-oxo-1-(tetrahydro-2H-pyran-4-yl)-1,2-dihydropyridine-4-carboxylic acid (860 mg, 2.66 mmol, 82% yield) as a light yellow solid. MS obsd. (ESI⁺): 324.3 [(M+H)⁺].

### Step C: tert-butyl 4-(2-(tert-butoxycarbonyl)-1-methylhydrazine-1-carbonyl)-6-oxo-1-(tetrahydro-2H-pyran-4-yl)-1,6-dihydropyridine-3-carboxylate

A mixture of 5-(tert-butoxycarbonyl)-2-oxo-1-(tetrahydro-2H-pyran-4-yl)-1,2-dihydropyridine-4-carboxylic acid (700 mg, 2.17 mmol, 1.0 eq), HATU (988 mg, 2.60 mmol, 1.2 eq) and N,N-diisopropylethylamine (336 mg, 2.6 mmol, 1.2 eq) in THE (10 mL) was treated with tert-butyl 2-methylhydrazine-1-carboxylate (380 mg, 2.6 mmol, 1.2 eq) and the mixture was stirred at RT overnight and then concentrated. The crude residue was partitioned between ethyl acetate (150 mL) and sodium bicarbonate saturated solution (50 mL). The organic phase was washed with brine (20 mL), dried over sodium sulfate, concentrated, and purified by flash column chromatography (1% MeOH in DCM) to afford tert-butyl 4-(2-(tert-butoxycarbonyl)-1-methylhydrazine-1-carbonyl)-6-oxo-1-(tetrahydro-2H-pyran-4-yl)-1,6-dihydropyridine-3-carboxylate (760 mg, 1.68 mmol, 78% yield) as a light yellow solid. MS obsd. (ESI⁺): 452.3 [(M+H)⁺].

### Step D: 2-methyl-6-(tetrahydro-2H-pyran-4-yl)-2,3-dihydropyrido[3,4-d]pyridazine-1,4,7(6H)-trione

4-(2-(tert-butoxycarbonyl)-1-methylhydrazine-1-carbonyl)-6-oxo-1-(tetrahydro-2H-pyran-4-yl)-1,6-dihydropyridine-3-carboxylate (700 mg, 1.55 mmol, 1.0 eq) was dissolved in hydrochloric acid (15 mL, 4M in 1,4-dioxane). The mixture was stirred at RT for 30 min. and then heated to 100° C. and stirred for 30 min. The mixture was cooled to RT and the resulting solid was filtered under reduced pressure to afford 2-methyl-6-(tetrahydro-2H-pyran-4-yl)-2,3-dihydropyrido[3,4-d]pyridazine-1,4,7(6H)-trione (328 mg, 1.18 mmol, 76% yield) as a light yellow solid. MS obsd. (ESI⁺): 278.1 [(M+H)⁺].

### Step E: 2-methyl-1,7-dioxo-6-(tetrahydro-2H-pyran-4-yl)-1,2,6,7-tetrahydropyrido[3,4-d]pyridazin-4-vl 2,4,6-triisopropylbenzenesulfonate

2-methyl-6-(tetrahydro-2H-pyran-4-yl)-2,3-dihydropyrido[3,4-d]pyridazine-1,4,7(6H)-trione (100 mg, 0.44 mmol, 1.0 eq) and N,N-diisopropylethylamine (170 mg, 1.32 mmol, 3.0 eq) were dissolved in DCM (5 mL), and 2,4,6-triisopropylbenzenesulfonyl chloride (148 mg, 0.48 mmol, 1.1 eq) was added at 0° C. under N₂. The mixture was warmed to RT and stirred for 2 hrs and concentrated. The crude mixture was partitioned between sodium bicarbonate saturated solution (50 mL) and DCM (150 mL). The organic phase was dried, concentrated, and purified by flash column chromatography (10-20% ethyl acetate and 1% DCM in Petroleum ether) to afford 2-methyl-1,7-dioxo-6-(tetrahydro-2H-pyran-4-yl)-1,2,6,7-tetrahydropyrido[3,4-d]pyridazin-4-yl 2,4,6-triisopropylbenzenesulfonate (159 mg, 0.29 mmol, 66% yield) as a yellow solid. MS obsd. (ESI⁺): 544.5 [(M+H)⁺].

### Step F: (R)-2-methyl-4-((1-(2-methyl-3-(trifluoromethyl)phenyl)ethyl)amino)-6-(tetrahydro-2H-pyran-4-yl)-2,6-dihydropyrido[3,4-d]pyridazine-1,7-dione

2-methyl-1,7-dioxo-6-(tetrahydro-2H-pyran-4-yl)-1,2,6,7-tetrahydropyrido[3,4-d]pyridazin-4-yl 2,4,6-triisopropylbenzenesulfonate (100 mg, 0.18 mmol, 1.0 eq), (R)-1-(2-methyl-3-(trifluoromethyl)phenyl)ethan-1-amine (43 mg, 0.216 mmol, 1.2 eq), XantPhos Pd G3 (15.13 mg, 0.018 mmol, 0.1 eq) and Cs₂CO₃ (180 mg, 0.55 mmol, 2.0 eq) were dissolved in 1,4-dioxane (8 mL) under N₂. The mixture was stirred at 100° C. for 16 hrs. The mixture was then filtered and concentrated. The crude residue was pre-purified by preparative TLC (3% MeOH in DCM) to obtain an impure product (40 mg, yellow solid). The product was further purified by reverse phase HPLC to afford (R)-2-methyl-4-((1-(2-methyl-3-(trifluoromethyl)phenyl)ethyl)amino)-6-(tetrahydro-2H-pyran-4-yl)-2,6-dihydropyrido[3,4-d]pyridazine-1,7-dione (25 mg, 0.054 mmol, 29% yield) as a yellow solid. ¹H NMR (400 MHz, DMSO-d6) dppm: 8.88 (s, 1H), 7.71 (d, J=7.8 Hz, 1H), 7.53 (d, J=7.8 Hz, 1H), 7.38 (d, J=7.8 Hz, 1H), 7.30 (d, J=6.4 Hz, 1H), 6.86 (s, 1H), 5.16 (m, 2H), 4.19-4.03 (m, 2H), 3.55 (s, 2H), 3.24 (s, 3H), 2.54 (s, 3H), 2.10 (m, 2H), 1.82 (m, 2H), 1.48 (d, J=6.8 Hz, 3H). MS obsd. (ESI⁺): 463.4 [(M+H)⁺].

### Example 5: (R)-2-methyl-6-(tetrahydro-2H-pyran-4-yl)-4-((1-(3-(trifluoromethyl)phenyl)ethyl)amino)-2,6-dihydropyrido[3,4-d]pyridazine-1,7-dione

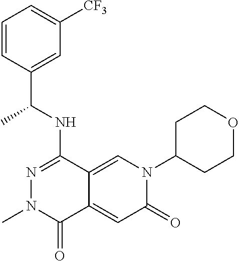 [SMILES: C[C@@H](Nc1nn(C)c(=O)c2cc(=O)n(C3CCOCC3)cc12)c1cccc(C(F)(F)F)c1]

A solution of (2-methyl-1,7-dioxo-6-tetrahydropyran-4-yl-pyrido[3,4-d]pyridazin-4-yl) 2,4,6-triisopropylbenzenesulfonate (150 mg, 0.28 mmol, 1.0 eq), Cs₂CO₃ (269.83 mg, 0.83 mmol, 3.0 eq), Xantphos Pd G3 (26.17 mg, 0.03 mmol, 0.1 eq), and (1R)-1-[3-(trifluoromethyl)phenyl]ethanamine (78.29 mg, 0.41 mmol, 1.5 eq) in 1,4-dioxane (5 mL) was stirred at 100° C. for 16 hrs. The mixture was then cooled to RT, filtered through a pad of celite and concentrated, and purified by preparative HPLC to afford (R)-2-methyl-6-(tetrahydro-2H-pyran-4-yl)-4-((1-(3-(trifluoromethyl)phenyl)ethyl)amino)-2,6-dihydropyrido[3,4-d]pyridazine-1,7-dione (34 mg, 0.07 mmol, 28% yield). 1H NMR (400 MHz, CDCl3) δ 8.01 (s, 1H), 7.66 (s, 1H), 7.59 (d, J=7.4 Hz, 1H), 7.52 (d, J=7.6 Hz, 1H), 7.45 (t, J=7.6 Hz, 1H), 7.37 (s, 1H), 5.34 (m, 1H), 5.03 (m, 1H), 4.13 (m, 2H), 3.62 (m, 2H), 3.46 (s, 3H), 1.96 (s, 3H), 1.61 (d, J=6.6 Hz, 5H). MS obsd. (ESI⁺): 449.5 [(M+H)⁺].

### Example 6: (R)-6-cyclopropyl-8-methoxy-2-methyl-4-((1-(2-methyl-3-(trifluoromethyl)phenyl)ethyl)amino)-2,6-dihydropyrido[3,4-d]pyridazine-1,7-dione

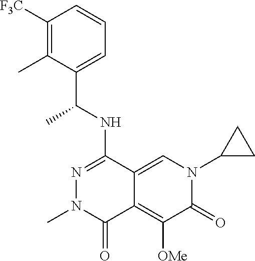 [SMILES: COc1c(=O)n(C2CC2)cc2c(N[C@H](C)c3cccc(C(F)(F)F)c3C)nn(C)c(=O)c12]

### Step A: 8-bromo-6-cyclopropyl-2-methyl-2,3-dihydropyrido[3,4-d]pyridazine-1,4,7(6H)-trione

6-cyclopropyl-2-methyl-2,3-dihydropyrido[3,4-d]pyridazine-1,4,7(6H)-trione (2 g, 8.6 mmol, 1.0 eq) was dissolved in acetic acid (40 mL). To the reaction was added potassium acetate (706 mg, 8.6 mmol, 1.0 eq) and bromine (1.6 g, 10.3 mmol, 1.2 eq). The solution was stirred at 70° C. for 3 hrs. The solution was concentrated and the crude mixture was suspended in ethyl acetate (20 mL) and filtered to afford 8-bromo-6-cyclopropyl-2-methyl-2,3-dihydropyrido[3,4-d]pyridazine-1,4,7(6H)-trione (1.66 g, 2.11 mmol, 62% yield) as a yellow solid, which was used without further purification. MS obsd. (ESI⁺): 312.2/314.2 (⁷⁹Br/⁸¹Br) [(M+H)⁺]

### Step B: 6-cyclopropyl-8-methoxy-2-methyl-2,3-dihydropyrido[3,4-d]pyridazine-1,4,7(6H)-trione

8-bromo-6-cyclopropyl-2-methyl-2,3-dihydropyrido[3,4-d]pyridazine-1,4,7(6H)-trione (100 mg, 0.32 mmol, 1.0 eq) was dissolved in MeOH (5 mL) and sodium methoxide (259.2 mg, 4.8 mmol, 15.0 eq) was added. The solution was stirred at 90° C. for 15 hrs. The mixture was then filtered, the organic phase was concentrated, and purified by flash column chromatography (10% MeOH in DCM) to afford the product 6-cyclopropyl-8-methoxy-2-methyl-2,3-dihydropyrido[3,4-d]pyridazine-1,4,7(6H)-trione (70 mg, 0.27 mmol, 83% yield) as a yellow solid. MS obsd. (ESI⁺): 264.3 [(M+H)⁺].

### Step C: 6-cyclopropyl-8-methoxy-2-methyl-1,7-dioxo-1,2,6,7-tetrahydropyrido[3,4-d]pyridazin-4-yl 2,4,6-triisopropylbenzenesulfonate

6-cyclopropyl-8-methoxy-2-methyl-2,3-dihydropyrido[3,4-d]pyridazine-1,4,7(6H)-trione (1 g, 3.80 mmol, 1.0 eq) and N,N-diisopropylethylamine (7.60 mmol, 982 mg, 2 eq) were dissolved in DCM (20 mL), and 2,4,6-triisopropylbenzenesulfonyl chloride (1.265 g, 4.18 mmol, 1.1 eq) was added at 0° C. under N₂. The mixture was warmed to RT and stirred for 2 hrs. The solution was concentrated and the crude mixture was washed with sodium bicarbonate saturated solution (50 mL) and extracted with DCM (150 mL). The organic phase was dried, concentrated, and purified by flash column chromatography (10-20% ethyl acetate and 1% DCM in petroleum ether) to afford 6-cyclopropyl-8-methoxy-2-methyl-1,7-dioxo-1,2,6,7-tetrahydropyrido[3,4-d]pyridazin-4-yl 2,4,6-triisopropylbenzenesulfonate (1.228 g, 2.32 mmol, 61% yield) as a yellow solid. MS obsd. (ESI⁺): 530.2 [(M+H)⁺].

### Step D: (R)-6-cyclopropyl-8-methoxy-2-methyl-4-((1-(2-methyl-3-(trifluoromethyl)phenyl)ethyl)amino)-2,6-dihydropyrido[3,4-d]pyridazine-1,7-dione

6-cyclopropyl-8-methoxy-2-methyl-1,7-dioxo-1,2,6,7-tetrahydropyrido[3,4-d]pyridazin-4-yl 2,4,6-triisopropylbenzenesulfonate (600 mg, 1.13 mmol, 1.0 eq), (R)-1-(2-methyl-3-(trifluoromethyl)phenyl)ethan-1-amine (276 mg, 1.36 mmol, 1.2 eq), XantPhos Pd G3 (189 mg, 0.23 mmol, 0.2 eq) and Cs₂CO₃ (1.11 g, 3.40 mmol, 3.0 eq) were dissolved in 1,4-dioxane (25 mL) under N₂. The mixture was stirred at 110° C. for 16 hrs. The resulting mixture was filtered and the organic phase was concentrated. The crude residue was pre-purified by preparative-TLC (3% MeOH in DCM) to afford an impure product residue (110 mg). The product was further purified by reverse phase HPLC to afford (R)-6-cyclopropyl-8-methoxy-2-methyl-4-((1-(2-methyl-3-(trifluoromethyl)phenyl)ethyl)amino)-2,6-dihydropyrido[3,4-d]pyridazine-1,7-dione (59 mg, 0.13 mmol, 12% yield) as a yellow solid. ¹H NMR (400 MHz, DMSO-d6) dppm: 8.37 (s, 1H), 7.71 (d, J=7.8 Hz, 1H), 7.52 (d, J=7.4 Hz, 1H), 7.34 (t, J=7.8 Hz, 1H), 7.05 (d, J=6.4 Hz, 1H), 5.22-5.03 (m, 1H), 3.75 (s, 3H), 3.60-3.45 (m, 1H), 3.17 (s, 3H), 2.52 (s, 3H), 1.45 (d, J=6.8 Hz, 3H), 1.18-1.10 (m, 2H), 1.08 (m, 2H). MS obsd. (ESI⁺): 449.2 [(M+H)⁺].

### Example 7: (R)-6-cyclopropyl-2,8-dimethyl-4-((1-(2-methyl-3-(trifluoromethyl)phenyl)ethyl)amino)-2,6-dihydropyrido[3,4-d]pyridazine-1,7-dione

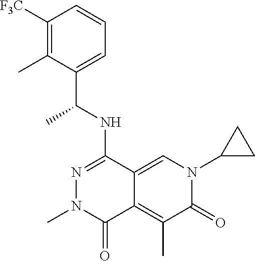 [SMILES: Cc1c([C@@H](C)Nc2nn(C)c(=O)c3c(C)c(=O)n(C4CC4)cc23)cccc1C(F)(F)F]

### Step A: 1,6-cyclopropyl-2,8-dimethyl-2,3-dihydropyrido[3,4-d]pyridazine-1,4,7(6H)-trione

8-bromo-6-cyclopropyl-2-methyl-2,3-dihydropyrido[3,4-d]pyridazine-1,4,7-(6H)-trione (200 mg, 0.64 mmol, 1.0 eq), 2,4,6-trimethyl-1,3,5,2,4,6-trioxatriborinane (482.6 mg, 1.92 mmol, 3.0 eq), Pd(PPh₃)₄(74.1 mg, 0.06 mmol, 0.1 eq), and Cs₂CO₃ (626.3 mg, 1.92 mmol, 3.0 eq) were dissolved in anhydrous 1,4-dioxane (5.5 mL) under N₂. The mixture was stirred at 100° C. for 16 hrs under N₂. The mixture was cooled to RT, diluted with ethyl acetate (20 mL) and washed sequentially with H₂O (15 mL*3) and brine (15 mL*3). The organic phase was dried, concentrated, and purified by flash column chromatography (9% MeOH in DCM) to afford 6-cyclopropyl-2,8-dimethyl-2,3-dihydropyrido[3,4-d]pyridazine-1,4,7(6H)-trione (80 mg, 0.32 mmol, 46% yield) as a yellow solid. MS obsd. (ESI⁺): 248.1 [(M+H)⁺].

### Step B: 6-cyclopropyl-2,8-dimethyl-1,7-dioxo-1,2,6,7-tetrahydropyrido[3,4-d]pyridazin-4-yl 2,4,6-triisopropylbenzenesulfonate

To a mixture of 6-cyclopropyl-2,8-dimethyl-2,3-dihydropyrido[3,4-d]pyridazine-1,4,7(6H)-trione (90 mg, 0.36 mmol, 1.0 eq) in DCM (4 mL) was added DIPEA (141.13 mg, 1.09 mmol, 3.0 eq) and 2,4,6-triisopropylbenzenesulfonyl chloride (110.24 mg, 0.36 mmol, 1.0 eq) at 0° C. under N₂. The mixture was stirred at RT for 2 hrs. The mixture was diluted by DCM (10 mL), washed with H₂O (8 ml*3), brine (6 mL*3). The organic phase was dried, concentrated, and purified by preparative-TLC (25% ethyl actetate in petroleum ether, Rf=0.4) to provide 6-cyclopropyl-2,8-dimethyl-1,7-dioxo-1,2,6,7-tetrahydropyrido[3,4-d]pyridazin-4-yl 2,4,6-triisopropylbenzenesulfonate (110 mg, 0.21 mmol, 59% yield) as a yellow gum. MS obsd. (ESI⁺): 514.4 [(M+H)⁺].

### Step C: (R)-6-cyclopropyl-2,8-dimethyl-4-((1-(2-methyl-3-(trifluoromethyl)phenyl)ethyl)amino)-2,6-dihydropyrido[3,4-d]pyridazine-1,7-dione

A mixture of 6-cyclopropyl-2,8-dimethyl-1,7-dioxo-1,2,6,7-tetrahydropyrido[3,4-d]pyridazin-4-yl 2,4,6-triisopropylbenzenesulfonate (60 mg, 0.12 mmol, 1.0 eq), (R)-1-(2-methyl-3-(trifluoromethyl)phenyl)ethan-1-amine hydrochloride (28.48 mg, 0.14 mmol, 1.2 eq), XantPhos-Pd-G3 (11.23 mg, 0.01 mmol, 0.1 eq), and Cs₂CO₃ (114.18 mg, 0.35 mmol, 3.0 eq) was dissolved in dry 1,4-dioxane (0.4 mL) under N₂. The mixture was stirred at 100° C. for 16 hrs. The mixture was cooled to RT, diluted with ethyl acetate (3 mL), and washed sequentially with H₂O (3 mL*3) and brine (2 mL*3). The organic layer was dried, concentrated, and purified by preparative-TLC (3% MeOH in DCM, Rf=0.2) followed by reverse phase HPLC to provide (R)-6-cyclopropyl-2,8-dimethyl-4-((1-(2-methyl-3-(trifluoromethyl)phenyl)ethyl)amino)-2,6-dihydropyrido[3,4-d]pyridazine-1,7-dione (5 mg, 0.01 mmol, 10% yield) as a yellow solid. MS obsd. (ESI⁺): 433.3 [(M+H)⁺].

### Example 8: (R)-6-cyclopropyl-2-ethyl-4-((1-(2-methyl-3-(trifluoromethyl)phenyl)ethyl)amino)-2,6-dihydropyrido[3,4-d]pyridazine-1,7-dione

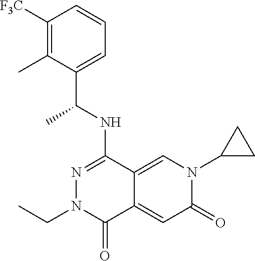 [SMILES: CCn1nc(N[C@H](C)c2cccc(C(F)(F)F)c2C)c2cn(C3CC3)c(=O)cc2c1=O]

### Step A: lithium 5-(tert-butoxycarbonyl)-1-cyclopropyl-2-oxo-1,2-dihydropyridine-4-carboxylate

To a solution of 3-(tert-butyl) 4-methyl 1-cyclopropyl-6-oxo-1,6-dihydropyridine-3,4-dicarboxylate (150 mg, 0.51 mmol, 1.0 eq.) in MeOH (5.0 mL) and THE (5.0 mL) was added lithium hydroxide (37 mg, 1.53 mmol, 3.0 eq.) in water (5.0 mL) at RT. The mixture was stirred for 16 hrs at RT. The mixture was concentrated to obtain lithium 5-(tert-butoxycarbonyl)-1-cyclopropyl-2-oxo-1,2-dihydropyridine-4-carboxylate (190 mg, crude) as a yellow solid. The residue was used for the next step without further purification. MS obsd. (ESI⁺): 280.3 [(M+H)⁺].

### Step B: Tert-butyl 4-(2-(tert-butoxycarbonyl)-1-ethylhydrazine-1-carbonyl)-1-cyclopropyl-6-oxo-1,6-dihydropyridine-3-carboxylate

To a solution of lithium 5-(tert-butoxycarbonyl)-1-cyclopropyl-2-oxo-1,2-dihydropyridine-4-carboxylate (190 mg, crude) in N,N-dimethylformamide (3.0 mL) was added N-ethyl-N-isopropyl-propan-2-amine (264 mg, 2.04 mmol, 355.48 uL) and HATU (390 mg, 1.02 mmol). The mixture was stirred for 15 minutes at RT and then tert-butyl 2-ethylhydrazine-1-carboxylate (142 mg, 0.89 mmol) was added. The mixture was stirred for 16 hrs at RT. Water was added and the mixture was extracted with DCM (10 mL×3). The combined organic phases were washed with water (10 mL), brine (10 mL), and then dried over sodium sulfate. The organic phase was filtered, concentrated and purified by flash column chromatography (0-70% ethyl acetate in petroleum ether) to afford tert-butyl 4-(2-(tert-butoxycarbonyl)-1-ethylhydrazine-1-carbonyl)-1-cyclopropyl-6-oxo-1,6-dihydropyridine-3-carboxylate (150 mg, 0.36 mmol, 52% yield) as a light-yellow solid. MS obsd. (ESI⁺): 422.4 [(M+H)⁺].

### Step C: 6-cyclopropyl-2-ethyl-2,3-dihydropyrido[3,4-d]pyridazine-1,4,7(6H)-trione

To a solution of tert-butyl 4-(2-(tert-butoxycarbonyl)-1-ethylhydrazine-1-carbonyl)-1-cyclopropyl-6-oxo-1,6-dihydropyridine-3-carboxylate (150 mg, 0.36 mmol, 1.0 eq.) in 1,4-dioxane (1.0 mL) was added HCl (4 M in 1,4-dioxane, 3.0 mL) at RT. The mixture was stirred for 1 hr at RT. The temperature was raised to 100° C. and stirred for 0.5 hrs. The mixture was cooled to RT and the solid was filtered to afford 6-cyclopropyl-2-ethyl-2,3-dihydropyrido[3,4-d]pyridazine-1,4,7(6H)-trione (60 mg, 0.24 mmol, 68% yield) as a yellow solid. MS obsd. (ESI⁺): 248.4 [(M+H)⁺].

### Step D: 6-cyclopropyl-2-ethyl-1,7-dioxo-1,2,6,7-tetrahydropyrido[3,4-d]pyridazin-4-yl 2,4,6-triisopropylbenzenesulfonate

To a solution of 6-cyclopropyl-2-ethyl-2,3-dihydropyrido[3,4-d]pyridazine-1,4,7(6H)-trione (60 mg, 0.24 mmol, 1.0 eq.) in DCM (12.0 mL) was added N,N-Diisopropylethylamine (125 mg, 0.97 mmol, 169.07 uL, 4.0 eq.) and 2,4,6-triisopropylbenzenesulfonyl chloride (81 mg, 0.27 mmol, 1.1 eq.). The mixture was stirred for 3 hrs at RT. The reaction was concentrated and diluted with DCM and water. The mixture was extracted with DCM (10 mL×3). The combined organic phases were washed with water (20 mL), brine (20 mL), and dried over sodium sulfate. The organic phase was filtered, concentrated and purified by flash column chromatography (0-40% ethyl acetate in petroleum ether) to afford 6-cyclopropyl-2-ethyl-1,7-dioxo-1,2,6,7-tetrahydropyrido[3,4-d]pyridazin-4-yl 2,4,6-triisopropylbenzenesulfonate (120 mg, 0.23 mmol, 96% yield) as a yellow solid. MS obsd. (ESI⁺): 514.6 [(M+H)⁺].

### Step E: (R)-6-cyclopropyl-2-ethyl-4-((1-(2-methyl-3-(trifluoromethyl)phenyl)ethyl)amino)-2,6-dihydropyrido[3,4-d]pyridazine-1,7-dione

A mixture of 6-cyclopropyl-2-ethyl-1,7-dioxo-1,2,6,7-tetrahydropyrido[3,4-d]pyridazin-4-yl 2,4,6-triisopropylbenzenesulfonate (100 mg, 0.19 mmol, 1.0 eq.), (1R)-1-[2-methyl-3-(trifluoromethyl)phenyl]ethanamine hydrochloride (119 mg, 0.58 mmol, 3.0 eq.), Xantphos-Pd-G3 (64 mg, 0.08 mmol, 0.4 eq.) and Cs₂CO₃ (571 mg, 1.75 mmol, 9.0 eq.) was dissolved in 1,4-dioxane (16.0 mL) under N₂. The mixture was stirred at 110° C. for 16 hrs. The mixture was and purified by flash column chromatography (0-15% MeOH in DCM) to obtain a crude product. The crude product was furthur purified by reverse phase HPLC to afford (R)-6-cyclopropyl-2-ethyl-4-((1-(2-methyl-3-(trifluoromethyl)phenyl)ethyl)amino)-2,6-dihydropyrido[3,4-d]pyridazine-1,7-dione (3.5 mg, 0.008 mmol, 4% yield) as a yellow solid. MS obsd. (ESI⁺): 433.3 [(M+H)⁺].

### Example 9: (R)-6-(1-acetylpiperidin-4-yl)-2-methyl-4-((1-(2-methyl-3-(trifluoromethyl)phenyl)ethyl)amino)-2,6-dihydropyrido[3,4-d]pyridazine-1,7-dione

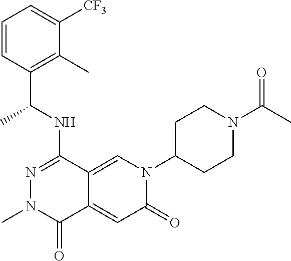 [SMILES: CC(=O)N1CCC(n2cc3c(N[C@H](C)c4cccc(C(F)(F)F)c4C)nn(C)c(=O)c3cc2=O)CC1]

### Step A: 3-(tert-butyl) 4-methyl 1-(1-acetylpiperidin-4-yl)-6-oxo-1,6-dihydropyridine-3,4-dicarboxylate

A mixture of 1-(4-amino-1-piperidyl)ethanone (1 g, 7.03 mmol, 1.21 mL) and tert-butyl prop-2-ynoate (887.16 mg, 7.03 mmol) in ethanol (40 mL) and water (0.040 mL) was stirred at RT for 18 hrs. Then, dimethyl but-2-ynedioate (999.37 mg, 7.03 mmol) was added dropwise and the mixture was heated to 85° C. for 2 days. The crude mixture was cooled to RT and was treated with sodium methoxide (378.17 mg, 7.00 mmol). The resulting dark suspension was stirred at RT for 15 min., concentrated, and purified by flash column chromatography (0-100% ethyl acetate in cyclohexane, followed by 0-10% MeOH in ethyl acetate) to give 3-(tert-butyl) 4-methyl 1-(1-acetylpiperidin-4-yl)-6-oxo-1,6-dihydropyridine-3,4-dicarboxylate as a yellow foam (0.95 g, 74% purity) which was used as is for the following step assuming 100% purity. MS obsd. (ESI⁺): 323.2 [(M-tBu)⁺].

### Step B: 1-(1-acetylpiperidin-4-yl)-5-(tert-butoxycarbonyl)-2-oxo-1,2-dihydropyridine-4-carboxylic acid

3-(tert-Butyl) 4-methyl 1-(1-acetylpiperidin-4-yl)-6-oxo-1,6-dihydropyridine-3,4-dicarboxylate (0.95 g, 74% purity) and lithium hydroxide (1 M aqueous solution, 5.02 mL) in MeOH (10 mL) were stirred at RT for 1 hr. The MeOH was evaporated under reduced pressure and the residue was washed with ethyl acetate (20 mL) and water (20 mL). The aqueous phase was acidified with 1 M HCl solution (5 mL) and extracted with ethyl acetate (2×20 mL). The organic phase was dried over sodium sulfate and concentrated to afford 1-(1-acetylpiperidin-4-yl)-5-(tert-butoxycarbonyl)-2-oxo-1,2-dihydropyridine-4-carboxylic acid as a light yellow solid (540 mg, 1.48 mmol, 21% over 2 steps). MS obsd. (ESI⁻): 363.2 [(M−H)⁻].

### Step C: tert-Butyl 1-(1-acetylpiperidin-4-yl)-4-(2-(tert-butoxycarbonyl)-1-methylhydrazine-1-carbonyl)-6-oxo-1,6-dihydropyridine-3-carboxylate

A mixture of 1-(1-acetylpiperidin-4-yl)-5-(tert-butoxycarbonyl)-2-oxo-1,2-dihydropyridine-4-carboxylic acid (0.54 g, 1.48 mmol), N,N-diisopropylethylamine (229.83 mg, 1.78 mmol, 309.74 uL) and HATU (676.16 mg, 1.78 mmol) in 2-Me-THF (10 mL) was treated with tert-butyl 2-methylhydrazine-1-carboxylate (259.97 mg, 1.78 mmol) and the mixture was stirred at RT for 2 hrs. The solution was concentrated and the residue was washed with ethyl acetate (150 mL) and sodium bicarbonate saturated solution (50 mL). The organic phase was washed with brine (20 mL), dried, concentrated, and purified by flash column chromatography (10-100% ethyl acetate in cyclohexane) to afford tert-butyl 1-(1-acetylpiperidin-4-yl)-4-(2-(tert-butoxycarbonyl)-1-methylhydrazine-1-carbonyl)-6-oxo-1,6-dihydropyridine-3-carboxylate as a light yellow solid (360 mg, 0.73 mmol, 49% yield). MS obsd. (ESI⁻): 491.4 [(M−H)⁻].

### Step D: 6-(1-acetylpiperidin-4-yl)-2-methyl-2,3-dihydropyrido[3,4-d]pyridazine-1,4,7(6H)-trione

A mixture of tert-butyl 1-(1-acetylpiperidin-4-yl)-4-(2-(tert-butoxycarbonyl)-1-methylhydrazine-1-carbonyl)-6-oxo-1,6-dihydropyridine-3-carboxylate (360 mg, 0.73 mmol) and hydrogen chloride in 1,4-dioxane (4 M, 14.6 mmol, 3.65 mL) was stirred at RT for 15 minutes then heated at 100° C. for 1 hr. The mixture was cooled to RT and filtered to give 6-(1-acetylpiperidin-4-yl)-2-methyl-2,3-dihydropyrido[3,4-d]pyridazine-1,4,7(6H)-trione (165 mg, 0.52 mmol, 71% yield) as a yellow solid. MS obsd. (ESI⁺): 319.2 [(M+H)⁺].

### Step E: 6-(1-acetylpiperidin-4-yl)-2-methyl-1,7-dioxo-1,2,6,7-tetrahydropyrido[3,4-d]pyridazin-4-yl trifluoromethanesulfonate

A mixture of 6-(1-acetylpiperidin-4-yl)-2-methyl-2,3-dihydropyrido[3,4-d]pyridazine-1,4,7(6H)-trione (80 mg, 0.25 mmol) and pyridine (29.82 mg, 0.38 mmol, 30.49 uL) in DCM (1.5 mL) was cooled to 0° C. under N₂ atmosphere. A solution of triflic anhydride (106.36 mg, 0.38 mmol, 63.31 uL) in DCM (1.5 mL) was added and the mixture was stirred at 0° C. for 30 min. At this time, additional pyridine (29.82 mg, 0.38 mmol, 30.49 uL) and triflic anhydride (106.36 mg, 0.38 mmol, 63.31 uL) were added and the mixture was stirred at 0° C. for an additional 30 min. The mixture was diluted with DCM (20 mL) and quenched with water (5 mL). The organic phase was dried, concentrated, and purified by flash column chromatography (0-100% ethyl acetate in cyclohexane, then 0-10% MeOH in ethyl acetate) to yield 6-(1-acetylpiperidin-4-yl)-2-methyl-1,7-dioxo-1,2,6,7-tetrahydropyrido[3,4-d]pyridazin-4-yl trifluoromethanesulfonate as a yellow solid (31 mg, 0.07 mmol, 27%). MS obsd. (ESI⁺): 451.3 [(M+H)⁺].

### Step F: (R)-6-(1-acetylpiperidin-4-yl)-2-methyl-4-((1-(2-methyl-3-(trifluoromethyl)phenyl)ethyl)amino)-2,6-dihydropyrido[3,4-d]pyridazine-1,7-dione

A mixture of tris(dibenzylideneacetone)dipalladium(0) (6.89 mg, 6.66 μmol), Cs₂CO₃ (43.41 mg, 133.22 μmol) and Xantphos (7.7 mg) in 1,4-dioxane (1 mL) was degassed with N₂ for 5 min., then stirred at 100° C. for 1 hr before a mixture of 6-(1-acetylpiperidin-4-yl)-2-methyl-1,7-dioxo-1,2,6,7-tetrahydropyrido[3,4-d]pyridazin-4-yl trifluoromethanesulfonate (30 mg, 0.067 mmol) and (R)-1-(2-methyl-3-(trifluoromethyl)phenyl)ethan-1-amine (16.24 mg, 0.080 mmol) in 1,4-dioxane (1 mL) was added and the mixture was stirred at 100° C. for a further 19 hrs. The mixture was cooled to RT, concentrated and purified by preparative HPLC to afford (R)-6-(1-acetylpiperidin-4-yl)-2-methyl-4-((1-(2-methyl-3-(trifluoromethyl)phenyl)ethyl)amino)-2,6-dihydropyrido[3,4-d]pyridazine-1,7-dione as a yellow solid (1.5 mg, 0.003 mmol, 4%). MS obsd. (ESI⁺) 504.3 [(M+H)⁺].

### Example 10: (R)-6-cyclopropyl-2-methyl-4-((1-(2-methyl-3-(trifluoromethyl)phenyl)ethyl)amino)-1,7-dioxo-1,2,6,7-tetrahydropyrido[3,4-d]pyridazine-8-carbonitrile

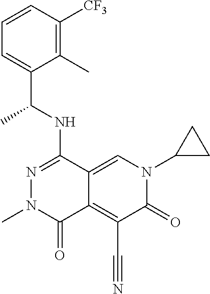 [SMILES: Cc1c([C@@H](C)Nc2nn(C)c(=O)c3c(C#N)c(=O)n(C4CC4)cc23)cccc1C(F)(F)F]

A mixture of (R)-8-bromo-6-cyclopropyl-2-methyl-4-((1-(2-methyl-3-(trifluoromethyl)phenyl)ethyl)amino)-2,6-dihydropyrido[3,4-d]pyridazine-1,7-dione (20 mg, 40 umol, 1 equiv), 1,1′-Bis(diphenylphosphino)ferrocene (22.2 mg, 40 umol, 1 equiv), Tris(dibenzylideneacetone)dipalladium (0) (36.7 mg, 40 umol, 1 equiv), and zinc cyanide (7 mg, 60 umol, 1.2 equiv) in DMF (0.2 mL) was stirred at 120° C. under nitrogen for 16 hours. The mixture was then cooled to room temperature, and partitioned between water and ethyl acetate. The organic phase was dried over sodium sulfate, filtered, and concentrated. The crude product was combined with the crude product derived from an identical reaction starting with 35 mg (R)-8-bromo-6-cyclopropyl-2-methyl-4-((1-(2-methyl-3-(trifluoromethyl)phenyl)ethyl)amino)-2,6-dihydropyrido[3,4-d]pyridazine-1,7-dione. The combined crude material was purified by preparative TLC (30:1 DCM/MeOH) and further purified via reverse phase HPLC to obtain (R)-6-cyclopropyl-2-methyl-4-((1-(2-methyl-3-(trifluoromethyl)phenyl)ethyl)amino)-1,7-dioxo-1,2,6,7-tetrahydropyrido[3,4-d]pyridazine-8-carbonitrile (8 mg, 17.4 umol, 16% yield). MS obsd. (ESI+) 444.2 [(M+H)⁺].

### Example 11: (R)-8-chloro-6-cyclopropyl-2-methyl-4-((1-(2-methyl-3-(trifluoromethyl)phenyl)ethyl)amino)-2,6-dihydropyrido[3,4-d]pyridazine-1,7-dione

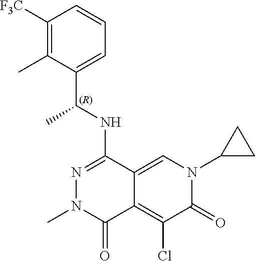

(R)-6-cyclopropyl-2-methyl-4-((1-(2-methyl-3-(trifluoromethyl)phenyl)ethyl)amino)-2,6-dihydropyrido[3,4-d]pyridazine-1,7-dione (70 mg, 167 umol) and 1-chloropyrrolidine-2,5-dione (24.57 mg, 184 umol) were dissolved in CH₃CN (5 mL). The mixture was heated to 70° C. and stirred for 1 hr. The reaction mixture was concentrated under reduced pressure and purified by reverse phase HPLC to afford (R)-8-chloro-6-cyclopropyl-2-methyl-4-((1-(2-methyl-3-(trifluoromethyl)phenyl)ethyl)amino)-2,6-dihydropyrido[3,4-d]pyridazine-1,7-dione (example 11, 47 mg, 62% yield). MS obsd. (ESI+): 453.2 (M+H)+.

### Example 12: (R)-8-amino-6-cyclopropyl-2-methyl-4-((1-(2-methyl-3-(trifluoromethyl)phenyl)ethyl)amino)-2,6-dihydropyrido[3,4-d]pyridazine-1,7-dione

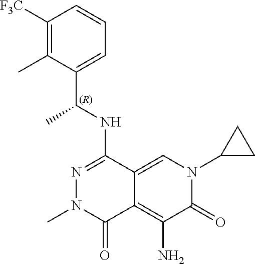

(R)-8-bromo-6-cyclopropyl-2-methyl-4-((1-(2-methyl-3-(trifluoromethyl)phenyl)ethyl)amino)-2,6-dihydropyrido[3,4-d]pyridazine-1,7-dione (30 mg, 60 umol) was dissolved in NH₃ (7M in MeOH, 5 mL). The mixture was heated to 110° C. and stirred for 16 hrs. The solvent was removed under reduced pressure and the crude compound was purified by reverse phase HPLC to afford (R)-8-amino-6-cyclopropyl-2-methyl-4-((1-(2-methyl-3(trifluoromethyl)phenyl)ethyl)amino)-2,6-dihydropyrido[3,4-d]pyridazine-1,7-dione (5 mg, 19% yield). MS obsd. (ESI⁺): 434.2 (M+H)⁺.

### Example 13: 6-((1r,3r)-3-hydroxycyclobutyl)-2-methyl-4-(((R)-1-(2-methyl-3-(trifluoromethyl)phenyl)ethyl)amino)-2,6-dihydropyrido[3,4-d]pyridazine-1,7-dione

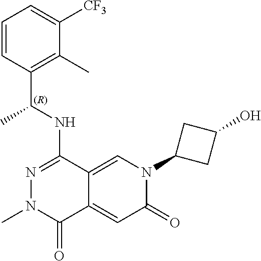

### Step A: 3-(tert-butyl) 4-methyl 1-((1r,3r)-3-hydroxycyclobutyl)-6-oxo-1,6-dihydropyridine-3,4-dicarboxylate

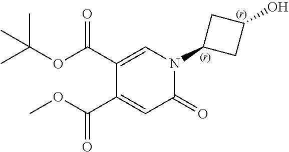 [SMILES: COC(=O)c1cc(=O)n([C@H]2C[C@H](O)C2)cc1C(=O)OC(C)(C)C]

To a solution of trans-3-aminocyclobutanol (2.00 g, 22.96 mmol) in EtOH (50 mL) was added tert-butyl prop-2-ynoate (3.04 g, 24.10 mmol, 3.31 mL) and DIPEA (12.0 mL, 8.90 g, 68.87 mmol). The mixture was stirred at room temperature for 16 hrs and dimethyl but-2-ynedioate (3.59 g, 25.25 mmol, 3.09 mL) was added to the reaction. The reaction mixture was stirred at 80° C. for a further 16 hrs. The reaction mixture was concentrated under vacuum and the residue was purified by silica gel chromatography (eluting with PE:EtOAc=3:1, v/v) to afford 3-(tert-butyl) 4-methyl 1-((1r,3r)-3-hydroxycyclobutyl)-6-oxo-1,6-dihydropyridine-3,4-dicarboxylate (3.00 g, 34% yield, 85% purity) MS obsd. (ESI⁺): 324.4 (M+H)⁺.

### Step B: 5-(tert-butoxycarbonyl)-1-((1r,3r)-3-hydroxycyclobutyl)-2-oxo-1,2-dihydropyridine-4-carboxylic acid

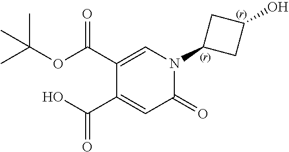 [SMILES: CC(C)(C)OC(=O)c1cn([C@H]2C[C@H](O)C2)c(=O)cc1C(=O)O]

To a solution of 3-(tert-butyl) 4-methyl 1-((1r,3r)-3-hydroxycyclobutyl)-6-oxo-1,6-dihydropyridine-3,4-dicarboxylate (2.5 g, 6.56 mmol, 85% purity) in MeOH (20 mL), THE (20 mL) and H₂O (20 mL) was added LiOH (555 mg, 23.20 mmol). The mixture was stirred at 20° C. for 3 hrs. The mixture was concentrated in vacuum to afford 5-(tert-butoxycarbonyl)-1-((1r,3r)-3-hydroxycyclobutyl)-2-oxo-1,2-dihydropyridine-4-carboxylic acid (2.1 g, crude) as yellow solid, which was used for the next step without further purification. MS obsd. (ESI+): 310.4 (M+H)⁺.

### Step C: tert-butyl 4-(2-(tert-butoxycarbonyl)-1-methylhydrazine-1-carbonyl)-1-((1r,3r)-3-hydroxycyclobutyl)-6-oxo-1,6-dihydropyridine-3-carboxylate

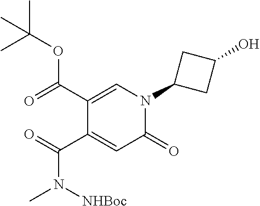 [SMILES: CN(NC(=O)OC(C)(C)C)C(=O)c1cc(=O)n([C@H]2C[C@H](O)C2)cc1C(=O)OC(C)(C)C]

To a solution of 5-(tert-butoxycarbonyl)-1-((1r,3r)-3-hydroxycyclobutyl)-2-oxo-1,2-dihydropyridine-4-carboxylic acid (2.4 g, crude) in DMF (3 mL) was added tert-butyl N-(methylamino)carbamate (1.59 g, 10.86 mmol), DIPEA (3.00 g, 23.28 mmol) and HATU (5.90 g, 15.52 mmol). The mixture was stirred at room temperature for 16 hrs. The reaction mixture was quenched with water (60 mL) and extracted with EtOAc (80 mL×3). The organic layers were washed with water (60 mL×3), dried over Na₂SO₄ and filtered. The filtrate was concentrated under vacuum and the residue was purified by flash column (eluting with PE:EtOAc=form 100:0 to 50:50, v/v) to afford tert-butyl 4-(2-(tert-butoxycarbonyl)-1-methylhydrazine-1-carbonyl)-1-((1r,3r)-3-hydroxycyclobutyl)-6-oxo-1,6-dihydropyridine-3-carboxylate (2 g, 50% yield, 85% purity). MS obsd. (ESI+): 438.5 (M+H)⁺.

### Step D: tert-butyl 1-((1r,3r)-3-hydroxycyclobutyl)-4-(1-methylhydrazine-1-carbonyl)-6-oxo-1,6-dihydropyridine-3-carboxylate Hydrochloride

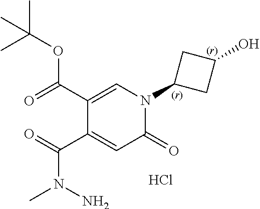 [SMILES: CN(N)C(=O)c1cc(=O)n([C@H]2C[C@H](O)C2)cc1C(=O)OC(C)(C)C ; Cl]

Tert-butyl 4-(2-(tert-butoxycarbonyl)-1-methylhydrazine-1-carbonyl)-1-((1r,3r)-3-hydroxycyclobutyl)-6-oxo-1,6-dihydropyridine-3-carboxylate (500 mg, 0.97 mmol, 85% purity) was dissolved in HCl/dioxane (4 M, 20 mL) at 0° C. The mixture was then stirred at rt for 3 hrs. The reaction mixture was concentrated under vacuum to afford tert-butyl 1-((1r,3r)-3-hydroxycyclobutyl)-4-(1-methylhydrazine-1-carbonyl)-6-oxo-1,6-dihydropyridine-3-carboxylate hydrochloride (385 mg, crude) as a yellow solid, which was used for the next step without further purification. MS obsd. (ESI+): 338.4 (M+H)⁺.

### Step E: 6-((1r,3r)-3-hydroxycyclobutyl)-2-methyl-2,3-dihydropyrido[3,4-d]pyridazine-1,4,7(6H)-trione

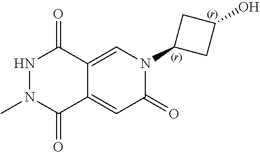 [SMILES: Cn1[nH]c(=O)c2cn([C@H]3C[C@H](O)C3)c(=O)cc2c1=O]

Tert-butyl 1-((1r,3r)-3-hydroxycyclobutyl)-4-(1-methylhydrazine-1-carbonyl)-6-oxo-1,6-dihydropyridine-3-carboxylate hydrochloride (385 mg, crude) was dissolved in NH₃/MeOH (7 M, 20 mL) at 0° C. The reaction was stirred at 20° C. for 48 hrs. The reaction mixture was concentrated in vacuum to give the residue, which was purified by flash column (eluted with PE:EtOAc=4:6, v/v) to afford 6-((1r,3r)-3-hydroxycyclobutyl)-2-methyl-2,3-dihydropyrido[3,4-d]pyridazine-1,4,7(6H)-trione (240 mg, 93% yield over 2 steps) as a yellow solid. MS obsd. (ESI+): 264.3 (M+H)⁺.

### Step F: 6-((1r,3r)-3-hydroxycyclobutyl)-2-methyl-1,7-dioxo-1,2,6,7-tetrahydropyrido[3,4-d]pyridazin-4-yl 2,4,6-triisopropylbenzenesulfonate

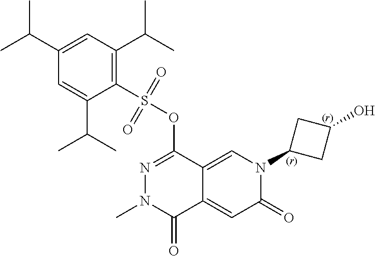 [SMILES: CC(C)c1cc(C(C)C)c(S(=O)(=O)Oc2nn(C)c(=O)c3cc(=O)n([C@H]4C[C@H](O)C4)cc23)c(C(C)C)c1]

To a solution of 6-((1r,3r)-3-hydroxycyclobutyl)-2-methyl-2,3-dihydropyrido[3,4-d]pyridazine-1,4,7(6H)-trione (100 mg, 380 umol, 1.0 eq) in DCM (5 mL) was added DIPEA (147 mg, 1.14 mmol) and 2,4,6-triisopropylbenzenesulfonyl chloride (138.0 mg, 456 umol) at 0° C. The reaction was stirred at rt for 2 hrs. The reaction was quenched with water (10 mL) and extracted with DCM (15 mL×3). The organic layers were dried over Na2SO4 and filtered. The filtrate was concentrated under vacuum and the residue was purified by flash column chromatography (eluted with PE:EtOAC=1:2, v/v) to afford 6-((1r,3r)-3-hydroxycyclobutyl)-2-methyl-1,7-dioxo-1,2,6,7-tetrahydropyrido[3,4-d]pyridazin-4-yl 2,4,6-triisopropylbenzenesulfonate (150 mg, 283 umol, 74% yield). MS obsd. (ESI+): 530.6 (M+H)+.

### Step G: (1r,3r)-3-(2-methyl-1,7-dioxo-4-(((2,4,6-triisopropylphenyl)sulfonyl)oxy)-1,7-dihydropyrido[3,4-d]pyridazin-6(2H)-yl)cyclobutyl acetate

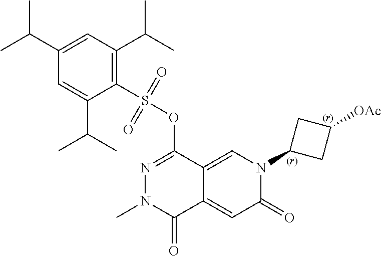 [SMILES: CC(=O)O[C@H]1C[C@H](n2cc3c(OS(=O)(=O)c4c(C(C)C)cc(C(C)C)cc4C(C)C)nn(C)c(=O)c3cc2=O)C1]

To a solution of 6-((1r,3r)-3-hydroxycyclobutyl)-2-methyl-1,7-dioxo-1,2,6,7-tetrahydropyrido[3,4-d]pyridazin-4-yl 2,4,6-triisopropylbenzenesulfonate (140 mg, 264 umol) in DCM was added DMAP (161 mg, 1.32 mmol) and Ac₂O (135 mg, 1.32 mmol) at 0° C. The mixture was stirred at 20° C. for 2 hrs. The reaction mixture was concentrated under vacuum and the residue was purified by flash column chromatography (PE:EtOAC=1:2, v/v) to afford (1r,3r)-3-(2-methyl-1,7-dioxo-4-(((2,4,6-triisopropylphenyl)sulfonyl)oxy)-1,7-dihydropyrido[3,4-d]pyridazin-6(2H)-yl)cyclobutyl acetate (120 mg, 79% yield). MS obsd. (ESI+): 572.2 (M+H)⁺.

### Step H: (1r,3r)-3-(2-methyl-4-(((R)-1-(2-methyl-3-(trifluoromethyl)phenyl)ethyl)amino)-1,7-dioxo-1,7-dihydropyrido[3,4-d]pyridazin-6(2H)-yl)cyclobutyl acetate

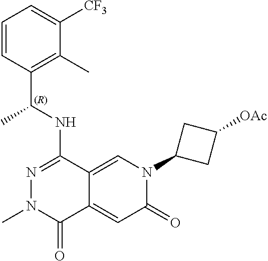

To a solution of (1r,3r)-3-(2-methyl-1,7-dioxo-4-(((2,4,6-triisopropylphenyl)sulfonyl)oxy)-1,7-dihydropyrido[3,4-d]pyridazin-6(2H)-yl)cyclobutyl acetate (64 mg, 113 umol, 1.0 eq) and (1R)-1-[2-methyl-3-(trifluoromethyl)phenyl]ethanamine (34.53 mg, 169 umol) in 1,4-dioxane (5 mL) was added Cs₂CO₃ (110.5 mg, 339 umol) and xantphos-Pd-G3 (21.5 mg, 22 umol). The mixture was then stirred at 105° C. for 1 hr under nitrogen. The reaction mixture was filtered. The filtrate was concentrated and purified by flash column chromatography (PE:EtOAC=1:2 to EtOAc, v/v) to afford (1r,3r)-3-(2-methyl-4-(((R)-1-(2-methyl-3-(trifluoromethyl)phenyl)ethyl)amino)-1,7-dioxo-1,7-dihydropyrido[3,4-d]pyridazin-6(2H)-yl)cyclobutyl acetate (30 mg, 54% yield, 84% purity). MS obsd. (ESI+): 491.5 (M+H)⁺.

### Step I: 6-((1r,3r)-3-hydroxycyclobutyl)-2-methyl-4-(((R)-1-(2-methyl-3-(trifluoromethyl)phenyl)ethyl)amino)-2,6-dihydropyrido[3,4-d]pyridazine-1,7-dione (Example 13)

To a solution of (1r,3r)-3-(2-methyl-4-(((R)-1-(2-methyl-3-(trifluoromethyl)phenyl)ethyl)amino)-1,7-dioxo-1,7-dihydropyrido[3,4-d]pyridazin-6(2H)-yl)cyclobutyl acetate (30 mg, 51 umol, 84% purity) in MeOH (3 mL) was added K₂CO₃ (17 mg, 122 umol). The mixture was stirred at 20° C. for 1 hr. The reaction mixture was then filtered. The filtrate was purified by reverse phase HPLC to afford 6-((1r,3r)-3-hydroxycyclobutyl)-2-methyl-4-(((R)-1-(2-methyl-3-(trifluoromethyl)phenyl)ethyl)amino)-2,6-dihydropyrido[3,4-d]pyridazine-1,7-dione (example 13, 6.5 mg, 28% yield). MS obsd. (ESI+): 449.2 (M+H)⁺.

### Example 14: 6-((1s,3s)-3-hydroxycyclobutyl)-2-methyl-4-(((R)-1-(2-methyl-3-(trifluoromethyl)phenyl)ethyl)amino)-2,6-dihydropyrido[3,4-d]pyridazine-1,7-dione

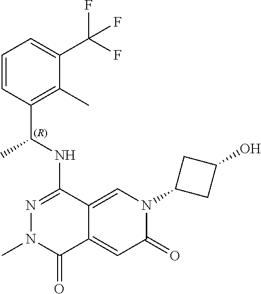

Prepared according to an essential analogous route to example 13. MS obsd. (ESI+): 449.3 (M+H)⁺.

### Example 15: (R)-2-methyl-4-((1-(2-methyl-3-(trifluoromethyl)phenyl)ethyl)amino)-6-(1-methylcyclopropyl)-2,6-dihydropyrido[3,4-d]pyridazine-1,7-dione

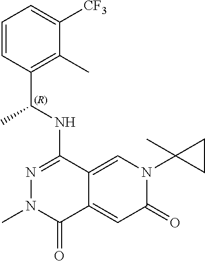

### Step A: 3-(tert-butyl) 4-methyl 1-(1-methylcyclopropyl)-6-oxo-1,6-dihydropyridine-3,4-dicarboxylate

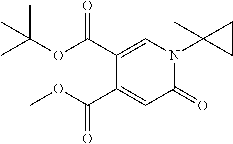 [SMILES: COC(=O)c1cc(=O)n(C2(C)CC2)cc1C(=O)OC(C)(C)C]

To a solution of 1-methylcyclopropanamine; hydrochloride (500 mg, 4.65 mmol) in EtOH (30 mL) was added sodium ethoxide (1.58 g, 4.65 mmol, 20 wt % in ethanol). The reaction was stirred for 5 h at rt. Then water (0.3 mL) and tert-butyl prop-2-ynoate (586 mg, 4.65 mmol) were added. The reaction was stirred for 48 h at rt. Then dimethyl but-2-ynedioate (1.30 g, 9.30 mmol) was added and the mixture was heated to 85° C. for 16 hrs. The solvent was removed in vacuo and the residue was purified by flash column chromatography (eluting with 0%-50% EA in PE) to afford 3-(tert-butyl) 4-methyl 1-(1-methylcyclopropyl)-6-oxo-1,6-dihydropyridine-3,4-dicarboxylate (565 mg, 40% yield, 80% purity), which was used without further purification. MS obsd. (ESI+): 308.2 (M+H)⁺.

### Step B: (R)-2-methyl-4-((1-(2-methyl-3-(trifluoromethyl)phenyl)ethyl)amino)-6-(1-methylcyclopropyl)-2,6-dihydropyrido[3,4-d]pyridazine-1,7-dione (example 15)

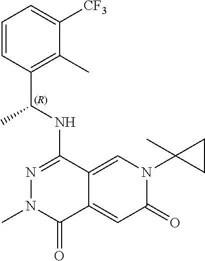

Prepared according to an analogous route as example 2. (R)-2-methyl-4-((1-(2-methyl-3-(trifluoromethyl)phenyl)ethyl)amino)-6-(1-methylcyclopropyl)-2,6-dihydropyrido[3,4-d]pyridazine-1,7-dione. MS obsd. (ESI+): 433.3 (M+H)⁺.

### Example 16: (R)-6-(bicyclo[1.1.1]pentan-1-yl)-2-methyl-4-((1-(2-methyl-3-(trifluoromethyl)phenyl)ethyl)amino)-2,6-dihydropyrido[3,4-d]pyridazine-1,7-dione

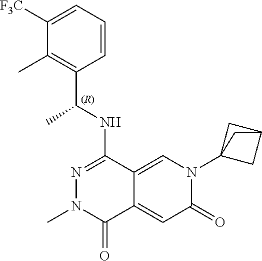

### Step A: tert-butyl (E)-3-(bicyclo[1.1.1]pentan-1-ylamino)acrylate

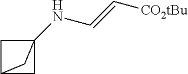 [SMILES: CC(C)(C)OC(=O)/C=C/NC12CC(C1)C2]

To a solution of bicyclo[1.1.1]pentan-1-amine HCl salt (1.22 g, 10.20 mmol) in ethanol (10 mL) was added sodium ethoxide (3.47 g, 10.20 mmol, 20 wt %,) at 0° C. and the mixture was stirred at 0° C. for 2.5 hrs. Then tert-butyl prop-2-ynoate (1.29 g, 10.20 mmol, 1.40 mL) was added and the reaction was stirred for 36 hrs at rt. Upon completion, the mixture was concentrated and purified by flash column chromatography (EA in PE=40%) to afford tert-butyl (E)-3-(bicyclo[1.1.1]pentan-1-ylamino)acrylate (621 mg, 29% yield). MS obsd. (ESI+): 210.2 (M+H)⁺.

### Step B: 3-(tert-butyl) 4-ethyl 1-(bicyclo[1.1.1]pentan-1-yl)-6-oxo-1,6-dihydropyridine-3,4-dicarboxylate

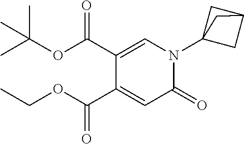 [SMILES: CCOC(=O)c1cc(=O)n(C23CC(C2)C3)cc1C(=O)OC(C)(C)C]

To a solution of tert-butyl (E)-3-(1-bicyclo[1.1.1]pentanylamino)prop-2-enoate (621 mg, 2.97 mmol, 1.0 eq) in EtOH (30 mL) was added dimethyl but-2-ynedioate (421.67 mg, 2.97 mmol, 363.51 uL, 1.0 eq) at rt and the mixture was stirred at 90° C. for 16 hrs. Then the reaction was cooled to rt and sodium methanolate (160.29 mg, 2.97 mmol, 1 eq) was added and stirred at rt for 2 hrs. After completion, the mixture was concentrated and purified by flash column (EA:PE=1:4, V/V) to afford 3-(tert-butyl) 4-ethyl 1-(bicyclo[1.1.1]pentan-1-yl)-6-oxo-1,6-dihydropyridine-3,4-dicarboxylate (258 mg, 26% yield). MS obsd. (ESI+): 334.2 (M+H)⁺.

### Step C: (R)-6-(bicyclo[1.1.1]pentan-1-yl)-2-methyl-4-((1-(2-methyl-3-(trifluoromethyl)phenyl)ethyl)amino)-2,6-dihydropyrido[3,4-d]pyridazine-1,7-dione (example 16)

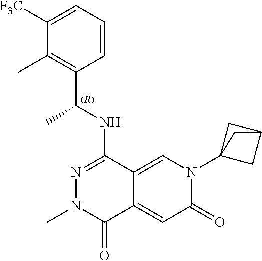

Prepared according to an analogous route as described for example 2. MS obsd. (ESI⁺): 445.3 (M+H)⁺.

### Example 17: (R)-6-cyclopropyl-4-((1-(3-(difluoromethyl)-2-fluorophenyl)ethyl)amino)-2-methyl-2,6-dihydropyrido[3,4-d]pyridazine-1,7-dione

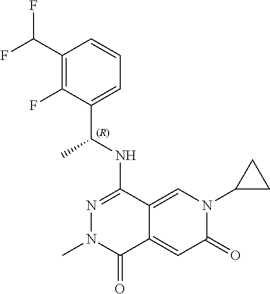

Prepared according to an analogous route as described for example 2 using (R)-1-(3-(difluoromethyl)-2-fluorophenyl)ethan-1-amine (preparation described in WO/2018/115380). MS obsd. (ESI⁺): 405.3 (M+H)⁺.

### Example 18: (R)-4-((1-(3-(difluoromethyl)-2-fluorophenyl)ethyl)amino)-2-methyl-6-(tetrahydro-2H-pyran-4-yl)-2,6-dihydropyrido[3,4-d]pyridazine-1,7-dione

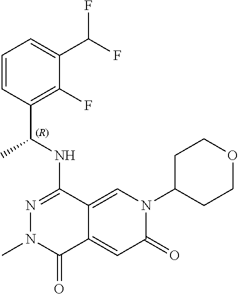

Prepared according to an analogous route as described for example 4. MS obsd. (ESI⁺): 449.5 (M+H)⁺.

### Example 19: (R)-6-cyclopropyl-8-ethoxy-2-methyl-4-((1-(2-methyl-3-(trifluoromethyl)phenyl)ethyl)amino)-2,6-dihydropyrido[3,4-d]pyridazine-1,7-dione

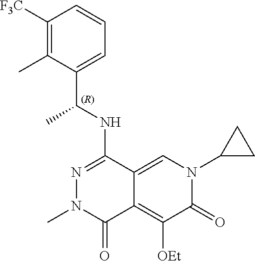

### Step A: 6-cyclopropyl-8-ethoxy-2-methyl-2,3-dihydropyrido[3,4-d]pyridazine-1,4,7(6H)-trione

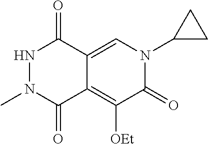 [SMILES: CCOc1c(=O)n(C2CC2)cc2c(=O)[nH]n(C)c(=O)c12]

8-bromo-6-cyclopropyl-2-methyl-2,3-dihydropyrido[3,4-d]pyridazine-1,4,7(6H)-trione (100 mg, 0.32 mmol) was dissolved in EtOH (5 mL), followed by the addition of sodium ethoxide (436 mg, 6.41 mmol). The whole mixture was stirred at 90° C. for 15 hrs. After the reaction was finished, the mixture filtered and the solvent was removed under reduced pressure. And then the crude residue was purified by flash column chromatography (MeOH:DCM=1:5, V/V) to afford 6-cyclopropyl-8-ethoxy-2-methyl-2,3-dihydropyrido[3,4-d]pyridazine-1,4,7(6H)-trione (63 mg, 67% yield). MS obsd. (ESI+): 278.2 (M+H)⁺.

### Step B: 6-cyclopropyl-8-ethoxy-2-methyl-1,7-dioxo-1,2,6,7-tetrahydropyrido[3,4-d]pyridazin-4-yl 2,4,6-triisopropylbenzenesulfonate

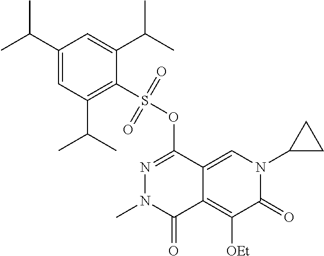 [SMILES: CCOc1c(=O)n(C2CC2)cc2c(OS(=O)(=O)c3c(C(C)C)cc(C(C)C)cc3C(C)C)nn(C)c(=O)c12]

6-cyclopropyl-8-ethoxy-2-methyl-2,3-dihydropyrido[3,4-d]pyridazine-1,4,7(6H)-trione (290 mg, 0.63 mmol) and N,N-diisopropylethylamine (1.26 mmol, 162 mg) were dissolved in DCM (10 mL) and 2,4,6-triisopropylbenzenesulfonyl chloride (209 mg, 0.69 mmol) was added at 0° C. The reaction was warmed to room temperature and stirred for 16 hrs. The solvent was concentrated under reduced pressure and the crude mixture was washed with sodium saturated bicarbonate solution (50 mL) and extracted with dichloromethane (150 mL). The organic phase was dried over anhydrous sodium sulfate, concentrated and purified by silica gel chromatography (PE:EtOAc=10:1 to 0:100, v/v) to afford 6-cyclopropyl-8-ethoxy-2-methyl-1,7-dioxo-pyrido[3,4-d]pyridazin-4-yl)2,4,6-triisopropylbenzene sulfate (120 mg, 34% yield). MS obsd. (ESI+): 544.4 (M+H)⁺.

### Step C: (R)-6-cyclopropyl-8-ethoxy-2-methyl-4-((1-(2-methyl-3-(trifluoromethyl)phenyl)ethyl)amino)-2,6-dihydropyrido[3,4-d]pyridazine-1,7-dione (example 19)

(6-cyclopropyl-8-ethoxy-2-methyl-1,7-dioxo-pyrido[3,4-d]pyridazin-4-yl)2,4,6-triisopropylbenzene sulfate (30 mg, 0.055 mmol), (R)-1-(2-methyl-3-(trifluoromethyl)phenyl)ethan-1-amine (16.8 mg, 0.082 mmol), XantPhos-Pd-G3 (5.2 mg, 5.52 umol, 0.1 eq) and dicesium carbonate (54 mg, 165.54 umol) were dissolved in 1,4-dioxane (5 mL). Then the mixture was stirred at 110° C. for 16 hrs. The mixture was filtered and the solvent was removed under reduced pressure. The crude residue was purified by preparative TLC (3% methanol in dichloromethane) and further purified by reverse phase HPLC to afford (R)-6-cyclopropyl-8-ethoxy-2-methyl-4-((1-(2-methyl-3-(trifluoromethyl)phenyl)ethyl)amino)-2,6-dihydropyrido[3,4-d]pyridazine-1,7-dione (0.3 mg, 1.2% yield). MS obsd. (ESI+): 463.3 (M+H)⁺.

### Example 20: 8-((R)-3-aminopyrrolidin-1-yl)-6-cyclopropyl-2-methyl-4-(((R)-1-(2-methyl-3-(trifluoromethyl)phenyl)ethyl)amino)-2,6-dihydropyrido[3,4-d]pyridazine-1,7-dione

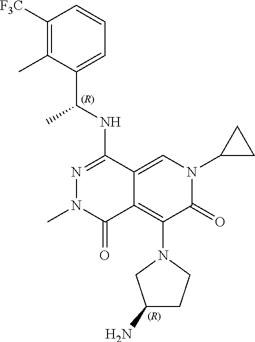

### Step A: tert-butyl ((R)-1-(6-cyclopropyl-2-methyl-4-(((R)-1-(2-methyl-3-(trifluoromethyl)phenyl)ethyl)amino)-1,7-dioxo-1,2,6,7-tetrahydropyrido[3,4-d]pyridazin-8-yl)pyrrolidin-3-yl)carbamate

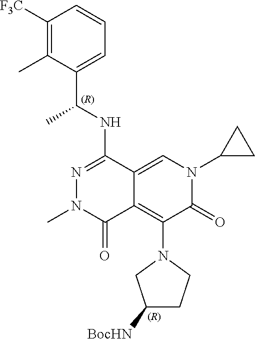

Tert-butyl N-[(3R)-pyrrolidin-3-yl]carbamate (804 umol, 149.8 mg) and (R)-8-bromo-6-cyclopropyl-2-methyl-4-((1-(2-methyl-3-(trifluoromethyl)phenyl)ethyl)amino)-2,6-dihydropyrido[3,4-d]pyridazine-1,7-dione (20 mg, 40 umol) were dissolved in dioxane (1 mL) and stirred at room temperature for 1 h. The reaction mixture was concentrated under reduced pressure to afford crude tert-butyl((R)-1-(6-cyclopropyl-2-methyl-4-(((R)-1-(2-methyl-3-(trifluoromethyl)phenyl)ethyl)amino)-1,7-dioxo-1,2,6,7-tetrahydropyrido[3,4-d]pyridazin-8-yl)pyrrolidin-3-yl)carbamate (20 mg, crude). MS obsd. (ESI+): 603.6 (M+H)⁺.

### Step B: 8-((R)-3-aminopyrrolidin-1-yl)-6-cyclopropyl-2-methyl-4-(((R)-1-(2-methyl-3-(trifluoromethyl)phenyl)ethyl)amino)-2,6-dihydropyrido[3,4-d]pyridazine-1,7-dione (Example 20)

Tert-butyl ((R)-1-(6-cyclopropyl-2-methyl-4-(((R)-1-(2-methyl-3-(trifluoromethyl)phenyl)ethyl)amino)-1,7-dioxo-1,2,6,7-tetrahydropyrido[3,4-d]pyridazin-8-yl)pyrrolidin-3-yl)carbamate (20 mg, crude) was dissolved in HCl (4M in 1,4-dioxane, 1.16 mL, 4.64 mmol). The solution was stirred at room temperature for 1 hours. The reaction mixture was concentrated under reduced pressure and purified by reverse phase HPLC to afford 8-((R)-3-aminopyrrolidin-1-yl)-6-cyclopropyl-2-methyl-4-(((R)-1-(2-methyl-3-(trifluoromethyl)phenyl)ethyl)amino)-2,6-dihydropyrido[3,4-d]pyridazine-1,7-dione (5.5 mg, 18% yield over two steps). MS obsd. (ESI+): 503.5 (M+H)⁺.

### Example 21: 8-((S)-3-aminopyrrolidin-1-yl)-6-cyclopropyl-2-methyl-4-(((R)-1-(2-methyl-3-(trifluoromethyl)phenyl)ethyl)amino)-2,6-dihydropyrido[3,4-d]pyridazine-1,7-dione

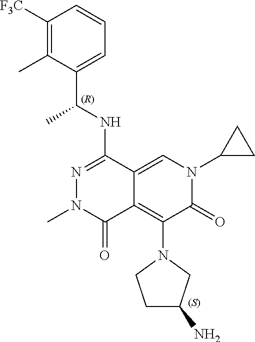

Prepared according to an analogous route as example 20. MS obsd. (ESI+): 503.2 (M+H)⁺.

### Example 22: (R)-2-methyl-4-((1-(2-methyl-3-(trifluoromethyl)phenyl)ethyl)amino)-6-(piperidin-4-yl)-2,6-dihydropyrido[3,4-d]pyridazine-1,7-dione

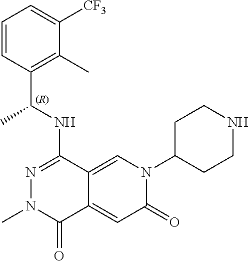

### Step A: 3-(tert-butyl) 4-methyl 1-(1-(tert-butoxycarbonyl)piperidin-4-yl)-6-oxo-1,6-dihydropyridine-3,4-dicarboxylate

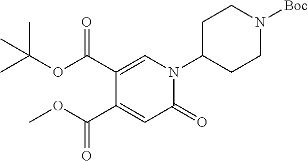 [SMILES: COC(=O)c1cc(=O)n(C2CCN(C(=O)OC(C)(C)C)CC2)cc1C(=O)OC(C)(C)C]

To a solution of tert-butyl 4-aminopiperidine-1l-carboxylate (3.0 g, 14.98 mmol, 1.0 eq.) and tert-butyl prop-2-ynoate (1.9 g, 14.98 mmol, 1.0 eq.) in EtOH (50 mL) was added H₂O (0.5 mL). The reaction was stirred for 24 hrs at 25° C. Then dimethyl but-2-ynedioate (2.1 g, 14.98 mmol, 1.0 eq.) was added and the reaction was heated to 85° C. for 24 hrs. After cooling to room temperature, the reaction was concentrated in vacuo and the residue was dissolved in MeOH (50.0 mL). NH₃ (7 M in MeOH, 5.0 mL) was then added and stirred for 4 hrs at 25° C. The solvent was removed in vacuo and the residue was purified by flash column chromatography (eluting with 0-50% EA in PE) to afford 3-(tert-butyl) 4-methyl 1-(1-(tert-butoxycarbonyl)piperidin-4-yl)-6-oxo-1,6-dihydropyridine-3,4-dicarboxylate (2.0 g, 30% yield). MS obsd. (ESI⁺): 381.3 (M-tBu)+.

Step B: 5-(tert-butoxycarbonyl)-1-(1-(tert-butoxycarbonyl)piperidin-4-yl)-2-oxo-1,2-dihydropyridine-4-carboxylic acid

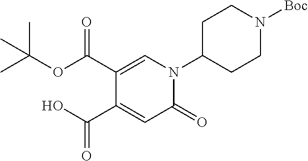 [SMILES: CC(C)(C)OC(=O)c1cn(C2CCN(C(=O)OC(C)(C)C)CC2)c(=O)cc1C(=O)O]

To a solution of 3-(tert-butyl) 4-methyl 1-(1-(tert-butoxycarbonyl)piperidin-4-yl)-6-oxo-1,6-dihydropyridine-3,4-dicarboxylate (2.0 g, 4.49 mmol) in THF (30 mL) and MeOH (30 mL) was added lithium hydroxide (439 mg, 18.33 mmol). The reaction was stirred for 16 hrs at 25° C. Then the mixture was concentrated in vacuo to give 5-(tert-butoxycarbonyl)-1-(1-(tert-butoxycarbonyl)piperidin-4-yl)-2-oxo-1,2-dihydropyridine-4-carboxylic acid (1.94 g, crude) which was used without further purification. MS obsd. (ESI+): 311.3 (M−2tBu)⁺.

### Step C: tert-butyl 4-(2-(tert-butoxycarbonyl)-1-methylhydrazine-1-carbonyl)-1-(1-(tert-butoxycarbonyl)piperidin-4-yl)-6-oxo-1,6-dihydropyridine-3-carboxylate

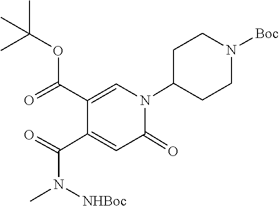 [SMILES: CN(NC(=O)OC(C)(C)C)C(=O)c1cc(=O)n(C2CCN(C(=O)OC(C)(C)C)CC2)cc1C(=O)OC(C)(C)C]

To a solution of 5-tert-butoxycarbonyl-1-(1-tert-butoxycarbonyl-4-piperidyl)-2-oxo-pyridine-4-carboxylic acid (1.94 g, crude, assumed 4.49 mmol) and tert-butyl N-(methylamino)carbamate (1.0 g, 6.89 mmol) in DMF (15 mL) was added HATU (3.5 g, 9.18 mmol) and N-ethyl-N-isopropyl-propan-2-amine (2.4 g, 18.37 mmol). The reaction was stirred for 16 hrs at 25° C. Then the mixture was quenched with H₂O (30 mL) and extracted with EA (30 mL×3). The combined organic layers were dried over MgSO₄, filtered and concentrated in vacuo. The residue was purified by flash chromatography (eluting with 0-80% EA in PE) to afford tert-butyl 4-(2-(tert-butoxycarbonyl)-1-methylhydrazine-1-carbonyl)-1-(1-(tert-butoxycarbonyl)piperidin-4-yl)-6-oxo-1,6-dihydropyridine-3-carboxylate (806 mg, 31% yield). MS obsd. (ESI+): 383.2 (M−3tBu)⁺.

### Step D: 2-methyl-6-(piperidin-4-yl)-2,3-dihydropyrido[3,4-d]pyridazine-1,4,7(6H)-trione

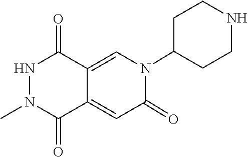 [SMILES: Cn1[nH]c(=O)c2cn(C3CCNCC3)c(=O)cc2c1=O]

To a solution of tert-butyl 4-(2-(tert-butoxycarbonyl)-1-methylhydrazine-1-carbonyl)-1-(1-(tert-butoxycarbonyl)piperidin-4-yl)-6-oxo-1,6-dihydropyridine-3-carboxylate (804 mg, 1.50 mmol) in 1,4-dioxane (1.0 mL) was added HCl (4 M in 1,4-dioxane, 10 mL). The reaction was stirred for 0.5 h at 25° C. and then heated to 100° C. for 2 hrs. The mixture was concentrated in vacuo and purified by flash chromatography (eluting with 0-20% DCM in MeOH) to afford 2-methyl-6-(piperidin-4-yl)-2,3-dihydropyrido[3,4-d]pyridazine-1,4,7(6H)-trione (275 mg, 66% yield). MS obsd. (ESI+): 277.4 (M+H)⁺.

### Step E: tert-butyl 4-(2-methyl-1,4,7-trioxo-1,3,4,7-tetrahydropyrido[3,4-d]pyridazin-6(2H)-yl)piperidine-1-carboxylate

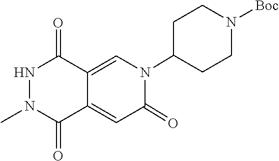 [SMILES: Cn1[nH]c(=O)c2cn(C3CCN(C(=O)OC(C)(C)C)CC3)c(=O)cc2c1=O]

To a solution of 2-methyl-6-(piperidin-4-yl)-2,3-dihydropyrido[3,4-d]pyridazine-1,4,7(6H)-trione (275 mg, 1.00 mmol) in DCM (10 mL) was added tert-butoxycarbonyl tert-butyl carbonate (219 mg, 1.00 mmol) and N, N-diethylethanamine (507 mg, 5.01 mmol) and the reaction was stirred for 2 hrs at 25° C. The mixture was concentrated in vacuo and purified by flash chromatography (eluting with 0-10% MeOH in DCM) to afford tert-butyl 4-(2-methyl-1,4,7-trioxo-1,3,4,7-tetrahydropyrido[3,4-d]pyridazin-6(2H)-yl)piperidine-1-carboxylate (323 mg, 86% yield). MS obsd. (ESI+): 321.3 (M−tBu)⁺.

### Step F: tert-butyl 4-(2-methyl-1,7-dioxo-4-(((2,4,6-triisopropylphenyl)sulfonyl)oxy)-1,7-dihydropyrido[3,4-d]pyridazin-6(2H)-yl)piperidine-1-carboxylate

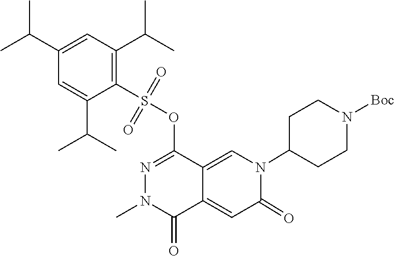 [SMILES: CC(C)c1cc(C(C)C)c(S(=O)(=O)Oc2nn(C)c(=O)c3cc(=O)n(C4CCN(C(=O)OC(C)(C)C)CC4)cc23)c(C(C)C)c1]

To a solution of tert-butyl 4-(2-methyl-1,4,7-trioxo-3H-pyrido[3,4-d]pyridazin-6-yl)piperidine-1-carboxylate (323 mg, 0.86 mmol) in DCM (10 mL) was added N-ethyl-N-isopropyl-propan-2-amine (444 mg, 3.43 mmol) and 2,4,6-triisopropylbenzenesulfonyl chloride (520 mg, 1.72 mmol) and the reaction was stirred for 2 hrs at 25° C. Then the mixture was concentrated in vacuo and purified by flash chromatography (eluting with 0-10% MeOH in DCM) to afford tert-butyl 4-(2-methyl-1,7-dioxo-4-(((2,4,6-triisopropylphenyl)sulfonyl)oxy)-1,7-dihydropyrido[3,4-d]pyridazin-6(2H)-yl)piperidine-1-carboxylate (365 mg, 66% yield). MS obsd. (ESI+): 587.4 (M−tBu)⁺.

### Step G: tert-butyl (R)-4-(2-methyl-4-((1-(2-methyl-3-(trifluoromethyl)phenyl)ethyl)amino)-1,7-dioxo-1,7-dihydropyrido[3,4-d]pyridazin-6(2H)-yl)piperidine-1-carboxylate

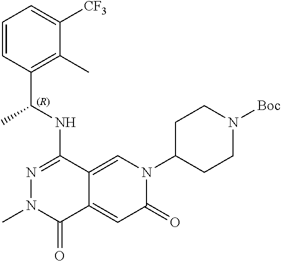

To a solution of tert-butyl 4-(2-methyl-1,7-dioxo-4-(((2,4,6-triisopropylphenyl)sulfonyl)oxy)-1,7-dihydropyrido[3,4-d]pyridazin-6(2H)-yl)piperidine-1-carboxylate (150 mg, 0.23 mmol) and (1R)-1-[2-methyl-3-(trifluoromethyl)phenyl]ethanamine (119 mg, 0.58 mmol) in 1,4-dioxane (4 mL) was added cesium carbonate (304 mg, 0.93 mmol, 4.0 eq.) and XantPhos-Pd-G3 (72 mg, 0.08 mmol). The reaction was stirred for 16 hrs at 110° C. Then the mixture was concentrated in vacuo and purified by silica gel chromatography (eluting with 0-10% MeOH in DCM) to afford tert-butyl (R)-4-(2-methyl-4-((1-(2-methyl-3-(trifluoromethyl)phenyl)ethyl)amino)-1,7-dioxo-1,7-dihydropyrido[3,4-d] pyridazin-6(2H)-yl)piperidine-1-carboxylate (65 mg, 50% yield, 89% purity). MS obsd. (ESI+): 562.4 (M+H)⁺.

### Step H: (R)-2-methyl-4-((1-(2-methyl-3-(trifluoromethyl)phenyl)ethyl)amino)-6-(piperidin-4-yl)-2,6-dihydropyrido[3,4-d]pyridazine-1,7-dione (example 22)

To a solution of tert-butyl 4-[2-methyl-4-[[(1R)-1-[2-methyl-3-(trifluoromethyl)phenyl]ethyl]amino]-1,7-dioxo-pyrido[3,4-d]pyridazin-6-yl]piperidine-1-carboxylate (65 mg, 0.11 mmol, 89% purity) in 1,4-dioxane (1.0 mL) was added hydrochloric acid (4 M in 1,4-dioxane, 2.0 mL) and the reaction was stirred for 1 hr at 25° C. The mixture was concentrated in vacuo and the residue was purified by reverse phase HPLC to afford (R)-2-methyl-4-((1-(2-methyl-3-(trifluoromethyl)phenyl)ethyl)amino)-6-(piperidin-4-yl)-2,6-dihydropyrido[3,4-d]pyridazine-1,7-dione (40 mg, 78% yield). MS obsd. (ESI+):462.5 (M+H)⁺.

### Example 23: (R)-2-methyl-4-((1-(2-methyl-3-(trifluoromethyl)phenyl)ethyl)amino)-6-(1-methylpiperidin-4-yl)-2,6-dihydropyrido[3,4-d]pyridazine-1,7-dione

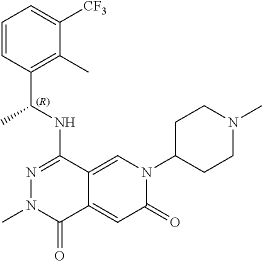

To a solution of (R)-2-methyl-4-((1-(2-methyl-3-(trifluoromethyl)phenyl)ethyl)amino)-6-(piperidin-4-yl)-2,6-dihydropyrido[3,4-d]pyridazine-1,7-dione (example 22) (70 mg, 0.02 mmol) in MeOH (8.0 mL) was added polyoxymethylene (36 mg, 3.03 mmol) and acetic acid (2 mg, 0.03 mmol) and the reaction was stirred for 1 hr at 25° C. Then sodium cyanoborohydride (191 mg, 3.03 mmol) was added and the reaction was stirred for 16 hrs at 25° C. The mixture was then concentrated in vacuo and purified by reverse phase HPLC to afford (R)-2-methyl-4-((1-(2-methyl-3-(trifluoromethyl)phenyl)ethyl)amino)-6-(1-methylpiperidin-4-yl)-2,6-dihydropyrido[3,4-d]pyridazine-1,7-dione (22 mg, 30% yield). MS obsd. (ESI+): 476.4 (M+H)⁺.

### Example 24: (R)-2-(2-aminoethyl)-6-cyclopropyl-4-((1-(2-methyl-3-(trifluoromethyl)phenyl)ethyl)amino)-2,6-dihydropyrido[3,4-d]pyridazine-1,7-dione

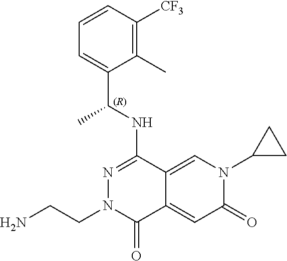

### Step A: benzyl (2-oxoethyl)carbamate

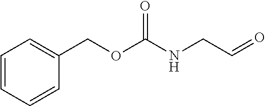 [SMILES: O=CCNC(=O)OCc1ccccc1]

To a solution of benzyl N-(2-hydroxyethyl)carbamate (4.0 g, 20.49 mmol) and N-ethyl-N-isopropyl-propan-2-amine (5.3 g, 40.98 mmol) in DCM (15.0 mL) was added pyridine sulfur trioxide (6.5 g, 40.98 mmol) in DMSO (3.0 mL) at −20° C. The reaction was stirred for 4 hrs at −20° C., then warmed to 25° C. and diluted with DCM. The organic layer was washed with 1.0 M HCl followed by brine, then dried over MgSO₄ and concentrated in vacuo. The residue was purified by flash chromatography (eluting with 0-80% EA in PE) to afford benzyl (2-oxoethyl)carbamate (1.4 g, 35% yield) as a colorless oil. MS obsd. (ESI+): 194.2 (M+H)⁺.

### Step B: tert-butyl 2-(2-(((benzyloxy)carbonyl)amino)ethyl)hydrazine-1-carboxylate

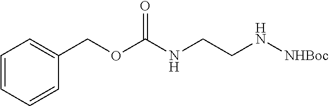 [SMILES: CC(C)(C)OC(=O)NNCCNC(=O)OCc1ccccc1]

To a solution of benzyl N-(2-oxoethyl)carbamate (1.4 g, 9.06 mmol) and tert-butyl N-aminocarbamate (1.2 g, 9.06 mmol) in MeOH (20.0 mL) was added acetic acid (105 mg, 1.75 mmol) and the reaction was stirred for 30 min at 25° C. Then sodium cyanoborohydride (1.7 g, 27.17 mmol) was added and the mixture was allowed to stir for 3 hrs at 25° C. The mixture was concentrated and 50 mL of DCM was added. The organic layer was washed with water followed by brine, dried over MgSO₄ and concentrated in vacuo. The residue was then purified by flash chromatography (eluting with 0-80% EA in PE) to afford tert-butyl 2-(2-(((benzyloxy)carbonyl)amino)ethyl)hydrazine-1-carboxylate (1.7 g, 76% yield). MS obsd. (ESI+): 210.2 (M−Boc)⁺.

### Step C: tert-butyl 1-cyclopropyl-4-((11,11-dimethyl-3,9-dioxo-1-phenyl-2,10-dioxa-4,7,8-triazadodecan-7-yl)carbonyl)-6-oxo-1,6-dihydropyridine-3-carboxylate

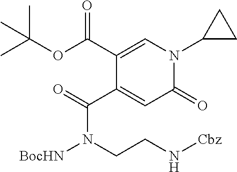 [SMILES: CC(C)(C)OC(=O)NN(CCNC(=O)OCc1ccccc1)C(=O)c1cc(=O)n(C2CC2)cc1C(=O)OC(C)(C)C]

To a solution of tert-butyl N-[2-(benzyloxycarbonylamino)ethylamino]carbamate (1.7 g, 5.37 mmol) and 5-tert-butoxycarbonyl-1-cyclopropyl-2-oxo-pyridine-4-carboxylic acid (1.0 g, 3.58 mmol) in DMF (6.0 mL) was added N,N,N′,N′-tetramethyl-1-(3-oxido-2,3-dihydrotriazolo[4,5-b]pyridin-3-ium-1-yl)methanediamine; hexafluorophosphate (2.7 g, 7.16 mmol) and N-ethyl-N-isopropyl-propan-2-amine (1.9 g, 14.32 mmol) at 0° C. The reaction was stirred for 16 hrs at 25° C. Then the mixture was quenched with H₂O (50 mL) and extracted with EA (30 mL×3). The organic layer was then dried over MgSO₄, filtered and concentrated in vacuo. The residue was purified by flash chromatography (eluting with 0-80% EA in PE) to afford tert-butyl 1-cyclopropyl-4-((11,11-dimethyl-3,9-dioxo-1-phenyl-2,10-dioxa-4,7,8-triazadodecan-7-yl)carbonyl)-6-oxo-1,6-dihydropyridine-3-carboxylate (548 mg, 27% yield). MS obsd. (ESI+): 459.5 (M−(2×tBu))⁺.

### Step D: benzyl (2-(6-cyclopropyl-4-hydroxy-1,7-dioxo-6,7-dihydropyrido[3,4-d]pyridazin-2(1H)-yl)ethyl)carbamate

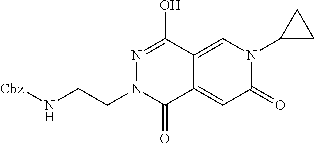 [SMILES: O=C(NCCn1nc(O)c2cn(C3CC3)c(=O)cc2c1=O)OCc1ccccc1]

To a solution of tert-butyl 1-cyclopropyl-4-((11,11-dimethyl-3,9-dioxo-1-phenyl-2,10-dioxa-4,7,8-triazadodecan-7-yl)carbonyl)-6-oxo-1,6-dihydropyridine-3-carboxylate (548 mg, 0.96 mmol) in DCM (4.0 mL) was added 2,2,2-trifluoroacetic acid (3.0 g, 25.96 mmol, 2.0 mL) and the reaction was stirred for 0.5 h at 25° C. and then heated to 50° C. for 2 hrs. The reaction was concentrated in vacuo to give benzyl (2-(6-cyclopropyl-4-hydroxy-1,7-dioxo-6,7-dihydropyrido[3,4-d]pyridazin-2(1H)-yl)ethyl)carbamate (415 mg, crude) as a brown oil. The crude product was used for next step without further purification. MS obsd. (ESI+): 397.2 (M+H)⁺.

### Step E: 2-(2-(((benzyloxy)carbonyl)amino)ethyl)-6-cyclopropyl-1,7-dioxo-1,2,6,7-tetrahydropyrido[3,4-d]pyridazin-4-yl 2,4,6-triisopropylbenzenesulfonate

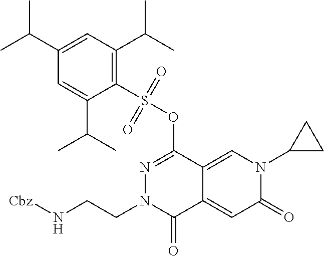 [SMILES: CC(C)c1cc(C(C)C)c(S(=O)(=O)Oc2nn(CCNC(=O)OCc3ccccc3)c(=O)c3cc(=O)n(C4CC4)cc23)c(C(C)C)c1]

To a solution of benzyl N-[2-(6-cyclopropyl-4-hydroxy-1,7-dioxo-pyrido[3,4-d]pyridazin-2-yl)ethyl]carbamate (415 mg, crude, assumed 0.96 mmol) in DCM (20.0 mL) was added N-ethyl-N-isopropyl-propan-2-amine (677 mg, 5.23 mmol) and 2,4,6-triisopropylbenzenesulfonyl chloride (476 mg, 1.57 mmol) and the reaction was stirred for 2 hrs at 25° C. The mixture was concentrated in vacuo and purified by flash chromatography (eluting with 0-80% EA in PE) to afford 2-(2-(((benzyloxy)carbonyl)amino)ethyl)-6-cyclopropyl-1,7-dioxo-1,2,6,7-tetrahydropyrido[3,4-d]pyridazin-4-yl 2,4,6-triisopropylbenzenesulfonate (430 mg, 68% yield over two steps). MS obsd. (ESI+): 663.5 (M+H)⁺.

### Step F: benzyl (R)-(2-(6-cyclopropyl-4-((1-(2-methyl-3-(trifluoromethyl)phenyl)ethyl)amino)-1,7-dioxo-6,7-dihydropyrido[3,4-d]pyridazin-2(1H)-yl)ethyl)carbamate

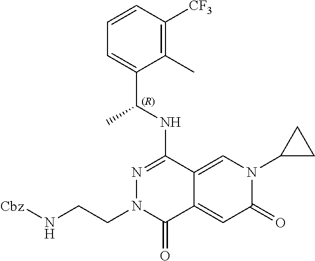

To a solution of 2-(2-(((benzyloxy)carbonyl)amino)ethyl)-6-cyclopropyl-1,7-dioxo-1,2,6,7-tetrahydropyrido[3,4-d]pyridazin-4-yl 2,4,6-triisopropylbenzenesulfonate (170 mg, 0.26 mmol) and (1R)-1-[2-methyl-3-(trifluoromethyl)phenyl]ethanamine (78 mg, 0.38 mmol) in dioxane (6.0 mL) was added cesium carbonate (334 mg, 1.03 mmol) and XantPhos-Pd-G3 (49 mg, 0.05 mmol). The reaction was stirred for 16 hrs at 110° C. Then the mixture was quenched with H₂O (30 mL). The mixture was extracted with EA (20 mL×3) and dried over MgSO₄, filtered and concentrated in vacuo. The residue was purified by flash chromatography (eluting with 0-10% MeOH in DCM) to afford benzyl (R)-(2-(6-cyclopropyl-4-((1-(2-methyl-3-(trifluoromethyl)phenyl)ethyl)amino)-1,7-dioxo-6,7-dihydropyrido[3,4-d]pyridazin-2(1H)-yl)ethyl)carbamate (26 mg, 17% yield). MS obsd. (ESI+): 582.3 (M+H)⁺.

### Step G: (R)-2-(2-aminoethyl)-6-cyclopropyl-4-((1-(2-methyl-3-(trifluoromethyl)phenyl)ethyl)amino)-2,6-dihydropyrido[3,4-d]pyridazine-1,7-dione (example 24)

To a solution of benzyl N-[2-[6-cyclopropyl-4-[[(1R)-1-[2-methyl-3-(trifluoromethyl)phenyl]ethyl]amino]-1,7-dioxo-pyrido[3,4-d]pyridazin-2-yl]ethyl]carbamate (30 mg, 0.05 mmol) in EA (1.5 mL) was added Hydrobromic acid (33% in acetic acid, 8.29 mmol, 1.50 mL) at 0° C. and the reaction was heated to 50° C. for 2 h. Then the mixture was concentrated in vacuo and purified by reverse phase HPLC to afford (R)-2-(2-aminoethyl)-6-cyclopropyl-4-((1-(2-methyl-3-(trifluoromethyl)phenyl)ethyl)amino)-2,6-dihydropyrido[3,4-d]pyridazine-1,7-dione (6 mg, 26% yield). MS obsd. (ESI+): 448.5 (M+H)⁺.

### Example 25 and 26: (R)-6-(1-((6-cyclopropyl-2-methyl-1,7-dioxo-1,2,6,7-tetrahydropyrido[3,4-d]pyridazin-4-yl)amino)ethyl)-1-methyl-1H-indazole-3-carbonitrile (Example 25, stereochemistry is arbitrarily assigned) and (S)-6-(1-((6-cyclopropyl-2-methyl-1,7-dioxo-1,2,6,7-tetrahydropyrido[3,4-d]pyridazin-4-yl)amino)ethyl)-1-methyl-1H-indazole-3-carbonitrile (example 26, stereochemistry is arbitrarily assigned)

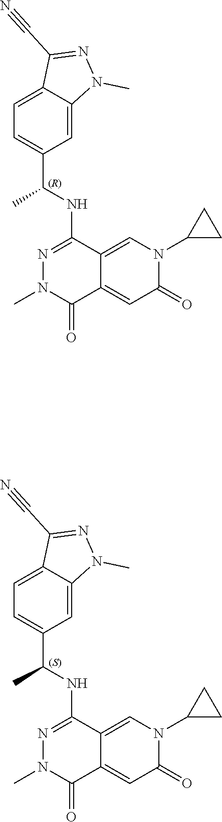

### Step A: 6-bromo-1-methyl-indazole-3-carbonitrile

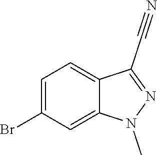

To a solution of 6-bromo-1H-indazole-3-carbonitrile (2.3 g, 10.36 mmol) and potassium carbonate (4.29 g, 31.08 mmol) in MeCN (30 mL) was added iodomethane (2.94 g, 20.72 mmol, 1.29 mL). The mixture was stirred at rt for 18 hrs. The reaction was then quenched with ice-water (50 mL) and the mixture was extracted with EtOAc (70 mL×3). The combined organic layers were dried over magnesium sulfate and concentrated under vacuum. The crude product was purified by flash column chromatography (eluted with PE:EtOAc=4:1, v/v) to afford 6-bromo-1-methyl-indazole-3-carbonitrile (1.6 g, 64%, yield). ¹H NMR (400 MHz, CDCl₃) δ: 7.71 (d, J=8.8 Hz, 2H), 7.46 (dd, J=8.6, 1.6 Hz, 1H), 4.13 (s, 3H).

### Step B: 6-acetyl-1-methyl-indazole-3-carbonitrile

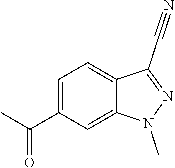

A mixture of 6-bromo-1-methyl-indazole-3-carbonitrile (1.5 g, 6.35 mmol), tributyl(1-ethoxyvinyl)stannane (3.44 g, 9.53 mmol, 3.21 mL), bis(Triphenylphosphine)palladium (II) chloride (669 mg, 9.53 mmol) and triethylamine (1.29 g, 12.71 mmol, 1.77 mL) in dioxane (30 mL) was heated to 80° C. for 16 hrs. Ice-water (100 mL) was added and the mixture was extracted with EtOAc (100 mL×3). The combined organic layers were dried over magnesium sulfate and concentrated under vacuum. The crude product was purified by flash column (eluted with PE:EtOAc=4:1) to afford 6-acetyl-1-methyl-indazole-3-carbonitrile (830 mg, 62% yield). ¹H NMR (400 MHz, CDCl₃) δ: 8.17 (s, 1H), 7.93 (dd, J=2.8, 1.0 Hz, 2H), 4.25 (s, 3H), 2.74 (s, 3H).

### Step C: N-(1-(3-cyano-1-methyl-1H-indazol-6-yl)ethylidene)-2-methylpropane-2-sulfinamide

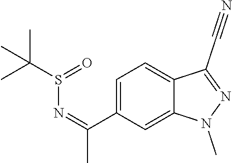 [SMILES: C/C(=N/S(=O)C(C)(C)C)c1ccc2c(C#N)nn(C)c2c1]

A mixture of 6-acetyl-1-methyl-indazole-3-carbonitrile (500 mg, 2.51 mmol), 2-methylpropane-2-sulfinamide (456.31 mg, 3.76 mmol) and titanium ethoxide (1.43 g, 6.27 mmol, 1.31 mL) in THE (20 mL) was heated to 80° C. for 16 hrs. The reaction was quenched with ice-water (100 mL) and the mixture was extracted with ethyl acetate (100 mL×3). The combined organic layers were dried over magnesium sulfate and concentrated under vacuum. The crude product was purified by flash column (eluted with PE:EtOAc=4:1 to 1:1) to give N-(1-(3-cyano-1-methyl-1H-indazol-6-yl)ethylidene)-2-methylpropane-2-sulfinamide (626 mg, 82% yield). MS obsd. (ESI+): 303.4 (M+H)⁺.

### Step D: N-(1-(3-cyano-1-methyl-1H-indazol-6-yl)ethyl)-2-methylpropane-2-sulfinamide (diastereomeric mixture)

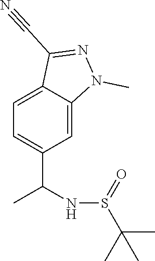 [SMILES: CC(NS(=O)C(C)(C)C)c1ccc2c(C#N)nn(C)c2c1]

To a solution of N-(1-(3-cyano-1-methyl-1H-indazol-6-yl)ethylidene)-2-methylpropane-2-sulfinamide (626 mg, 2.07 mmol) in MeOH (15 mL) was added sodium borohydride (234.96 mg, 6.21 mmol) at 0° C. and the whole mixture was stirred at this temperature for 2 hrs. The reaction was quenched with ice-water (30 mL). The mixture was extracted with EA (70 mL×3) and the combined organic layers were dried over magnesium sulfate and concentrated under vacuum. The crude product was purified by flash column chromatography (eluted with PE:EtOAc=4:1 to 1:2, v/v) to afford N-(1-(3-cyano-1-methyl-1H-indazol-6-yl)ethyl)-2-methylpropane-2-sulfinamide (diastereomeric mixture) (570 mg, 85% yield). ¹H NMR (400 MHz, DMSO-d6) δ: 7.88-7.82 (m, 2H), 7.45 (d, J=8.0 Hz, 1H), 5.83-5.49 (m, 1H), 4.66-4.53 (m, 1H), 4.18 (s, 3H), 1.51 (d, J=8.0 Hz, 3H), 1.12 (s, 9H).

### Step E: 5,6-(1-aminoethyl)-1-methyl-1H-indazole-3-carbonitrile hydrochloride

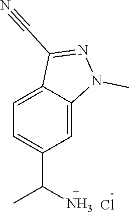

To a mixture of N-(1-(3-cyano-1-methyl-1H-indazol-6-yl)ethyl)-2-methylpropane-2-sulfinamide (500 mg, 1.64 mmol) in dioxane (5 mL) was added HCl (4M in 1,4-Dioxane, 10 mL, 40 mmol) at 0° C. The mixture was stirred at 0° C. for 2 hrs. The mixture was then concentrated under vacuum. The crude product was suspended in MeCN (15 mL) and the solid was filtered to give 6-(1-aminoethyl)-1-methyl-1H-indazole-3-carbonitrile hydrochloride (326 mg, 82% yield).¹H NMR (400 MHz, D₂O) δ: 7.68 (d, J=8.0 Hz, 2H), 7.36 (d, J=8.8 Hz, 1H), 4.65 (m, 1H), 4.01 (s, 3H), 1.67 (d, J=6.8 Hz, 3H).

### Step F: (R)-6-(1-((6-cyclopropyl-2-methyl-1,7-dioxo-1,2,6,7-tetrahydropyrido[3,4-d]pyridazin-4-yl)amino)ethyl)-1-methyl-1H-indazole-3-carbonitrile (Example 25, stereochemistry is arbitrarily assigned) and (R)-6-(1-((6-cyclopropyl-2-methyl-1,7-dioxo-1,2,6,7-tetrahydropyrido[3,4-d]pyridazin-4-yl)amino)ethyl)-1-methyl-1H-indazole-3-carbonitrile (example 26, stereochemistry is arbitrarily assigned)

Prepared according to an analogous procedure as described for example 2 using racemic 6-(1-aminoethyl)-1-methyl-1H-indazole-3-carbonitrile hydrochloride. Separation of isomers via chiral SFC afforded (R)-6-(1-((6-cyclopropyl-2-methyl-1,7-dioxo-1,2,6,7-tetrahydropyrido[3,4-d]pyridazin-4-yl)amino)ethyl)-1-methyl-1H-indazole-3-carbonitrile (example 25) and (S)-6-(1-((6-cyclopropyl-2-methyl-1,7-dioxo-1,2,6,7-tetrahydropyrido[3,4-d]pyridazin-4-yl)amino)ethyl)-1-methyl-1H-indazole-3-carbonitrile (example 26), Example 25: MS obsd. (ESI+): 416.3 (M+H)+, Example 26: MS obsd. (ESI+): 416.3 (M+H)⁺.

### Example 27 and 28: (R)-6-(1-((6-cyclopropyl-2-methyl-1,7-dioxo-1,2,6,7-tetrahydropyrido[3,4-d]pyridazin-4-yl)amino)ethyl)-1,4-dimethyl-1H-indazole-3-carbonitrile (example 27, stereochemistry is arbitrarily assigned) and (S)-6-(1-((6-cyclopropyl-2-methyl-1,7-dioxo-1,2,6,7-tetrahydropyrido[3,4-d]pyridazin-4-yl)amino)ethyl)-1,4-dimethyl-1H-indazole-3-carbonitrile (example 28, stereochemistry is arbitrarily assigned)

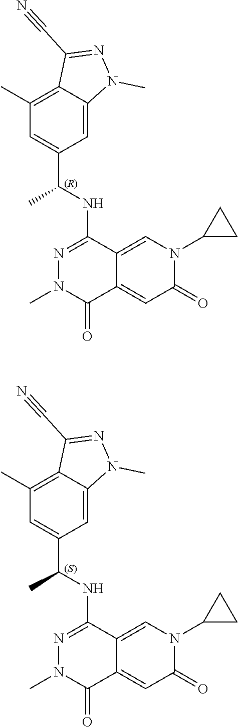

### Step A: 6-bromo-1,4-dimethyl-1H-indazole

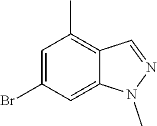

To a mixture of 6-bromo-4-methyl-1H-indazole (3 g, 14.21 mmol), Potassium carbonate (5.89 g, 42.64 mmol) in DMF (50 mL) was added iodomethane (4.04 g, 28.43 mmol, 1.77 mL). The mixture was stirred at rt for 2 hrs. The mixture was diluted with EtOAc (160 mL), and washed with H₂O (80 mL×3) and brine (80 mL×3). The organic layer was dried over sodium sulfate, filtered and concentrated. The residue was purified by silica gel chromatography (PE:EA=5:1) to provide 6-bromo-1,4-dimethyl-indazole (1.72 g, 52% yield). MS obsd. (ESI+): ^{79/81}Br 225.0/227.0 (M+H)⁺.

### Step B: 6-bromo-3-iodo-1,4-dimethyl-1H-indazole

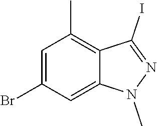

To a solution of 6-bromo-1,4-dimethyl-indazole (1.72 g, 7.64 mmol, 1.0 eq) in DMSO (30 mL) was added N-Iodosuccinimide (2.58 g, 11.46 mmol, 1.5 eq). The mixture was stirred at 90° C. for 16 hrs. The mixture was cooled to rt, diluted with EtOAc (20 mL). The organic layer was washed with saturated Na₂S₂O₃ (10 mL×2), H₂O (20 mL×3), brine (15 mL×3), dried over sodium sulfate and concentrated. The residue was purified by chromatography on silica gel column (PE:EA=7:1) to provide the title compound 6-bromo-3-iodo-1,4-dimethyl-indazole (0.7 g, 26% yield). MS obsd. (ESI+): ^{79/81}Br 350.8/352.8 (M+H)+.

### Step C: 6-bromo-1,4-dimethyl-1H-indazole-3-carbonitrile

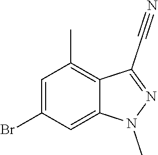

The mixture of 6-bromo-3-iodo-1,4-dimethyl-indazole (400 mg, 1.14 mmol), copper(I) cyanide (112.28 mg, 1.25 mmol, 38.45 uL) in DMSO (6 mL) was stirred at 150° C. for 2 hrs. The mixture was concentrated and purified by silica gel chromatography column (PE:EA=4:1) to provide the title compound 6-bromo-1,4-dimethyl-indazole-3-carbonitrile (190 mg, 23% yield). MS obsd. (ESI+): ^{79/81}Br 250.2/252.2 (M+H)+

### Step D: (R)-6-(1-((6-cyclopropyl-2-methyl-1,7-dioxo-1,2,6,7-tetrahydropyrido[3,4-d]pyridazin-4-yl)amino)ethyl)-1,4-dimethyl-1H-indazole-3-carbonitrile (example 27) and (S)-6-(1-((6-cyclopropyl-2-methyl-1,7-dioxo-1,2,6,7-tetrahydropyrido[3,4-d]pyridazin-4-yl)amino)ethyl)-1,4-dimethyl-1H-indazole-3-carbonitrile (example 28)

Title compounds were prepared using 6-bromo-1,4-dimethyl-1H-indazole-3-carbonitrile and procedures analogous to Examples 25 and 26 Steps (B-F). Example 27 (stereochemistry arbitrarily assigned): MS obsd. (ESI+): 430.2 (M+H)⁺. Example 28 (stereochemistry arbitrarily assigned): MS obsd. (ESI+): 430.2 (M+H)⁺.

### Example 29: 6-((1r,3R)-3-(1H-imidazol-1-yl)cyclobutyl)-2-methyl-4-(((R)-1-(2-methyl-3-(trifluoromethyl)phenyl)ethyl)amino)-2,6-dihydropyrido[3,4-d]pyridazine-1,7-dione

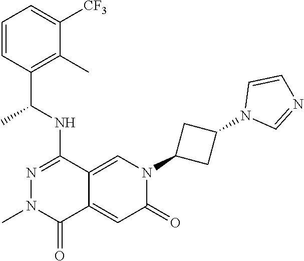 [SMILES: Cc1c([C@@H](C)Nc2nn(C)c(=O)c3cc(=O)n([C@H]4C[C@H](n5ccnc5)C4)cc23)cccc1C(F)(F)F]

### Step A: (1s,3s)-3-aminocyclobutan-1-ol hydrochloride

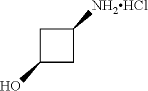

To a solution of tert-butyl ((1s,3s)-3-hydroxycyclobutyl)carbamate (6.0 g, 32.05 mmol, 1.0 eq.) in dioxane (15.0 mL) was added HCl (4.0 M in 1,4-dioxane, 15.0 mL) and the mixture was stirred for 4 h at 25° C. Then the mixture was concentrated to give (1s,3s)-3-aminocyclobutan-1-ol hydrochloride (3.95 g, 32.05 mmol, crude) which was used for the next setp without further purification. MS obsd. (ESI+): 88.1 (M+H)⁺.

### Step B: 3-(tert-butyl) 4-methyl 1-((1s,3s)-3-hydroxycyclobutyl)-6-oxo-1,6-dihydropyridine-3,4-dicarboxylate and 3-(tert-butyl) 4-ethyl 1-((1s,3s)-3-hydroxycyclobutyl)-6-oxo-1,6-dihydropyridine-3,4-dicarboxylate

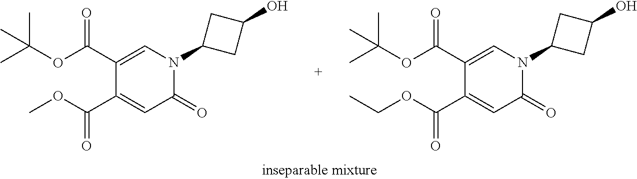 [SMILES: COC(=O)c1cc(=O)n([C@H]2C[C@@H](O)C2)cc1C(=O)OC(C)(C)C ; CCOC(=O)c1cc(=O)n([C@H]2C[C@@H](O)C2)cc1C(=O)OC(C)(C)C]

To the solution of (1s,3s)-3-aminocyclobutan-1-ol hydrochloride (3 g, 24.28 mmol) and tert-butyl propiolate (3.1 g, 24.28 mmol) in EtOH (40.0 mL) was added N-ethyl-N-isopropyl-propan-2-amine (12.6 g, 97.10 mmol) and H₂O (88 mg, 4.86 mmol). The mixture was stirred for 16 h at 25° C. Then to the mixture was added dimethyl but-2-ynedioate (3.5 g, 24.28 mmol.) and the reaction was stirred for 48 h at 85° C. The mixture was concentrated and directly purified by flash column chromatography (eluting with 0-100% EA in PE) to afford an inseparable mixture of 3-(tert-butyl) 4-methyl 1-((1s,3s)-3-hydroxycyclobutyl)-6-oxo-1,6-dihydropyridine-3,4-dicarboxylate and 3-(tert-butyl) 4-ethyl 1-((1s,3s)-3-hydroxycyclobutyl)-6-oxo-1,6-dihydropyridine-3,4-dicarboxylate (2.0 g, 5.93 mmol, 24% yield, ˜78% purity), which was used without further purification. MS obsd. (ESI+): 324.4, 338.4 (M+H)⁺.

### Step C: 3-(tert-butyl) 4-methyl 6-oxo-1-((1s,3s)-3-(tosyloxy)cyclobutyl)-1,6-dihydropyridine-3,4-dicarboxylate and 3-(tert-butyl) 4-ethyl 6-oxo-1-((1s,3s)-3-(tosyloxy)cyclobutyl)-1,6-dihydropyridine-3,4-dicarboxylate

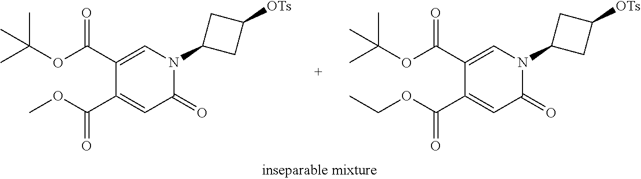 [SMILES: COC(=O)c1cc(=O)n([C@H]2C[C@@H](OS(=O)(=O)c3ccc(C)cc3)C2)cc1C(=O)OC(C)(C)C ; CCOC(=O)c1cc(=O)n([C@H]2C[C@@H](OS(=O)(=O)c3ccc(C)cc3)C2)cc1C(=O)OC(C)(C)C]

To a solution of the afforementioned mixture of 3-(tert-butyl) 4-methyl 1-((1s,3s)-3-hydroxycyclobutyl)-6-oxo-1,6-dihydropyridine-3,4-dicarboxylate and 3-(tert-butyl) 4-ethyl 1-((1s,3s)-3-hydroxycyclobutyl)-6-oxo-1,6-dihydropyridine-3,4-dicarboxylate (2.0 g, 4.8 mmol, ˜78% purity) in DCM (50) was added 4-methylbenzenesulfonyl chloride (2.3 g, 11.86 mmol), N,N-diethylethanamine (1.8 g, 17.78 mmol) and DMAP (724 mg, 5.93 mmol). The reaction was stirred for 3 h at 50° C. Then the mixture was concentrated and purified by flash chromatography (eluting with 0-100% EA in PE) to afford a mixture of 3-(tert-butyl) 4-methyl 6-oxo-1-((1s,3s)-3-(tosyloxy)cyclobutyl)-1,6-dihydropyridine-3,4-dicarboxylate and 3-(tert-butyl) 4-ethyl 6-oxo-1-((1s,3s)-3-(tosyloxy)cyclobutyl)-1,6-dihydropyridine-3,4-dicarboxylate (1.6 g, 3.2 mmol, 66% yield). MS obsd. (ESI+): 478.1, 492.1 (M+H)⁺.

### Step D: 1-((1r,3r)-3-(1H-imidazol-1-yl)cyclobutyl)-5-(tert-butoxycarbonyl)-2-oxo-1,2-dihydropyridine-4-carboxylic acid

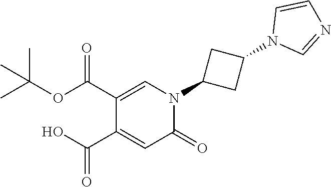 [SMILES: CC(C)(C)OC(=O)c1cn([C@H]2C[C@H](n3ccnc3)C2)c(=O)cc1C(=O)O]

A mixture of 3-(tert-butyl) 4-methyl 6-oxo-1-((1s,3s)-3-(tosyloxy)cyclobutyl)-1,6-dihydropyridine-3,4-dicarboxylate and 3-(tert-butyl) 4-ethyl 6-oxo-1-((1s,3s)-3-(tosyloxy)cyclobutyl)-1,6-dihydropyridine-3,4-dicarboxylate (1.0 g, 2.0 mmol) and imidazole (3.3 g, 49.0 mmol) was stirred for 20 h at 120° C. Then the mixture was directly purified by flash chromatography (eluting with 0-60% MeOH in DCM) to afford 1-((1r,3r)-3-(1H-imidazol-1-yl)cyclobutyl)-5-(tert-butoxycarbonyl)-2-oxo-1,2-dihydropyridine-4-carboxylic acid (420.0 mg, 1.17 mmol, 57% yield). MS obsd. (ESI+): 360.1 (M+H)⁺.

### Step E: tert-butyl 1-((1r,3r)-3-(1H-imidazol-1-yl)cyclobutyl)-4-(2-(tert-butoxycarbonyl)-1-methylhydrazine-1-carbonyl)-6-oxo-1,6-dihydropyridine-3-carboxylate

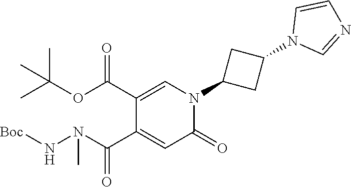 [SMILES: CN(NC(=O)OC(C)(C)C)C(=O)c1cc(=O)n([C@H]2C[C@H](n3ccnc3)C2)cc1C(=O)OC(C)(C)C]

To a solution of 1-((1r,3r)-3-(1H-imidazol-1-yl)cyclobutyl)-5-(tert-butoxycarbonyl)-2-oxo-1,2-dihydropyridine-4-carboxylic acid (420 mg, 1.17 mmol) in DMF (8.0 mL) was added HATU (894 mg, 2.34 mmol), N-ethyl-N-isopropyl-propan-2-amine (755 mg, 5.84 mmol, 1.0 mL) and tert-butyl N-(methylamino)carbamate (342 mg, 2.34 mmol) at 0° C. The mixture was stirred for 16 h at 25° C. Then the mixture was quenched with H₂O (100 mL), extracted with ethyl acetate (30 mL×3), and dried over MgSO₄. The combined organic phases were concentrated and purified by flash chromatography (eluting with 0-20% MeOH in DCM) to afford tert-butyl 1-((1r,3r)-3-(1H-imidazol-1-yl)cyclobutyl)-4-(2-(tert-butoxycarbonyl)-1-methylhydrazine-1-carbonyl)-6-oxo-1,6-dihydropyridine-3-carboxylate (498 mg, 1.0 mmol, 87% yield). MS obsd. (ESI+): 488.3 (M+H)⁺.

### Step F: 6-((1r,3r)-3-(1H-imidazol-1-yl)cyclobutyl)-2-methyl-2,3-dihydropyrido[3,4-d]pyridazine-1,4,7(6H)-trione

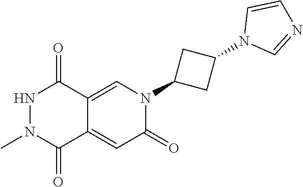 [SMILES: Cn1[nH]c(=O)c2cn([C@H]3C[C@H](n4ccnc4)C3)c(=O)cc2c1=O]

To the solution of tert-butyl 1-((1r,3r)-3-(1H-imidazol-1-yl)cyclobutyl)-4-(2-(tert-butoxycarbonyl)-1-methylhydrazine-1-carbonyl)-6-oxo-1,6-dihydropyridine-3-carboxylate (498 mg, 1.0 mmol) in DCM (20.0 mL) was added 2,2,2-trifluoroacetic acid (11.8 g, 103.84 mmol, 8.0 mL) at 0° C. and the reaction was stirred for 16 h at 25° C. Then the mixture was concentrated and purified by flash chromatography (eluting with 0-100% MeOH in DCM) to afford 6-((1r,3r)-3-(1H-imidazol-1-yl)cyclobutyl)-2-methyl-2,3-dihydropyrido[3,4-d]pyridazine-1,4,7(6H)-trione (290 mg, 0.93 mmol, 91% yield). MS obsd. (ESI+): 314.1 (M+H)⁺.

### Step G: 6-((1r,3r)-3-(1H-imidazol-1-yl)cyclobutyl)-2-methyl-1,7-dioxo-1,2,6,7-tetrahydropyrido[3,4-d]pyridazin-4-yl trifluoromethanesulfonate

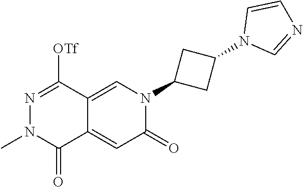 [SMILES: Cn1nc(OS(=O)(=O)C(F)(F)F)c2cn([C@H]3C[C@H](n4ccnc4)C3)c(=O)cc2c1=O]

To a solution of 6-((1r,3r)-3-(1H-imidazol-1-yl)cyclobutyl)-2-methyl-2,3-dihydropyrido[3,4-d]pyridazine-1,4,7(6H)-trione (290 mg, 0.93 mmol, 1.0 eq.) in dry pyridine (4.0 mL) was added 1,1,1-trifluoro-N-phenyl-N-(trifluoromethylsulfonyl)methanesulfonamide (992 mg, 2.78 mmol, 3.0 eq.) and the reaction was stirred for 48 h at 25° C. Then the mixture was concentrated and purified by flash chromatography (eluting with 0-15% MeOH in DCM) to afford 6-((1r,3r)-3-(1H-imidazol-1-yl)cyclobutyl)-2-methyl-1,7-dioxo-1,2,6,7-tetrahydropyrido[3,4-d]pyridazin-4-yl trifluoromethanesulfonate (120 mg, 0.27 mmol, 29% yield). MS obsd. (ESI+): 446.1 (M+H)⁺.

### Step H: 6-((1r,3R)-3-(1H-imidazol-1-yl)cyclobutyl)-2-methyl-4-(((R)-1-(2-methyl-3-(trifluoromethyl)phenyl)ethyl)amino)-2,6-dihydropyrido[3,4-d]pyridazine-1,7-dione (example 29)

A neat mixture of 6-((1r,3r)-3-(1H-imidazol-1-yl)cyclobutyl)-2-methyl-1,7-dioxo-1,2,6,7-tetrahydropyrido[3,4-d]pyridazin-4-yl trifluoromethanesulfonate (90 mg, 0.20 mmol, 1.0 eq.) and (1R)-1-[2-methyl-3-(trifluoromethyl)phenyl]ethanamine (493 mg, 2.42 mmol) was stirred for 2 h at 100° C. in a microwave reactor. Then the mixture was purified by flash chromatography (eluting with 0-15% MeOH in DCM) and further purified by preparative HPLC to afford 6-((1r,3R)-3-(1H-imidazol-1-yl)cyclobutyl)-2-methyl-4-(((R)-1-(2-methyl-3-(trifluoromethyl)phenyl)ethyl)amino)-2,6-dihydropyrido[3,4-d]pyridazine-1,7-dione (8 mg, 0.02 mmol, 8% yield). MS obsd. (ESI+): 499.3 (M+H)⁺.

### Examples 30 and 31: 4-(((R)-1-(3-(difluoromethyl)-2-fluorophenyl)ethyl)amino)-6-((3R,4S)-3-fluorotetrahydro-2H-pyran-4-yl)-2-methyl-2,6-dihydropyrido[3,4-d]pyridazine-1,7-dione (example 30, stereochemistry at tetrahydropyran is arbitrarily assigned) and 4-(((R)-1-(3-(difluoromethyl)-2-fluorophenyl)ethyl)amino)-6-((3S,4R)-3-fluorotetrahydro-2H-pyran-4-yl)-2-methyl-2,6-dihydropyrido[3,4-d]pyridazine-1,7-dione (example 31, stereochemistry at tetrahydropyran is arbitrarily assigned)

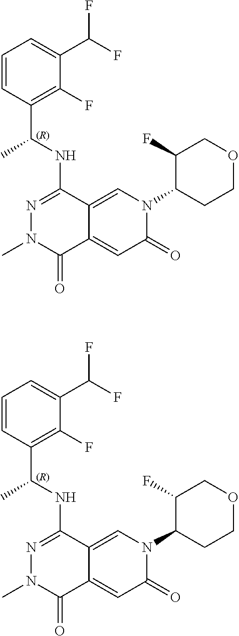

Prepared in an essentially analogous manner to example 4, starting with racemic trans-3-fluorotetrahydro-2H-pyran-4-amine (prepared according to the method of WO/2013/020062). Chiral separation at the last step afforded two unassigned tetrahydropyran stereoisomers.

Isomer 1: 4-(((R)-1-(3-(difluoromethyl)-2-fluorophenyl)ethyl)amino)-6-((3R,4S)-3-fluorotetrahydro-2H-pyran-4-yl)-2-methyl-2,6-dihydropyrido[3,4-d]pyridazine-1,7-dione (example 30, stereochemistry at tetrahydropyran is arbitrarily assigned). MS obsd. (ESI+): 467.0 (M+H)⁺. Chiral HPLC: (Column: (R,R)Whelk-O1, 4.6*100 mm 3.5 um, Flow rate: 3.0 mL/min, Eluent: MeOH (0.2% 7M NH3 in MeOH), Temp 40° C.) Retention time=3.42 min.

Isomer 2: 4-(((R)-1-(3-(difluoromethyl)-2-fluorophenyl)ethyl)amino)-6-((3S,4R)-3-fluorotetrahydro-2H-pyran-4-yl)-2-methyl-2,6-dihydropyrido[3,4-d]pyridazine-1,7-dione (example 31, stereochemistry at tetrahydropyran is arbitrarily assigned). MS obsd. (ESI+): 467.0 (M+H)⁺. Chiral HPLC: (Column: (R,R)Whelk-O1, 4.6*100 mm 3.5 um, Flow rate: 3.0 mL/min, Eluent: MeOH (0.2% 7M NH3 in MeOH), Temp 40° C.) Retention time=3.05 min.

### Examples 32 and 33: 4-(((R)-1-(3-(difluoromethyl)-2-fluorophenyl)ethyl)amino)-6-((3R,4R)-3-fluorotetrahydro-2H-pyran-4-yl)-2-methyl-2,6-dihydropyrido[3,4-d]pyridazine-1,7-dione (example 32, tetrahydropyran absolute stereochemistry arbitrarily assigned) and 4-(((R)-1-(3-(difluoromethyl)-2-fluorophenyl)ethyl)amino)-6-((3S,4S)-3-fluorotetrahydro-2H-pyran-4-yl)-2-methyl-2,6-dihydropyrido[3,4-d]pyridazine-1,7-dione (example 33, tetrahydropyran absolute stereochemistry arbitrarily assigned)

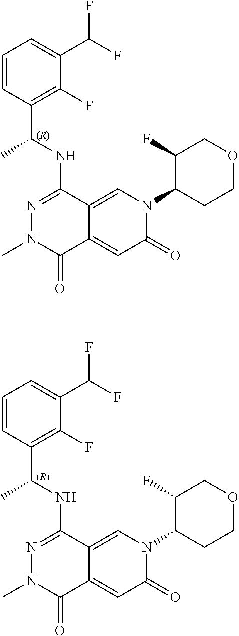

Prepared in an essentially analogous manner to example 4, starting with racemic cis-3-fluorotetrahydro-2H-pyran-4-amine (prepared according to the method of WO/2013/020062). Chiral separation at the last step afforded two tetrahydropyran stereoisomers.

Isomer 1: 4-(((R)-1-(3-(difluoromethyl)-2-fluorophenyl)ethyl)amino)-6-((3R,4R)-3-fluorotetrahydro-2H-pyran-4-yl)-2-methyl-2,6-dihydropyrido[3,4-d]pyridazine-1,7-dione (example 32, tetrahydropyran absolute stereochemistry arbitrarily assigned). MS obsd. (ESI+): 467.4 (M+H)⁺. Chiral HPLC: (Column: IC-3, 4.6*100 mm 3 um, Flow rate: 3.0 mL/min, Eluent: EtOH (1% 7M NH3 in MeOH), Temp 40° C.) Retention time=2.48 min.

Isomer 2: 4-(((R)-1-(3-(difluoromethyl)-2-fluorophenyl)ethyl)amino)-6-((3S,4S)-3-fluorotetrahydro-2H-pyran-4-yl)-2-methyl-2,6-dihydropyrido[3,4-d]pyridazine-1,7-dione (example 33, tetrahydropyran absolute stereochemistry arbitrarily assigned). MS obsd. (ESI+): 467.4 (M+H)⁺. Chiral HPLC: (Column: IC-3, 4.6*100 mm 3 um, Flow rate: 3.0 mL/min, Eluent: EtOH (1% 7M NH3 in MeOH), Temp 40° C.) Retention time=2.99 min.

### Examples 34 and 35: 6-cyclopropyl-2-methyl-4-(((R)-1-(2-methyl-3-(trifluoromethyl)phenyl)ethyl)amino)-8-((S)-pyrrolidin-2-yl)-2,6-dihydropyrido[3,4-d]pyridazine-1,7-dione (example 34, Absolute stereochemistry at pyrrolidine arbitrarily defined) and 6-cyclopropyl-2-methyl-4-(((R)-1-(2-methyl-3-(trifluoromethyl)phenyl)ethyl)amino)-8-((R)-pyrrolidin-2-yl)-2,6-dihydropyrido[3,4-d]pyridazine-1,7-dione (example 35, Absolute stereochemistry at pyrrolidine arbitrarily defined)

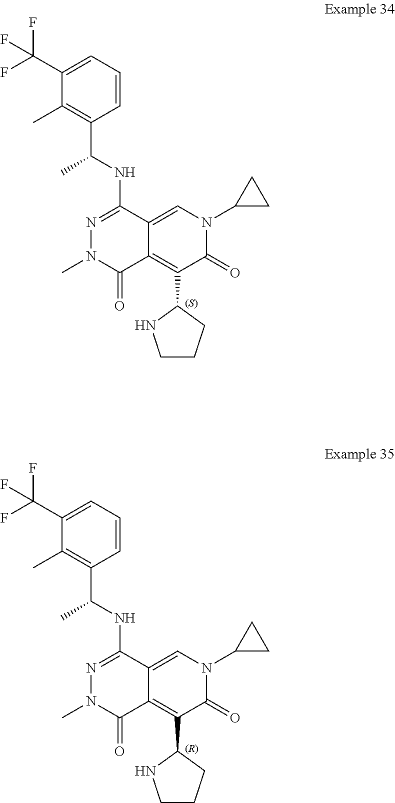

### Step A: tert-butyl 2-(6-cyclopropyl-2-methyl-4-(((R)-1-(2-methyl-3-(trifluoromethyl)phenyl)ethyl)amino)-1,7-dioxo-1,2,6,7-tetrahydropyrido[3,4-d]pyridazin-8-yl)-2,5-dihydro-1H-pyrrole-1-carboxylate

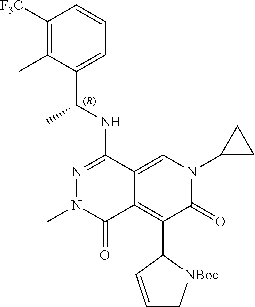

To a mixture of (R)-8-bromo-6-cyclopropyl-2-methyl-4-((1-(2-methyl-3-(trifluoromethyl)phenyl)ethyl)amino)-2,6-dihydropyrido[3,4-d]pyridazine-1,7-dione (230 mg, 462 umol), tert-butyl 2,3-dihydropyrrole-1-carboxylate (156 mg, 923 umol), palladium(II) acetate (21 mg, 92 umol), potassium carbonate (192 mg, 1.39 mmol) in 1,4-Dioxane (10 mL) was added triphenylphosphine (49 mg, 185 umol) under N₂. The mixture was heated to 60° C. for 16 h. The mixture was then concentrated, adsorbed on silica gel and was purified by preparative TLC (eluting with 10% MeOH in DCM) to afford tert-butyl 2-(6-cyclopropyl-2-methyl-4-(((R)-1-(2-methyl-3-(trifluoromethyl)phenyl)ethyl)amino)-1,7-dioxo-1,2,6,7-tetrahydropyrido[3,4-d]pyridazin-8-yl)-2,5-dihydro-1H-pyrrole-1-carboxylate (220 mg, ˜80% purity, 74% yield) MS obsd. (ESI+): 586.5 (M+H)⁺.

### Step B: tert-butyl 2-(6-cyclopropyl-2-methyl-4-(((R)-1-(2-methyl-3-(trifluoromethyl)phenyl)ethyl)amino)-1,7-dioxo-1,2,6,7-tetrahydropyrido[3,4-d]pyridazin-8-yl)pyrrolidine-1-carboxylate

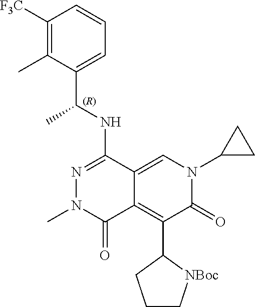

To a solution of tert-butyl tert-butyl 2-(6-cyclopropyl-2-methyl-4-(((R)-1-(2-methyl-3-(trifluoromethyl)phenyl)ethyl)amino)-1,7-dioxo-1,2,6,7-tetrahydropyrido[3,4-d]pyridazin-8-yl)-2,5-dihydro-1H-pyrrole-1-carboxylate (208 mg, 285 umol, 80% purity) in MeOH (11 mL) was added Pd(OH)₂ (40 mg, 285 umol). The mixture was stirred at room temperature for 2 hours. The mixture was concentrated under vacuum to obtain the crude product. The crude product was purified by preparative HPLC eluting to afford tert-butyl 2-(6-cyclopropyl-2-methyl-4-(((R)-1-(2-methyl-3-(trifluoromethyl)phenyl)ethyl)amino)-1,7-dioxo-1,2,6,7-tetrahydropyrido[3,4-d]pyridazin-8-yl)pyrrolidine-1-carboxylate (30 mg, 51 umol, 17% yield). MS obsd. (ESI+): 588.6 (M+H)⁺

### Step C: 6-cyclopropyl-2-methyl-4-(((R)-1-(2-methyl-3-(trifluoromethyl)phenyl)ethyl)amino)-8-(pyrrolidin-2-yl)-2,6-dihydropyrido[3,4-d]pyridazine-1,7-dione

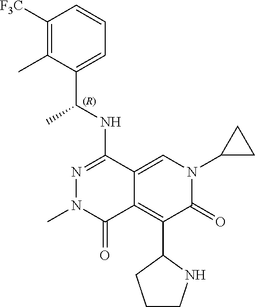

To a solution of tert-butyl 2-(6-cyclopropyl-2-methyl-4-(((R)-1-(2-methyl-3-(trifluoromethyl)phenyl)ethyl)amino)-1,7-dioxo-1,2,6,7-tetrahydropyrido[3,4-d]pyridazin-8-yl)pyrrolidine-1-carboxylate (50 mg, 85 umol) in DCM (6 mL) was added 4 M HCl in 1,4-Dioxane (4 M in 1,4-dioxane, 2.13 mL) at 0° C. The mixture was stirred at room temperature for 16 hours. The mixture was concentrated under vacuum. The crude product was purified by preparative HPLC to give 6-cyclopropyl-2-methyl-4-(((R)-1-(2-methyl-3-(trifluoromethyl)phenyl)ethyl)amino)-8-(pyrrolidin-2-yl)-2,6-dihydropyrido[3,4-d]pyridazine-1,7-dione (13 mg, 27 umol, 24% yield). MS obsd. (ESI+): 488.2 (M+H)⁺.

### Step D: 6-cyclopropyl-2-methyl-4-(((R)-1-(2-methyl-3-(trifluoromethyl)phenyl)ethyl)amino)-8-((S)-pyrrolidin-2-yl)-2,6-dihydropyrido[3,4-d]pyridazine-1,7-dione (example 34) and 6-cyclopropyl-2-methyl-4-(((R)-1-(2-methyl-3-(trifluoromethyl)phenyl)ethyl)amino)-8-((R)-pyrrolidin-2-yl)-2,6-dihydropyrido[3,4-d]pyridazine-1,7-dione (example 35)

6-cyclopropyl-2-methyl-4-(((R)-1-(2-methyl-3-(trifluoromethyl)phenyl)ethyl)amino)-8-(pyrrolidin-2-yl)-2,6-dihydropyrido[3,4-d]pyridazine-1,7-dione was separated by chiral SFC to afford: 6-cyclopropyl-2-methyl-4-(((R)-1-(2-methyl-3-(trifluoromethyl)phenyl)ethyl)amino)-8-((S)-pyrrolidin-2-yl)-2,6-dihydropyrido[3,4-d]pyridazine-1,7-dione (example 34, pyrrolidine stereochemistry arbitrarily assigned) and 6-cyclopropyl-2-methyl-4-(((R)-1-(2-methyl-3-(trifluoromethyl)phenyl)ethyl)amino)-8-((R)-pyrrolidin-2-yl)-2,6-dihydropyrido[3,4-d]pyridazine-1,7-dione (example 35, pyrrolidine stereochemistry arbitrarily assigned),

Example 34: MS obsd. (ESI+): 488.2 (M+H)⁺. Chiral HPLC: (Column: Cellulose-SC, 4.6*100 mm 3.5 um, Flow rate: 3.0 mL/min, Eluent: MeOH (0.2% 7M NH3 in MeOH), Temp 40° C.) Retention time=2.07 min.

Example 35: MS obsd. (ESI+): 488.2 (M+H)⁺. Chiral HPLC: (Column: Cellulose-SC, 4.6*100 mm 3.5 um, Flow rate: 3.0 mL/min, Eluent: MeOH (0.2% 7M NH3 in MeOH), Temp 40° C.) Retention time=1.95 min.

### Example 36: (R)-4-((1-(3-(1,1-difluoroethyl)-2-fluorophenyl)ethyl)amino)-2-methyl-6-(tetrahydro-2H-pyran-4-yl)-2,6-dihydropyrido[3,4-d]pyridazine-1,7-dione

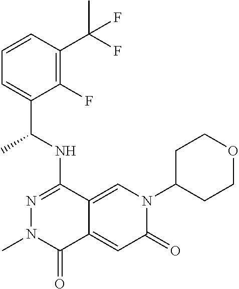 [SMILES: C[C@@H](Nc1nn(C)c(=O)c2cc(=O)n(C3CCOCC3)cc12)c1cccc(C(C)(F)F)c1F]

To a mixture of 2-methyl-1,7-dioxo-6-(tetrahydro-2H-pyran-4-yl)-1,2,6,7-Tetrahydropyrido[3,4-d]pyridazin-4-yl 2,4,6-triisopropylbenzenesulfonate (57 mg, 105), (R)-1-(3-(1,1-difluoroethyl)-2-fluorophenyl)ethan-1-amine hydrochloride (26 mg, 126 umol, prepared according to the procedure of WO2019122129) in dioxane (0.5 mL) was added cesium carbonate (102 mg, 315 umol), and XantPhos-PdG3 (10.0 mg, 10.5 umol). The mixture was stirred at 110° C. for 3 hr. Upon cooling to room temperature, the solution was then concentrated and the resulting residue was purified by flash column chromatography (MeOH in DCM=2%). Further purification with preparative HPLC afforded (R)-4-((1-(3-(1,1-difluoroethyl)-2-fluorophenyl)ethyl)amino)-2-methyl-6-(tetrahydro-2H-pyran-4-yl)-2,6-dihydropyrido[3,4-d]pyridazine-1,7-dione (11.0 mg, 24 umol, 23% yield). MS obsd. (ESI+): 463.2 (M+H)+.

### Example 37: (R)-4-((1-(3-(1,1-difluoroethyl)-2-fluorophenyl)ethyl)amino)-8-methoxy-2-methyl-6-(tetrahydro-2H-pyran-4-yl)-2,6-dihydropyrido[3,4-d]pyridazine-1,7-dione

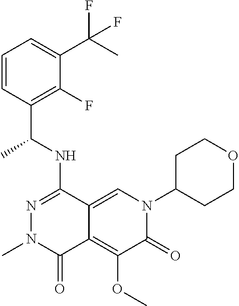 [SMILES: COc1c(=O)n(C2CCOCC2)cc2c(N[C@H](C)c3cccc(C(C)(F)F)c3F)nn(C)c(=O)c12]

### Step A: (8-bromo-2-methyl-1,7-dioxo-6-tetrahydropyran-4-yl-pyrido[3,4-d]pyridazin-4-yl) 2,4,6-triisopropylbenzenesulfonate

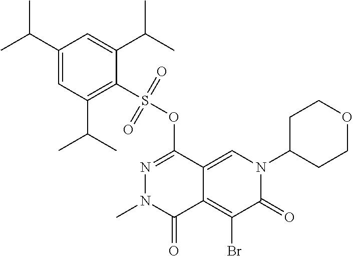 [SMILES: CC(C)c1cc(C(C)C)c(S(=O)(=O)Oc2nn(C)c(=O)c3c(Br)c(=O)n(C4CCOCC4)cc23)c(C(C)C)c1]

A mixture of 2-methyl-1,7-dioxo-6-(tetrahydro-2H-pyran-4-yl)-1,2,6,7-tetrahydropyrido[3,4-d]pyridazin-4-yl 2,4,6-triisopropylbenzenesulfonate (3.11 g, 5.72 mmol), potassium acetate (842 mg, 8.58 mmol) and molecular bromine (1.37 g, 8.58 mmol) in acetic acid (55 mL) was heated to 90° C. for 16 hours. The mixture then cooled to room temperature and concentrated under reduced pressure. The residue was purified by flash column (PE:EA=2:1) to afford (8-bromo-2-methyl-1,7-dioxo-6-tetrahydropyran-4-yl-pyrido[3,4-d]pyridazin-4-yl) 2,4,6-triisopropylbenzenesulfonate (3.32 g, 4.5 mmol, 79% yield, 85% purity). MS obsd. (ESI+): ^{79/81}Br 622.4/624.4 (M+H)⁺

### Step C: (8-methoxy-2-methyl-1,7-dioxo-6-tetrahydropyran-4-yl-pyrido[3,4-d]pyridazin-4-yl) 2,4,6-triisopropylbenzenesulfonate

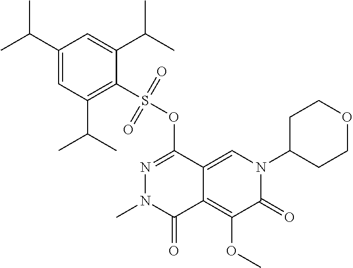 [SMILES: COc1c(=O)n(C2CCOCC2)cc2c(OS(=O)(=O)c3c(C(C)C)cc(C(C)C)cc3C(C)C)nn(C)c(=O)c12]

To a mixture of (8-bromo-2-methyl-1,7-dioxo-6-tetrahydropyran-4-yl-pyrido[3,4-d]

pyridazin-4-yl) 2,4,6-triisopropylbenzenesulfonate (400 mg, 546 umol, 85% purity), MeOH (440 mg, 13.73 mmol) in dioxane (4 mL) was added cesium carbonate (520 mg, 1.60 mmol) and Xantphos Pd G3 (110 mg, 116.29 umol) under N₂ atmosphere. The reaction mixture was stirred at 50° C. for 4 hours, cooled to room temperature and concentrated in vacuum. The resulting residue was dissolved in 10 ml DCM, 360 mg DIPEA, and 360 mg 2,4,6-triisopropylbenzenesulfonyl chloride were added and stirred at room temperature for 3 hours. The mixture was concentrated in vacuum and the residue was purified by flash chromatography (DCM:MeOH=25:1) to afford (8-methoxy-2-methyl-1,7-dioxo-6-tetrahydropyran-4-yl-pyrido[3,4-d]pyridazin-4-yl) 2,4,6-triisopropylbenzenesulfonate (272 mg, 403 umol, 73% yield). MS obsd. (ESI+): 574.4 (M+H)⁺.

### Step D: (R)-4-((1-(3-(1,1-difluoroethyl)-2-fluorophenyl)ethyl)amino)-8-methoxy-2-methyl-6-(tetrahydro-2H-pyran-4-yl)-2,6-dihydropyrido[3,4-d]pyridazine-1,7-dione (example 37)

To a mixture of (8-methoxy-2-methyl-1,7-dioxo-6-tetrahydropyran-4-yl-pyrido [3,4-d]pyridazin-4-yl) 2,4,6-triisopropylbenzenesulfonate (222 mg, 387 umol), (1R)-1-[3-(1,1-difluoroethyl)-2-fluoro-phenyl]ethanamine hydrochloride (94 mg, 460 umol), XantphosPdG3 (37 mg, 39 umol) in 1,4-dioxane (2.5 mL) was added cesium carbonate (378 mg, 1.16 mmol). The mixture was stirred at 110° C. for 16 hr, cooled to room temperature and concentrated under vacuum. The residue was purified by flash chromatography (2% MeOH in DCM), followed by preparative HPLC to afford (R)-4-((1-(3-(1,1-difluoroethyl)-2-fluorophenyl)ethyl)amino)-8-methoxy-2-methyl-6-(tetrahydro-2H-pyran-4-yl)-2,6-dihydropyrido[3,4-d]pyridazine-1,7-dione (77.3 mg, 157 umol, 41% yield). MS obsd. (ESI+): 493.2 (M+H)⁺.

### Example 38 and 39: 4-(((R)-1-(3-(difluoromethyl)-2-fluorophenyl)ethyl)amino)-2-methyl-6-((S)-tetrahydro-2H-pyran-3-yl)-2,6-dihydropyrido[3,4-d]pyridazine-1,7-dione (example 38, tetrahydropyran absolute stereochemistry arbitrarily assigned) and 4-(((R)-1-(3-(difluoromethyl)-2-fluorophenyl)ethyl)amino)-2-methyl-6-((R)-tetrahydro-2H-pyran-3-yl)-2,6-dihydropyrido[3,4-d]pyridazine-1,7-dione (example 39, tetrahydropyran absolute stereochemistry arbitrarily assigned)

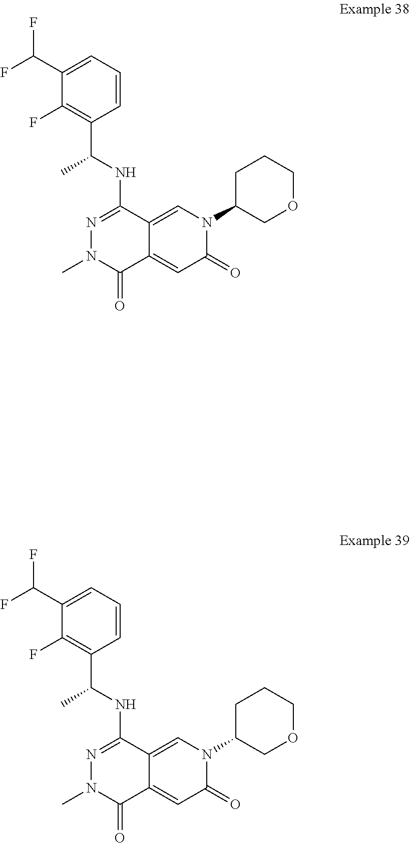 [SMILES: C[C@@H](Nc1nn(C)c(=O)c2cc(=O)n([C@H]3CCCOC3)cc12)c1cccc(C(F)F)c1F ; C[C@@H](Nc1nn(C)c(=O)c2cc(=O)n([C@@H]3CCCOC3)cc12)c1cccc(C(F)F)c1F]

The title compounds were prepared in an analogous manner as example 4, using racemic tetrahydropyran-3-amine hydrochloride. Chiral SFC afforded (4-(((R)-1-(3-(difluoromethyl)-2-fluorophenyl)ethyl)amino)-2-methyl-6-((S)-tetrahydro-2H-pyran-3-yl)-2,6-dihydropyrido[3,4-d]pyridazine-1,7-dione (example 38, tetrahydropyran absolute stereochemistry arbitrarily assigned). MS obsd. (ESI+): 449.4 (M+H)⁺. Chiral HPLC: (Column: AD-3, 4.6*100 mm 3 um, Flow rate: 3.0 mL/min, Eluent: EtOH (1% 7M NH3 in MeOH), Temp 40° C.) Retention time=1.20 min. 4-(((R)-1-(3-(difluoromethyl)-2-fluorophenyl)ethyl)amino)-2-methyl-6-((R)-tetrahydro-2H-pyran-3-yl)-2,6-dihydropyrido[3,4-d]pyridazine-1,7-dione (example 39, tetrahydropyran absolute stereochemistry arbitrarily assigned). MS obsd. (ESI+): 449.4 (M+H)⁺. Chiral HPLC: (Column: AD-3, 4.6*100 mm 3 um, Flow rate: 3.0 mL/min, Eluent: EtOH (1% 7M NH3 in MeOH), Temp 40° C.) Retention time=1.47 min.

### Example 40: (R)-4-((1-(3-(1,1-difluoro-2-hydroxy-2-methylpropyl)-2-fluorophenyl)ethyl)amino)-2-methyl-6-(tetrahydro-2H-pyran-4-yl)-2,6-dihydropyrido[3,4-d]pyridazine-1,7-dione

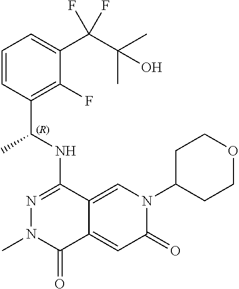

A mixture of (2-methyl-1,7-dioxo-6-tetrahydropyran-4-yl-pyrido[3,4-d]pyridazin-4-yl) 2,4,6-triisopropylbenzenesulfonate (70 mg, 129 umol), (R)-1-(3-(1-aminoethyl)-2-fluorophenyl)-1,1-difluoro-2-methylpropan-2-ol HCl (55 mg, 193 umol, prepared according to the procedure detailed in WO2019122129), XantPhos Pd G3 (32 mg, 39 umol) and cesium carbonate (126 mg, 386 umol) was dissolved in 1,4-dioxane (2 mL) and then stirred at 100° C. for 16 hrs. The mixture was then filtered and the crude filtrate was diluted in ethyl acetate and washed with NaHCO₃ (aq). The organic phase was dried over sodium sulfate and concentrated under reduced pressure. The crude residue was purified by preparative TLC (DCM/MeOH=30:1) followed by preparative HPLC to afford (R)-4-((1-(3-(1,1-difluoro-2-hydroxy-2-methylpropyl)-2-fluorophenyl)ethyl)amino)-2-methyl-6-(tetrahydro-2H-pyran-4-yl)-2,6-dihydropyrido[3,4-d]pyridazine-1,7-dione (24 mg, 47 umol, 37% yield). MS obsd. (ESI+): 507.4, [M+H]⁺.

### Example 41: (R)-4-((1-(3-(1,1-difluoro-2-hydroxy-2-methylpropyl)-2-fluorophenyl)ethyl)amino)-8-methoxy-2-methyl-6-(tetrahydro-2H-pyran-4-yl)-2,6-dihydropyrido[3,4-d]pyridazine-1,7-dione

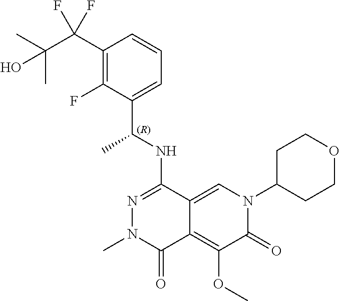

(R)-4-((1-(3-(1,1-difluoro-2-hydroxy-2-methylpropyl)-2-fluorophenyl)ethyl)amino)-8-methoxy-2-methyl-6-(tetrahydro-2H-pyran-4-yl)-2,6-dihydropyrido[3,4-d]pyridazine-1,7-dione (example 41) was prepared in an analogous manner to example 37. MS obsd. (ESI+): 537.4 [M+H]⁺.

### Example 42: (R)-6-cyclopropyl-4-((1-(3-(1,1-difluoro-2-hydroxy-2-methylpropyl)-2-fluorophenyl)ethyl)amino)-2-methyl-2,6-dihydropyrido[3,4-d]pyridazine-1,7-dione

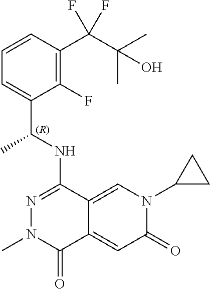

(R)-6-cyclopropyl-4-((1-(3-(1,1-difluoro-2-hydroxy-2-methylpropyl)-2-fluorophenyl)ethyl)amino)-2-methyl-2,6-dihydropyrido[3,4-d]pyridazine-1,7-dione (example 42) was prepared in an analogous manner to example 40. MS obsd. (ESI+): 463.4, [M+H]⁺.

### Example 43: (R)-4-((1-(3-(difluoromethyl)-2-fluorophenyl)ethyl)amino)-6-(1-(difluoromethyl)cyclopropyl)-2-methyl-2,6-dihydropyrido[3,4-d]pyridazine-1,7-dione

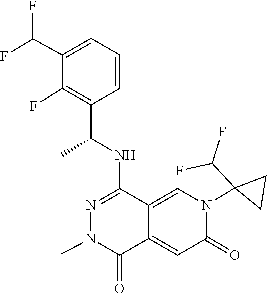 [SMILES: C[C@@H](Nc1nn(C)c(=O)c2cc(=O)n(C3(C(F)F)CC3)cc12)c1cccc(C(F)F)c1F]

### Step A: dimethyl 2-((dimethylamino)methylene)-3-oxopentanedioate

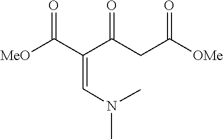 [SMILES: COC(=O)CC(=O)/C(=C\N(C)C)C(=O)OC]

To a solution of dimethyl 3-oxopentanedioate (5.0 g, 28.71 mmol) in MeOH (100 mL) was added 1,1-dimethoxy-N,N-dimethyl-methanamine (4.1 g, 34.45 mmol). The mixture was stirred at rt for 4 h. The reaction mixture was used directly in the next step. MS obsd. (ESI+): 230.2 (M+H)⁺.

### Step B: methyl 1-(1-(difluoromethyl)cyclopropyl)-4-hydroxy-6-oxo-1,6-dihydropyridine-3-carboxylate

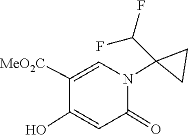 [SMILES: COC(=O)c1cn(C2(C(F)F)CC2)c(=O)cc1O]

To a crude solution of dimethyl 2-((dimethylamino)methylene)-3-oxopentanedioate (1.3 g, 5.43 mmol) in MeOH (25 mL) was added 1-(difluoromethyl)cyclopropanamine (640 mg, 5.98 mmol). The mixture was stirred at rt for 6 h and then the solvent was removed under reduced pressure. Water (50 mL) was added to the residue and the suspension was adjusted to pH=11. The solution was extracted with EA. The aqueous phase was acidified with saturated citric acid to pH 3. The aqueous phase was extracted with DCM. The organic layer was dried over anhydrous Na₂SO₄, concentrated to afford pure methyl 1-(1-(difluoromethyl)cyclopropyl)-4-hydroxy-6-oxo-1,6-dihydropyridine-3-carboxylate (980 mg, 3.66 mmol, 67% yield). MS obsd. (ESI+): 260.2 (M+H)⁺.

### Step C: Methyl 1-(1-(difluoromethyl)cyclopropyl)-6-oxo-4-(((trifluoromethyl)sulfonyl)oxy)-1,6-dihydropyridine-3-carboxylate

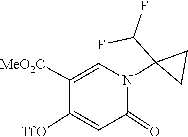 [SMILES: COC(=O)c1cn(C2(C(F)F)CC2)c(=O)cc1OS(=O)(=O)C(F)(F)F]

To a solution of methyl 1-(1-(difluoromethyl)cyclopropyl)-4-hydroxy-6-oxo-1,6-dihydropyridine-3-carboxylate (748 mg, 2.89 mmol) and 1,1,1-trifluoro-N-phenyl-N-((trifluoromethyl)sulfonyl)methanesulfonamide (1.6 g, 4.33 mmol) in dry DMF (30 mL) was added potassium carbonate (1.2 g, 8.66 mmol). The reaction mixture was stirred at rt for 3.5 hr and then quenched by adding aqueous saturated ammonium chloride solution (50 mL). The reaction mixture was extracted with three portions of 50 mL of ethyl acetate. The combined organic layers were washed with brine and dried over sodium sulfate, filtered and concentrated. The residue was purified by flash chromatography (eluting with 0-40% EA in PE) to afford methyl 1-(1-(difluoromethyl)cyclopropyl)-6-oxo-4-(((trifluoromethyl)sulfonyl)oxy)-1,6-dihydropyridine-3-carboxylate (880 mg, 2.25 mmol, 78% yield). MS obsd. (ESI+): 392.3 (M+H)⁺.

### Step D: 1-(1-(difluoromethyl)cyclopropyl)-5-(methoxycarbonyl)-2-oxo-1,2-dihydropyridine-4-carboxylic acid

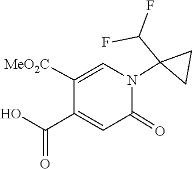 [SMILES: COC(=O)c1cn(C2(C(F)F)CC2)c(=O)cc1C(=O)O]

To a solution of methyl 1-(1-(difluoromethyl)cyclopropyl)-6-oxo-4-(((trifluoromethyl)sulfonyl)oxy)-1,6-dihydropyridine-3-carboxylate (835 mg, 2.13 mmol) in DMF (50.0 mL) and H₂O (2.5 mL) was added 1′-Bis(diphenylphosphino)ferrocene (354 mg, 640 umol), triethylamine (647 mg, 6.40 mmol) and Palladium (II) Acetate (95 mg, 427 umol). The reaction was stirred in an autoclave at 90° C. for 16 h under carbon monoxide (2.0 MPa). The solvent was then removed under reduced pressure. To the residue was added water (20 mL). The suspension was adjusted to pH 11 and extracted with ethyl acetate (20 mL). The water phase was isolated, acidified with saturated citric acid solution to pH 4, and then extracted with ethyl acetate (20 mL). The organic layer was dried over anhydrous Na₂SO₄, concentrated. The resulting material was combined with product from an identical experiment conducted on 0.29 mmol scale to afford pure 1-(1-(difluoromethyl)cyclopropyl)-5-(methoxycarbonyl)-2-oxo-1,2-dihydropyridine-4-carboxylic acid (523 mg, 1.82 mmol, 76% yield). MS obsd. (ESI+): 288.2 (M+H)⁺.

### Step E: methyl 4-(2-(tert-butoxycarbonyl)-1-methylhydrazine-1-carbonyl)-1-(1-(difluoromethyl)cyclopropyl)-6-oxo-1,6-dihydropyridine-3-carboxylate

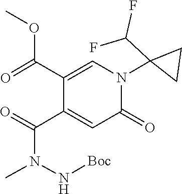 [SMILES: COC(=O)c1cn(C2(C(F)F)CC2)c(=O)cc1C(=O)N(C)NC(=O)OC(C)(C)C]

A solution of 1-(1-(difluoromethyl)cyclopropyl)-5-(methoxycarbonyl)-2-oxo-1,2-dihydropyridine-4-carboxylic acid (180 mg, 627 umol), 1-N-Boc-2-methylhydrazine (122 mg, 836b umol), DIPEA (233 mg, 1.81 mmol) and HATU (317 mg, 836 umol) in dry THE (5.0 mL) was stirred at rt for 2 h. The reaction mixture was then diluted with water and extracted with ethyl acetate. The organic layer was washed with brine and dried over sodium sulfate, filtered and concentrated. The residue was combined with material from another separate reaction run under identical conditions on a 1.01 mmol scale reaction and purified by flash chromatography on a silica gel column (eluting with 0-70% EA in PE) to afford methyl 4-(2-(tert-butoxycarbonyl)-1-methylhydrazine-1-carbonyl)-1-(1-(difluoromethyl)cyclopropyl)-6-oxo-1,6-dihydropyridine-3-carboxylate (388 mg, 934 mol, 57% yield). MS obsd. (ESI+): 360.4 (M+H−(Boc))⁺.

### Step F: 6-(1-(difluoromethyl)cyclopropyl)-2-methyl-2,3-dihydropyrido[3,4-d]pyridazine-1,4,7(6H)-trione

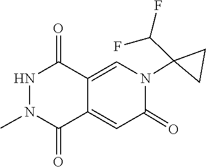 [SMILES: Cn1[nH]c(=O)c2cn(C3(C(F)F)CC3)c(=O)cc2c1=O]

A solution of methyl 4-(2-(tert-butoxycarbonyl)-1-methylhydrazine-1-carbonyl)-1-(1-(difluoromethyl)cyclopropyl)-6-oxo-1,6-dihydropyridine-3-carboxylate (388 mg, 934 umol) in HCl (4M in 1,4-dioxane, 10.0 mL) was stirred at 100° C. for 6 h. The resulting suspension was filtered and washed with ethyl acetate to give 6-(1-(difluoromethyl)cyclopropyl)-2-methyl-2,3-dihydropyrido[3,4-d]pyridazine-1,4,7(6H)-trione (151 mg, 534 umol, 57% yield). MS obsd. (ESI+): 284.3 (M+H)⁺.

### Step G: 6-(1-(difluoromethyl)cyclopropyl)-2-methyl-1,7-dioxo-1,2,6,7-tetrahydropyrido[3,4-d]pyridazin-4-yl 2,4,6-triisopropylbenzenesulfonate

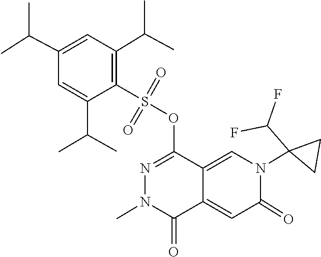 [SMILES: CC(C)c1cc(C(C)C)c(S(=O)(=O)Oc2nn(C)c(=O)c3cc(=O)n(C4(C(F)F)CC4)cc23)c(C(C)C)c1]

A solution of 6-(1-(difluoromethyl)cyclopropyl)-2-methyl-2,3-dihydropyrido[3,4-d]pyridazine-1,4,7(6H)-trione (45 mg, 160 μmol), DIPEA (68 mg, 530 μmol) and 2,4,6-triisopropylbenzenesulfonyl chloride (80 mg, 260 μmol) in DCM (1.0 mL) was stirred at rt for 2 hr. The reaction mixture was concentrated and purified by flash chromatography to afford 6-(1-(difluoromethyl)cyclopropyl)-2-methyl-1,7-dioxo-1,2,6,7-tetrahydropyrido[3,4-d]pyridazin-4-yl 2,4,6-triisopropylbenzenesulfonate (77 mg, 140 μmol, 88% yield). MS obsd. (ESI+): 550.7 (M+H)⁺.

### Step H: (R)-4-((1-(3-(difluoromethyl)-2-fluorophenyl)ethyl)amino)-6-(1-(difluoromethyl)cyclopropyl)-2-methyl-2,6-dihydropyrido[3,4-d]pyridazine-1,7-dione (example 43)

To a solution of 6-(1-(difluoromethyl)cyclopropyl)-2-methyl-1,7-dioxo-1,2,6,7-tetrahydropyrido[3,4-d]pyridazin-4-yl 2,4,6-triisopropylbenzenesulfonate (50 mg, 0.09 mmol) in 1,4-dioxane (0.5 mL) was added (R)-1-(3-(difluoromethyl)-2-fluorophenyl)ethan-1-amine hydrochloride (25 mg, 0.11 mmol), Cs₂CO₃ (177 mg, 0.55 mmol) and Xantphos-Pd-G3 (35 mg, 0.04 mmol). The reaction was irradiated in a microwave reactor at 110° C. for 3 h. The reaction was then concentrated and purified by flash chromatography (eluting with 3% MeOH in DCM). Further purification by reverse phase HPLC afforded (R)-4-((1-(3-(difluoromethyl)-2-fluorophenyl)ethyl)amino)-6-(1-(difluoromethyl)cyclopropyl)-2-methyl-2,6-dihydropyrido[3,4-d]pyridazine-1,7-dione (8 mg, 17 μmol, 19% yield). MS obsd. (ESI+): 455.1 (M+H)⁺.

### Examples 44 and 45: 4-(((S)-1-(3-(difluoro((S)-tetrahydrofuran-2-yl)methyl)-2-fluorophenyl)ethyl)amino)-2-methyl-6-(tetrahydro-2H-pyran-4-yl)-2,6-dihydropyrido[3,4-d]pyridazine-1,7-dione (example 44, stereochemistry at chiral methyl is arbitrarily assigned) and 4-(((R)-1-(3-(difluoro((S)-tetrahydrofuran-2-yl)methyl)-2-fluorophenyl)ethyl)amino)-2-methyl-6-(tetrahydro-2H-pyran-4-yl)-2,6-dihydropyrido[3,4-d]pyridazine-1,7-dione (example 45, stereochemistry at chiral methyl is arbitrarily assigned)

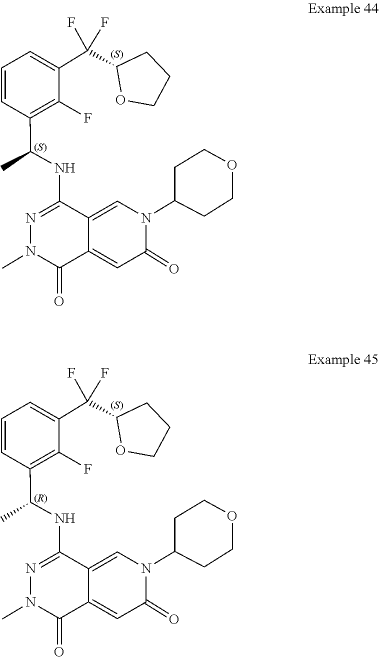

### Step A: 1-(3-(difluoro((S)-tetrahydrofuran-2-yl)methyl)-2-fluorophenyl)ethanamine

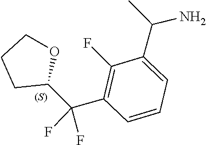

The title compound 1-(3-(difluoro((S)-tetrahydrofuran-2-yl)methyl)-2-fluorophenyl)ethanamine was synthesized according to methods described in WO/2019/122129 using racemic 2-methyl-2-propanesulfinamide. MS obsd. (ESI+): 260.2 (M+H)⁺.

Step B: 4-(((S)-1-(3-(difluoro((S)-tetrahydrofuran-2-yl)methyl)-2-fluorophenyl)ethyl)amino)-2-methyl-6-(tetrahydro-2H-pyran-4-yl)pyrido[3,4-d]pyridazine-1,7(2H,6H)-dione (example 44) and 4-(((R)-1-(3-(difluoro((S)-tetrahydrofuran-2-yl)methyl)-2-fluorophenyl)ethyl)amino)-2-methyl-6-(tetrahydro-2H-pyran-4-yl)pyrido[3,4-d]pyridazine-1,7(2H,6H)-dione (example 45).

To a solution of 1-(3-(difluoro((S)-tetrahydrofuran-2-yl)methyl)-2-fluorophenyl)ethanamine (358 mg, 1.38 mmol) in 1,4-dioxane (5.0 mL) was added (2-methyl-1,7-dioxo-6-tetrahydropyran-4-yl-pyrido[3,4-d]pyridazin-4-yl) 2,4,6-triisopropyl benzenesulfonate (500 mg, 919 umol), Cs₂CO₃ (1.8 g, 5.52 mmol) and Xantphos-Pd-G3 (349 mg, 368 umol). The reaction was irradiated in a microwave reactor at 110° C. for 3 h. The reaction was cooled to rt and then the solvent was removed under vacuum. The crude product was purified by reverse phase HPLC to afford 4-((1-(3-(difluoro((S)-tetrahydrofuran-2-yl)methyl)-2-fluorophenyl)ethyl)amino)-2-methyl-6-(tetrahydro-2H-pyran-4-yl)pyrido[3,4-d]pyridazine-1,7(2H,6H)-dione (208 mg, 0.40 mmol, 44% yield). The diastereomeric mixture was then subjected to chiral separation to afford 4-(((S)-1-(3-(difluoro((S)-tetrahydrofuran-2-yl)methyl)-2-fluorophenyl)ethyl)amino)-2-methyl-6-(tetrahydro-2H-pyran-4-yl)pyrido[3,4-d]pyridazine-1,7(2H,6H)-dione (example 44): MS obsd. (ESI+): 519.3 [(M+H)⁺]. Chiral HPLC: (Column: AS-3, 4.6*100 mm 3 um, Flow rate: 3.0 mL/min, Eluent: MeOH (0.2% 7M NH3 in MeOH), Temp 40° C.) Retention time=1.76 min. 4-(((R)-1-(3-(difluoro((S)-tetrahydrofuran-2-yl)methyl)-2-fluorophenyl)ethyl)amino)-2-methyl-6-(tetrahydro-2H-pyran-4-yl)pyrido[3,4-d]pyridazine-1,7(2H,6H)-dione (example 45): MS obsd. (ESI+): 519.3 [(M+H)⁺]. Chiral HPLC: (Column: IC-3, 4.6*100 mm 3 um, Flow rate: 3.0 mL/min, Eluent: EtOH (1% 7M NH3 in MeOH), Temp 40° C.) Retention time=2.17 min.

### Example 46: (R)-4-((1-(3-fluorobenzofuran-7-yl)ethyl)amino)-2-methyl-6-(tetrahydro-2H-pyran-4-yl)-2,6-dihydropyrido[3,4-d]pyridazine-1,7-dione

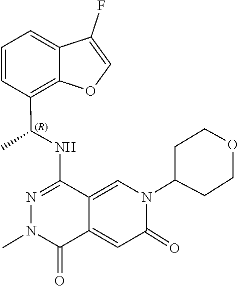

Prepared in an essentially analogous manner to example 45, starting with (R)-1-(3-fluorobenzofuran-7-yl)ethan-1-amine hydrochloride (prepared according to the method of WO/2019/122129). MS obsd. (ESI+): 439.1 (M+H)⁺.

### Example 47: (R)-4-((1-(3,3-difluoro-2,3-dihydrobenzofuran-7-yl)ethyl)amino)-2-methyl-6-(tetrahydro-2H-pyran-4-yl)-2,6-dihydropyrido[3,4-d]pyridazine-1,7-dione

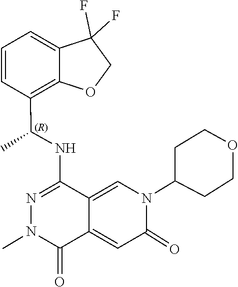

Prepared in an essentially analogous manner to example 45, starting with (R)-1-(3,3-difluoro-2,3-dihydrobenzofuran-7-yl)ethan-1-amine hydrochloride (prepared according to the method of WO/2019/122129). MS obsd. (ESI+): 459.2 (M+H)⁺.

### Example 48: (R)-8-acetyl-6-cyclopropyl-2-methyl-4-((1-(2-methyl-3-(trifluoromethyl)phenyl)ethyl)amino)-2,6-dihydropyrido[3,4-d]pyridazine-1,7-dione

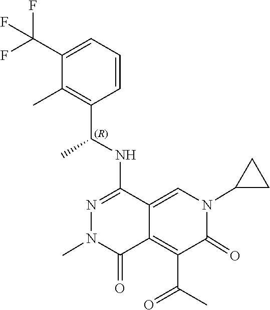

To a solution of (R)-8-bromo-6-cyclopropyl-2-methyl-4-((1-(2-methyl-3-(trifluoromethyl)phenyl)ethyl)amino)-2,6-dihydropyrido[3,4-d]pyridazine-1,7-dione (example 3, 196 mg, 0.39 mmol) in 1,4-dioxane (10 mL) was added tributyl(1-ethoxyvinyl)stannane (171 mg, 0.47 mmol), triethylamine (80 mg, 0.79 mmol) and Bis(triphenylphosphine)palladium (II) chloride (55 mg, 0.08 mmol). The mixture was stirred for 16 hr at 100° C. and treated with 0.5 mL HCl (4 M in 1,4-dioxane) for 2 h at rt. The reaction was quenched with saturated aqueous potassium fluoride. The mixture was filtered and the filtrate was extracted with DCM (3*100 mL). The organic layers were dried over Na₂SO₄ and concentrated. Purification by reverse phase HPLC to afford (R)-8-acetyl-6-cyclopropyl-2-methyl-4-((1-(2-methyl-3-(trifluoromethyl)phenyl)ethyl)amino)-2,6-dihydropyrido[3,4-d]pyridazine-1,7-dione (1.03 mg, 0.07 mmol, 0.5% yield). MS obsd. (ESI+): 461.2 (M+H)⁺.

### Example 49: (R)-6-cyclohexyl-4-((1-(3-(difluoromethyl)-2-fluorophenyl)ethyl)amino)-8-methoxy-2-methyl-2,6-dihydropyrido[3,4-d]pyridazine-1,7-dione

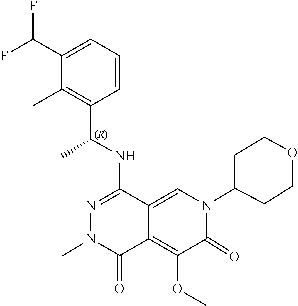

Prepared according to an analogous procedure as example 37. MS obsd. (ESI+): 477.3 (M+H)⁺.

### Example 50: (R)-4-((1-(3-(difluoromethyl)-2-fluorophenyl)ethyl)amino)-2-methyl-6-(3-morpholinobicyclo[1.1.1]pentan-1-yl)-2,6-dihydropyrido[3,4-d]pyridazine-1,7-dione

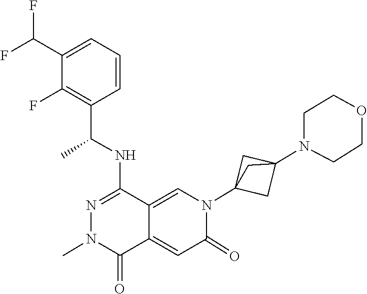 [SMILES: C[C@@H](Nc1nn(C)c(=O)c2cc(=O)n(C34CC(N5CCOCC5)(C3)C4)cc12)c1cccc(C(F)F)c1F]

### Step A: 4-bromo-6-chloro-5-methylpyridazin-3(2H)-one

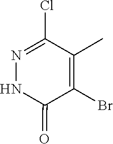

Charge 6-chloro-5-methylpyridazin-3(2H)-one (440 g, 3.05 mol) into a 10 L reaction vessel followed by water (4.4 L). To the suspension is added N-bromosuccinimide (649 g, 3.65 mol), and the suspension is stirred at 100° C. for 16 hr. The mixture is cooled to room temperature and filtered. The filter cake is washed with water (1.1 L×2) and dried under an infrared lamp to afford 4-bromo-6-chloro-5-methylpyridazin-3(2H)-one (644 g, 95% yield). ¹H NMR (400 MHz, DMSO-d₆) δ: 13.46 (s, 1H), 2.38 (s, 3H).

### Step B: 4-bromo-6-chloro-2,5-dimethylpyridazin-3(2H)-one

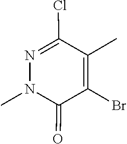

4-bromo-6-chloro-5-methylpyridazin-3(2H)-one (700 g, 3.14 mol) is charged to a 10 L reaction vessel. To the reactor is added N,N-dimethylacetamide (3.5 L) followed by potassium carbonate (653 g, 4.72 mol). Methyl iodide (493 g, 3.47 mol) is added dropwise over 1 hr at room temperature, and the mixture is stirred for 16 hours at room temperature. At this time, the reaction mixture is poured into ice water (7 L), and the resulting suspension is stirred for 30 minutes at 0° C. The mixture is filtered, and the filter cake is washed with water (1.75 L×2). The solids are dried under infrared lamp to afford 4-bromo-6-chloro-2,5-dimethylpyridazin-3(2H)-one (672 g, 2.82 mol, 91% yield). ¹H NMR (400 MHz, DMSO-d₆) δ: 3.45 (s, 3H), 2.39 (s, 3H).

### Step C: 4-bromo-5-(bromomethyl)-6-chloro-2-methylpyridazin-3(2H)-one

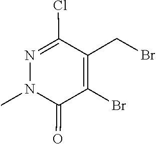

4-bromo-6-chloro-2,5-dimethylpyridazin-3(2H)-one (107 g, 450 mmol) is charged to a 2 L reactor. To the flask is added carbon tetrachloride (1.07 L), N-bromosuccinimide (160.5 g, 900 mmol), and benzoyl peroxide (22 g, 91 mmol). The mixture is stirred for 16 hr at 80° C. At this time, the solution is cooled to room temperature and filtered. The filtrate is washed with water (1 L) and extracted with DCM (500 mL). The organic phase is dried over sodium sulfate, filtered, and concentrated. The residue is then slurried in diethyl ether (1 L), and the solids are filtered and dried under vacuum to afford 4-bromo-5-(bromomethyl)-6-chloro-2-methylpyridazin-3(2H)-one (56 g, 177 mmol, 40% yield). ¹H NMR (400 MHz, CDCl₃) δ: 4.54 (s, 2H), 3.82 (s, 3H).

### Step D: (5-bromo-3-chloro-1-methyl-6-oxo-1,6-dihydropyridazin-4-yl)methyl acetate

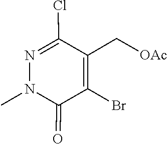

To a 2 L reactor is charged 4-bromo-5-(bromomethyl)-6-chloro-2-methylpyridazin-3(2H)-one (56 g, 177 mmol) followed by DMF (840 mL). To the mixture is added sodium acetate (29.2 g, 356 mmol). The mixture is stirred at room temperature for 3 hr, and is then diluted with water (1.68 L). The aqueous mixture is extracted with ethyl acetate (840 mL×2), and the combined organic phases are washed with water (840 mL), brine (840 mL), dried over sodium sulfate, filtered and concentrated to afford (5-bromo-3-chloro-1-methyl-6-oxo-1,6-dihydropyridazin-4-yl)methyl acetate (44 g, 149 mmol, 84% yield). ¹H NMR (400 MHz, CDCl₃) δ: 5.24 (s, 2H), 3.85 (s, 3H), 2.14 (s, 3H).

### Step E: dimethyl 2-(5-(acetoxymethyl)-6-chloro-2-methyl-3-oxo-2,3-dihydropyridazin-4-yl)malonate

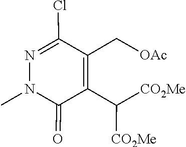 [SMILES: COC(=O)C(C(=O)OC)c1c(COC(C)=O)c(Cl)nn(C)c1=O]

To a 1 L flask containing (5-bromo-3-chloro-1-methyl-6-oxo-1,6-dihydropyridazin-4-yl)methyl acetate (26.0 g, 88 mmol) is added THE (520 mL). To the mixture is added dimethyl malonate (19.85 g, 150 mmol) and the solution is cooled to 0° C. To the mixture is charged NaH (60% in mineral oil, 7.10 g, 177 mmol) portionwise. The reaction is stirred at room temperature for 16 hours, and is then diluted with ice water (520 mL). The aqueous mixture is extracted into ethyl acetate (520 mL×2), and the combined organic layers are washed with brine (520 mL), dried over sodium sulfate, filtered and concentrated to afford dimethyl 2-(5-(acetoxymethyl)-6-chloro-2-methyl-3-oxo-2,3-dihydropyridazin-4-yl)malonate (31 g, crude), which is used without further purification. MS obsd. (ESI+): 347.0 (M+H)⁺.

### Step F: 4-chloro-2-methyl-7-methylene-5,6,7,8-tetrahydrophthalazin-1(2H)-one

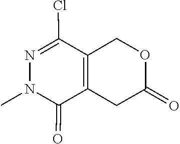

To a 500 mL flask is added dimethyl 2-(5-(acetoxymethyl)-6-chloro-2-methyl-3-oxo-2,3-dihydropyridazin-4-yl)malonate (31 g, crude) followed by ethanol (310 mL). Concentrated HCl (31 mL) is added and the mixture is stirred for 16 hours at 70° C. The mixture is cooled to room temperature and concentrated. The residue is slurried with PE/EtOAc (10/1, 310 mL) and the solids are collected to afford 4-chloro-2-methyl-7-methylene-5,6,7,8-tetrahydrophthalazin-1(2H)-one (11 g, 52.2 mmol, 58% yield over two steps). ¹HNMR (400 MHz, DMSO-d₆) δ: 5.35 (s, 2H), 3.67 (s, 3H), 3.54 (s, 2H).

### Step G: tert-butyl (3-morpholinobicyclo[1.1.1]pentan-1-yl)carbamate

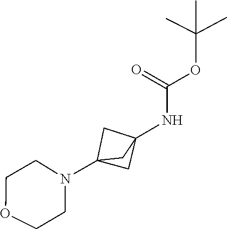 [SMILES: CC(C)(C)OC(=O)NC12CC(N3CCOCC3)(C1)C2]

To a solution of tert-butyl (3-aminobicyclo[1.1.1]pentan-1-yl)carbamate (3.0 g, 15.14 mmol) and 1-chloro-2-(2-chloroethoxy)ethane (2.6 g, 18.17 mmol) in DMF (10.0 mL) was added potassium carbonate (4.2 g, 30.28 mmol) and potassium iodide (2.5 g, 15.14 mmol). Then the reaction was stirred for 5 h at 90° C. The mixture was quenched with H2O (200 mL), extracted with EA (300 mL) and dried over MgSO4. The organic layers were concentrated and purified by flash chromatography (eluting with 0-75% EA in PE) to afford tert-butyl (3-morpholinobicyclo[1.1.1]pentan-1-yl)carbamate (4.0 g, 14.92 mmol, 98% yield). MS obsd. (ESI+): 269.1 (M+H)+.

### Step H: 3-morpholinobicyclo[1.1.1]pentan-1-amine hydrochloride

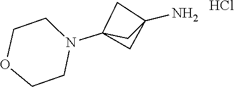 [SMILES: NC12CC(N3CCOCC3)(C1)C2 ; Cl]

To a solution of tert-butyl (3-morpholinobicyclo[1.1.1]pentan-1-yl)carbamate (2.7 g, 9.88 mmol) in dioxane (20.0 mL) was added HCl (4.0 M in 1,4-dioxane, 20 mL) and the mixture was stirred for 1 h at 25° C. The mixture was concentrated to afford 3-morpholinobicyclo[1.1.1]pentan-1-amine hydrochloride (1.69 g, crude), which was used in next step without further purification. MS obsd. (ESI+): 169.1 (M+H)+.

### Step I: 2-(6-chloro-5-(hydroxymethyl)-2-methyl-3-oxo-2,3-dihydropyridazin-4-yl)-N-(3-morpholinobicyclo[1.1.1]pentan-1-yl)acetamide

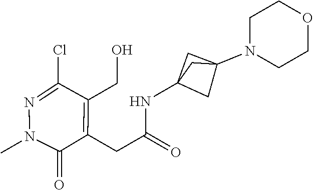 [SMILES: Cn1nc(Cl)c(CO)c(CC(=O)NC23CC(N4CCOCC4)(C2)C3)c1=O]

To a solution of 3-morpholinobicyclo[1.1.1]pentan-1-amine hydrochloride (376 mg, 1.83 mmol) in EtOH (20.0 mL) was added 4-chloro-2-methyl-5,8-dihydro-2H-pyrano[3,4-d]pyridazine-1,7-dione (480 mg, 2.24 mmol) and N,N-Diisopropylethylamine (866 mg, 6.70 mmol). The reaction was stirred for 4 h at 80° C. The mixture was concentrated and purified by flash chromatography (eluting with 0-15% MeOH in DCM) to afford 2-(6-chloro-5-(hydroxymethyl)-2-methyl-3-oxo-2,3-dihydropyridazin-4-yl)-N-(3-morpholinobicyclo[1.1.1]pentan-1-yl)acetamide (300 mg, 0.79 mmol, 43% yield). MS obsd. (ESI+): 383.2 (M+H)⁺.

### Step J: 4-chloro-2-methyl-6-(3-morpholinobicyclo[1.1.1]pentan-1-yl)-2,6-dihydropyrido[3,4-d]pyridazine-1,7-dione

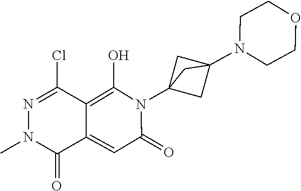 [SMILES: Cn1nc(Cl)c2c(O)n(C34CC(N5CCOCC5)(C3)C4)c(=O)cc2c1=O]

To a solution of 2-(6-chloro-5-(hydroxymethyl)-2-methyl-3-oxo-2,3-dihydropyridazin-4-yl)-N-(3-morpholinobicyclo[1.1.1]pentan-1-yl)acetamide (300 mg, 0.79 mmol) in DCM (30.0 mL) was added pyridinium chlorochromate (220 mg, 1.02 mmol) and the mixture was stirred for 16 h at 25° C. The mixture was concentrated and purified by flash chromatography (eluting with 0-15% MeOH in DCM) to afford 4-chloro-2-methyl-6-(3-morpholinobicyclo[1.1.1]pentan-1-yl)-2,6-dihydropyrido[3,4-d]pyridazine-1,7-dione (112 mg, 0.31 mmol, 39% yield. MS obsd. (ESI+): 363.2 (M+H)+.

### Step K: (R)-4-((1-(3-(difluoromethyl)-2-fluorophenyl)ethyl)amino)-2-methyl-6-(3-morpholinobicyclo[1.1.1]pentan-1-yl)-2,6-dihydropyrido[3,4-d]pyridazine-1,7-dione

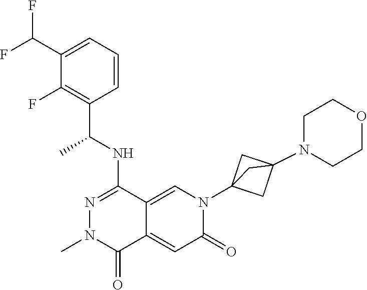 [SMILES: C[C@@H](Nc1nn(C)c(=O)c2cc(=O)n(C34CC(N5CCOCC5)(C3)C4)cc12)c1cccc(C(F)F)c1F]

To a solution of 4-chloro-2-methyl-6-(3-morpholinobicyclo[1.1.1]pentan-1-yl)-2,6-dihydropyrido[3,4-d]pyridazine-1,7-dione (80 mg, 0.22 mmol) and (1R)-1-[3-(difluoromethyl)-2-fluoro-phenyl]ethanamine (63 mg, 0.33 mmol) in 1,4-dioxane (1.0 mL) was added Xantphos-Pd-G3 (42 mg, 0.04 mmol) and cesium carbonate (216 mg, 0.66 mmol). The reaction was stirred for 9 h at 120° C. in a microwave reactor. The mixture was quenched with water (10 mL), extracted with DCM (20 mL) and dried over MgSO4. The organic layers were concentrated and purified by flash chromatography (eluting with 0-15% MeOH in DCM) followed by reverse phase HPLC to afford (R)-4-((1-(3-(difluoromethyl)-2-fluorophenyl)ethyl)amino)-2-methyl-6(3-morpholinobicyclo[1.1.1]pentan-1-yl)-2,6-dihydropyrido[3,4-d]pyridazine-1,7-dione (example 50, 17 mg, 0.03 mmol, 15% yield) as a yellow solid. MS obsd. (ESI+): 516.3 (M+H)⁺

### Example 51: (R)-4-((1-(3-(difluoromethyl)-2-fluorophenyl)ethyl)amino)-2-methyl-6-(2-oxaspiro[3.3]heptan-6-yl)pyrido[3,4-d]pyridazine-1,7(2H,6H)-dione

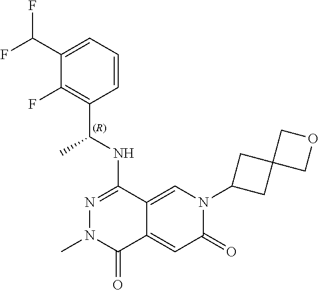

Prepared in an analgous manner as described for example 50 [steps I-K]. MS obsd. (ESI+): 461.2 (M+H)⁺.

### Examples 52 and 53: 4-(((R)-1-(3-(difluoromethyl)-2-fluorophenyl)ethyl)amino)-2-methyl-6-((S)-3-methyltetrahydrofuran-3-yl)-2,6-dihydropyrido[3,4-d]pyridazine-1,7-dione (example 52, absolute stereochemistry at tetrahydrofuran ring is arbitrarily assigned) and 4-(((R)-1-(3-(difluoromethyl)-2-fluorophenyl)ethyl)amino)-2-methyl-6-((R)-3-methyltetrahydrofuran-3-yl)-2,6-dihydropyrido[3,4-d]pyridazine-1,7-dione (example 53, absolute stereochemistry at tetrahydrofuran ring is arbitrarily assigned)

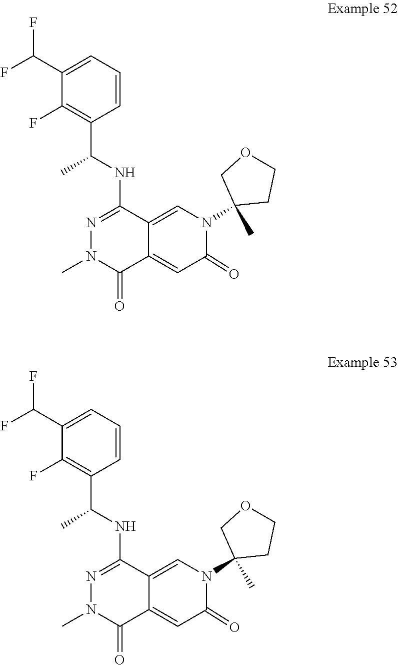 [SMILES: C[C@@H](Nc1nn(C)c(=O)c2cc(=O)n([C@@]3(C)CCOC3)cc12)c1cccc(C(F)F)c1F ; C[C@@H](Nc1nn(C)c(=O)c2cc(=O)n([C@]3(C)CCOC3)cc12)c1cccc(C(F)F)c1F]

Prepared in an analogous manner as example 43. Example 52: MS obsd. (ESI+): 449.3 (M+H)+. Chiral HPLC: (Column: OJ-3, 4.6*100 mm 3 um, Flow rate: 3.0 mL/min, Eluent: MeOH (0.2% 7M NH3 in MeOH), Temp 40° C.) Retention time=1.23 min. Example 53: MS obsd. (ESI+): 449.3 (M+H)⁺. Chiral HPLC: (Column: OJ-3, 4.6*100 mm 3 um, Flow rate: 3.0 mL/min, Eluent: MeOH (0.2% 7M NH3 in MeOH), Temp 40° C.) Retention time=1.69 min.

### Examples 54 and 55: 2-methyl-4-(((R)-1-(2-methyl-3-(trifluoromethyl)phenyl)ethyl)amino)-6-((S)-3-methyltetrahydrofuran-3-yl)-2,6-dihydropyrido[3,4-d]pyridazine-1,7-dione (example 54, stereochemistry at tetrahydrofuran ring is arbitrarily assigned) and 2-methyl-4-(((R)-1-(2-methyl-3-(trifluoromethyl)phenyl)ethyl)amino)-6-((R)-3-methyltetrahydrofuran-3-yl)-2,6-dihydropyrido[3,4-d]pyridazine-1,7-dione (example 55, stereochemistry at tetrahydrofuran ring is arbitrarily assigned)

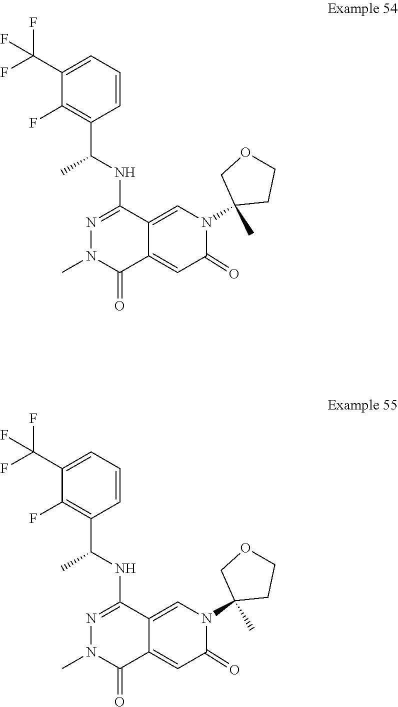 [SMILES: C[C@@H](Nc1nn(C)c(=O)c2cc(=O)n([C@@]3(C)CCOC3)cc12)c1cccc(C(F)(F)F)c1F ; C[C@@H](Nc1nn(C)c(=O)c2cc(=O)n([C@]3(C)CCOC3)cc12)c1cccc(C(F)(F)F)c1F]

Prepared in an analogous manner as example 43. Example 54: MS obsd. (ESI+): 463.5 (M+H)+. Chiral HPLC: (Column: OJ-3, 4.6*100 mm 3 um, Flow rate: 3.0 mL/min, Eluent: MeOH (0.2% 7M NH3 in MeOH), Temp 40° C.) Retention time=1.06 min. Example 55: MS obsd. (ESI+): 463.5 (M+H)+. Chiral HPLC: (Column: OJ-3, 4.6*100 mm 3 um, Flow rate: 3.0 mL/min, Eluent: MeOH (0.2% 7M NH3 in MeOH), Temp 40° C.) Retention time=1.29 min

### Example 56: (R)-4-((1-(4-bromothiophen-2-yl)ethyl)amino)-6-cyclopropyl-2-methyl-2,6-dihydropyrido[3,4-d]pyridazine-1,7-dione

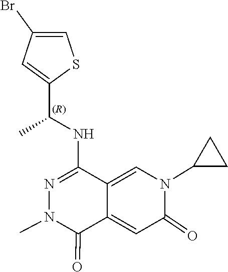

### Step A: (R)—N-(1-(4-bromothiophen-2-yl)ethylidene)-2-methylpropane-2-sulfinamide

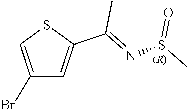

To a solution of 1-(4-bromo-2-thienyl)ethanone (4.7 g, 22.9 mmol) in THE (50 mL) was added (R)-2-methylpropane-2-sulfinamide (5.56 g, 46 mmol) and titanium tetraethoxide (15.68 g, 68.8 mmol). The reaction was stirred for 3 h at 80° C. The reaction was quenched with H₂O (30 mL) and the mixture was filtered. The filtrate was extracted with DCM (5*50 mL) and the combined organic layers were dried over Na₂SO₄, filtered and concentrated to afford (R)—N-(1-(4-bromothiophen-2-yl)ethylidene)-2-methylpropane-2-sulfinamide (7 g, crude). The crude product was used for the next step without further purification. MS obsd. (ESI+): ^{79/81}Br 308.1/310.0[(M+H)⁺].

### Step B: (R)—N—((R)-1-(4-bromothiophen-2-yl)ethyl)-2-methylpropane-2-sulfinamide

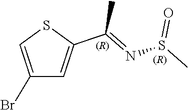

To a solution of (R)—N-(1-(4-bromothiophen-2-yl)ethylidene)-2-methylpropane-2-sulfinamide (7 g, 22.71 mmol) in THF (50 mL) was added sodium borohydride (1.03 g, 27.3 mmol). The reaction was stirred for 2 hr at rt. The reaction was quenched with H₂O (20 mL) and the mixture was extracted with DCM (3*40 mL). The combined organic layers were dried over Na₂SO₄ and the solvent was removed in vacuo. The residue was purified by flash column chromatography (eluting with 0%-70% EA in PE) to afford (R)—N—((R)-1-(4-bromothiophen-2-yl)ethyl)-2-methylpropane-2-sulfinamide (6.7 g, 21.59 mmol, 95% yield). MS obsd. (ESI⁺): ^{79/81}Br 310.3, 312.3 [(M+H)⁺].

### Step C: (R)-1-(4-bromothiophen-2-yl)ethan-1-amine hydrochloride

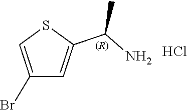

To a solution of ((R)—N—((R)-1-(4-bromothiophen-2-yl)ethyl)-2-methylpropane-2-sulfinamide (6.7 g, 21.59 mmol, 1.0 eq.) in dioxane (40 mL) was added HCl (4 M, 20 mL). The reaction was stirred for 2 h at rt. The solvent was concentrated in vacuo and the residue was recrystallized by ethyl ether. The mixture was filtered to afford (R)-1-(4-bromothiophen-2-yl)ethan-1-amine hydrochloride (4.4 g, 18.14 mmol, 84% yield). MS obsd. (ESI⁺): ^{79/81}Br 188.9/190.9 [(M−NH2)⁺].

### Step D: (R)-4-((1-(4-bromothiophen-2-yl)ethyl)amino)-6-cyclopropyl-2-methyl-2,6-dihydropyrido[3,4-d]pyridazine-1,7-dione (example 56)

To a solution of (R)-1-(4-bromothiophen-2-yl)ethan-1-amine hydrochloride (1 g, 4.13 mmol) in water (20 mL) was added K₂CO₃ until pH>12. The mixture was stirred for 1 h. Then the mixture was extracted with DCM (3*20 mL) and the combined organic layers were dried over Na₂SO₄. The solvent was removed in vacuo to afford the freebase (R)-1-(4-bromothiophen-2-yl)ethan-1-amine (846 mg, 4.11 mmol) as a light-yellow oil. The crude product was used for the next step without further purification.

To a solution of (6-cyclopropyl-2-methyl-1,7-dioxo-pyrido[3,4-d]pyridazin-4-yl)trifluoromethanesulfonate (500 mg, 1.37 mmol) in NMP (0.5 mL) was added ((R)-1-(4-bromothiophen-2-yl)ethan-1-amine (846 mg, 4.11 mmol). The reaction was stirred for 6 h at 95° C. The mixture was poured into saturated aqueous citric acid (10 mL) and stirred for 10 mins. The mixture was extracted with DCM (4*30 mL) and the combined organic layers were dried over Na₂SO₄. The solvent was removed in vacuo and the residue was purified by flash column chromatography (eluting with 0%-7% MeOH in DCM) and reverse phase HPLC afford (R)-4-((1-(4-bromothiophen-2-yl)ethyl)amino)-6-cyclopropyl-2-methyl-2,6-dihydropyrido[3,4-d]pyridazine-1,7-dione (340 mg, 0.81 mmol, 59% yield). MS obsd. (ESI⁺): ^{79/81}Br 421.1/423.1 [(M+H)⁺].

### Example 57: (R)-6-cyclopropyl-2-methyl-4-((1-(4-(2-((methylamino)methyl)phenyl)thiophen-2-yl)ethyl)amino)-2,6-dihydropyrido[3,4-d]pyridazine-1,7-dione

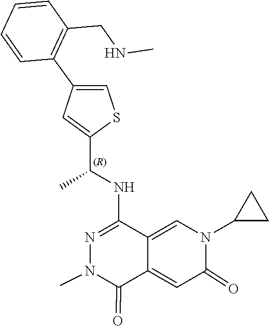

To a solution of (R)-4-((1-(4-bromothiophen-2-yl)ethyl)amino)-6-cyclopropyl-2-methyl-2,6-dihydropyrido[3,4-d]pyridazine-1,7-dione (example 56, 100 mg, 0.24 mmol) in dioxane (5 mL) and water (1 mL) was added N-methyl-1-(2-(4,4,5,5-tetramethyl-1,3,2-dioxaborolan-2-yl)phenyl)methanamine (117 mg, 0.48 mmol), potassium carbonate (98 mg, 0.71 mmol), and tetrakis(triphenylphosphine)palladium (55 mg, 0.05 mmol). The reaction was stirred for 3 hr at 110° C. in a microwave reactor. The reaction was cooled, diluted with water and extracted with DCM (4*30 mL). The combined organic layers were dried over Na₂SO₄ and the solvent was removed in vacuo. The residue was purified by flash column chromatography (eluting with 0%-7% MeOH in DCM) followed by preparative HPLC to afford (R)-6-cyclopropyl-2-methyl-4-((1-(4-(2-((methylamino)methyl)phenyl)thiophen-2-yl)ethyl)amino)-2,6-dihydropyrido[3,4-d]pyridazine-1,7-dione (example 57, 23.1 mg, 0.05 mmol, 20% yield). MS obsd. (ESI⁺): 462.3 [(M+H)⁺].

### Examples 58 and 59: 4-(((R)-1-(3-(difluoromethyl)-2-fluorophenyl)ethyl)amino)-6-((3R,4R)-3-fluorotetrahydro-2H-pyran-4-yl)-8-methoxy-2-methyl-2,6-dihydropyrido[3,4-d]pyridazine-1,7-dione (example 58, absolute stereochemistry at tetrahydropyran is arbitrarily assigned) & 4-(((R)-1-(3-(difluoromethyl)-2-fluorophenyl)ethyl)amino)-6-((3S,4S)-3-fluorotetrahydro-2H-pyran-4-yl)-8-methoxy-2-methyl-2,6-dihydropyrido[3,4-d]pyridazine-1,7-dione (example 59, absolute stereochemistry at tetrahydropyran is arbitrarily assigned)

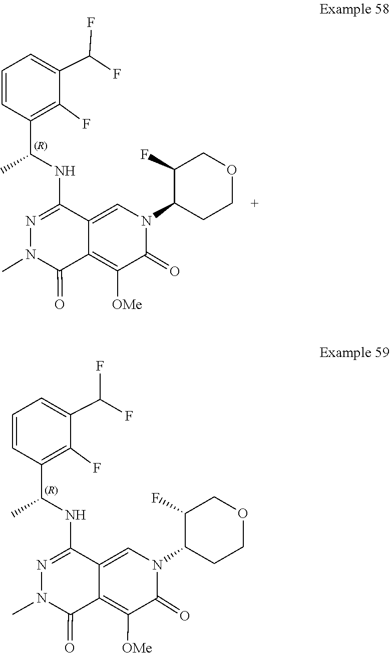

### Step A: 8-bromo-4-(((R)-1-(3-(difluoromethyl)-2-fluorophenyl)ethyl)amino)-6-((3R,4R)-3-fluorotetrahydro-2H-pyran-4-yl)-2-methyl-2,6-dihydropyrido[3,4-d]pyridazine-1,7-dione and 8-bromo-4-(((R)-1-(3-(difluoromethyl)-2-fluorophenyl)ethyl)amino)-6-((3S,4S)-3-fluorotetrahydro-2H-pyran-4-yl)-2-methyl-2,6-dihydropyrido[3,4-d]pyridazine-1,7-dione

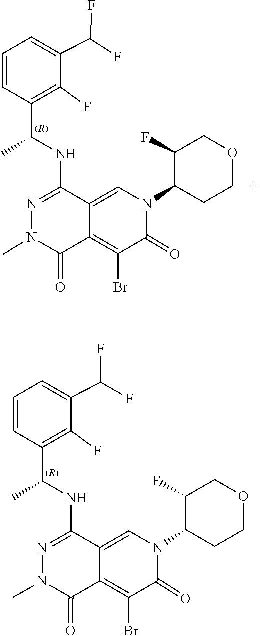

To a diastereomeric mixture of 4-(((R)-1-(3-(difluoromethyl)-2-fluorophenyl)ethyl)amino)-6-((3R,4R)-3-fluorotetrahydro-2H-pyran-4-yl)-2-methyl-2,6-dihydropyrido[3,4-d]pyridazine-1,7-dione (example 32) and 4-(((R)-1-(3-(difluoromethyl)-2-fluorophenyl)ethyl)amino)-6-((3S,4S)-3-fluorotetrahydro-2H-pyran-4-yl)-2-methyl-2,6-dihydropyrido[3,4-d]pyridazine-1,7-dione (example 33) (238 mg, 510.26 μmol) in CH₃COOH (2.03 mL) was added potassium acetate (75 mg, 765 μmol) and bromine (46 mg, 561 μmol). The mixture was heated to 25° C. and stirred for 2 hrs. The solvent was removed under reduced pressure and the crude residue was purified by preparative TLC (DCM/MeOH=30:1) to afford a diastereomeric mixture of 8-bromo-4-(((R)-1-(3-(difluoromethyl)-2-fluorophenyl)ethyl)amino)-6-((3R,4R)-3-fluorotetrahydro-2H-pyran-4-yl)-2-methyl-2,6-dihydropyrido[3,4-d]pyridazine-1,7-dione and 8-bromo-4-(((R)-1-(3-(difluoromethyl)-2-fluorophenyl)ethyl)amino)-6-((3S,4S)-3-fluorotetrahydro-2H-pyran-4-yl)-2-methyl-2,6-dihydropyrido[3,4-d]pyridazine-1,7-dione (160 mg, 293 μmol, 58% yield). MS obsd. (ESI⁺): ^{79/81}Br 545.3/547.3 [M+H]⁺

### Step B: 4-(((R)-1-(3-(difluoromethyl)-2-fluorophenyl)ethyl)amino)-6-((3R,4R)-3-fluorotetrahydro-2H-pyran-4-yl)-8-methoxy-2-methyl-2,6-dihydropyrido[3,4-d]pyridazine-1,7-dione (example 58) & 4-(((R)-1-(3-(difluoromethyl)-2-fluorophenyl)ethyl)amino)-6-((3S,4S)-3-fluorotetrahydro-2H-pyran-4-yl)-8-methoxy-2-methyl-2,6-dihydropyrido[3,4-d]pyridazine-1,7-dione (example 59)

Methanol (94 mg, 2.93 mmol, 119 μL), XantPhos-PdG3 (73 mg, 88 μmol), a diastereomeric mixture of 8-bromo-4-(((R)-1-(3-(difluoromethyl)-2-fluorophenyl)ethyl)amino)-6-((3R,4R)-3-fluorotetrahydro-2H-pyran-4-yl)-2-methyl-2,6-dihydropyrido[3,4-d]pyridazine-1,7-dione and 8-bromo-4-(((R)-1-(3-(difluoromethyl)-2-fluorophenyl)ethyl)amino)-6-((3S,4S)-3-fluorotetrahydro-2H-pyran-4-yl)-2-methyl-2,6-dihydropyrido[3,4-d]pyridazine-1,7-dione (160 mg, 293 μmol), and cesium carbonate (287 mg, 880 μmol) were dissolved in 1,4-dioxane (4 mL). The mixture was stirred at 50° C. for 16 hours. The mixture was then filtered and the filtrate was partitioned between NaHCO₃ (aq) and ethyl acetate. The organic layers were combined and dried over sodium sulfate, and the solvent was removed under reduced pressure. The crude residue was purified by preparative TLC (DCM/MeOH=30:1) followed by reverse phase HPLC to afford a diastereomeric mixture 4-(((R)-1-(3-(difluoromethyl)-2-fluorophenyl)ethyl)amino)-6-(cis-3-fluorotetrahydro-2H-pyran-4-yl)-8-methoxy-2-methyl-2,6-dihydropyrido[3,4-d]pyridazine-1,7-dione (60 mg, 117 μmol, 40% yield). Further separation of diastereomers via chiral SFC afforded 4-(((R)-1-(3-(difluoromethyl)-2-fluorophenyl)ethyl)amino)-6-((3R,4R)-3-fluorotetrahydro-2H-pyran-4-yl)-8-methoxy-2-methyl-2,6-dihydropyrido[3,4-d]pyridazine-1,7-dione (example 58, absolute stereochemistry at tetrahydropyran is arbitrarily assigned) and 4-(((R)-1-(3-(difluoromethyl)-2-fluorophenyl)ethyl)amino)-6-((3S,4S)-3-fluorotetrahydro-2H-pyran-4-yl)-8-methoxy-2-methyl-2,6-dihydropyrido[3,4-d]pyridazine-1,7-dione (example 59, absolute stereochemistry at tetrahydropyran is arbitrarily). Example 58: MS obsd. (ESI⁺): 497.3, [M+H]⁺. Chiral HPLC: (Column: OD-3, 4.6*100 mm 3 um, Flow rate: 3.0 mL/min, Eluent: MeOH (0.2% 7M NH3 in MeOH), Temp 40° C.) Retention time=1.56 min. Example 59: MS obsd. (ESI⁺): 497.3, [M+H]+. Chiral HPLC: (Column: OD-3, 4.6*100 mm 3 um, Flow rate: 3.0 mL/min, Eluent: MeOH (0.2% 7M NH3 in MeOH), Temp 40° C.) Retention time=2.22 min.

### Examples 60 and 61: 4-(((R)-1-(3-(difluoromethyl)-2-fluorophenyl)ethyl)amino)-6-((3R,4S)-3-fluorotetrahydro-2H-pyran-4-yl)-8-methoxy-2-methyl-2,6-dihydropyrido[3,4-d]pyridazine-1,7-dione (example 60, absolute stereochemistry at tetrahydropyran is arbitrarily assigned) 4-(((R)-1-(3-(difluoromethyl)-2-fluorophenyl)ethyl)amino)-6-((3S,4R)-3-fluorotetrahydro-2H-pyran-4-yl)-8-methoxy-2-methyl-2,6-dihydropyrido[3,4-d]pyridazine-1,7-dione (example 61, absolute stereochemistry at tetrahydropyran is arbitrarily assigned)

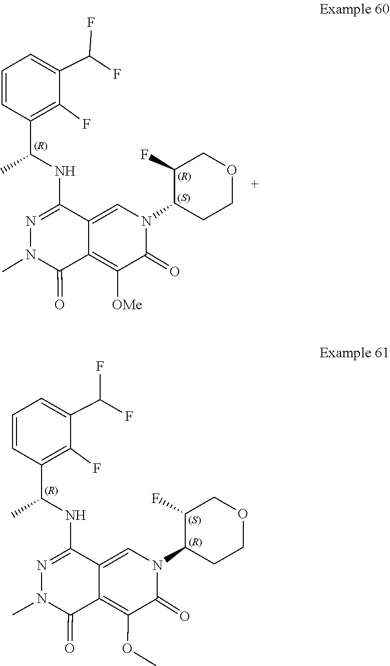

Prepared in an analogous fashion to examples 58 and 59, starting with a diastereomeric mixture of examples 30 and 31. Example 60: MS obsd. (ESI⁺): 497.0 (M+H)⁺. Chiral HPLC: (Column: OZ-3, 4.6*100 mm 3 um, Flow rate: 3.0 mL/min, Eluent: MeOH (0.2% 7M NH3 in MeOH), Temp 40° C.) Retention time=1.36 min. Example 61: MS obsd. (ESI⁺): 497.0 (M+H)⁺. Chiral HPLC: (Column: OZ-3, 4.6*100 mm 3 um, Flow rate: 3.0 mL/min, Eluent: MeOH (0.2% 7M NH3 in MeOH), Temp 40° C.) Retention time=1.75 min.

### Examples 62 and 63: (R)-4-((1-(benzo[b]thiophen-4-yl)ethyl)amino)-2-methyl-6-(tetrahydro-2H-pyran-4-yl)-2,6-dihydropyrido[3,4-d]pyridazine-1,7-dione (example 62) and (S)-4-((1-(benzo[b]thiophen-4-yl)ethyl)amino)-2-methyl-6-(tetrahydro-2H-pyran-4-yl)-2,6-dihydropyrido[3,4-d]pyridazine-1,7-dione (example 63)

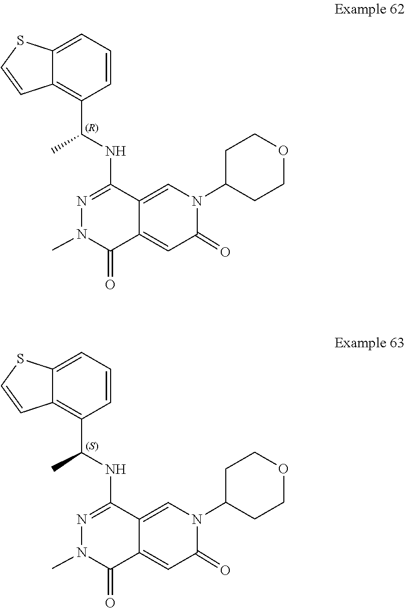

Prepared in an analogous fashion to example 4 using (R/S)-1-(benzo[b]thiophen-4-yl)ethan-1-amine (synthesis described in WO/2018/192250). Separation of individual enantiomers via chiral SFC afforded (R)-4-((1-(benzo[b]thiophen-4-yl)ethyl)amino)-2-methyl-6-(tetrahydro-2H-pyran-4-yl)-2,6-dihydropyrido[3,4-d]pyridazine-1,7-dione (example 62) and (S)-4-((1-(benzo[b]thiophen-4-yl)ethyl)amino)-2-methyl-6-(tetrahydro-2H-pyran-4-yl)-2,6-dihydropyrido[3,4-d]pyridazine-1,7-dione (example 63). Example 62: MS obsd. (ESI⁺): 437.2 (M+H)⁺. Chiral HPLC: (Column: AS-3, 4.6*100 mm 3 um, Flow rate: 3.0 mL/min, Eluent: MeOH (0.2% 7M NH3 in MeOH), Temp 40° C.) Retention time=2.83 min. Example 63: MS obsd. (ESI⁺): 437.2 (M+H)⁺. Chiral HPLC: (Column: AS-3, 4.6*100 mm 3 um, Flow rate: 3.0 mL/min, Eluent: MeOH (0.2% 7M NH3 in MeOH), Temp 40° C.) Retention time=2.18 min

### Examples 64 and 65: (R)-4-((1-(benzo[b]thiophen-4-yl)ethyl)amino)-6-cyclopropyl-2-methyl-2,6-dihydropyrido[3,4-d]pyridazine-1,7-dione (example 64) and (S)-4-((1-(benzo[b]thiophen-4-yl)ethyl)amino)-6-cyclopropyl-2-methyl-2,6-dihydropyrido[3,4-d]pyridazine-1,7-dione (example 65)

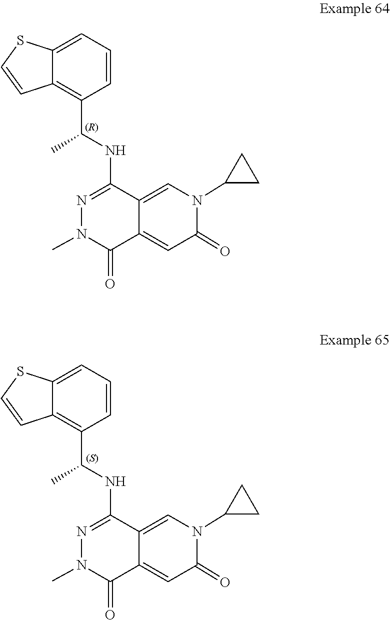

Prepared in an analogous fashion to examples 4, 62 and 63. Example 64: MS obsd. (ESI⁺): 393.2 (M+H)⁺, Example 65: MS obsd. (ESI⁺): 393.2 (M+H)⁺.

### Example 66: (R)-1-(4-((1-(3-(difluoromethyl)-2-fluorophenyl)ethyl)amino)-2-methyl-1,7-dioxo-1,7-dihydropyrido[3,4-d]pyridazin-6(2H)-yl)cyclopropane-1-carbonitrile

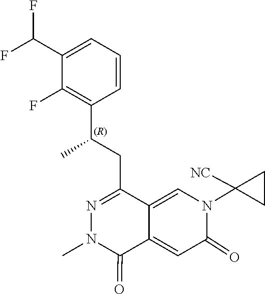

### Step A: methyl 4-(2-(tert-butoxycarbonyl)-1-methylhydrazine-1-carbonyl)-1-(1-cyanocyclopropyl)-6-oxo-1,6-dihydropyridine-3-carboxylate

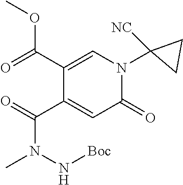 [SMILES: COC(=O)c1cn(C2(C#N)CC2)c(=O)cc1C(=O)N(C)NC(=O)OC(C)(C)C]

Prepared in an analogous fashion to example 43 [steps A-E]. LCMS: m/z 391.4 (M+H)⁺.

### Step B: methyl 1-(1-cyanocyclopropyl)-4-(1-methylhydrazine-1-carbonyl)-6-oxo-1,6-dihydropyridine-3-carboxylate

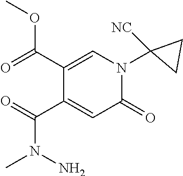 [SMILES: COC(=O)c1cn(C2(C#N)CC2)c(=O)cc1C(=O)N(C)N]

To a solution of methyl 4-(2-(tert-butoxycarbonyl)-1-methylhydrazine-1-carbonyl)-1-(1-cyanocyclopropyl)-6-oxo-1,6-dihydropyridine-3-carboxylate (20 mg, 51 umol) in 1,1,1,3,3,3-Hexafluoro-2-propanol (1 mL) was added TsOH (5 mg, 25 umol). The reaction was stirred for 1 h at room temperature. The reaction was concentrated to dryness to afford 14 mg crude product. A parallel reaction batch performed on 250 umol scale was also carried out to give 74 mg crude product. The two batches were combined to give methyl 1-(1-cyanocyclopropyl)-4-(1-methylhydrazine-1-carbonyl)-6-oxo-1,6-dihydropyridine-3-carboxylate (88 mg, crude). The crude material was used in the next step without further purification. MS obsd. (ESI⁺): 291.5 (M+H)⁺

### Step C: 1-(2-methyl-1,4,7-trioxo-1,3,4,7-tetrahydropyrido[3,4-d]pyridazin-6(2H)-yl)cyclopropane-1-carbonitrile

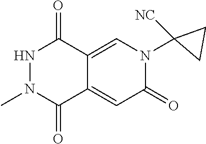 [SMILES: Cn1[nH]c(=O)c2cn(C3(C#N)CC3)c(=O)cc2c1=O]

The aforementioned methyl 1-(1-cyanocyclopropyl)-4-(1-methylhydrazine-1-carbonyl)-6-oxo-1,6-dihydropyridine-3-carboxylate (88 mg, crude) was dissolved in NH3/MeOH (7 M, 3 mL). The mixture was stirred for 1 h. The reaction was concentrated to dryness to afford 1-(2-methyl-1,4,7-trioxo-1,3,4,7-tetrahydropyrido[3,4-d]pyridazin-6(2H)-yl)cyclopropane-1-carbonitrile (70 mg, crude), which was used in the next step without further purification. MS obsd. (ESI⁺): 259.3 (M+H)⁺.

### Step D: 6-(1-cyanocyclopropyl)-2-methyl-1,7-dioxo-1,2,6,7-tetrahydropyrido[3,4-d]pyridazin-4-yl 2,4,6-triisopropylbenzenesulfonate

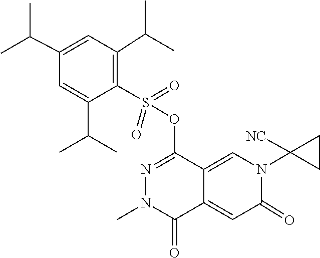 [SMILES: CC(C)c1cc(C(C)C)c(S(=O)(=O)Oc2nn(C)c(=O)c3cc(=O)n(C4(C#N)CC4)cc23)c(C(C)C)c1]

The aforementioned crude 1-(2-methyl-1,4,7-trioxo-1,3,4,7-tetrahydropyrido[3,4-d]pyridazin-6(2H)-yl)cyclopropane-1-carbonitrile (70 mg, crude) was dissolved in DCM (5 mL) followed by the addition of DIPEA (70 mg, 542 μmol) and 2,4,6-triisopropylbenzenesulfonyl chloride (90 mg, 298 μmol). The solution was stirred at rt for 1 hour. The mixture was purified by flash column chromatography (PE:EA=3:1) to afford 6-(1-cyanocyclopropyl)-2-methyl-1,7-dioxo-1,2,6,7-tetrahydropyrido[3,4-d]pyridazin-4-yl 2,4,6-triisopropylbenzenesulfonate (85 mg, 0.16 mmol, 21% yield over 3 steps). MS obsd. (ESI⁺): 525.6 (M+H)⁺.

### Step E: (R)-1-(4-((1-(3-(difluoromethyl)-2-fluorophenyl)ethyl)amino)-2-methyl-1,7-dioxo-1,7-dihydropyrido[3,4-d]pyridazin-6(2H)-yl)cyclopropane-1-carbonitrile (example 66)

A mixture of 6-(1-cyanocyclopropyl)-2-methyl-1,7-dioxo-1,2,6,7-tetrahydropyrido[3,4-d]pyridazin-4-yl 2,4,6-triisopropylbenzenesulfonate (70 mg, 133 umol), (1R)-1-[3-(difluoromethyl)-2-fluoro-phenyl]ethanamine (38 mg, 200 μmol), XantPhos-PdG3 (13 mg, 13 umol), Cs₂CO₃ (131 mg, 400 umol) in 1,4-dioxane (0.7 mL) was stirred under N₂ at 110° C. for 1 hour. The reaction mixture was filtered and purified by preparative TLC followed by reverse phase HPLC to obtain (R)-1-(4-((1-(3-(difluoromethyl)-2-fluorophenyl)ethyl)amino)-2-methyl-1,7-dioxo-1,7-dihydropyrido[3,4-d]pyridazin-6(2H)-yl)cyclopropane-1-carbonitrile (3.8 mg, 9 umol, 7% yield). MS obsd. (ESI⁺): 430.3 (M+H)⁺.

### Examples 67 and 68: (R)-4-((1-(1,1-difluoro-2,3-dihydro-1H-inden-4-yl)ethyl)amino)-2-methyl-6-(tetrahydro-2H-pyran-4-yl)pyrido[3,4-d]pyridazine-1,7(2H,6H)-dione (example 67) and (S)-4-((1-(1,1-difluoro-2,3-dihydro-1H-inden-4-yl)ethyl)amino)-2-methyl-6-(tetrahydro-2H-pyran-4-yl)pyrido[3,4-d]pyridazine-1,7(2H,6H)-dione (example 68)

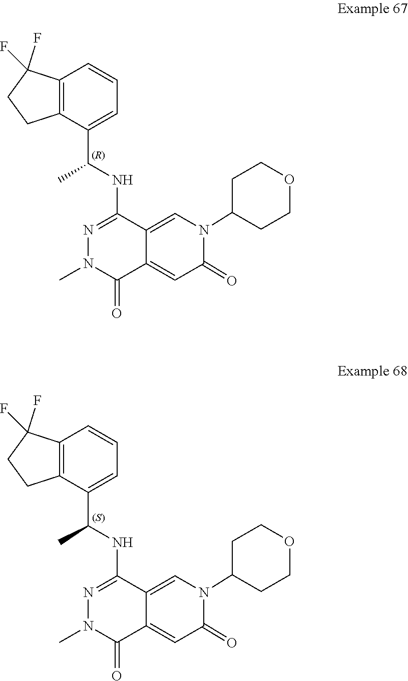

Prepared in an analogous fashion to examples 44 and 45, using (R/S)-1-(1,1-difluoro-2,3-dihydro-1H-inden-4-yl)ethanamine hydrochloride (preparation described in WO/2019/122129). Example 67: MS obsd. (ESI⁺): 457.2 (M+H)⁺. Chiral HPLC: (Column: IG-3, 4.6*100 mm 3 um, Flow rate: 3.0 mL/min, Eluent: MeOH (0.2% 7M NH3 in MeOH), Temp 40° C.) Retention time=1.45 min. Example 68: MS obsd. (ESI⁺): 457.2 (M+H)⁺. Chiral HPLC: (Column: IG-3, 4.6*100 mm 3 um, Flow rate: 3.0 mL/min, Eluent: MeOH (0.2% 7M NH3 in MeOH), Temp 40° C.) Retention time=1.09 min.

### Example 69: (R)-3-(4-((1-(3-(difluoromethyl)-2-fluorophenyl)ethyl)amino)-2-methyl-1,7-dioxo-1,7-dihydropyrido[3,4-d]pyridazin-6(2H)-yl)-N,N-dimethylbicyclo[1.1.1]pentane-1-carboxamide

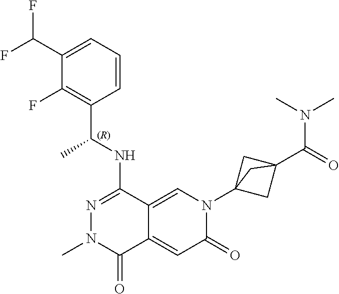

### Step A: tert-butyl (3-(dimethylcarbamoyl)bicyclo[1.1.1]pentan-1-yl)carbamate

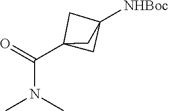 [SMILES: CN(C)C(=O)C12CC(NC(=O)OC(C)(C)C)(C1)C2]

To a solution of 3-((tert-butoxycarbonyl)amino)bicyclo[1.1.1]pentane-1-carboxylic acid (4.0 g, 17.62 mmol) in THE (60.0 mL) was added dimethylamine (2.0 M in THF, 17.6 mL, 35.24 μmmol) and 2-(7-Aza-1H-benzotriazole-1-yl)-1,1,3,3-tetramethyluronium hexafluorophosphate (20.1 g, 52.86 mmol) and stirred for 15 min at 25° C. To the mixture was then added N-ethyl-N-isopropyl-propan-2-amine (11.4 g, 88.10 mmol) at 0° C. and the reaction was stirred for 16 h at 25° C. The mixture was concentrated and purified by flash chromatography (eluting with 0-100% EA in PE) to afford tert-butyl (3-(dimethylcarbamoyl)bicyclo[1.1.1]pentan-1-yl)carbamate (4.0 g, 15.73 mmol, 89% yield). MS obsd. (ESI⁺): 255.4 (M+H)⁺.

### Step B: 3-amino-N,N-dimethylbicyclo[1.1.1]pentane-1-carboxamide hydrochloride

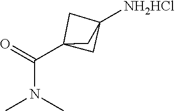

To a solution of tert-butyl (3-(dimethylcarbamoyl)bicyclo[1.1.1]pentan-1-yl)carbamate (3.6 g, 14.17 mmol) in 1,4-dioxane (20.0 mL) was added HCl (4.0 M in 1,4-dioxane, 20.0 mL) and the mixture was stirred for 1 h at 25° C. The mixture was concentrated to afford 3-amino-N,N-dimethylbicyclo[1.1.1]pentane-1-carboxamide hydrochloride (2.7 g, crude) which was used in the next step without further purification. MS obsd. (ESI⁺): 155.4 (M+H)⁺.

### Step C: methyl 1-(3-(dimethylcarbamoyl)bicyclo[1.1.1]pentan-1-yl)-4-hydroxy-6-oxo-1,6-dihydropyridine-3-carboxylate

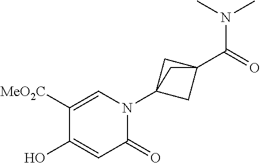 [SMILES: COC(=O)c1cn(C23CC(C(=O)N(C)C)(C2)C3)c(=O)cc1O]

To a solution of dimethyl 3-oxopentanedioate (2.4 g, 13.90 mmol) in MeOH (50 mL) was added N,N-Dimethylformamide dimethyl acetal (1.8 g, 15.05 mmol) and the reaction was stirred for 5 h at 25° C. To the mixture was added 3-amino-N,N-dimethylbicyclo[1.1.1]pentane-1-carboxamide hydrochloride (2.2 g, 11.58 mmol) and stirred for 20 h at 25° C. Then to the mixture was added Sodium methoxide (813 mg, 15.05 mmol) and the reaction was stirred for 2 h at 25° C. The mixture was concentrated and purified by flash chromatography (eluting with 0-100% EA in PE) to give methyl 1-(3-(dimethylcarbamoyl)bicyclo[1.1.1]pentan-1-yl)-4-hydroxy-6-oxo-1,6-dihydropyridine-3-carboxylate (2.1 g, 6.86 mmol, 59% yield). MS obsd. (ESI⁺): 307.4 (M+H)⁺.

### Step D: (R)-3-(4-((1-(3-(difluoromethyl)-2-fluorophenyl)ethyl)amino)-2-methyl-1,7-dioxo-1,7-dihydropyrido[3,4-d]pyridazin-6(2H)-yl)-N,N-dimethylbicyclo[1.1.1]pentane-1-carboxamide (example 69)

Synthesized in an analogous manner to example 43 steps [c-h] starting with methyl 1-(3-(dimethylcarbamoyl)bicyclo[1.1.1]pentan-1-yl)-4-hydroxy-6-oxo-1,6-dihydropyridine-3-carboxylate. MS obsd. (ESI⁺): 502.3 (M+H)⁺.

### Example 70: (R)-4-((1-(3-(1,1-difluoro-2-hydroxy-2-methylpropyl)-2-fluorophenyl)ethyl)amino)-6-(1-(difluoromethyl)cyclopropyl)-2-methyl-2,6-dihydropyrido[3,4-d]pyridazine-1,7-dione

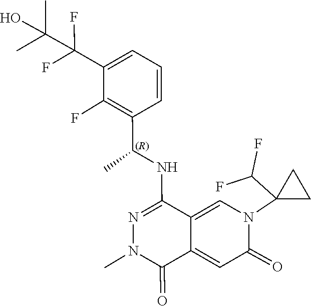

### Step A: 2-(6-chloro-5-(hydroxymethyl)-2-methyl-3-oxo-2,3-dihydropyridazin-4-yl)-N-(1-(difluoromethyl)cyclopropyl)acetamide

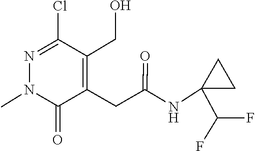 [SMILES: Cn1nc(Cl)c(CO)c(CC(=O)NC2(C(F)F)CC2)c1=O]

To a stirring solution of 1-(difluoromethyl)cyclopropanamine; hydrochloride (501.72 mg, 3.49 mmol) in Toluene (15 mL) was added 4-chloro-2-methyl-5,8-dihydropyrano[3,4-d]pyridazine-1,7-dione (500 mg, 2.33 mmol) followed by trimethylaluminum (1.6 M in toluene, 4.37 mL) The resulting mixture was stirred at 75° C. for 16 hr. The reaction mixture was then cooled to room temperature and diluted with ethyl acetate (10 mL). The organic layer was washed with water (3×5 mL), 10% sodium chloride solution (5 mL), dried over sodium sulfate and concentrated under reduced pressure. The crude product was purified by flash chromatography eluting with 0-5% MeOH in DCM to afford 2-(6-chloro-5-(hydroxymethyl)-2-methyl-3-oxo-2,3-dihydropyridazin-4-yl)-N-(1-(difluoromethyl)cyclopropyl)acetamide (300 mg, 932 μmol, 40% yield). MS obsd. (ESI⁺): 322.4 (M+H)⁺.

### Step B: 4-chloro-6-(1-(difluoromethyl)cyclopropyl)-2-methyl-2,6-dihydropyrido[3,4-d]pyridazine-1,7-dione

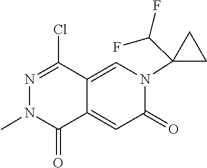 [SMILES: Cn1nc(Cl)c2cn(C3(C(F)F)CC3)c(=O)cc2c1=O]

To a solution of 2-(6-chloro-5-(hydroxymethyl)-2-methyl-3-oxo-2,3-dihydropyridazin-4-yl)-N-(1-(difluoromethyl)cyclopropyl)acetamide e (300 mg, 932 μmol) in chloroform (12 mL) was added PCC (502 mg, 2.33 mmol) at 0° C., then the reaction was stirred at rt for 16 hr. The reaction was concentrated in vacuo. The crude mixture was purified by flash chromatography eluting with (0-30% EA in PE) to afford 4-chloro-6-(1-(difluoromethyl)cyclopropyl)-2-methyl-2,6-dihydropyrido[3,4-d]pyridazine-1,7-dione (55 mg, 182 μmol, 58% yield). MS obsd. (ESI⁺): 302.3 (M+H)⁺.

### Step C: (R)-4-((1-(3-(1,1-difluoro-2-hydroxy-2-methylpropyl)-2-fluorophenyl)ethyl)amino)-6-(1-(difluoromethyl)cyclopropyl)-2-methyl-2,6-dihydropyrido[3,4-d]pyridazine-1,7-dione (example 70)

To a solution of 4-chloro-6-(1-(difluoromethyl)cyclopropyl)-2-methyl-2,6-dihydropyrido[3,4-d]pyridazine-1,7-dione (53 mg, 176 μmol) in 1,4-dioxane (0.5 mL) was added 1-[3-[(1R)-1-aminoethyl]-2-fluoro-phenyl]-1,1-difluoro-2-methyl-propan-2-ol (52 mg, 211 mol), Xantphos-Pd-G3 (33.3 mg, 35 μmol) and Cs2CO3 (171.3 mg, 527. μmol). The reaction was irradiated in a microwave reactor at 130° C. for 3 hr. The solvent was then removed in vacuo. The crude product was purified by reverse phase HPLC to afford (R)-4-((1-(3-(1,1-difluoro-2-hydroxy-2-methylpropyl)-2-fluorophenyl)ethyl)amino)-6-(1-(difluoromethyl)cyclopropyl)-2-methyl-2,6-dihydropyrido[3,4-d]pyridazine-1,7-dione (28 mg, 52 μmol, 30% yield). MS obsd. (ESI⁺): 513.7 (M+H)⁺.

### Example 71: (R)-4-((1-(3-(difluoromethyl)-2-fluorophenyl)ethyl)amino)-2-methyl-6-(1-(trifluoromethyl)cyclopropyl)-2,6-dihydropyrido[3,4-d]pyridazine-1,7-dione

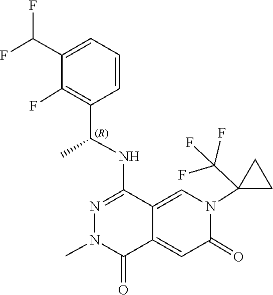

Prepared via an analogous route to example 70. MS obsd. (ESI⁺): 473.2 (M+H)⁺.

### Example 72: (R)-8-bromo-4-((1-(3-(difluoromethyl)-2-fluorophenyl)ethyl)amino)-6-(1-(difluoromethyl)cyclopropyl)-2-methyl-2,6-dihydropyrido[3,4-d]pyridazine-1,7-dione

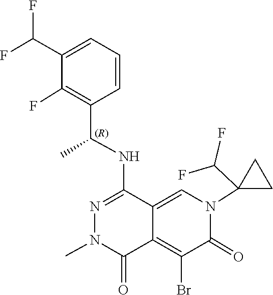

To a solution of (R)-4-((1-(3-(difluoromethyl)-2-fluorophenyl)ethyl)amino)-6-(1-(difluoromethyl)cyclopropyl)-2-methyl-2,6-dihydropyrido[3,4-d]pyridazine-1,7-dione (323 mg, 711 μmol) in AcOH (15 mL) was added potassium acetate (105 mg, 1.07 mmol). The reaction was stirred at rt for 1.5 hr. The reaction mixture was concentrated to dryness and the residue was purified by silica gel chromatography (eluting with EA in PE) to afford (R)-8-bromo-4-((1-(3-(difluoromethyl)-2-fluorophenyl)ethyl)amino)-6-(1-(difluoromethyl)cyclopropyl)-2-methyl-2,6-dihydropyrido[3,4-d]pyridazine-1,7-dione (305 mg, 571 μmol, 80% yield). MS obsd. (ESI⁺): ^{79/81}Br 533.1/535.1 (M+H)⁺.

### Example 73: (R)-4-((1-(3-(difluoromethyl)-2-fluorophenyl)ethyl)amino)-6-(1-(difluoromethyl)cyclopropyl)-8-methoxy-2-methyl-2,6-dihydropyrido[3,4-d]pyridazine-1,7-dione

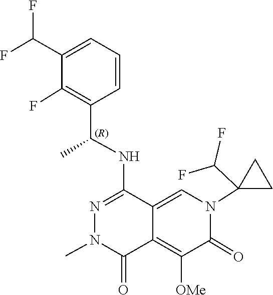

To a solution of (R)-8-bromo-4-((1-(3-(difluoromethyl)-2-fluorophenyl)ethyl)amino)-6-(1-(difluoromethyl)cyclopropyl)-2-methyl-2,6-dihydropyrido[3,4-d]pyridazine-1,7-dione (example 72, 110 mg, 206 μmol) in MeOH (11 mL) was added MeONa (111 mg, 2.06 mmol) at rt. The mixture was heated in a microwave reactor at 80° C. for 2 hr. The mixture was quenched with NH4Cl (aq.) and extracted with ethyl acetate. The organic layers were dried over sodium sulfate and concentrated under vacuum. The residue was purified by flash chromatography (eluted with 0-2% MeOH in DCM) to give (R)-4-((1-(3-(difluoromethyl)-2-fluorophenyl)ethyl)amino)-6-(1-(difluoromethyl)cyclopropyl)-8-methoxy-2-methyl-2,6-dihydropyrido[3,4-d]pyridazine-1,7-dione (30 mg, 61 μmol, 30% yield). MS obsd. (ESI⁺): 485.2 (M+H)⁺.

### Example 74: (R)-4-((1-(3-(difluoromethyl)-2-fluorophenyl)ethyl)amino)-6-(1-(difluoromethyl)cyclopropyl)-2,8-dimethyl-2,6-dihydropyrido[3,4-d]pyridazine-1,7-dione

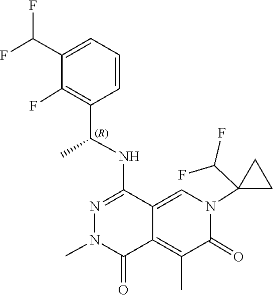

To a solution of (R)-8-bromo-4-((1-(3-(difluoromethyl)-2-fluorophenyl)ethyl)amino)-6-(1-(difluoromethyl)cyclopropyl)-2-methyl-2,6-dihydropyrido[3,4-d]pyridazine-1,7-dione (example 72, 80 mg, 150 μmol) in 1,4-dioxane (2 mL) was added Pd(PPh4)3 (17 mg, 15 μmol), Cs2CO3 (146 mg, 450 μmol) and 2,4,6-trimethyl-1,3,5,2,4,6-trioxatriborinane (113 mg, 450 μmol) at rt. The mixture was stirred at 100° C. for 16 hr. The mixture was quenched with NH₄Cl (aq.), and extracted with ethyl acetate. The organic layers were dried over sodium sulfate and concentrated. The residue was purified by flash chromatography (eluted with 0-250% EA in PE) to give (R)-4-((1-(3-(difluoromethyl)-2-fluorophenyl)ethyl)amino)-6-(1-(difluoromethyl)cyclopropyl)-2,8-dimethyl-2,6-dihydropyrido[3,4-d]pyridazine-1,7-dione (40 mg, 85 μmol, 57% yield). MS obsd. (ESI⁺): 469.1 (M+H)⁺.

### Examples 75 and 76: (1S,3s)-3-(4-(((R)-1-(3-(difluoromethyl)-2-fluorophenyl)ethyl)amino)-2-methyl-1,7-dioxo-1,7-dihydropyrido[3,4-d]pyridazin-6(2H)-yl)cyclobutane-1-carbonitrile (example 75, stereochemistry about cyclobutyl ring is arbitrarily assigned) and (1r,3s)-3-(4-(((R)-1-(3-(difluoromethyl)-2-fluorophenyl)ethyl)amino)-2-methyl-1,7-dioxo-1,7-dihydropyrido[3,4-d]pyridazin-6(2H)-yl)cyclobutane-1-carbonitrile (example 76, stereochemistry about cyclobutyl ring is arbitrarily assigned)

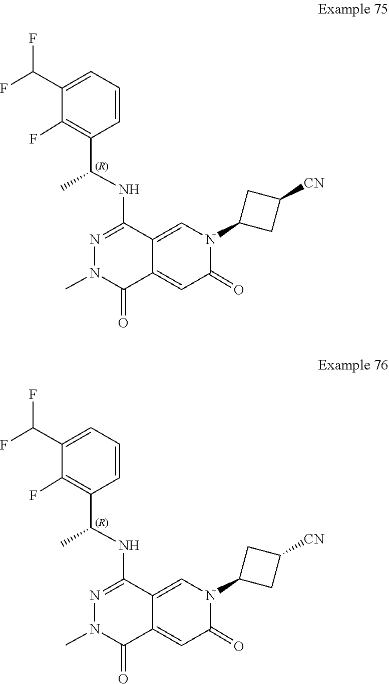

Prepared according to an analogous route as described for example 50 [steps I-K], starting with a mixture of cis/trans 3-aminocyclobutane-1-carbonitrile hydrochloride. Cis/trans stereochemistry on the cyclobutyl ring is arbitrarily assigned. Example 75: MS obsd. (ESI⁺): 444.3 (M+H)⁺. Chiral IPLC: (Column: AS-3, 4.6*100 mm 3 um, Flow rate: 3.0 mL/min, Eluent: MeOH (0.2% 7M NH3 in MeOH), Temp 40° C.) Retention time=1.35 min. Example 76: MS obsd. (ESI⁺): 444.3 (M+H)⁺. Chiral IPLC: (Column: AS-3, 4.6*100 mm 3 um, Flow rate: 3.0 mL/min, Eluent: MeOH (0.2% 7M NH3 in MeOH), Temp 40° C.) Retention time=1.83 min.

### Example 77: (R)-6-cyclopropyl-2-(2-hydroxyethyl)-4-((1-(2-methyl-3-(trifluoromethyl)phenyl)ethyl)amino)-2,6-dihydropyrido[3,4-d]pyridazine-1,7-dione

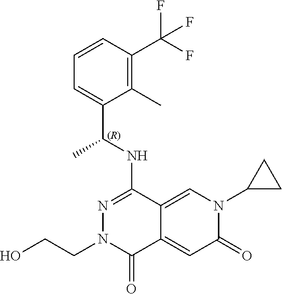

### Step A: (R)-2-(2-(benzyloxy)ethyl)-6-cyclopropyl-4-((1-(2-methyl-3-(trifluoromethyl)phenyl)ethyl)amino)-2,6-dihydropyrido[3,4-d]pyridazine-1,7-dione

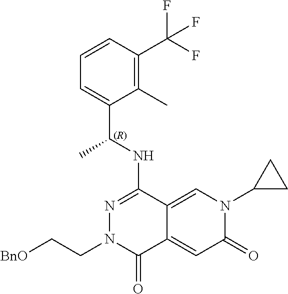

Synthesized according to an analogous procedure as example 24 [steps A-F]starting with 2-(benzyloxy)acetaldehyde. MS obsd. (ESI⁺): 539.4 (M+H)⁺.

### Step B: (R)-6-cyclopropyl-2-(2-hydroxyethyl)-4-((1-(2-methyl-3-(trifluoromethyl)phenyl)ethyl)amino)-2,6-dihydropyrido[3,4-d]pyridazine-1,7-dione

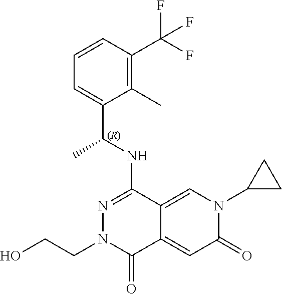

To a solution of ((R)-2-(2-(benzyloxy)ethyl)-6-cyclopropyl-4-((1-(2-methyl-3-(trifluoromethyl)phenyl)ethyl)amino)-2,6-dihydropyrido[3,4-d]pyridazine-1,7-dione was added boron trichloride (1M in DCM, 0.62 mL, 0.62 mmol). The mixture was stirred for 0.5 h at rt. The solvent was removed in vacuo. For use as an intermediate in compound synthesis, the material was used without further purification. For biological testing, the residue was purified by reverse phase HPLC to afford (R)-6-cyclopropyl-2-(2-hydroxyethyl)-4-((1-(2-methyl-3-(trifluoromethyl)phenyl)ethyl)amino)-2,6-dihydropyrido[3,4-d]pyridazine-1,7-dione (2.0 mg, 4.42 umol, 4% yield). MS obsd. (ESI⁺): 449.2 (M+H)⁺.

### Example 78: (R)-6-cyclopropyl-2-(2-(3-methoxy-3-methylazetidin-1-yl)ethyl)-4-((1-(2-methyl-3-(trifluoromethyl)phenyl)ethyl)amino)-2,6-dihydropyrido[3,4-d]pyridazine-1,7-dione

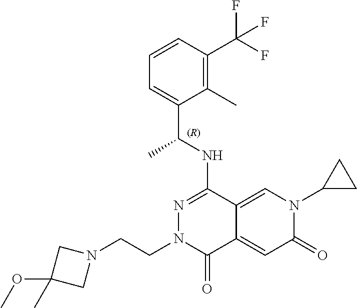

### Step A: (R)-2-(2-chloroethyl)-6-cyclopropyl-4-((1-(2-methyl-3-(trifluoromethyl)phenyl)ethyl)amino)-2,6-dihydropyrido[3,4-d]pyridazine-1,7-dione

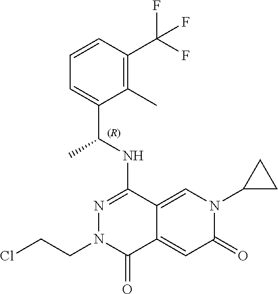

To a solution of crude (R)-6-cyclopropyl-2-(2-hydroxyethyl)-4-((1-(2-methyl-3-(trifluoromethyl)phenyl)ethyl)amino)-2,6-dihydropyrido[3,4-d]pyridazine-1,7-dione (example 77, step b, 100 mg, assumed 0.22 mmol) in DCM (10 mL) was added para-toluenesulfonyl chloride (64 mg, 0.33 mmol), DMAP (27.2 mg, 0.22 mmol) and triethylamine (45 mg, 0.45 mmol). The mixture was stirred for 2 h at 50° C. The solvent was removed in vacuo and the residue was purified by flash column (eluting with 10% MeOH in DCM) to afford (R)-2-(2-chloroethyl)-6-cyclopropyl-4-((1-(2-methyl-3-(trifluoromethyl)phenyl)ethyl)amino)-2,6-dihydropyrido[3,4-d]pyridazine-1,7-dione (74 mg, 0.16 mmol, 72% yield). MS obsd. (ESI⁺): 467.3 (M+H)⁺.

### Step B: (R)-6-cyclopropyl-2-(2-(3-methoxy-3-methylazetidin-1-yl)ethyl)-4-((1-(2-methyl-3-(trifluoromethyl)phenyl)ethyl)amino)-2,6-dihydropyrido[3,4-d]pyridazine-1,7-dione (example 78)

To a solution of 3-methoxy-3-methylazetidine; hydrochloride (42 mg, 0.3 mmol) in CH₃CN (5 mL) was added (R)-2-(2-chloroethyl)-6-cyclopropyl-4-((1-(2-methyl-3-(trifluoromethyl)phenyl)ethyl)amino)-2,6-dihydropyrido[3,4-d]pyridazine-1,7-dione (47 mg, 0.1 mmol), potassium carbonate (56 mg, 0.4 mmol) and potassium iodide (3 mg, 0.02 mmol). The reaction was stirred for 16 h at 80° C. The solvent was removed in vacuo and the residue was purified by reverse phase HPLC to afford (R)-6-cyclopropyl-2-(2-(3-methoxy-3-methylazetidin-1-yl)ethyl)-4-((1-(2-methyl-3-(trifluoromethyl)phenyl)ethyl)amino)-2,6-dihydropyrido[3,4-d]pyridazine-1,7-dione (12.6 mg, 0.02 mmol, 23% yield). MS obsd. (ESI⁺): 532.2 (M+H)⁺.

### Example 79: (R)-6-cyclopropyl-2-(2-(3-fluoro-3-methylazetidin-1-yl)ethyl)-4-((1-(2-methyl-3-(trifluoromethyl)phenyl)ethyl)amino)-2,6-dihydropyrido[3,4-d]pyridazine-1,7-dione

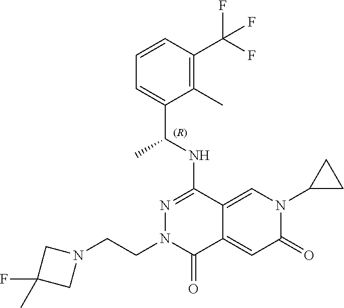

Synthesized according the an analogous route as example 78. MS obsd. (ESI⁺): 520.2 (M+H)⁺.

### Example 80: (R)-6-cyclopropyl-8-(dimethylamino)-2-methyl-4-((1-(2-methyl-3-(trifluoromethyl)phenyl)ethyl)amino)-2,6-dihydropyrido[3,4-d]pyridazine-1,7-dione

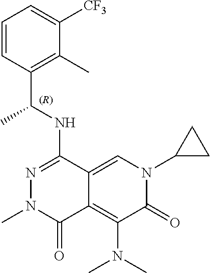

(R)-8-bromo-6-cyclopropyl-2-methyl-4-((1-(2-methyl-3-(trifluoromethyl)phenyl)ethyl)amino)-2,6-dihydropyrido[3,4-d]pyridazine-1,7-dione (50 mg, 100 umol) was dissolved in dimethylamine (2 M in tetrahydrofuran, 2 mL). The reaction mixture was stirred at 25° C. for 3 hrs. The solvent was removed under reduced pressure and the crude product was purified by reverse phase HPLC to afford (R)-6-cyclopropyl-8-(dimethylamino)-2-methyl-4-((1-(2-methyl-3-(trifluoromethyl)phenyl)ethyl)amino)-2,6-dihydropyrido[3,4-d]pyridazine-1,7-dione (30 mg, 64 umol, 64% yield). MS obsd. (ESI⁺): 462.5 (M+H)⁺.

### Example 81: (R)-6-cyclopropyl-8-isopropoxy-2-methyl-4-((1-(2-methyl-3-(trifluoromethyl)phenyl)ethyl)amino)-2,6-dihydropyrido[3,4-d]pyridazine-1,7-dione

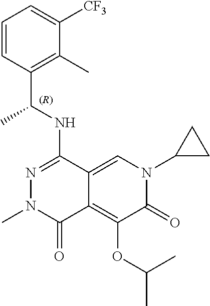

In a flask, isopropanol (37 mg, 603 umol), XantPhos-Pd-G3 (12 mg, 12 umol), (R)-8-bromo-6-cyclopropyl-2-methyl-4-((1-(2-methyl-3-(trifluoromethyl)phenyl)ethyl)amino)-2,6-dihydropyrido[3,4-d]pyridazine-1,7-dione (30 mg, 60 umol), and Cesium Carbonate (59 mg, 181 umol) were dissolved in 1,4-dioxane (1 mL). The mixture was stirred at 50° C. for 16 hrs. The mixture was filtered and the filtrate was concentrated. The crude residue was purified by preparative TLC (MeOH/DCM=1/30) followed by further purification via reverse phase HPLC to afford (R)-6-cyclopropyl-8-isopropoxy-2-methyl-4-((1-(2-methyl-3-(trifluoromethyl)phenyl)ethyl)amino)-2,6-dihydropyrido[3,4-d]pyridazine-1,7-dione (10.0 mg, 21 umol, 34% yield). MS obsd. (ESI⁺): 477.4 [M+H]⁺.

### Example 82: (R)-6-cyclopropyl-8-(cyclopropylmethoxy)-2-methyl-4-((1-(2-methyl-3-(trifluoromethyl)phenyl)ethyl)amino)-2,6-dihydropyrido[3,4-d]pyridazine-1,7-dione

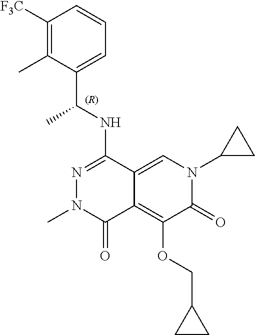

Prepared via an analogous procedure to example 81, using cyclopropylmethanol in place of 2-propanol. MS obsd. (ESI⁺): 489.5 (M+H)⁺.

### Examples 83 and 84: 4-(((R)-1-(3-(difluoromethyl)-2-fluorophenyl)ethyl)amino)-6-((S)-2,2-dimethyltetrahydro-2H-pyran-4-yl)-2-methyl-2,6-dihydropyrido[3,4-d]pyridazine-1,7-dione (example 83, stereochemistry at tetrahydropyran arbitrarily assigned) and 4-(((R)-1-(3-(difluoromethyl)-2-fluorophenyl)ethyl)amino)-6-((R)-2,2-dimethyltetrahydro-2H-pyran-4-yl)-2-methyl-2,6-dihydropyrido[3,4-d]pyridazine-1,7-dione (example 84, stereochemistry at tetrahydropyran arbitrarily assigned)

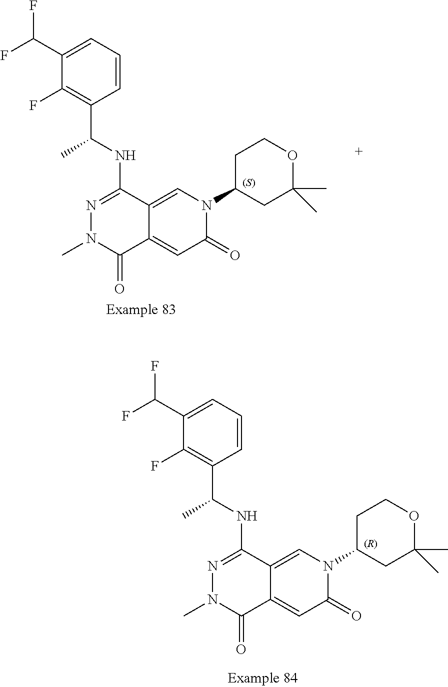

Prepared according to an analogous route as example 4 using racemic 2,2-dimethyltetrahydropyran-4-amine in step A. Example 83: MS obsd. (ESI⁺): 477.3 (M+H)⁺. Chiral HPLC: (Column: IG-3, 4.6*100 mm 3 um, Flow rate: 3.0 mL/min, Eluent: EtOH (1% 7M NH3 in MeOH), Temp 40° C.) Retention time=2.34 min. Example 84: MS obsd. (ESI⁺): 477.3 (M+H)⁺. Chiral HPLC: (Column: IG-3, 4.6*100 mm 3 um, Flow rate: 3.0 mL/min, Eluent: EtOH (1% 7M NH3 in MeOH), Temp 40° C.) Retention time=1.93 min.

### Example 85: (R)-6-cyclopropyl-4-((1-(3-(difluoromethyl)-2-fluorophenyl)ethyl)amino)-8-methoxy-2-methyl-2,6-dihydropyrido[3,4-d]pyridazine-1,7-dione

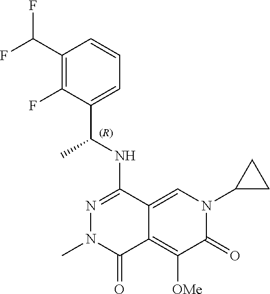

Prepared via an analogous route to example 6. MS obsd. (ESI⁺): 435.2 (M+H)⁺.

### Example 86: (R)-4-((1-(3-(difluoromethyl)-2-fluorophenyl)ethyl)amino)-6-(1-(difluoromethyl)cyclopropyl)-8-(2-(dimethylamino)ethoxy)-2-methyl-2,6-dihydropyrido[3,4-d]pyridazine-1,7-dione

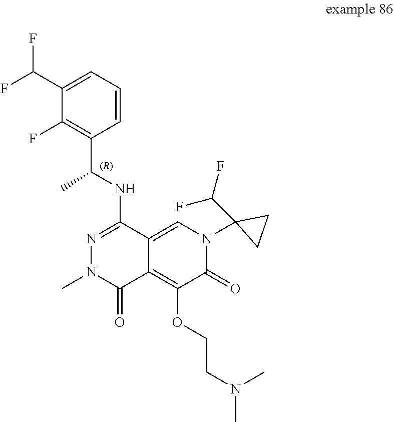

### Step A: (R)-8-bromo-4-((1-(3-(difluoromethyl)-2-fluorophenyl)ethyl)amino)-6-(1-(difluoromethyl)cyclopropyl)-2-methyl-2,6-dihydropyrido[3,4-d]pyridazine-1,7-dione

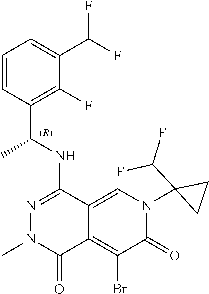

To a solution of (R)-4-((1-(3-(difluoromethyl)-2-fluorophenyl)ethyl)amino)-6-(1-(difluoromethyl)cyclopropyl)-2-methyl-2,6-dihydropyrido[3,4-d]pyridazine-1,7-dione (Example 43) (323 mg, 0.71 mmol) in AcOH (15.0 mL) was added KOAc (105 mg, 1.07 mmol) and Br₂ (171 mg, 1.07 mmol). The reaction was purged with N₂ and stirred at rt for 1 hr. The reaction was concentrated to dryness and the residue was purified by silica gel chromatography (eluted with 0-40% EA in PE) to give (R)-8-bromo-4-((1-(3-(difluoromethyl)-2-fluorophenyl)ethyl)amino)-6-(1-(difluoromethyl)cyclopropyl)-2-methyl-2,6-dihydropyrido[3,4-d]pyridazine-1,7-dione (305 mg, 80% yield). MS obsd. (ESI⁺): 534.1(M+H)⁺.

### Step B: (R)-4-((1-(3-(difluoromethyl)-2-fluorophenyl)ethyl)amino)-6-(1-(difluoromethyl)cyclopropyl)-8-(2-(dimethylamino)ethoxy)-2-methylpyrido[3,4-d]pyridazine-1,7(2H,6H)-dione (example 86)

To a solution of (R)-8-bromo-4-((1-(3-(difluoromethyl)-2-fluorophenyl)ethyl)amino)-6-(1-(difluoromethyl)cyclopropyl)-2-methylpyrido[3,4-d]pyridazine-1,7(2H,6H)-dione (100 mg, 187 μmol) in dioxane (4.0 mL) was added 2-(dimethylamino)ethanol (84 mg, 937 μmol), Cs₂CO₃ (122 mg, 375 μmol) and Xantphos-Pd-G₃ (36 mg, 37 μmol) at room temperature. The mixture was purged with N₂, heated under microwave irradiation at 50° C. for 4 h. The mixture was concentrated under vacuum. The residue was purified by flash column (eluted with 0-2% MeOH in DCM) to give an impure product. The crude product was combined with 10 mg desired product from a previous reaction, and further purified by reverse column to give (R)-4-((1-(3-(difluoromethyl)-2-fluorophenyl)ethyl)amino)-6-(1-(difluoromethyl)cyclopropyl)-8-(2-(dimethylamino)ethoxy)-2-methylpyrido[3,4-d]pyridazine-1,7(2H,6H)-dione (33 mg). MS obsd. (ESI⁺): 542.2(M+H)⁺.

### Example 87: (R)-8-(3,3-difluorocyclobutoxy)-4-((1-(3-(difluoromethyl)-2-fluorophenyl)ethyl)amino)-6-(1-(difluoromethyl)cyclopropyl)-2-methylpyrido[3,4-d]pyridazine-1,7(2H,6H)-dione

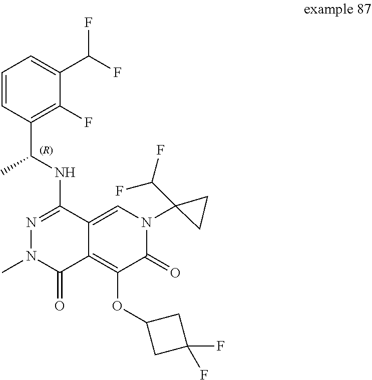

To a solution of (R)-8-bromo-4-((1-(3-(difluoromethyl)-2-fluorophenyl)ethyl)amino)-6-(1-(difluoromethyl)cyclopropyl)-2-methyl-2,6-dihydropyrido[3,4-d]pyridazine-1,7-dione (30 mg, 56 μmol) in dioxane (1.0 mL) was added 3,3-difluorocyclobutanol (30 mg, 281 μmol), Cs₂CO₃ (37 mg, 112 μmol) and Xantphos-Pd-G3 (11 mg, 11 μmol) at room temperature. The mixture was purged with N₂ and heated under microwave reactor at 50° C. for 3 h. The mixture reaction was concentrated and the residue was purified by flash chromatography (eluting with 2% MeOH in DCM) to give the crude product which was further purified by reverse column (C18, MeCN/H₂O) to afford (R)-8-(3,3-difluorocyclobutoxy)-4-((1-(3-(difluoromethyl)-2-fluorophenyl)ethyl)amino)-6-(1-(difluoromethyl)cyclopropyl)-2-methylpyrido [3,4-d]pyridazine-1,7(2H,6H)-dione (10 mg, 32% yield). MS obsd. (ESI⁺): 561.2(M+H)⁺.

### Example 88: (R)-4-((1-(3-(difluoromethyl)-2-fluorophenyl)ethyl)amino)-6-(1-(difluoromethyl)cyclopropyl)-2-methyl-8-(oxetan-3-yloxy)pyrido[3,4-d]pyridazine-1,7(2H,6H)-dione

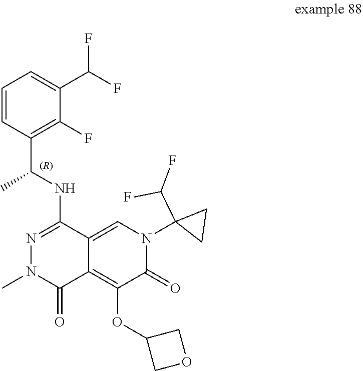

To a solution of (R)-8-bromo-4-((1-(3-(difluoromethyl)-2-fluorophenyl)ethyl)amino)-6-(1-(difluoromethyl)cyclopropyl)-2-methylpyrido[3,4-d]pyridazine-1,7(2H,6H)-dione (40 mg, 75 μmol) in dioxane (2.0 mL) was added oxetan-3-ol (23.8 μL, 375 mol), Cs₂CO₃ (49 mg, 150 μmol) and Xantphos-Pd-G₃ (14 mg, 15 μmol) at room temperature. The mixture was purged with N₂, heated under microwave irradiation at 50° C. for 4 h. The mixture was concentrated. The residue was purified by flash column (eluted with 0-2% MeOH in DCM), and further purified by reverse column to give (R)-4-((1-(3-(difluoromethyl)-2-fluorophenyl)ethyl)amino)-6-(1-(difluoromethyl)cyclopropyl)-2-methyl-8-(oxetan-3-yloxy)pyrido[3,4-d]pyridazine-1,7(2H,6H)-dione (15 mg, 25% yield). MS obsd. (ESI⁺): 527.2(M+H)⁺.

### Example 89: 4-(((R)-1-(3-(difluoromethyl)-2-fluorophenyl)ethyl)amino)-6-(1-(difluoromethyl)cyclopropyl)-8-((1r,3R)-3-fluorocyclobutoxy)-2-methylpyrido[3,4-d]pyridazine-1,7(2H,6H)-dione

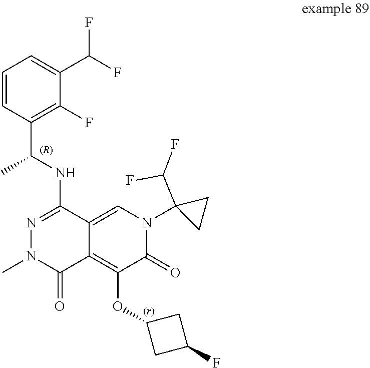

To a solution of (R)-8-bromo-4-((1-(3-(difluoromethyl)-2-fluorophenyl)ethyl)amino)-6-(1-(difluorom ethyl)cyclopropyl)-2-methylpyrido[3,4-d]pyridazine-1,7(2H,6H)-dione (20 mg, 37 μmol) in dioxane (1.0 mL) was added trans-3-fluorocyclobutanol (17 mg, 188 μmol), Cs₂CO₃ (24 mg, 75 μmol) and Xantphos-Pd-G3 (7 mg, 7 μmol) at room temperature. The mixture was purged with N₂, heated under microwave irradiation at 50° C. for 3 h. The mixture reaction was concentrated and the residue was purified by flash chromatography (eluting with 2% MeOH in DCM), followed by reverse phase column (C18, MeCN/H₂O) to afford the title compound (9 mg, 32% yield). MS obsd. (ESI+): 543.3(M+H)⁺.

### Example 90: (R)-4-((1-(3-(difluoromethyl)-2-fluorophenyl)ethyl)amino)-6-(1-(difluoromethyl)cyclopropyl)-2-methyl-8-(oxetan-3-ylmethoxy)-2,6-dihydropyrido[3,4-d]pyridazine-1,7-dione

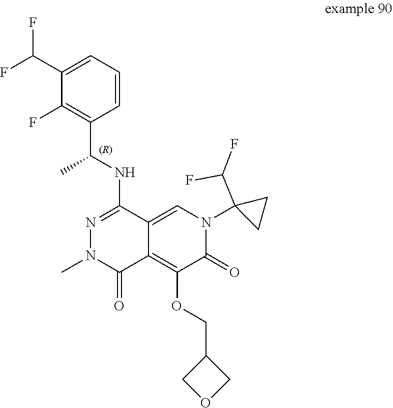

To a solution of (R)-8-bromo-4-((1-(3-(difluoromethyl)-2-fluorophenyl)ethyl)amino)-6-(1-(difluoromethyl)cyclopropyl)-2-methyl-2,6-dihydropyrido[3,4-d]pyridazine-1,7-dione (40 mg, 0.08 mmol) in dioxane (2.0 mL) was added oxetan-3-ylmethanol (33 mg, 0.38 mmol), Cs₂CO₃ (49 mg, 0.15 mmol) and Xantphos-Pd-G3 (14 mg, 0.02 mmol) at rt. The mixture was purged with N₂, heated under microwave irradiation at 50° C. for 4 hr, after which the mixture was cooled and concentrated. The residue was purified by flash column chromatography (eluted with 0-10% MeOH in DCM) to give and impure product. The crude product was combined with 10 mg of the title compound from a similar procedure and further purified by reverse column [ACN/H₂O] to afford the title compound (14 mg). MS obsd. (ESI+): 541.2(M+H)⁺.

### Examples 91 and 92: 6-[(1S,2R)-2-(5-fluoro-3-pyridyl)cyclopropyl]-2-methyl-4-[[(1R)-1-[2-methyl-3-(trifluoromethyl)phenyl]ethyl]amino]pyrido[3,4-d]pyridazine-1,7-dione (example 91, absolute stereochemistry at cyclopropyl is arbitrarily assigned) and 6-[(1R,2S)-2-(5-fluoro-3-pyridyl)cyclopropyl]-2-methyl-4-[[(1R)-1-[2-methyl-3-(trifluoromethyl)phenyl]ethyl]amino]pyrido[3,4-d]pyridazine-1,7-dione (example 92, absolute stereochemistry at cyclopropyl is arbitrarily assigned)

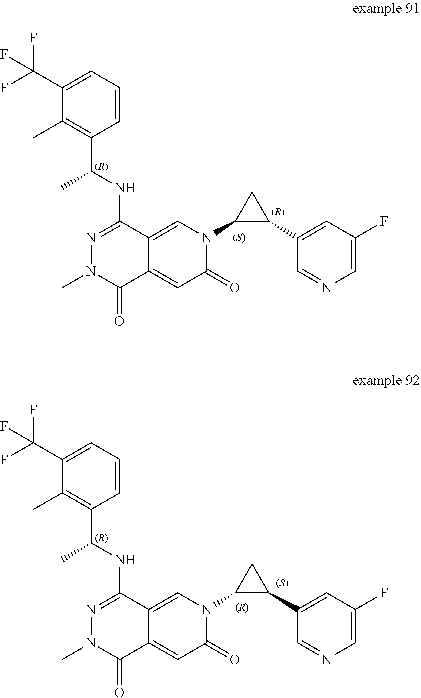

### Step A: tert-butyl (E)-3-(5-fluoro-3-pyridyl)prop-2-enoate

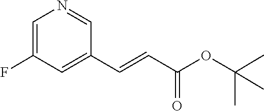 [SMILES: CC(C)(C)OC(=O)/C=C/c1cncc(F)c1]

A mixture of 3-bromo-5-fluoro-pyridine (2.1 g, 11.93 mmol), potassium carbonate (4.95 g, 35.80 mmol), triphenylphosphine (939 mg, 3.58 mmol), Palladium(II)acetate (536 mg, 2.39 mmol) in DMF (30 mL) was stirred at 120° C. for 1 hr. The reaction mixture was cooled to rt and concentrated under vacuum. The residue was purified by flash chromatography (eluting with 10% EA in PE) to afford the title compound (1.77 g, 63% yield). MS obsd. (ESI+): 224.3(M+H)⁺.

### Step B: tert-butyl 2-(5-fluoro-3-pyridyl)cyclopropanecarboxylate

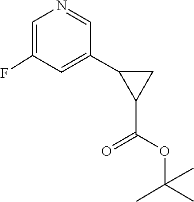 [SMILES: CC(C)(C)OC(=O)C1CC1c1cncc(F)c1]

A mixture of NaH (1.97 g, 49.27 mmol, 60% in mineral oil) and trimethylsulfoxonium iodide (5.91 g, 26.88 mmol) in DMSO (50 mL) was stirred at rt for 30 min, then tert-butyl (E)-3-(5-fluoro-3-pyridyl)prop-2-enoate (5 g, 22.40 mmol) was added to the mixture and stirred at rt for 30 min. To the reaction was added 100 mL ice water. The mixture was extracted with EtOAc, the organic layer was dried over Na₂SO₄, filtered and concentrated under reduced pressure. The residue was purified by flash chromatography (eluting with 5% EA in PE) to afford the title compound (1.83 g, 36% yield). MS obsd. (ESI+): 224.3(M+H)⁺.

### Step C: 2-(5-fluoro-3-pyridyl)cyclopropanecarboxylic acid

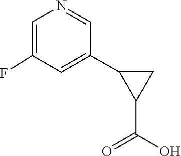 [SMILES: O=C(O)C1CC1c1cncc(F)c1]

A mixture of tert-butyl 2-(5-fluoro-3-pyridyl)cyclopropanecarboxylate (3.23 g, 14.7 mmol) in trifluoroacetic acid (24 mL, 312 mmol) and DCM (100 mL) was stirred at rt for 16 hr. The reaction was concentrated to afford the title compound (3.86 g, 87% yield). MS obsd. (ESI+): 182.1 (M+H)⁺.

### Step D: tert-butyl N-[(1S)-2-(5-fluoro-3-pyridyl)cyclopropyl]carbamate

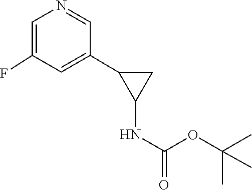 [SMILES: CC(C)(C)OC(=O)NC1CC1c1cncc(F)c1]

A mixture of 2-(5-fluoro-3-pyridyl)cyclopropanecarboxylic acid (6.14 g, 33.89 mmol), DPPA (8.24 g, 33.89 mmol), and TEA (13.72 g, 135.57 mmol, 18.90 mL) in 2-methyl-2-propanol (60 mL) under N₂ atmosphere was stirred at 90° C. for 16 hr. The mixture was concentrated and purified by flash chromatography (eluting with 30% EA in PE) to afford the title compound (869 mg, 9.6% yield). MS obsd. (ESI+): 252.3 (M+H)⁺.

### Step E: (1R)-2-(5-fluoro-3-pyridyl)cyclopropanamine hydrochloride salt

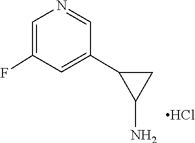 [SMILES: NC1CC1c1cncc(F)c1 ; Cl]

A mixture of tert-butyl N-[(1R)-2-(5-fluoro-3-pyridyl)cyclopropyl]carbamate (850 mg, 3.37 mmol) in 4 N HCl in dioxane (174 mL) was stirred at rt for 3 h. The reaction was concentrated to afford the title compound (800 mg, crude, HCl).

### Step F: 2-[6-chloro-5-(hydroxymethyl)-2-methyl-3-oxo-pyridazin-4-yl]-N-[2-(5-fluoro-3-pyridyl)cyclopropyl]acetamide

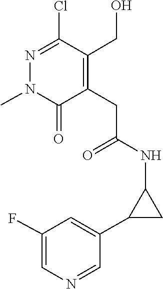 [SMILES: Cn1nc(Cl)c(CO)c(CC(=O)NC2CC2c2cncc(F)c2)c1=O]

To a mixture of 2-(5-fluoro-3-pyridyl)cyclopropanamine hydrochloride salt (650 mg, 4.2 mmol) and 4-chloro-2-methyl-5,8-dihydropyrano[3,4-d]pyridazine-1,7-dione (764 mg, 3.56 mmol) in toluene (26 mL) under a N₂ atmosphere was added trimethylaluminum (5.70 mmol, 2M in toluene) and the mixture was stirred at 75° C. for 4 hr. The mixture was quenched with water and extracted with EtOAc. The organic layer was dried over Na₂SO₄, filtered and concentrated. Purification by flash chromatography (eluting with 50% MeOH in DCM) afforded the title compound (297 mg, 23% yield). MS obsd. (ESI+): 367.2 (M+H)⁺.

### Step G: 4-chloro-6-[(trans)-2-(5-fluoro-3-pyridyl)cyclopropyl]-2-methyl-pyrido[3,4-d]pyridazine-1,7-dione (racemic)

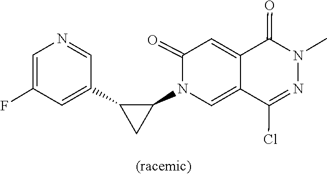 [SMILES: Cn1nc(Cl)c2cn([C@H]3C[C@@H]3c3cncc(F)c3)c(=O)cc2c1=O]

A mixture of 2-[6-chloro-5-(hydroxymethyl)-2-methyl-3-oxo-pyridazin-4-yl]-N-[2-(5-fluoro-3-pyridyl)cyclopropyl]acetamide (262 mg, 714 μmol), Dess-Martin Periodinane (1.06 g, 2.50 mmol) in CHCl₃ (4 mL) under N₂ atmosphere was stirred at rt for 16 h. The reaction was quenched by NaHCO₃ (aq) and extracted into EtOAc. The organic layer was dried over sodium sulfate, filtered and concentrated. The residue was purified by preparative TLC (MeOH:DCM=1:20) to afford 4-chloro-6-[(trans)-2-(5-fluoro-3-pyridyl)cyclopropyl]-2-methyl-pyrido[3,4-d]pyridazine-1,7-dione (70 mg, 28% yield). MS obsd. (ESI+): 347.3 (M+H)⁺.

### Step H: 6-[(1S,2R)-2-(5-fluoro-3-pyridyl)cyclopropyl]-2-methyl-4-[[(1R)-1-[2-methyl-3-(trifluoromethyl)phenyl]ethyl]amino]pyrido[3,4-d]pyridazine-1,7-dione (example 91, absolute stereochemistry at cyclopropyl is arbitrarily assigned) and 6-[(1R,2S)-2-(5-fluoro-3-pyridyl)cyclopropyl]-2-methyl-4-[[(1R)-1-[2-methyl-3-(trifluoromethyl)phenyl]ethyl]amino]pyrido[3,4-d]pyridazine-1,7-dione (example 92, absolute stereochemistry at cyclopropyl is arbitrarily assigned)

A racemic mixture of 4-chloro-6-[(trans)-2-(5-fluoro-3-pyridyl)cyclopropyl]-2-methyl-pyrido[3,4-d]pyridazine-1,7-dione (70 mg, 202 μmol), (1R)-1-[2-methyl-3-(trifluoromethyl)phenyl]ethanamine (49 mg, 242 μmol), Xantphos Pd G3 (19 mg, 20.2 μmol) and Cs₂CO₃ (197 mg, 605 μmol) in dioxane (0.7 mL) under N₂ atmosphere was stirred at 130° C. in a microwave reactor for 5 hr. The mixture was concentrated and purified by preparative TLC (DCM:MeOH=20:1), followed by chiral SFC to give 6-[(1S,2R)-2-(5-fluoro-3-pyridyl)cyclopropyl]-2-methyl-4-[[(1R)-1-[2-methyl-3-(trifluoromethyl)phenyl]ethyl]amino]pyrido[3,4-d]pyridazine-1,7-dione (example 91, absolute stereochemistry at cyclopropyl is arbitrarily assigned) (11.5 mg) and 6-[(1R,2S)-2-(5-fluoro-3-pyridyl)cyclopropyl]-2-methyl-4-[[(1R)-1-[2-methyl-3-(trifluoromethyl)phenyl]ethyl]amino]pyrido[3,4-d]pyridazine-1,7-dione (example 92, absolute stereochemistry at cyclopropyl is arbitrarily assigned) (10.9 mg). Chiral HPLC: (Column: OD-3 (4.6*100 mm, 3 um), Flow rate: 3.0 mL/min, Eluent:MeOH (0.2% 7M NH3 in MeOH), Temp 400C).

Example 91: Chiral HPLC retention time 1.13 min. MS obsd. (ESI+): 514.3 (M+H)⁺.

Example 92: Chiral HPLC retention time 1.72 min. MS obsd. (ESI⁺): 514.3 (M+H)⁺.

### Examples 93 and 94: 4-[[(1R)-1-[3-(difluoromethyl)-2-fluoro-phenyl]ethyl]amino]-6-[(1S,2R)-2-(5-fluoro-3-pyridyl)cyclopropyl]-2-methyl-pyrido[3,4-d]pyridazine-1,7-dione (absolute stereochemistry at cyclopropyl is arbitrarily assigned) and 4-[[(1R)-1-[3-(difluoromethyl)-2-fluoro-phenyl]ethyl]amino]-6-[(1R,2S)-2-(5-fluoro-3-pyridyl)cyclopropyl]-2-methyl-pyrido[3,4-d]pyridazine-1,7-dione (absolute stereochemistry at cyclopropyl is arbitrarily assigned)

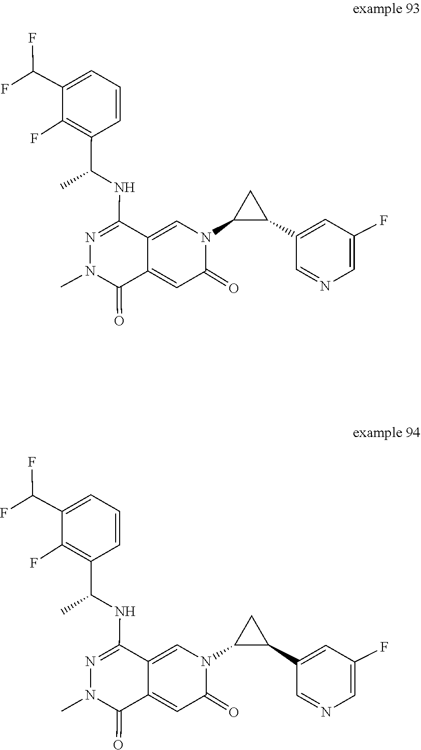 [SMILES: C[C@@H](Nc1nn(C)c(=O)c2cc(=O)n([C@H]3C[C@@H]3c3cncc(F)c3)cc12)c1cccc(C(F)F)c1F ; C[C@@H](Nc1nn(C)c(=O)c2cc(=O)n([C@@H]3C[C@H]3c3cncc(F)c3)cc12)c1cccc(C(F)F)c1F]

Prepared in an analogous manner as described for examples 91/92 step H, starting with (1R)-1-[3-(difluoromethyl)-2-fluoro-phenyl]ethanamine. Chiral HPLC: (Column: OD-3 (4.6*100 mm, 3 um), Flow rate: 3.0 mL/min, Eluent:MeOH (0.2% 7M NH3 in MeOH), Temp 400C).

Example 93. Chiral HPLC retention time=1.09 min. MS obsd. (ESI+): 500.3 (M+H)⁺.

Example 94. Chiral HPLC retention time=1.66 min. MS obsd. (ESI+): 500.3 (M+H)⁺.

### Examples 95 and 96: 4-(((R)-1-(3-(difluoromethyl)-2-fluorophenyl)ethyl)amino)-2-methyl-6-((R)-3-(trifluoromethyl)pyrrolidin-3-yl)-2,6-dihydropyrido[3,4-d]pyridazine-1,7-dione (example 95, absolute stereochemistry at pyrrolidine is arbitrarily assigned) and 4-(((R)-1-(3-(difluoromethyl)-2-fluorophenyl)ethyl)amino)-2-methyl-6-((S)-3-(trifluoromethyl)pyrrolidin-3-yl)-2,6-dihydropyrido[3,4-d]pyridazine-1,7-dione (example 96, absolute stereochemistry at pyrrolidine is arbitrarily assigned)

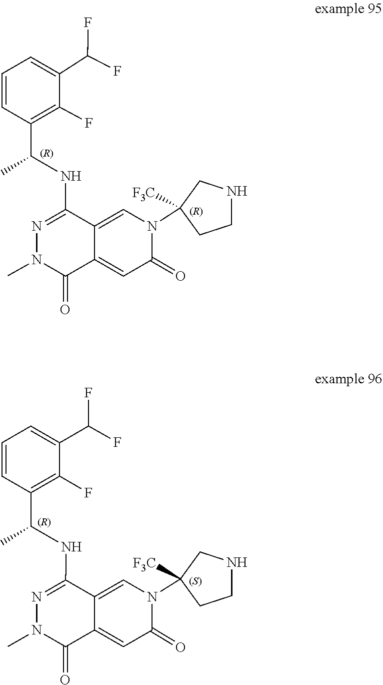

### Step A: tert-butyl 3-(2-(6-chloro-5-(hydroxymethyl)-2-methyl-3-oxo-2,3-dihydropyridazin-4-yl)acetamido)-3-(trifluoromethyl)pyrrolidine-1-carboxylate

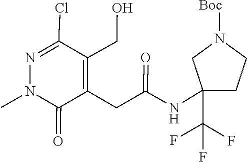 [SMILES: Cn1nc(Cl)c(CO)c(CC(=O)NC2(C(F)(F)F)CCN(C(=O)OC(C)(C)C)C2)c1=O]

A microwave tube was charged with tert-butyl 3-amino-3-(trifluoromethyl)pyrrolidine-1-carboxylate (889 mg, 3.49 mmol), 4-chloro-2-methyl-5,8-dihydropyrano[3,4-d]pyridazine-1,7-dione (500 mg, 2.33 mmol) and acetic acid (210 mg, 3.49 mmol, 201 μL). The tube was capped and heated to 120° C. for 16 hr. The reaction was cooled to room temperature and the residue was purfied by silica gel chromatography (eluting with 2% to 5% MeOH in DCM) to give tert-butyl 3-[[2-[6-chloro-5-(hydroxymethyl)-2-methyl-3-oxo-pyridazin-4-yl]acetyl]amino]-3-(trifluoromethyl) pyrrolidine-1-carboxylate (182 mg, 21% yield). MS obsd. (ESI+): 369.4, 371.4 (M-Boc)⁺.

### Step B: tert-butyl 3-(2-(6-chloro-5-formyl-2-methyl-3-oxo-2,3-dihydropyridazin-4-yl)acetamido)-3-(trifluoromethyl)pyrrolidine-1-carboxylate

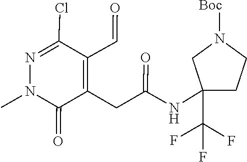 [SMILES: Cn1nc(Cl)c(C=O)c(CC(=O)NC2(C(F)(F)F)CCN(C(=O)OC(C)(C)C)C2)c1=O]

To a solution of tert-butyl 3-[[2-[6-chloro-5-(hydroxymethyl)-2-methyl-3-oxo-pyridazin-4-yl]acetyl]amino]-3-(trifluoromethyl)pyrrolidine-1-carboxylate (260 mg, 554 μmol) in MeCN (10 mL) was added IBX (311 mg, 1.11 mmol). The mixture reaction was stirred for 3 hr at 80° C. The reaction was filtered and the filtrate was collected and concentrated in vacuo to give tert-butyl 3-[[2-(6-chloro-5-formyl-2-methyl-3-oxo-pyridazin-4-yl)acetyl]amino]-3-(trifluoromethyl)pyrrolidine-1-carboxylate (260 mg, crude). The crude was used for the next step without further purification.

### Step C: tert-butyl 3-(4-chloro-2-methyl-1,7-dioxo-1,7-dihydropyrido[3,4-d]pyridazin-6(2H)-yl)-3-(trifluoromethyl)pyrrolidine-1-carboxylate

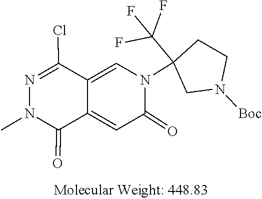 [SMILES: Cn1nc(Cl)c2cn(C3(C(F)(F)F)CCN(C(=O)OC(C)(C)C)C3)c(=O)cc2c1=O]

A mixture of tert-butyl 3-[[2-(6-chloro-5-formyl-2-methyl-3-oxo-pyridazin-4-yl)acetyl]amino]-3-(trifluoromethyl)pyrrolidine-1-carboxylate (260 mg, crude) in AcOH (10 mL) was stirred for 2 hr at 70° C. The reaction was concentrated to dryness and the residue was purified by silica gel chromatography (eltuing with 20% to 50% EA in PE) to afford the title compound (125 mg, 50% yield for two steps). MS obsd. (ESI+): 393.3, 395.3 (M+tBu)⁺.

### Step D: tert-butyl 3-(4-(((R)-1-(3-(difluoromethyl)-2-fluorophenyl)ethyl)amino)-2-methyl-1,7-dioxo-1,7-dihydropyrido[3,4-d]pyridazin-6(2H)-yl)-3-(trifluoromethyl)pyrrolidine-1-carboxylate

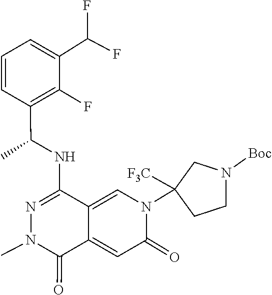 [SMILES: C[C@@H](Nc1nn(C)c(=O)c2cc(=O)n(C3(C(F)(F)F)CCN(C(=O)OC(C)(C)C)C3)cc12)c1cccc(C(F)F)c1F]

A mixture of (1R)-1-[3-(difluoromethyl)-2-fluoro-phenyl]ethanamine (58 mg, 307 μmol), tert-butyl 3-(4-chloro-2-methyl-1,7-dioxo-pyrido[3,4-d]pyridazin-6-yl)-3-(trifluoromethyl)pyrrolidine-1-carboxylate (115 mg, 256 μmol), cesium carbonate (251 mg, 767 μmol), BINAP (32 mg, 51 μmol) and palladium acetate (12 mg, 51 μmol) in toluene (1.5 mL) was heated to 100° C. for 16 hr. The reaction was concentrated to dryness and the residue was purified by silica gel chromatography (eluting with 0% to 4% MeOH in DCM) to afford the title compound (62 mg, 40% yield). MS obsd. (ESI+): 602.5 (M+H)⁺.

### Step E: 4-(((R)-1-(3-(difluoromethyl)-2-fluorophenyl)ethyl)amino)-2-methyl-6-(3-(trifluoromethyl) pyrrolidin-3-yl)-2,6-dihydropyrido[3,4-d]pyridazine-1,7-dione

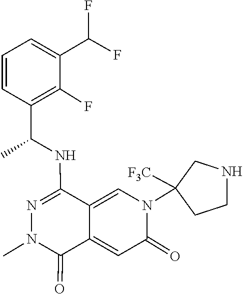 [SMILES: C[C@@H](Nc1nn(C)c(=O)c2cc(=O)n(C3(C(F)(F)F)CCNC3)cc12)c1cccc(C(F)F)c1F]

To a solution of tert-butyl 3-[4-[[(1R)-1-[3-(difluoromethyl)-2-fluoro-phenyl]ethyl]amino]-2-methyl-1,7-dioxo-pyrido[3,4-d]pyridazin-6-yl]-3-(trifluoromethyl)pyrrolidine-1-carboxylate (62 mg, 103 μmol) in DCM (3 mL) was added TFA (0.5 mL). The reaction was stirred for 2 hr at rt. The reaction was concentrated in vacuo. The residue was dissolved in 5 mL NH₃/MeOH (7M) and stirred for 10 min. Then the reaction was concentrated to dryness and purified by silica gel chromatography (eluting with 2% to 10% MeOH in DCM) to afford the title compound (40 mg, 77% yield). MS obsd. (ESI+): 502.3 (M+H)⁺.

### Step F: 4-(((R)-1-(3-(difluoromethyl)-2-fluorophenyl)ethyl)amino)-2-methyl-6-((R)-3-(trifluoromethyl)pyrrolidin-3-yl)-2,6-dihydropyrido[3,4-d]pyridazine-1,7-dione (example 95) and 4-(((R)-1-(3-(difluoromethyl)-2-fluorophenyl)ethyl)amino)-2-methyl-6-((S)-3-(trifluoromethyl)pyrrolidin-3-yl)-2,6-dihydropyrido[3,4-d]pyridazine-1,7-dione (example 96)

A diastereomeric mixture of 4-[[(1R)-1-[3-(difluoromethyl)-2-fluoro-phenyl]ethyl]amino]-2-methyl-6-[3-(trifluoromethyl) pyrrolidin-3-yl]pyrido[3,4-d]pyridazine-1,7-dione (26 mg, 51.85 μmol) was separated by chiral SFC: ((R,R)Whelk-O1 4.6*100 mm 3.5 um,CO2/IPA[1% NH3(7M in MeOH)]=55/45) to give 4-(((R)-1-(3-(difluoromethyl)-2-fluorophenyl)ethyl)amino)-2-methyl-6-((R)-3-(trifluoromethyl)pyrrolidin-3-yl)-2,6-dihydropyrido[3,4-d]pyridazine-1,7-dione (example 95, absolute stereochemistry at pyrrolidine is arbitrarily assigned) (6.63 mg, 26% yield) and 4-(((R)-1-(3-(difluoromethyl)-2-fluorophenyl)ethyl)amino)-2-methyl-6-((S)-3-(trifluoromethyl)pyrrolidin-3-yl)-2,6-dihydropyrido[3,4-d]pyridazine-1,7-dione (example 96, absolute stereochemistry at pyrrolidine is arbitrarily assigned) (4.01 mg, 15% yield). Chiral HPLC: (Column: (R,R)Whelk-O1 (4.6*100 mm, 3.5 um), Flow rate: 3.0 mL/min, Eluent:MeOH (1% 7M NH3 in MeOH), Temp 400C).

Example 95. Chiral HPLC retention time=3.0 min. MS obsd. (ESI⁺): 502.5 (M+H)⁺. H NMR (400 MHz, DMSO-d6) δ 8.53 (1H), 7.60 (1H), 7.49 (1H), 7.37-7.27 (2H), 7.16 (1H), 6.86 (1H), 5.28-5.15 (1H), 4.06 (1H), 3.49 (1H), 3.23 (3H), 3.00 (2H), 2.91 (2H), 2.59 (1H), 1.53 (3H).

Example 96. Chiral HPLC retention time=3.7 min. MS obsd. (ESI⁺): 502.6 (M+H)⁺. H NMR (400 MHz, DMSO-d6) δ 8.55 (1H), 7.61 (1H), 7.48 (1H), 7.29 (2H), 7.16 (1H), 6.87 (1H), 5.20 (1H), 4.08 (1H), 3.51 (1H), 3.23 (3H), 3.02 (2H), 2.96-2.86 (1H), 2.69-2.56 (1H), 1.54 (3H).

### Examples 97 and 98: 4-(((R)-1-(3-(difluoromethyl)-2-fluorophenyl)ethyl)amino)-2-methyl-6-((S)-1-methyl-3-(trifluoromethyl)pyrrolidin-3-yl)-2,6-dihydropyrido[3,4-d]pyridazine-1,7-dione (example 97, absolute stereochemistry at pyrrolidine is arbitrarily assigned) and 4-(((R)-1-(3-(difluoromethyl)-2-fluorophenyl)ethyl)amino)-2-methyl-6-((R)-1-methyl-3-(trifluoromethyl)pyrrolidin-3-yl)-2,6-dihydropyrido[3,4-d]pyridazine-1,7-dione (example 98, absolute stereochemistry at pyrrolidine is arbitrarily assigned)

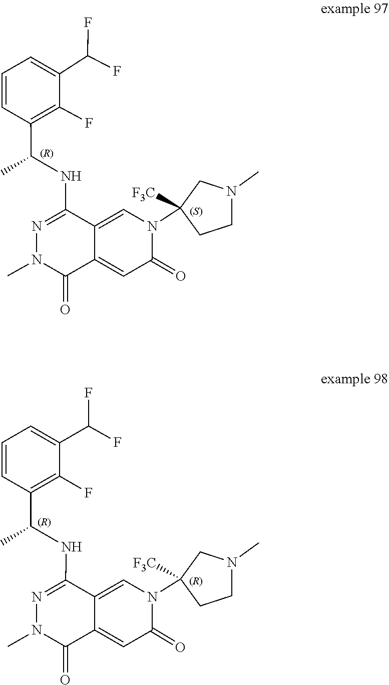

### Step A: 1-benzyl-3-(trifluoromethyl)pyrrolidine-3-carboxylic acid

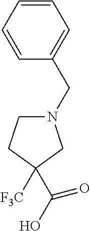 [SMILES: O=C(O)C1(C(F)(F)F)CCN(Cc2ccccc2)C1]

To a solution of 2-(trifluoromethyl)prop-2-enoic acid (6.0 g, 43 mmol) in DCM (100 mL) was slowly added (2-benzyl-3-methoxy-propyl)-trimethyl-silane (10.0 g, 42.8 mmol) at 0° C. Then TFA (772 mg, 6.77 mmol, 521 μL) was added and the reaction was warmed to room temperature and stirred for 16 hr. The resulting precipitate was collected and washed with DCM to give the title compound (9.0 g, 77% yield). MS obsd. (ESI+): 274.3 (M+H)⁺.

### Step B: tert-butyl (1-benzyl-3-(trifluoromethyl)pyrrolidin-3-yl)carbamate

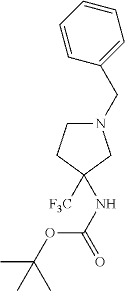 [SMILES: CC(C)(C)OC(=O)NC1(C(F)(F)F)CCN(Cc2ccccc2)C1]

To a solution of 1-benzyl-3-(trifluoromethyl)pyrrolidine-3-carboxylic acid (1 g, 3.66 mmol) in tert-butanol (20 mL) was added triethylamine (1.11 g, 11.0 mmol) and DPPA (979 mg, 4.03 mmol). The reaction was purged with N2 and heated to 90° C. for 1 hr, then heated to 105° C. for 16 hr. The reaction was cooled to room temperature. The reaction was concentrated to dryness under reduce pressure. The residue was purified by silica gel chromatography (eluting with 5% EA in PE) to give the title compound (500 mg, 1.45 mmol, 40% yield). An analogous procedure was used to prepare a larger batch of the title compound (3.5 g, 41% yield). MS obsd. (ESI+): 245.2 (M-Boc+H)⁺.

### Step C: 1-benzyl-3-(trifluoromethyl)pyrrolidin-3-amine

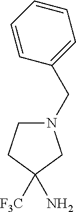 [SMILES: NC1(C(F)(F)F)CCN(Cc2ccccc2)C1]

To a solution of tert-butyl N-[1-benzyl-3-(trifluoromethyl)pyrrolidin-3-yl]carbamate (4.1 g, 11.9 mmol) in dioxane (10 mL) was added HCl (4 M dioxane, 20 mL). The reaction was stirred for 2 hr at rt. The reaction was concentrated to dryness and the residue was dissolved in water (100 mL) and washed with DCM three times. To the aqueous phase was added NaOH (2 M, 50 mL) and stirred for another 1 h. Then the aqueous layer was extracted with DCM. The combined organic layers were washed with brine, dried over Na2SO4, filtered and concentrated in vacuo to give the title compound (2.7 g, 93% yield). MS obsd. (ESI+): 245.3 (M+H)⁺.

### Step D: N-(1-benzyl-3-(trifluoromethyl)pyrrolidin-3-yl)-2-(6-chloro-5-(hydroxymethyl)-2-methyl-3-oxo-2,3-dihydropyridazin-4-yl)acetamide

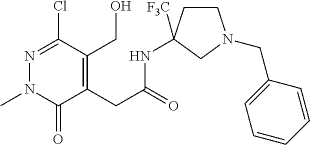 [SMILES: Cn1nc(Cl)c(CO)c(CC(=O)NC2(C(F)(F)F)CCN(Cc3ccccc3)C2)c1=O]

A mixture of 1-benzyl-3-(trifluoromethyl)pyrrolidin-3-amine (1.09 g, 4.47 mmol), 4-chloro-2-methyl-5,8-dihydropyrano[3,4-d]pyridazine-1,7-dione (800 mg, 3.73 mmol) and acetic acid (223.85 mg, 3.73 mmol) was heated to 120° C. for 1 hr. The reaction was cooled to room temperature and concentrated. The residue was purified by reverse phase column (C18, eluting with 10% to 55% MeCN in water/0.1% NH4HCO3) followed by silica gel chromatography (eluting with 0% to 3% MeOH in DCM) to give the title compound (365 mg, 21% yield). MS obsd. (ESI+): 459.5, 461.5 (M+H)⁺.

### Step E: 6-(1-benzyl-3-(trifluoromethyl)pyrrolidin-3-yl)-4-chloro-2-methyl-2,6-dihydropyrido[3,4-d]pyridazine-1,7-dione

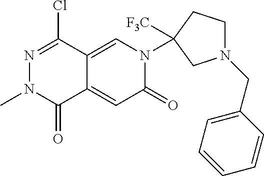 [SMILES: Cn1nc(Cl)c2cn(C3(C(F)(F)F)CCN(Cc4ccccc4)C3)c(=O)cc2c1=O]

To a solution of N-[1-benzyl-3-(trifluoromethyl)pyrrolidin-3-yl]-2-[6-chloro-5-(hydroxymethyl)-2-methyl-3-oxo-pyridazin-4-yl]acetamide (725 mg, 1.58 mmol) in MeCN (15 mL) was added IBX (885 mg, 3.16 mmol). The reaction was heated to 80° C. for 5 hr. The reaction was filtered and the filtrate was concentrated to dryness. The residue was purified by silica gel chromatography (eluting with 0% to 2% MeOH in DCM) to give the title compound (415 mg, 60% yield). MS obsd. (ESI+): 439.4, 441.4 (M+H)⁺.

### Step F: 4-chloro-2-methyl-6-(3-(trifluoromethyl)pyrrolidin-3-yl)-2,6-dihydropyrido[3,4-d]pyridazine-1,7-dione

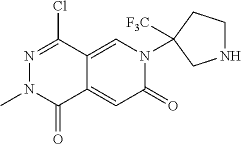 [SMILES: Cn1nc(Cl)c2cn(C3(C(F)(F)F)CCNC3)c(=O)cc2c1=O]

To a solution of 6-(1-benzyl-3-(trifluoromethyl)pyrrolidin-3-yl)-4-chloro-2-methyl-2,6-dihydropyrido[3,4-d]pyridazine-1,7-dione (415 mg, 946 μmol) in DCM (10 mL) was added 1-chloroethyl chloroformate (10 mL, 92 mmol). The reaction was stirred for 5 hr at 80° C. Then the reaction was concentrated to dryness under reduced pressure. The residue was dissolved in MeOH (10 mL) and heated to 80° C. for 1 hr. The reaction was concentrated to dryness and 20 mL NH₃/MeOH (7M) was added. The mixture was concentrated to dryness and the resulting residue was purified by silica gel chromatography (eluting with 2% to 6% MeOH in DCM) to give the title compound (166 mg, 50% yield). MS obsd. (ESI+): 349.4, 351.4 (M+H)⁺.

### Step G: 4-chloro-2-methyl-6-(1-methyl-3-(trifluoromethyl)pyrrolidin-3-yl)-2,6-dihydropyrido[3,4-d]pyridazine-1,7-dione

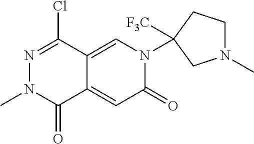

To a solution of 4-chloro-2-methyl-6-[3-(trifluoromethyl)pyrrolidin-3-yl]pyrido[3,4-d]pyridazine-1,7-dione (85 mg, 244 μmol) in MeOH (10 mL) was added paraformaldehyde (73 mg, 2.44 mmol). The mixture was stirred for 10 min at room temperature. Then sodium cyanoborohydride (23 mg, 366 μmol) was added and stirred for 1 hr. The reaction was quenched with aqueous NH₄Cl and extracted with DCM. The combined organic layers were washed with brine, dried over Na₂SO₄, filtered and concentrated to dryness. The residue was purified by silica gel chromatography (eluting with 1% to 3% MeOH in DCM) to give the title compound (29 mg, 33% yield). MS obsd. (ESI+): 363.2, 365.2 (M+H)⁺.

### Step H: 4-(((R)-1-(3-(difluoromethyl)-2-fluorophenyl)ethyl)amino)-2-methyl-6-((S)-1-methyl-3-(trifluoromethyl)pyrrolidin-3-yl)-2,6-dihydropyrido[3,4-d]pyridazine-1,7-dione (example 97, absolute stereochemistry at pyrrolidine is arbitrarily assigned) and 4-(((R)-1-(3-(difluoromethyl)-2-fluorophenyl)ethyl)amino)-2-methyl-6-((R)-1-methyl-3-(trifluoromethyl)pyrrolidin-3-yl)-2,6-dihydropyrido[3,4-d]pyridazine-1,7-dione (example 98, absolute stereochemistry at pyrrolidine is arbitrarily assigned)

A mixture of (1R)-1-[3-(difluoromethyl)-2-fluoro-phenyl]ethanamine (23 mg, 120 μmol), 4-chloro-2-methyl-6-[1-methyl-3-(trifluoromethyl)pyrrolidin-3-yl]pyrido[3,4-d]pyridazine-1,7-dione (29 mg, 78 μmol), cesium carbonate (78 mg, 240 μmol) and Xantphos Pd G3 (15 mg, 16 μmol) in 1,4-dioxane (1.5 mL) was purged with N₂ and then heated to 120° C. in a microwave reactor for 3 hr. The reaction was concentrated to dryness and the residue was purified by silica gel chromatography (eluting with 0˜4% MeOH in DCM) followed by prep-TLC (MeOH/DCM=1/20) to give 4-[[(1R)-1-[3-(difluoromethyl)-2-fluoro-phenyl]ethyl]amino]-2-methyl-6-[(3S)-1-methyl-3-(trifluoromethyl)pyrrolidin-3-yl]pyrido[3,4-d]pyridazine-1,7-dione (example 97, absolute stereochemistry at pyrrolidine is arbitrarily assigned) (1.87 mg, 4.5% yield) and 4-[[(1R)-1-[3-(difluoromethyl)-2-fluoro-phenyl]ethyl]amino]-2-methyl-6-[(3R)-1-methyl-3-(trifluoromethyl)pyrrolidin-3-yl]pyrido[3,4-d]pyridazine-1,7-dione (example 98, absolute stereochemistry at pyrrolidine is arbitrarily assigned) (1.53 mg, 3.7% yield). Analytical chiral HPLC: (Column: Cellulose-SC (4.6*100 mm, 3.0 um), Flow rate: 3.0 mL/min, Eluent:EtOH (1% 7M NH3 in MeOH), Temp 400C).

Example 97. Chiral HPLC retention time=2.4 min. MS obsd. (ESI⁺): 516.4 (M+H)⁺. ¹H NMR (400 MHz, DMSO-d6) δ 8.57 (1H), 7.63 (1H), 7.48 (1H), 7.27 (3H), 6.87 (1H), 5.19 (1H), 3.90 (1H), 3.23 (3H), 3.07 (1H), 2.94 (1H), 2.81 (2H), 2.42 (1H), 2.32 (3H), 1.54 (3H).

Example 98. Chiral HPLC retention time=2.7 min. MS obsd. (ESI⁺): 516.4 (M+H)⁺. ¹H NMR (400 MHz, DMSO-d6) δ 8.56 (1H), 7.60 (1H), 7.48 (1H), 7.41-7.05 (3H), 6.87 (1H), 5.26-5.11 (1H), 3.89 (1H), 3.23 (3H), 3.09 (1H), 2.93 (1H), 2.87-2.73 (2H), 2.42 (1H), 2.33 (3H), 1.54 (3H).

### Examples 99 and 100: 6-((S)-1-acetyl-3-(trifluoromethyl)pyrrolidin-3-yl)-4-(((R)-1-(3-(difluoromethyl)-2-fluorophenyl)ethyl)amino)-2-methyl-2,6-dihydropyrido[3,4-d]pyridazine-1,7-dione (example 99, absolute stereochemistry at pyrrolidine is arbitrarily assigned) and 6-((R)-1-acetyl-3-(trifluoromethyl)pyrrolidin-3-yl)-4-(((R)-1-(3-(difluoromethyl)-2-fluorophenyl)ethyl)amino)-2-methyl-2,6-dihydropyrido[3,4-d]pyridazine-1,7-dione (example 100, absolute stereochemistry at pyrrolidine is arbitrarily assigned)

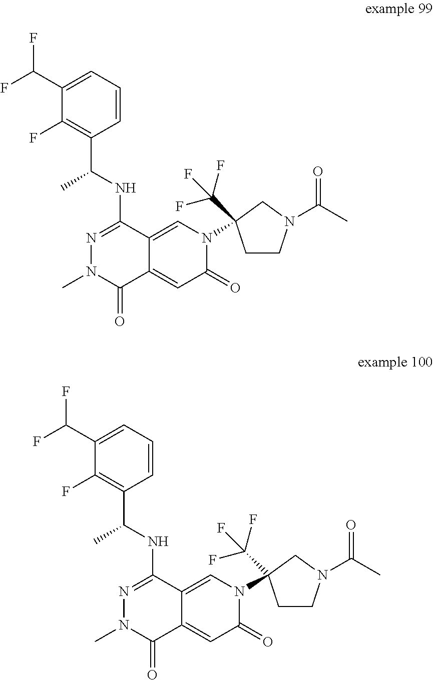

### Step A: 6-(1-acetyl-3-(trifluoromethyl)pyrrolidin-3-yl)-4-chloro-2-methyl-2,6-dihydropyrido[3,4-d]pyridazine-1,7-dione

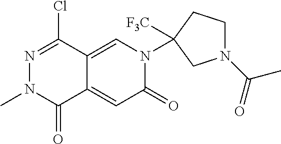 [SMILES: CC(=O)N1CCC(n2cc3c(Cl)nn(C)c(=O)c3cc2=O)(C(F)(F)F)C1]

To a solution of 4-chloro-2-methyl-6-[3-(trifluoromethyl)pyrrolidin-3-yl]pyrido[3,4-d]pyridazine-1,7-dione (example 97/98 step F, 50 mg, 143 μmol) in DCM (10 mL) was added TEA (30 μL, 215 μmol). Then acetyl chloride (13 μL, 215 μmol) was added at 0° C. The reaction was warmed to rt and stirred for 1 hr. The reaction was concentrated and the residue was purified by silica gel chromatography (eluting with 1%-3% MeOH in DCM) to give the title compound (56 mg, 99% yield). MS obsd. (ESI+): 391.0, 393.0 (M+H)⁺.

### Step B: 6-((S)-1-acetyl-3-(trifluoromethyl)pyrrolidin-3-yl)-4-(((R)-1-(3-(difluoromethyl)-2-fluorophenyl)ethyl)amino)-2-methyl-2,6-dihydropyrido[3,4-d]pyridazine-1,7-dione (example 99, absolute stereochemistry at pyrrolidine is arbitrarily assigned) and 6-((R)-1-acetyl-3-(trifluoromethyl)pyrrolidin-3-yl)-4-(((R)-1-(3-(difluoromethyl)-2-fluorophenyl)ethyl)amino)-2-methyl-2,6-dihydropyrido[3,4-d]pyridazine-1,7-dione (example 100, absolute stereochemistry at pyrrolidine is arbitrarily assigned)

Prepared in an essentially analogous manner to examples 97/98 step H, starting with 6-(1-acetyl-3-(trifluoromethyl)pyrrolidin-3-yl)-4-chloro-2-methyl-2,6-dihydropyrido[3,4-d]pyridazine-1,7-dione. Chiral HPLC: (Column: OD-3 (4.6*100 mm, 3 um), Flow rate: 3.0 mL/min, Eluent:MeOH (0.2% 7M NH3 in MeOH), Temp 40° C.).

Example 99. Chiral HPLC retention time=1.33 min. MS obsd. (ESI+): 544.4 (M+H)⁺.

Example 100. Chiral HPLC retention time=1.93 min. MS obsd. (ESI+): 544.4 (M+H)⁺.

### Examples 101 and 102: (R)-6-cyclopropyl-2-methyl-4-((1-(5,6,7,8-tetrahydronaphthalen-2-yl)ethyl)amino)-2,6-dihydropyrido[3,4-d]pyridazine-1,7-dione and (S)-6-cyclopropyl-2-methyl-4-((1-(5,6,7,8-tetrahydronaphthalen-2-yl)ethyl)amino)-2,6-dihydropyrido[3,4-d]pyridazine-1,7-dione

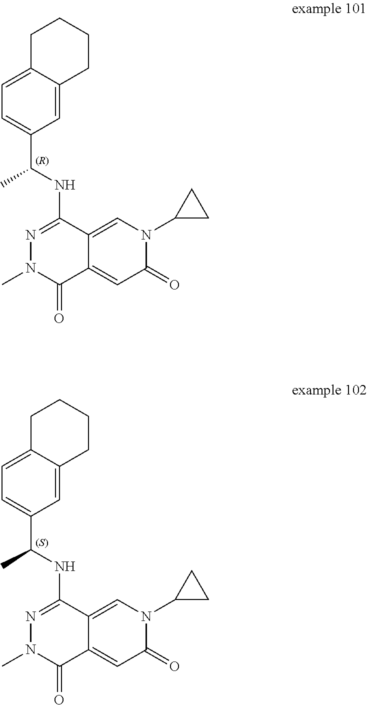

Prepared in an essentially analogous manner to example 2 step B, starting with 1-(5,6,7,8-tetrahydronaphthalen-2-yl)ethan-1-amine. The racemic mixture was separated using chiral HPLC. (Column Name: AS-3 4.6*100 mm 3 um, MeOH[0.2% NH3(7M in MeOH)] to give the titled compounds.

Example 101. First eluting isomer. MS obsd. (ESI+): 391.2 (M+H)⁺. ¹H NMR (400 MHz, DMSO-d6) δ 8.70 (1H), 7.12-7.04 (2H), 6.95 (2H), 6.83 (1H), 4.89 (1H), 3.53 (1H), 2.71-2.64 (4H), 1.71 (4H), 1.45 (3H), 1.12 (2H), 1.08-0.98 (2H).

Example 102. Second eluting isomer. MS obsd. (ESI+): 391.2 (M+H)⁺. ¹H NMR (400 MHz, DMSO-d6) δ 8.69 (1H), 7.11-7.05 (2H), 6.95 (2H), 6.83 (1H), 4.89 (1H), 3.53 (1H), 2.68 (4H), 1.71 (4H), 1.45 (3H), 1.15-1.10 (2H), 1.09-1.00 (2H).

### Examples 103 and 104: (S)-6-cyclopropyl-4-((1-(3-(1,1-difluoro-2-hydroxyethyl)-2-fluorophenyl)ethyl)amino)-2-methyl-2,6-dihydropyrido[3,4-d]pyridazine-1,7-dione and (R)-6-cyclopropyl-4-((1-(3-(1,1-difluoro-2-hydroxyethyl)-2-fluorophenyl)ethyl)amino)-2-methyl-2,6-dihydropyrido[3,4-d]pyridazine-1,7-dione

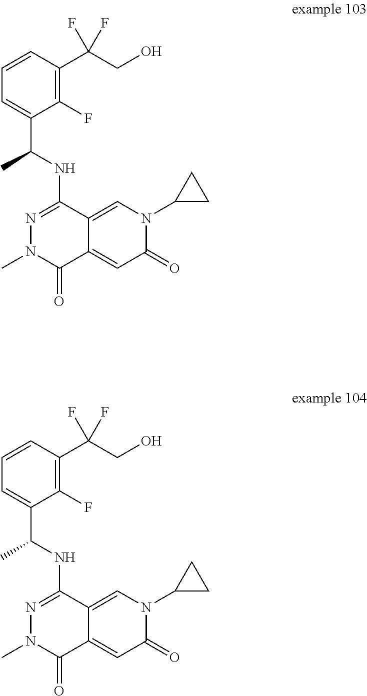 [SMILES: C[C@H](Nc1nn(C)c(=O)c2cc(=O)n(C3CC3)cc12)c1cccc(C(F)(F)CO)c1F ; C[C@@H](Nc1nn(C)c(=O)c2cc(=O)n(C3CC3)cc12)c1cccc(C(F)(F)CO)c1F]

Prepared in a similar manner to example 40, starting with 1-(3-(2-((tert-butyldimethylsilyl)oxy)-1,1-difluoroethyl)-2-fluorophenyl)ethan-1-amine (Prepared according to a procedure described in WO2022058344). Chiral HPLC: (Column Name: AS-3 4.6*100 mm 3 um, MeOH[0.2% NH3(7M in MeOH)], 3 mL/min, 40° C.).

Example 103: Chiral HPLC retention time=1.78 min. MS obsd. (ESI+): 435.4 (M+H)⁺.

Example 104: Chiral HPLC retention time=2.31 min. MS obsd. (ESI+): 435.4 (M+H)⁺.

### Examples 105 and 106: (S)-2-(3-(1-((6-cyclopropyl-2-methyl-1,7-dioxo-1,2,6,7-tetrahydropyrido[3,4-d]pyridazin-4-yl)amino)ethyl)-2-fluorophenyl)-2,2-difluoro-N,N-dimethylacetamide and (R)-2-(3-(1-((6-cyclopropyl-2-methyl-1,7-dioxo-1,2,6,7-tetrahydropyrido[3,4-d]pyridazin-4-yl)amino)ethyl)-2-fluorophenyl)-2,2-difluoro-N,N-dimethylacetamide

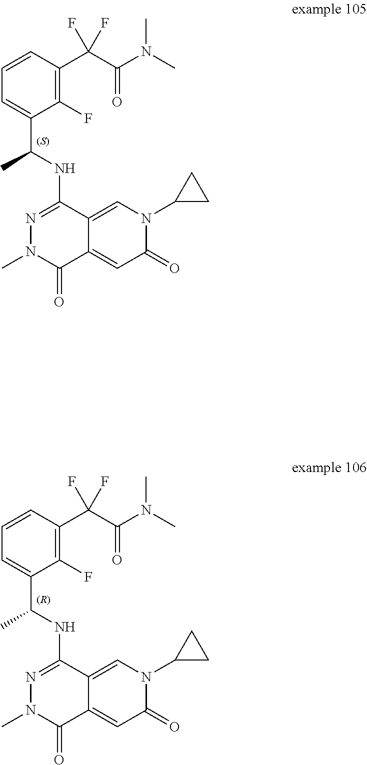

### Step A: Ethyl 2-(3-bromo-2-fluorophenyl)-2,2-difluoroacetate

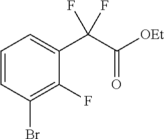 [SMILES: CCOC(=O)C(F)(F)c1cccc(Br)c1F]

A solution of ethyl bromodifluoroacetate (16.9 g, 83.1 mmol) in DMSO (160 mL) under N₂ was stirred at rt for 1 h. Then 1-bromo-2-fluoro-3-iodo-benzene (10 g, 33.23 mmol) was added and the mixture was heated to 70° C. for 3 h. Ice-water (240 mL) was added to the mixture, and the mixture was filtered through a celite pad. The aqueous mixture was extracted with EtOAc (3×200 mL). The combined organic layers were concentrated under vacuum to obtain the crude product and purified by flash chromatography column (eluting with 0-6% EA in PE) to give the title compound (8.4 g, 83% yield). ¹H NMR (400 MHz, DMSO-d6) δ: 8.05-7.97 (1H), 7.77-7.70 (1H), 7.38 (1H), 4.36 (2H), 1.24 (3H).

### Step B: 2-(3-bromo-2-fluorophenyl)-2,2-difluoro-N,N-dimethylacetamide

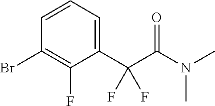 [SMILES: CN(C)C(=O)C(F)(F)c1cccc(Br)c1F]

A solution of ethyl 2-(3-bromo-2-fluoro-phenyl)-2,2-difluoro-acetate (1.5 g, 5.1 mmol) and dimethylamine solution (25 mL, 40 wt %) was heated to 70° C. for 18 h. The mixture was concentrated under vacuum to obtain the crude product and it was purified by flash chromatography column (eluting with 0-10% EA in PE) to give the title compound (1 g, 66% yield). MS obsd. (ESI+): 296.2, 298.2 (M+H)⁺.

### Step C: 2-(3-acetyl-2-fluorophenyl)-2,2-difluoro-N,N-dimethylacetamide

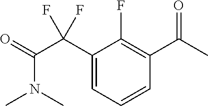 [SMILES: CC(=O)c1cccc(C(F)(F)C(=O)N(C)C)c1F]

To a mixture of 2-(3-bromo-2-fluoro-phenyl)-2,2-difluoro-N,N-dimethyl-acetamide (3 g, 10.1 mmol), tributyl(1-ethoxyvinyl)stannane (4.39 g, 12.1 mmol) and TEA (2.05 g, 20.2 mmol) in dioxane (30 mL) was added bis(Triphenylphosphine)palladium (II) chloride (711 mg, 1.01 mmol) under N₂. The mixture was heated to 90° C. for 16 h. Ice-water (30 mL) was then added and the mixture was extracted with EA (100 mL×3). The combined organic layers were concentrated under vacuum to obtain the crude product. The crude product was purified by flash chromatography column (eluting with 0%-25% EA in PE) to give the title compound (2.1 g, 78% yield). MS obsd. (ESI+): 260.3 (M+H)⁺.

### Step D: (R,E)-2-(3-(1-((tert-butylsulfinyl)imino)ethyl)-2-fluorophenyl)-2,2-difluoro-N,N-dimethylacetamide

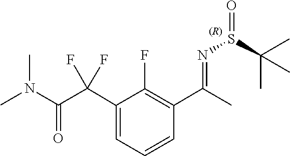

To a mixture of 2-(3-acetyl-2-fluoro-phenyl)-2,2-difluoro-N,N-dimethyl-acetamide (2.1 g, 8.10 mmol), (R)-2-methylpropane-2-sulfinamide (1.47 g, 12.2 mmol) in THE (21 mL) was added titanium ethoxide (4.62 g, 20.3 mmol). The mixture was heated to 65° C. under N₂ for 18 h. Ice-water (50 mL) was added and the mixture was extracted with EA (100 mL×3). The combined organic layers were concentrated under vacuum to obtain the crude product. The crude product was purified by flash chromatography column (eluting with 0%-50% EA in PE) to give the title compound (2.4 g, 80% yield). MS obsd. (ESI+): 363.4 (M+H)⁺.

### Step E: 2-(3-(1-(((R)-tert-butylsulfinyl)amino)ethyl)-2-fluorophenyl)-2,2-difluoro-N,N-dimethylacetamide

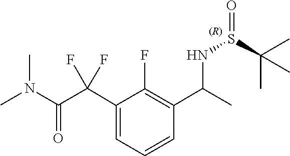

A solution of (R,E)-2-(3-(1-((tert-butylsulfinyl)imino)ethyl)-2-fluorophenyl)-2,2-difluoro-N,N-dimethylacetamide (1 g, 2.76 mmol) in water (0.2 mL) and THE (10 mL) was cooled to −70° C. NaBH₄ (188 mg, 4.97 mmol) was then added and the mixture was stirred for for 2 h. Ice-water (40 mL) was then added and the mixture was extracted with EA (80 mL×3). The combined organic layers were concentrated under vacuum to obtain the crude product. The crude product was purified by flash chromatography (eluting with 0%-68% EA in PE) to give the title compound (1 g, 97% yield). ¹H NMR (400 MHz, DMSO-d6) Diastereomeric mixture (˜2:1) δ 7.73 (1H), 7.50 (1H), 7.39-7.32 (1H), 5.87 (0.65H), 5.55 (0.37H), 4.68 (m, 1H), 2.98-2.94 (m, 6H), 1.49 (1H), 1.41 (2H), 1.10 (9H).

### Step F: 2-(3-(1-aminoethyl)-2-fluorophenyl)-2,2-difluoro-N,N-dimethylacetamide hydrochloric acid salt

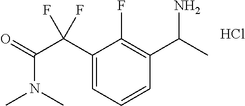 [SMILES: CC(N)c1cccc(C(F)(F)C(=O)N(C)C)c1F ; Cl]

To a solution of 2-[3-[1-[[(R)-tert-butylsulfinyl]amino]ethyl]-2-fluoro-phenyl]-2,2-difluoro-N,N-dimethyl-acetamide (1.00 g, 2.74 mmol) in DCM (21 mL) was slowly added 4 M HCl in 1,4-dioxane (6.86 mL). The mixture was stirred at rt for 2 h and then concentrated under vacuum to obtain the crude product. The crude product was purified by flash chromatography column (eluting with 0%-10% MeOH in DCM) to give the title compound (1.1 g, 76% yield). MS obsd. (ESI+): 261.3 (M+H)⁺.

Step G: (S)-2-(3-(1-((6-cyclopropyl-2-methyl-1,7-dioxo-1,2,6,7-tetrahydropyrido[3,4-d]pyridazin-4-yl)amino)ethyl)-2-fluorophenyl)-2,2-difluoro-N,N-dimethylacetamide(example 105) and (R)-2-(3-(1-((6-cyclopropyl-2-methyl-1,7-dioxo-1,2,6,7-tetrahydropyrido[3,4-d]pyridazin-4-yl)amino)ethyl)-2-fluorophenyl)-2,2-difluoro-N,N-dimethylacetamide(example 106).

To a mixture of 4-chloro-6-cyclopropyl-2-methyl-pyrido[3,4-d]pyridazine-1,7-dione (150 mg, 596 μmol), 2-[3-(1-aminoethyl)-2-fluoro-phenyl]-2,2-difluoro-N,N-dimethyl-acetamide hydrochloric acid salt (186 mg, 715 μmol), Xantphos (69 mg, 119.11 μmol, 0.2 eq.) and potassium carbonate (247 mg, 1.8 mmol) in DMF (2 mL) was added Pd₂(dba)₃ (55 mg, 59 μmol).The mixture was heated to 105° C. for 2 h. Ice-water (10 mL) was added and the mixture was extracted with EA (30 mL×3). The combined organic layers were concentrated under vacuum to obtain the crude product. The crude product was purified by preparative HPLC (MeCN/water/0.1% FA) followed by chiral SFC to give (S)-2-(3-(1-((6-cyclopropyl-2-methyl-1,7-dioxo-1,2,6,7-tetrahydropyrido[3,4-d]pyridazin-4-yl)amino)ethyl)-2-fluorophenyl)-2,2-difluoro-N,N-dimethylacetamide(example 105) and (R)-2-(3-(1-((6-cyclopropyl-2-methyl-1,7-dioxo-1,2,6,7-tetrahydropyrido[3,4-d]pyridazin-4-yl)amino)ethyl)-2-fluorophenyl)-2,2-difluoro-N,N-dimethylacetamide(example 106). Chiral HPLC:Column Name:OJ-3 4.6*100 mm 3 um, MeOH[0.2% NH3(7M in MeOH)], flow rate: 3 mL/min, column temp: 40° C.

Example 105. Chiral HPLC retention time=1.99 min. MS obsd. (ESI+): 476.0 (M+H)⁺. ¹H NMR (400 MHz, DMSO-d6) δ 8.72 (1H), 7.65 (1H), 7.45 (1H), 7.29 (1H), 7.16 (1H), 6.82 (1H), 5.15 (1H), 3.58-3.52 (1H), 3.23 (3H), 2.96 (6H), 1.50 (3H), 1.17-1.05 (4H).

Example 106. Chiral HPLC retention time=2.90 min. MS obsd. (ESI+): 476.0 (M+H)⁺. ¹H NMR (400 MHz, DMSO-d6) δ8.72 (1H), 7.65 (1H), 7.45 (1H), 7.29 (1H), 7.16 (1H), 6.82 (1H), 5.15 (1H), 3.59-3.52 (1H), 3.23 (3H), 2.96 (6H), 1.50 (3H), 1.12 (4H).

### Examples 107 and 108: (R)-2-(3-(1-((6-cyclopropyl-2-methyl-1,7-dioxo-1,2,6,7-tetrahydropyrido[3,4-d]pyridazin-4-yl)amino)ethyl)-2-fluorophenyl)-2,2-difluoro-N-methylacetamide and (S)-2-(3-(1-((6-cyclopropyl-2-methyl-1,7-dioxo-1,2,6,7-tetrahydropyrido[3,4-d]pyridazin-4-yl)amino)ethyl)-2-fluorophenyl)-2,2-difluoro-N-methylacetamide

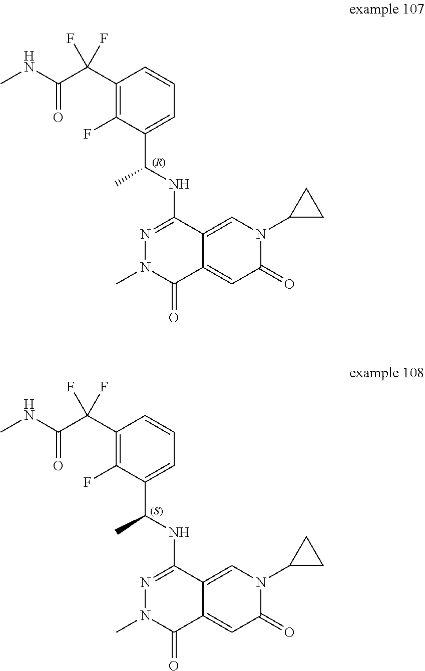

### Step A: 2-(3-bromo-2-fluorophenyl)-2,2-difluoro-N-methylacetamide

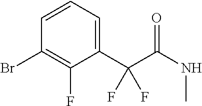

To a solution of ethyl 2-(3-bromo-2-fluoro-phenyl)-2,2-difluoro-acetate (1.5 g, 5.1 mmol) in methylamine (15 mL) was heated to 70° C. for 18 h. The mixture was concentrated under vacuum to obtain the crude product. The crude product was purified by flash chromatography column (eluting with 0%-50% EA in PE) to the title compound (1.1 g, 76% yield).

¹H NMR (400 MHz, DMSO) δ 9.04 (1H), 7.95 (1H), 7.65 (1H), 7.34 (1H), 2.73 (3H).

### Step B: (R)-2-(3-(1-((6-cyclopropyl-2-methyl-1,7-dioxo-1,2,6,7-tetrahydropyrido[3,4-d]pyridazin-4-yl)amino)ethyl)-2-fluorophenyl)-2,2-difluoro-N-methylacetamide(example 107) and (S)-2-(3-(1-((6-cyclopropyl-2-methyl-1,7-dioxo-1,2,6,7-tetrahydropyrido[3,4-d]pyridazin-4-yl)amino)ethyl)-2-fluorophenyl)-2,2-difluoro-N-methylacetamide (example 108)

Prepared in an essentially analogous manner to examples 105/6 steps C-G, starting with 2-(3-bromo-2-fluorophenyl)-2,2-difluoro-N-methylacetamide. Chiral HPLC: (Column Name: OD-3 4.6*100 mm 3 um, MeOH[0.2% NH3(7M in MeOH)], flow rate: 3.0 mL/min, column temp: 40° C.).

Example 107. Chiral HPLC retention time=1.24 min. MS obsd. (ESI+): 462.2 (M+H)⁺. ¹H NMR (400 MHz, DMSO-d6) δ 8.95 (1H), 8.71 (s, 1H), 7.64 (1H), 7.46 (1H), 7.28 (1H), 7.15 (1H), 6.81 (1H), 5.14 (1H), 3.59-3.51 (1H), 3.24 (3H), 2.73 (3H), 1.50 (3H), 1.10 (4H).

Example 108. Chiral HPLC retention time=1.53 min. MS obsd. (ESI+): 462.0 (M+H)⁺. ¹H NMR (400 MHz, DMSO-d6) δ 8.95 (1H), 8.71 (1H), 7.64 (1H), 7.46 (1H), 7.28 (1H), 7.15 (1H), 6.81 (1H), 5.14 (1H), 3.59-3.51 (1H), 3.24 (3H), 2.73 (3H), 1.50 (3H), 1.10 (4H).

### Example 109: (R)-6-cyclopropyl-4-((1-(3-(1,1-difluoro-2-methoxy-2-methylpropyl)-2-fluorophenyl)ethyl)amino)-2-methyl-2,6-dihydropyrido[3,4-d]pyridazine-1,7-dione

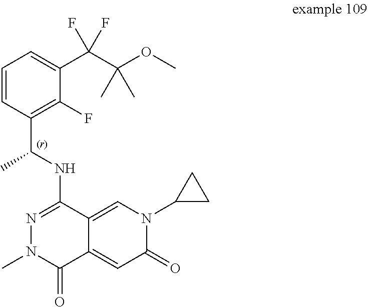 [SMILES: COC(C)(C)C(F)(F)c1cccc([C@@H](C)Nc2nn(C)c(=O)c3cc(=O)n(C4CC4)cc23)c1F]

### Step A: 1-bromo-3-(1,1-difluoro-2-methoxy-2-methylpropyl)-2-fluorobenzene

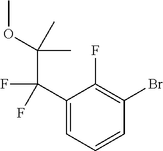 [SMILES: COC(C)(C)C(F)(F)c1cccc(Br)c1F]

To a solution of 1-(3-bromo-2-fluoro-phenyl)-1,1-difluoro-2-methyl-propan-2-ol (1.3 g, 4.59 mmol, prepared according to the procedure detailed in WO2019122129) in dioxane (20 mL), was added sodium hydride (121 mg, 5.05 mmol). The mixture was stirred for 30 min, followed by the addition of iodomethane (430 μL, 6.89 mmol). The mixture was stirred at 25° C. for 2 hrs. The mixture was poured into cold water and extracted with EA. The combined organic layers were dried over Na₂SO₄, filtered and concentrated. The residue was purified via flash chromatography (eluting with 3% to 5% EA in PE) to give the title compound (860 mg, 63% yield). MS obsd. (ESI+): 296.5, 297.5, [M+H]⁺.

### Step B: (R)-1-(3-(1,1-difluoro-2-methoxy-2-methylpropyl)-2-fluorophenyl)ethan-1-amine hydrochloride

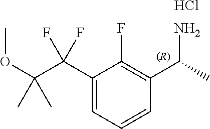

The title compound was prepared according to the procedure detailed in WO2019122129 for (R)-1-(3-(1-aminoethyl)-2-fluorophenyl)-1,1-difluoro-2-methylpropan-2-ol HCl, starting with 1-bromo-3-(1,1-difluoro-2-methoxy-2-methylpropyl)-2-fluorobenzene. MS obsd. (ESI+): 262.4.

### Step C: (R)-6-cyclopropyl-4-((1-(3-(1,1-difluoro-2-methoxy-2-methylpropyl)-2-fluorophenyl)ethyl)amino)-2-methyl-2,6-dihydropyrido[3,4-d]pyridazine-1,7-dione (example 109)

A solution of 4-chloro-6-cyclopropyl-2-methyl-pyrido[3,4-d]pyridazine-1,7-dione (60 mg, 238.41 μmol), (1R)-1-[3-(1,1-difluoro-2-methoxy-2-methyl-propyl)-2-fluoro-phenyl]ethanamine; hydrochloride (85 mg, 286.09 μmol), XantPhos (28 mg, 48 μmol), Pd₂(dba)₃ (22 mg, 24 μmol), and potassium carbonate (99 mg, 0.72 mmol) in DMF (500 μL) was stirred at 100° C. for 16 hrs. The mixture was filtered and diluted with NaHCO₃ (aq.). The mixture was extracted with EA,dried over Na₂SO₄, filtered and concentrated. The residue was purified by preparative HPLC (CH₃CN/0.1% NH₄HCO₃—Water=45-60%) to afford the title compound (38 mg, 33% yield).

MS obsd. (ESI+): 477.5 (M+H)⁺. ¹H NMR (400 MHz, DMSO-d₆) δ: 8.74 (1H), 7.57 (1H), 7.28 (1H), 7.21 (1H), 7.14 (1H), 6.82 (1H), 5.19 (1H), 3.62-3.49 (1H), 3.23 (3H), 3.19 (3H), 1.49 (3H), 1.26 (6H), 1.19-1.12 (2H), 1.11-1.04 (2H).

### Example 110: (R)-4-((1-(3-(1,1-difluoro-2-methoxy-2-methylpropyl)-2-fluorophenyl)ethyl)amino)-6-(1-(difluoromethyl)cyclopropyl)-2-methyl-2,6-dihydropyrido[3,4-d]pyridazine-1,7-dione

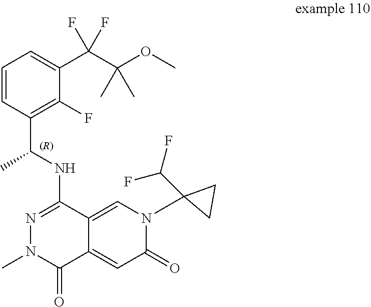

A solution of 4-chloro-6-(1-(difluoromethyl)cyclopropyl)-2-methyl-2,6-dihydropyrido[3,4-d]pyridazine-1,7-dione (20 mg, 66.3 μmol), (1R)-1-[3-(1,1-difluoro-2-methoxy-2-methyl-propyl)-2-fluoro-phenyl]ethanamine hydrochloride (24 mg, 79.6 μmol), RuPhos Pd G3 (11 mg, 13.3 μmol), potassium carbonate (9 mg, 66.3 μmol) in dioxane (0.4 mL) was stirred at 100° C. for 16 hrs. The reaction mixture was filtered under reduced pressure and washed with EA. The solution was concentrated and purified with prep-TLC (MeOH/DCM=1/30) and further purified by prep-HPLC (CH₃CN/0.1% NH₄HCO₃−Water=48-58%) to get the title compound (7.12 mg, 20% yield). MS obsd. (ESI+): 527.5 (M+H)⁺.

### Examples 111 and 112. (S)-6-cyclopropyl-4-((2,2-difluoro-1-(3-(trifluoromethyl)phenyl)ethyl)amino)-2-methylpyrido[3,4-d]pyridazine-1,7(2H,6H)-dione (example 111) and (R)-6-cyclopropyl-4-((2,2-difluoro-1-(3-(trifluoromethyl)phenyl)ethyl)amino)-2-methylpyrido[3,4-d]pyridazine-1,7(2H,6H)-dione (example 112)

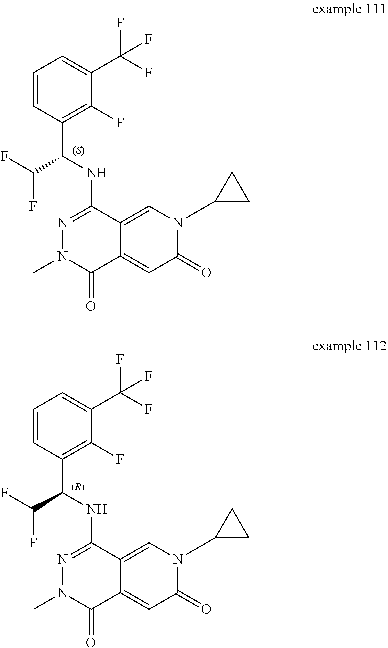

### Step A: (E)-2-methyl-N-(3-(trifluoromethyl)benzylidene)propane-2-sulfinamide

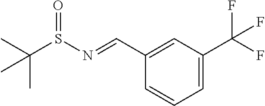 [SMILES: CC(C)(C)S(=O)/N=C/c1cccc(C(F)(F)F)c1]

To a solution of 2-methylpropane-2-sulfinamide (209 mg, 1.72 mmol) in THE (3.0 mL) was added 3-(trifluoromethyl)benzaldehyde (154 μL, 1.15 mmol) and titanium tetraethoxide (722 μL, 3.45 mmol) at room temperature. The mixture was stirred at 80° C. for 16 h. The mixture was quenched with water and extracted with DCM. The organic layers were concentrated and the residuepurified by flash chromatography (eluted with 0-15% EA in PE) to give the title compound (275 mg, 86% yield). MS obsd. (ESI+): 278.3 (M+H)⁺.

### Step B: N-(2,2-difluoro-1-(3-(trifluoromethyl)phenyl)ethyl)-2-methylpropane-2-sulfinamide

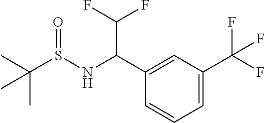 [SMILES: CC(C)(C)S(=O)NC(c1cccc(C(F)(F)F)c1)C(F)F]

To a −78° C. solution of (E)-2-methyl-N-(3-(trifluoromethyl)benzylidene)propane-2-sulfinamide (175 mg, 631 μmol) in THE (2.0 mL) was added difluoromethyl(trimethyl)silane (235 mg, 1.89 mmol). A solution of t-BuOK (212 mg, 1.89 mmol) in THE (1.5 mL) was added to the mixture and it was stirred at −78° C. for 1 h. The mixture was quenched with NH₄Cl (aq.) and extracted with EA. The organic layer was concentrated and purified by flash chromatography (eluted with 0-30% EA in PE) to give the title compound (150 mg, 72% yield).

¹H NMR (400 MHz, CDCl₃) δ: 7.66-7.61 (3H), 7.57-7.53 (1H), 6.15-6.87 (1H), 4.78-4.69 (1H), 1.25 (9H).

Step C: 2,2-difluoro-1-(3-(trifluoromethyl)phenyl)ethanamine hydrochloride

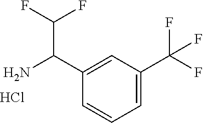 [SMILES: Cl ; NC(c1cccc(C(F)(F)F)c1)C(F)F]

A solution of N-(2,2-difluoro-1-(3-(trifluoromethyl)phenyl)ethyl)-2-methylpropane-2-sulfinamide (150 mg, 455 μmol) in HCl (4 M in dioxane, 5.0 mL) was stirred at room temperature for 2 h. The mixture was concentrated and suspended in diethyl ether. The mixture was filtered and the solids were washed with Et₂O and dried under vacuum to give the title compound (100 mg, 84% yield).

¹H NMR (400 MHz, DMSO-d6) δ: 9.28 (3H), 8.03 (1H), 7.92-7.86 (2H), 7.77 (1H), 6.71 (1H), 5.14-5.07 (1H).

### Step D: (S)-6-cyclopropyl-4-((2,2-difluoro-1-(3-(trifluoromethyl)phenyl)ethyl)amino)-2-methylpyrido[3,4-d]pyridazine-1,7(2H,6H)-dione (example 111) and (R)-6-cyclopropyl-4-((2,2-difluoro-1-(3-(trifluoromethyl)phenyl)ethyl)amino)-2-methylpyrido[3,4-d]pyridazine-1,7(2H,6H)-dione (example 112)

To a solution of 4-chloro-6-cyclopropyl-2-methyl-pyrido[3,4-d]pyridazine-1,7-dione (80 mg, 318.53 μmol) in dioxane (1.5 mL) was added [2,2-difluoro-1-(3-(trifluoromethyl)phenyl)ethanamine hydrochloride (100 mg, 382.24 μmol), Xantphos-Pd-G₃ (60 mg, 63.71 μmol) and Cs₂CO₃ (311 mg, 955.59 μmol) at room temperature under N₂ atmosphere. The mixture was heated under microwave irradiation at 130° C. for 3 h. The mixture was quenched with NH₄Cl (aq.) and extracted with EA. The organic layer was concentrated in vacuum and the resulting residue was purified by flash chromatography (eluted with 0-80% EA in PE). The racemic mixture was purified by chiral SFC (Daicel OD-3 (4.6*100 mm, 3 uM) (CO2/MeOH (0.2% methanolic ammonic). Analytical chiral HPLC: (Column Name:OD-3 4.6*100 mm 3 um, MeOH[0.2% NH3(7M in MeOH)], flow rate: 3 mL/min, temp: 40° C.).

Example 111. Chiral HPLC retention time=1.35 min. MS obsd. (ESI+): 441.4 (M+H)⁺. ¹H NMR (400 MHz, DMSO-d6) δ: 8.79 (1H), 7.92 (1H), 7.86 (1H), 7.73 (1H), 7.67 (1H), 7.50 (1H), 6.84 (1H), 6.61-6.33 (1H), 5.54-5.46 (1H), 3.58-3.52 (1H), 3.35 (3H), 1.16-1.12 (2H), 1.10-1.05 (2H).

Example 112. Chiral HPLC retention time=1.62 min. MS obsd. (ESI+): 441.4 (M+H)⁺. ¹H NMR (400 MHz, DMSO-d6) δ: 8.77 (1H), 7.90 (1H), 7.85 (1H), 7.71 (1H), 7.65 (1H), 7.47 (1H), 6.83 (1H), 6.59-6.31 (1H), 5.53-5.45 (1H), 3.56-3.50 (1H), 3.33 (3H), 1.16-1.10 (2H), 1.09-1.01 (2H).

### Examples 113 and 114: (S)-6-(1-(difluoromethyl)cyclopropyl)-4-((1-(indolizin-5-yl)ethyl)amino)-2-methyl-2,6-dihydropyrido[3,4-d]pyridazine-1,7-dione (example 113) and (R)-6-(1-(difluoromethyl)cyclopropyl)-4-((1-(indolizin-5-yl)ethyl)amino)-2-methyl-2,6-dihydropyrido[3,4-d]pyridazine-1,7-dione (example 114)

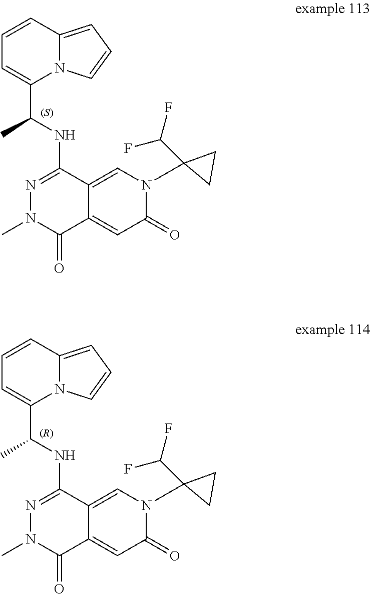

### Step A: 5-bromoindolizine

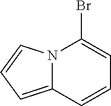

A solution of indolizine (1.5 g, 12.80 mmol) and N,N,N′,N′-tetramethylethylenediamine (1.64 g, 14.08 mmol, 2 mL) in THE (10 mL) was cooled down to −78° C. A solution of n-Butyllithium (14 mmol, 2.5 M in hexanes) was slowly added and the mixture was stirred at −78° C. for two hours. To the reaction was added 1,2-dibromo-1,1,2,2-tetrafluoro-ethane (3.7 g, 14.1 mmol) and the mixture was stirred at rt for 1 hr. The mixture was quenched with NH₄Cl (aq, 10 mL) and extracted with EA (10 mL×2). The organic phases were combined and dried over Na₂SO₄. The mixture was filtered and concentrated, and the crude residue was purified by flash column (elution with PE) to get the title compound (1.1 g, 44% yield). MS obsd. (ESI+): 196.1, 197.1 (M+H)⁺.

### Step B: 1-(indolizin-5-yl)ethan-1-one

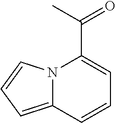

To a solution of 5-bromoindolizine (1.0 g, 5.10 mmol) in dioxane (5 mL) was added tributyl(1-ethoxyvinyl)stannane (2 mL, 6.1 mmol) followed by triethylamine (2 mL, 12.8 mmol). The reaction mixture was stirred at 100° C. for 16 hrs then filtered and diluted with EtOAc. The mixture was washed with KF (aq. 20 mL), H₂O (20 mL), and NaCl (aq., 20 mL).The organic layer was concentrated under reduced pressure and then dissolved in dioxane (5 mL) and treated with 2 mL HCl (2M aqueous). The reaction was further stirred at rt for 2 hrs. The solvent was removed under vacuum and the crude residue was purified by flash chromatography column (eluting with 3% to 5% EA in PE) to afford the title compound (400 mg, 49% yield). MS obsd. (ESI+): 160.3 (M+H)⁺.

### Step C: 1-(indolizin-5-yl)ethan-1-amine

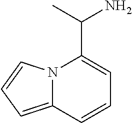

To a solution of 1-(indolizin-5-yl) ethan-1-one (300 mg, 1.88 mmol) in methanol (10 mL) was added NH₄OAc (1.5 g, 18.85 mmol) at rt. The mixture was stirred at 50° C. for 1 hr. Then NaBH₃CN (1.2 g, 18.85 mmol) was added to the mixture and it was stirred at 50° C. for another 48 hrs. The reaction was quenched with saturated aqueous NH₄Cl solution and extracted with EtOAc. The organic layer was dried over anhydrous Na₂SO₄, filtered and concentrated. The residue was purified by flash column (eluting with 0-3% MeOH in DCM) to afford the title compound (166 mg, 55% yield). MS obsd. (ESI+): 161.3 (M+H)⁺.

### Step D: (R)-6-(1-(difluoromethyl)cyclopropyl)-4-((1-(indolizin-5-yl)ethyl)amino)-2-methyl-2,6-dihydropyrido[3,4-d]pyridazine-1,7-dione (example 113) & (S)-6-(1-(difluoromethyl)cyclopropyl)-4-((1-(indolizin-5-yl)ethyl)amino)-2-methyl-2,6-dihydropyrido[3,4-d]pyridazine-1,7-dione (example 114)

A mixture of 4-chloro-6-[1-(difluoromethyl)cyclopropyl]-2-methyl-pyrido[3,4-d]pyridazine-1,7-dione (100 mg, 331 μmol), 1-(indolizin-5-yl)ethan-1-amine (64 mg, 398 μmol), Xantphos-Pd-G3 (94 mg, 99 μmol), cesium carbonate (216 mg, 663 μmol) and dioxane (1 mL) was stirred at 100° C. for 16 hrs. The reaction was filtered and diluted with water, and extracted into EtOAc. The organic layer was concentrated under reduced pressure and the crude product was purified by preparativeTLC (MeOH/DCM=1/30) followed by preparative-HPLC (CH₃CN/0.1% NH₄HCO₃−Water=45-60%) to afford a racemic mixture of the title compounds. The mixture was purified by chiral SFC with the following conditions ((Column Name: Daicel OD-3, 4.6*100 mm 3 μm, CO₂/MeOH(0.2% Methanol Ammonia)=80/20)) to get (R)-6-(1-(difluoromethyl)cyclopropyl)-4-((1-(indolizin-5-yl)ethyl)amino)-2-methyl-2,6-dihydropyrido[3,4-d]pyridazine-1,7-dione (example 113) and (S)-6-(1-(difluoromethyl)cyclopropyl)-4-((1-(indolizin-5-yl)ethyl)amino)-2-methyl-2,6-dihydropyrido[3,4-d]pyridazine-1,7-dione (example 114). Analytical chiral HPLC: Column Name: IG-3 4.6*100 mm 3 um,MeOH[0.2% NH3(7M in MeOH)], flow rate: 3 mL/min, temp: 40° C.

Example 113. Chiral HPLC retention time=1.04 min. MS obsd. (ESI+): 426.3 (M+H)⁺. ¹H NMR (400 MHz, DMSO-d₆) δ: 8.85 (1H), 7.50 (1H), 7.42 (1H), 7.17 (1H), 6.91 (1H), 6.83-6.80 (1H), 6.77 (1H), 6.70 (1H), 6.50-6.46 (1H), 6.28 (1H), 5.45-5.37 (1H), 3.33 (3H), 1.63 (3H), 1.46 (2H), 1.36 (2H).

Example 114. Chiral HPLC retention time=1.25 min. MS obsd. (ESI+): 426.3 (M+H)⁺. ¹H NMR (400 MHz, DMSO-d₆) δ: 8.85 (1H), 7.50 (1H), 7.42 (1H), 7.17 (1H), 6.91 (1H), 6.81 (1H), 6.77 (1H), 6.70 (1H), 6.48 (1H), 6.26 (1H), 5.45-5.37 (1H), 3.33 (3H), 1.63 (3H), 1.46 (2H), 1.36 (2H).

### Examples 115 and 116: (S)-6-(1-(difluoromethyl)cyclopropyl)-2-methyl-4-((1-(pyrazolo[1,5-a]pyridin-7-yl)ethyl)amino)-2,6-dihydropyrido[3,4-d]pyridazine-1,7-dione (example 115) & (R)-6-(1-(difluoromethyl)cyclopropyl)-2-methyl-4-((1-(pyrazolo[1,5-a]pyridin-7-yl)ethyl)amino)-2,6-dihydropyrido[3,4-d]pyridazine-1,7-dione (example 116)

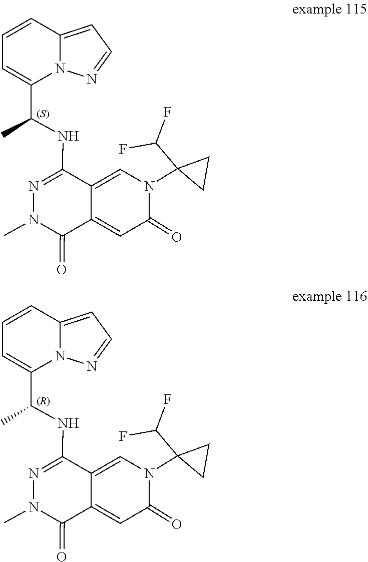

### Step A: 1-(pyrazolo[1,5-a]pyridin-7-yl)ethan-1-one

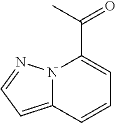

To a mixture of 7-bromopyrazolo[1,5-a]pyridine (1.5 g, 7.61 mmol), tributyl(1-ethoxyvinyl)stannane (3.30 g, 9.14 mmol) and TEA (1.54 g, 15.23 mmol) in dioxane (15 mL) was added bis(triphenylphosphine)palladium (II) chloride (534 mg, 761 μmol). The mixture was heated to 90° C. for 16 h. Ice-water (30 mL) was added and the mixture was extracted with EtOAc (50 mL×3). The combined organic layers were dried over sodium sulfate, filtered and concentrated. The crude product was purified by flash chromatography column (eluting with 0%-10% EA in PE) to give the title compound (900 mg, 69% yield). MS obsd. (ESI+): 161.1 (M+H)⁺.

### Step B: 2-methyl-N-(1-(pyrazolo[1,5-a]pyridin-7-yl)ethylidene)propane-2-sulfinamide

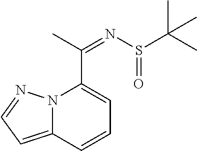 [SMILES: C/C(=N/S(=O)C(C)(C)C)c1cccc2ccnn12]

To a mixture of 1-pyrazolo[1,5-a]pyridin-7-ylethanone (650 mg, 4.06 mmol), tert-butanesulfinamide (738 mg, 6.09 mmol) in THE (7 mL) was added titanium ethoxide (2.31 g, 10.2 mmol). The mixture was heated to 65° C. under N₂ for 18 h. The reaction was quenched with ice-water (50 mL), the mixture was extracted with EA (100 mL×3), and the combined organic layers were dried over sodium sulfate, filtered and concentrated. The crude product was purified by flash chromatography (eluting with 0%-50% EA in PE) to give the title compound (800 mg, 75% yield). MS obsd. (ESI+): 264.3 (M+H)⁺.

### Step C: 2-methyl-N-(1-(pyrazolo[1,5-a]pyridin-7-yl)ethyl)propane-2-sulfinamide

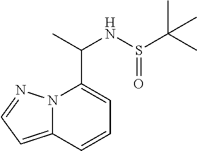 [SMILES: CC(NS(=O)C(C)(C)C)c1cccc2ccnn12]

A solution of 2-methyl-N-(1-(pyrazolo[1,5-a]pyridin-7-yl)ethylidene)propane-2-sulfinamide (800 mg, 3.04 mmol) in THE (8 mL) was cooled to −70° C. Sodium borohydride (207 mg, 5.47 mmol) was added and the mixture was stirred for 2 hr. Ice-water (20 mL) was added and the mixture was extracted with EA (50 mL×3). The combined organic layers were concentrated under vacuum to obtain the crude product. The crude product was purified by flash chromatography eluting with 0%-50% EA in PE to the title compound (700 mg, 87% yield). MS obsd. (ESI+): 266.3 (M+H)⁺.

### Step D: 1-(pyrazolo[1,5-a]pyridin-7-yl)ethan-1-amine hydrochloride

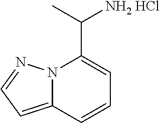

A solution of 2-methyl-N-(1-pyrazolo[1,5-a]pyridin-7-ylethyl)propane-2-sulfinamide (700 mg, 2.64 mmol) in DCM (10 mL) cooled to 0° C. To this solution was added 4 M HCl in 1,4-Dioxane (4 M, 3.30 mL) and the mixture was stirred for 2 h. The mixture was then concentrated under vacuum. The crude product was dissolved in 10 mL MeCN and filtered through a celite pad and concentrated to give 1-(pyrazolo[1,5-a]pyridin-7-yl)ethan-1-amine hydrochloride (400 mg, crude).

¹H NMR (400 MHz, D₂O) δ 7.94 (1H), 7.64 (1H), 7.17 (1H), 6.92 (1H), 6.63 (1H), 5.04 (1H), 1.71 (3H).

### Step E: (S)-6-(1-(difluoromethyl)cyclopropyl)-2-methyl-4-((1-(pyrazolo[1,5-a]pyridin-7-yl)ethyl)amino)-2,6-dihydropyrido[3,4-d]pyridazine-1,7-dione (example 115) & (R)-6-(1-(difluoromethyl)cyclopropyl)-2-methyl-4-((1-(pyrazolo[1,5-a]pyridin-7-yl)ethyl)amino)-2,6-dihydropyrido[3,4-d]pyridazine-1,7-dione (example 116)

Prepared in an essentially analogous manner to examples 113/114 step D, starting with 1-(pyrazolo[1,5-a]pyridin-7-yl)ethan-1-amine hydrochloride. Analytical chiral HPLC: Column Name: OJ-3 4.6*100 mm 3 um,EtOH[1% NH3(7M in MeOH)], flow rate: 3 mL/min, temp: 40° C.

Example 115. Retention time=1.33 min. MS obsd. (ESI+): 427.4 (M+H)⁺. ¹H NMR (400 MHz, DMSO) δ 8.99 (1H), 8.07 (1H), 7.65-7.57 (1H), 7.37 (1H), 7.21 (1H), 6.89 (2H), 6.67 (1H), 6.32 (1H), 5.79 (1H), 3.10 (3H), 1.65 (3H), 1.53 (2H), 1.45 (2H).

Example 116. Retention time=1.72 min. MS obsd. (ESI+): 427.4 (M+H)⁺. ¹H NMR (400 MHz, DMSO) δ 8.99 (1H), 8.07 (1H), 7.61 (1H), 7.38 (1H), 7.21 (1H), 6.89 (2H), 6.67 (1H), 6.32 (1H), 5.79 (1H), 3.10 (3H), 1.65 (3H), 1.53 (2H), 1.45 (2H).

### Examples 117 and 118: 6-cyclopropyl-2-methyl-4-[[(1S)-1-[3-methyl-5-(trifluoromethyl)phenyl]ethyl]amino]pyrido[3,4-d]pyridazine-1,7-dione (example 117) and 6-cyclopropyl-2-methyl-4-[[(1R)-1-[3-methyl-5-(trifluoromethyl)phenyl]ethyl]amino]pyrido[3,4-d]pyridazine-1,7-dione (example 118)

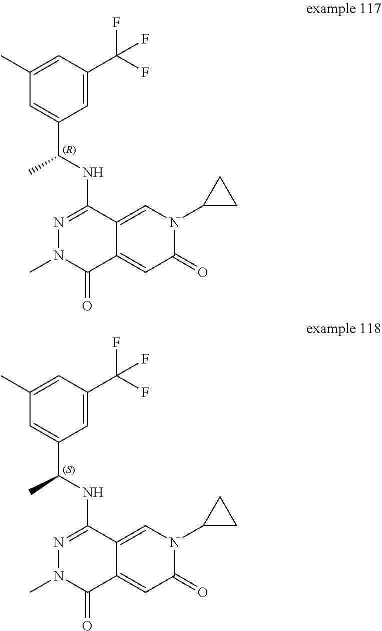

Prepared in an essentially analogous manner to example 2 step B, starting with 1-[3-methyl-5-(trifluoromethyl)phenyl]ethanamine hydrochloride (prepared according to the method of WO/2021/127429). Chiral HPLC: Column Name:AS-3 4.6*100 mm 3 um,IPA[1% NH3(7M in MeOH)], flow rate: 3 mL/min, temp: 40° C.

Example 117. Chiral HPLC retention time=3.04. MS obsd. (ESI+): 419.3 (M+H)⁺. ¹H NMR (400 MHz, DMSO-d6) δ 8.69 (1H), 7.52 (2H), 7.38 (1H), 7.08 (1H), 6.82 (1H), 4.96 (1H), 3.59-3.49 (1H), 3.29 (3H), 2.38 (3H), 1.51 (3H), 1.17-0.97 (4H).

Example 118. Chiral HPLC retention time=5.01. MS obsd. (ESI+): 419.3 (M+H)⁺. ¹H NMR (400 MHz, DMSO-d6) δ 8.69 (1H), 7.52 (2H), 7.38 (1H), 7.07 (1H), 6.82 (1H), 4.96 (1H), 3.59-3.48 (1H), 3.29 (3H), 2.38 (3H), 1.51 (3H), 1.18-0.98 (4H).

### Examples 119, 120: 4-(((R)-1-(3-(difluoromethyl)-2-fluorophenyl)ethyl)amino)-6-((S)-3-(difluoromethyl)tetrahydrofuran-3-yl)-2-methyl-2,6-dihydropyrido[3,4-d]pyridazine-1,7-dione (example 119, stereochemistry in tetrahydrofuran ring is arbitrarily assigned) and 4-(((R)-1-(3-(difluoromethyl)-2-fluorophenyl)ethyl)amino)-6-((R)-3-(difluoromethyl)tetrahydrofuran-3-yl)-2-methyl-2,6-dihydropyrido[3,4-d]pyridazine-1,7-dione (example 120, stereochemistry in tetrahydrofuran ring is arbitrarily assigned)

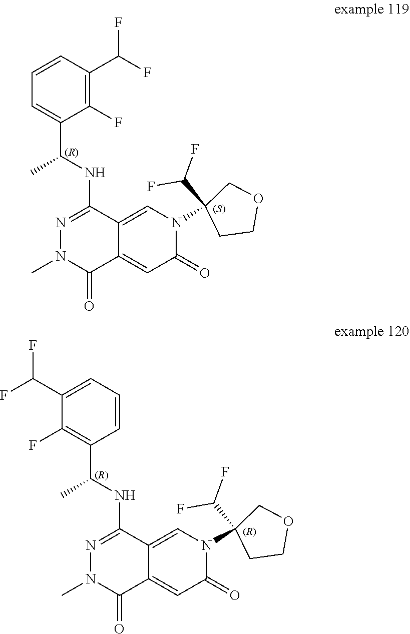

### Step A: N-(dihydrofuran-3(2H)-ylidene)-2-methylpropane-2-sulfinamide

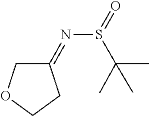

To a flask charged with tetrahydrofuran-3-one (10 g, 116 mmol) in THF (75 mL) was added Titanium ethoxide (48.7 mL, 232 mmol) and 2-methylpropane-2-sulfinamide (15.6 g, 127.8 mmol) under N₂ atmosphere at room temperature. The reaction mixture was stirred at 60° C. for 18 h. The reaction mixture was quenched with a saturated aq. solution of NaHCO₃ with vigorous stirring. The precipitate was filtered and washed with EtOAc and the aqueous mixture was extracted with EtOAc (100 mL×3). The combined organic layers were dried over sodium sulfate, filtered and concentrated. The crude product was purified by silica gel chromatography (120 g, 15-60% EA in PE) to afford the title compound (9.3 g, 42% yield). MS obsd. (ESI+): 190.3 (M+H)+.

### Step B: N-(3-(difluoro(phenylsulfonyl)methyl)tetrahydrofuran-3-yl)-2-methylpropane-2-sulfinamide

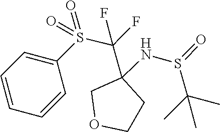 [SMILES: CC(C)(C)S(=O)NC1(C(F)(F)S(=O)(=O)c2ccccc2)CCOC1]

To a mixture of N-(dihydrofuran-3(2H)-ylidene)-2-methylpropane-2-sulfinamide (7.1 g, 37.51 mmol) and difluoromethylsulfonylbenzene (5.99 mL, 41.26 mmol) in tetrahydrofuran (150 mL) was added LiHMDS (1.0 M in THF, 56.3 mL) drop-wise at −78° C. under N₂ atmosphere. The resulting mixture was stirred for 5 h at this temperature. The mixture was poured into NH4Cl (sat., aq.) and extracted with EtOAc (100 mL×4). The combined organic layers were dried over sodium sulfate, filtered and concentrated in vacuo. The residue was purified by silica gel chromatography (15-75% EA in PE) to give the title compound (6.5 g, 45% yield). MS obsd. (ESI+): 382.4 (M+H)+.

### Step C: N-(3-(difluoromethyl)tetrahydrofuran-3-yl)-2-methylpropane-2-sulfinamide

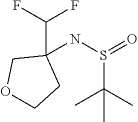

To a mixture of N-(3-(difluoro(phenylsulfonyl)methyl)tetrahydrofuran-3-yl)-2-methylpropane-2-sulfinamide (6.5 g, 17.0 mmol) in NaOAc/HOAc (8 M, 21.3 mL) and DMF (100 mL) was added Mg (1.24 g, 51.1 mmol) portion-wise at room temperature. The resulting mixture was stirred for 3 h at this temperature. The mixture was diluted with water (800 mL) and extracted with EA (200 mL×3). The combined organic layers were washed with brine and H₂O, dried over Na₂SO₄, filtered and concentrated. The residue was purified by column chromatography (15-80% EA in PE) to give the title compound (2.0 g, 49% yield). MS obsd. (ESI+): 242.3 (M+H)+.

### Step C: 3-(difluoromethyl)tetrahydrofuran-3-amine hydrochloride

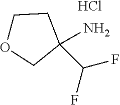

N-(3-(difluoromethyl)tetrahydrofuran-3-yl)-2-methylpropane-2-sulfinamide (2.0 g, 8.29 mmol) was dissolved in 1,4-Dioxane (10 mL). Then to the resulting mixture was added HCl/1,4-dioxane (4 M, 10.36 mL) at room temperature. The resulting mixture was stirred for 2 h at this temperature. The reaction mixture was concentrated in vacuo. The residue was triturated with ether (50 mL×2). The residue was dried under vacuum to give the title compound (1.4 g, crude).

¹H NMR (400 MHz, DMSO-d6) δ 9.15 (3H), 6.44 (1H), 3.98 (1H), 3.93 (1H), 3.87-3.79 (2H), 2.32-2.19 (1H), 2.15-2.02 (1H).

### Step D: 2-(6-chloro-5-(hydroxymethyl)-2-methyl-3-oxo-2,3-dihydropyridazin-4-yl)-N-(3-(difluoromethyl)tetrahydrofuran-3-yl)acetamide

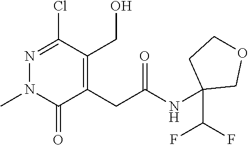 [SMILES: Cn1nc(Cl)c(CO)c(CC(=O)NC2(C(F)F)CCOC2)c1=O]

A mixture of 3-(difluoromethyl)tetrahydrofuran-3-amine (216 mg, 1.57 mmol), 4-chloro-2-methyl-5,8-dihydropyrano[3,4-d]pyridazine-1,7-dione (0.26 g, 1.21 mmol) and acetic acid (69 μL, 1.21 mmol) was sealed in microwave tube, and stirred at 110° C. for 1 h. The mixture was concentrated and purified by column chromatography (₀-5% MeOH in DCM) to give the title compound (0.11 g, 26% yield). MS obsd. (ESI+): 352.4 (M+H)+.

### Step E: 4-chloro-6-(3-(difluoromethyl)tetrahydrofuran-3-yl)-2-methyl-2,6-dihydropyrido[3,4-d]pyridazine-1,7-dione

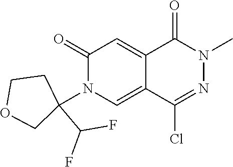 [SMILES: Cn1nc(Cl)c2cn(C3(C(F)F)CCOC3)c(=O)cc2c1=O]

To a mixture of 2-(6-chloro-5-(hydroxymethyl)-2-methyl-3-oxo-2,3-dihydropyridazin-4-yl)-N-(3-(difluoromethyl)tetrahydrofuran-3-yl)acetamide (0.11 g, 313 μmol) in acetonitrile (60 mL) was added 2-Iodoxybenzoic acid (175 mg, 625 μmol). The resulting mixture was stirred for 3 hr at 80° C. The mixture cooled to room temperature and filtered. The filtrate was concentrated in vacuo. The residue was purified by column chromatography (25 g, 0-4 MeOH in DCM) to give the title compound (60 mg, 58% yield). MS obsd. (ESI+): 332.2 (M+H)+.

### Step E: 4-(((R)-1-(3-(difluoromethyl)-2-fluorophenyl)ethyl)amino)-6-(3-(difluoromethyl)tetrahydrofuran-3-yl)-2-methyl-2,6-dihydropyrido[3,4-d]pyridazine-1,7-dione

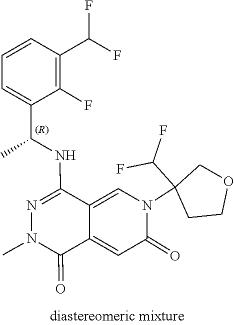

A mixture of 4-chloro-6-(3-(difluoromethyl)tetrahydrofuran-3-yl)-2-methyl-2,6-dihydropyrido[3,4-d]pyridazine-1,7-dione (60 mg, 181 μmol), (1R)-1-[3-(difluoromethyl)-2-fluoro-phenyl]ethanamine (51.3 mg, 271 μmol), Xantphos-Pd-G3 (34 mg, 36 μmol) and cesium carbonate (177 mg, 543 μmol) in 1,4-dioxane (1.0 mL) was stirred for 16 hr at 100° C. under N₂ atmosphere. The reaction mixture was filtered and the filtrate was concentrated in vacuo. The residue was purified by Preparative TLC (5% MeOH in DCM) to give the title compound (17 mg, 18% yield). MS obsd. (ESI+): 485.0 (M+H)+.

### Step F: 4-(((R)-1-(3-(difluoromethyl)-2-fluorophenyl)ethyl)amino)-6-((S)-3-(difluoromethyl)tetrahydrofuran-3-yl)-2-methyl-2,6-dihydropyrido[3,4-d]pyridazine-1,7-dione (example 119, tetrahydrofuran stereochemistry is arbitratily assigned) and 4-(((R)-1-(3-(difluoromethyl)-2-fluorophenyl)ethyl)amino)-6-((R)-3-(difluoromethyl)tetrahydrofuran-3-yl)-2-methyl-2,6-dihydropyrido[3,4-d]pyridazine-1,7-dione (example 120, tetrahydrofuran stereochemistry is arbitratily assigned)

4-(((R)-1-(3-(difluoromethyl)-2-fluorophenyl)ethyl)amino)-6-(3-(difluoromethyl)tetrahydrofuran-3-yl)-2-methyl-2,6-dihydropyrido[3,4-d]pyridazine-1,7-dione (14.15 mg, 29.21 μmol) was separated by chiral SFC (Column Name: Daicel OG-3, 4.6*100 mm 3 μm, CO₂/MeOH(0.2% Methanol Ammonia (7M in methanol))=85/15) to afford 4-[[(1R)-1-[3-(difluoromethyl)-2-fluoro-phenyl]ethyl]amino]-6-[(3S)-3-(difluoromethyl)tetrahydrofuran-3-yl]-2-methyl-pyrido[3,4-d]pyridazine-1,7-dione (example 119, tetrahydrofuran stereochemistry is arbitratily assigned) and 4-[[(1R)-1-[3-(difluoromethyl)-2-fluoro-phenyl]ethyl]amino]-6-[(3R)-3-(difluoromethyl)tetrahydrofuran-3-yl]-2-methyl-pyrido[3,4-d]pyridazine-1,7-dione (example 120, tetrahydrofuran stereochemistry is arbitratily assigned). Chiral HPLC: Column Name: OJ-3 4.6*100 mm 3 um,MeOH[0.2% NH3(7M in MeOH)], flow rate: 3 mL/min, temp: 40° C.

Example 119. Chiral HPLC retention time=0.72 min. MS obsd. (ESI+): 485.0 (M+H)+. ¹H NMR (400 MHz, DMSO-d6) δ 8.59 (1H), 7.61 (1H), 7.49 (1H), 7.38-7.06 (3H), 6.90 (1H), 6.56 (1H), 5.26-5.13 (1H), 4.66 (1H), 4.20-4.10 (1H), 3.99-3.85 (2H), 3.25 (3H), 2.88-2.71 (2H), 1.54 (3H).

Example 120. Chiral HPLC retention time=0.95 min. MS obsd. (ESI+): 485.0 (M+H)+. ¹H NMR (400 MHz, DMSO-d6) δ 8.61 (1H), 7.63 (1H), 7.48 (1H), 7.36-7.09 (3H), 6.90 (1H), 6.56 (1H), 5.26-5.15 (1H), 4.66 (1H), 4.23-4.07 (1H), 3.93 (2H), 3.24 (3H), 2.88-2.73 (2H), 1.54 (3H).

### Examples 121 and 122: 6-((S)-3,3-difluorocyclopentyl)-4-(((R)-1-(3-(difluoromethyl)-2-fluorophenyl)ethyl)amino)-2-methylpyrido[3,4-d]pyridazine-1,7(2H,6H)-dione (example 121, cyclopentyl stereochemistry is arbitrarily assigned) and 6-((R)-3,3-difluorocyclopentyl)-4-(((R)-1-(3-(difluoromethyl)-2-fluorophenyl)ethyl)amino)-2-methylpyrido[3,4-d]pyridazine-1,7(2H,6H)-dione (example 122, cyclopentyl stereochemistry is arbitrarily assigned)

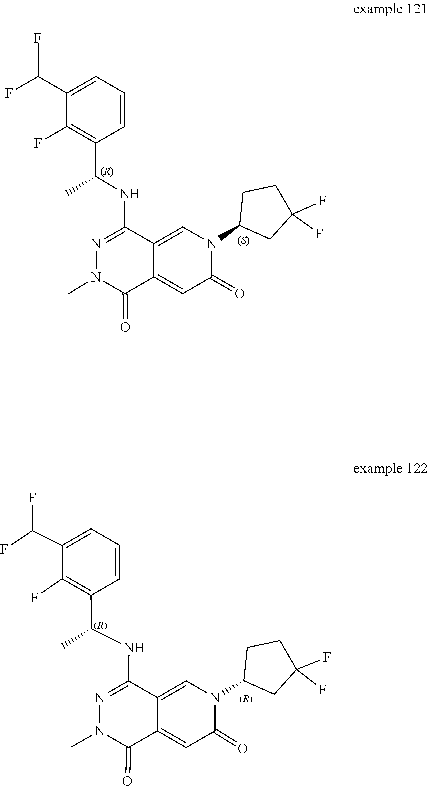

### Step A: 2-(6-chloro-5-(hydroxymethyl)-2-methyl-3-oxo-2,3-dihydropyridazin-4-yl)-N-(3,3-difluorocyclopentyl)acetamide

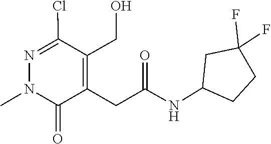 [SMILES: Cn1nc(Cl)c(CO)c(CC(=O)NC2CCC(F)(F)C2)c1=O]

To a solution of 4-chloro-2-methyl-5,8-dihydropyrano[3,4-d]pyridazine-1,7-dione (500 mg, 2.33 mmol) in toluene (5.0 mL) was added (3,3-difluorocyclopentyl)ammonium chloride (441 mg, 2.80 mmol) and triethylamine (390 μL, 2.80 mmol) at room temperature. The mixture was stirred at 80° C. for 56 h. The reaction was extracted with EtOAc and the combined organic layers were washed with brine, dried over anhydrous sodium sulfate, filtered and concentrated in vacuo. The residue was purified by flash chromatography (eluting with 5% MeOH in DCM) to give the title compound (543 mg, 69% yield). MS obsd. (ESI+): 336.1 (M+H)+.

### Step B: 4-chloro-6-(3,3-difluorocyclopentyl)-2-methylpyrido[3,4-d]pyridazine-1,7(2H,6H)-dione

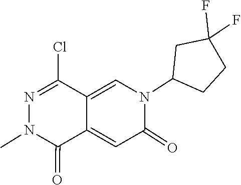 [SMILES: Cn1nc(Cl)c2cn(C3CCC(F)(F)C3)c(=O)cc2c1=O]

To a solution of 2-(6-chloro-5-(hydroxymethyl)-2-methyl-3-oxo-2,3-dihydropyridazin-4-yl)-N-(3,3-difluorocyclopentyl)acetamide (543 mg, 1.62 mmol) in MeCN (25 mL) was added IBX (906 mg, 3.23 mmol) at room temperature. The mixture was stirred at 80° C. for 3 h. The mixture was filtered, and filtrate was concentrated in vacuum. The residue was purified by flash chromatography (eluting with 10% EA in PE) to give the title compound (312 mg, 61% yield). MS obsd. (ESI+): 316.0 (M+H)⁺.

### Step C: 6-(3,3-difluorocyclopentyl)-4-(((R)-1-(3-(difluoromethyl)-2-fluorophenyl)ethyl)amino)-2-methylpyrido[3,4-d]pyridazine-1,7(2H,6H)-dione

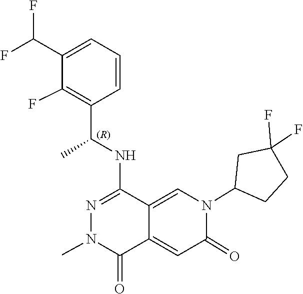

To a solution of 4-chloro-6-(3,3-difluorocyclopentyl)-2-methyl-pyrido[3,4-d]pyridazine-1,7-dione (100 mg, 317 μmol) in dioxane (2.5 mL) was added (1R)-1-[3-(difluoromethyl)-2-fluoro-phenyl]ethanaminechloride (85 mg, 380 μmol), Xantphos-Pd-G3 (60 mg, 63.4 μmol) and Cs₂CO₃ (309 mg, 950 μmol). The mixture was heated in a microwave reactor at 130° C. for 3 h. The mixture reaction was concentrated and the residue was purified by flash chromatography (eluting with 2% MeOH in DCM) followed by reverse column (C18, MeCN/H₂O) to give the title compound (53 mg, 36% yield). MS obsd. (ESI+): 469.2 (M+H)⁺

### Step D: 6-((S)-3,3-difluorocyclopentyl)-4-(((R)-1-(3-(difluoromethyl)-2-fluorophenyl)ethyl)amino)-2-methylpyrido[3,4-d]pyridazine-1,7(2H,6H)-dione (example 121, cyclopentyl stereochemistry is arbitrarily assigned) and 6-((R)-3,3-difluorocyclopentyl)-4-(((R)-1-(3-(difluoromethyl)-2-fluorophenyl)ethyl)amino)-2-methylpyrido[3,4-d]pyridazine-1,7(2H,6H)-dione (example 122, cyclopentyl stereochemistry is arbitrarily assigned)

Chiral SFC separation of the diastereomeric mixture of 6-(3,3-difluorocyclopentyl)-4-(((R)-1-(3-(difluoromethyl)-2-fluorophenyl)ethyl)amino)-2-methylpyrido[3,4-d]pyridazine-1,7(2H,6H)-dione afforded individual isomers. Chiral HPLC: Column Name: AD-3 4.6*100 mm 3 um,EtOH[1% NH3(7M in MeOH)], flow rate: 3 mL/min, temp: 40° C.

Example 121. Chiral HPLC retention time=0.83 min. MS obsd. (ESI+): 469.4 (M+H) ⁺. 1H NMR (400 MHz, DMSO-d6) δ ppm: 8.80 (1H), 7.66 (1H), 7.50 (1H), 7.36-7.06 (3H), 6.87 (1H), 5.25-5.18 (2H), 3.25 (3H), 2.71-2.50 (3H), 2.33-2.26 (3H), 1.55 (3H).

Example 122. Chiral HPLC retention time=1.19. MS obsd. (ESI⁺): 469.4 (M+H)⁺. 1H NMR (400 MHz, DMSO-d6) δ ppm: 8.80 (1H), 7.66 (1H), 7.50 (1H), 7.36-7.07 (3H), 6.87 (1H), 5.25-5.17 (2H), 3.25 (3H), 2.72-2.51 (3H), 2.32-2.27 (3H), 1.54 (3H).

### Example 123: (R)-4-((1-(3-(difluoromethyl)-2-fluorophenyl)ethyl)amino)-6-(1-(hydroxymethyl)cyclopropyl)-2-methylpyrido[3,4-d]pyridazine-1,7(2H,6H)-dione

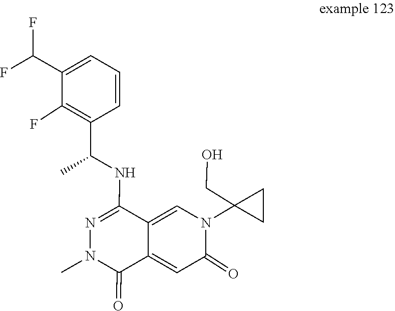 [SMILES: C[C@@H](Nc1nn(C)c(=O)c2cc(=O)n(C3(CO)CC3)cc12)c1cccc(C(F)F)c1F]

### Step A: 1-((benzyloxy)methyl)cyclopropanamine

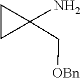 [SMILES: NC1(COCc2ccccc2)CC1]

To a solution of 2-(benzyloxy)acetonitrile (3.7 g, 25.1 mmol) in dry THF (70 mL) at 0° C. under Ar atmosphere was added titanium isopropoxide (7.9 g, 27.7 mmol) and EtMgBr (1 M in THF, 50.3 mL). The mixture was stirred at room temperature for 2 hr. BF₃·OEt₂ (6.0 mL) was added and stirred for 10 min. 10% NaOH (12.0 mL) was added and the mixture was extracted with EtOAc, dried over anhydrous sodium sulfate, filtered, concentrated and purified by flash chromatography (eluting with 0-5% MeOH in DCM) to afford the title compound (2.2 g, 45% yield).

¹H NMR (400 MHz, CDCl₃) δ ppm: 7.37-7.28 (5H), 4.58 (2H), 3.39 (2H), 0.74 (2H), 0.52 (2H).

### Step B: N-(1-((benzyloxy)methyl)cyclopropyl)-2-(6-chloro-5-(hydroxymethyl)-2-methyl-3-oxo-2,3-dihydropyridazin-4-yl)acetamide

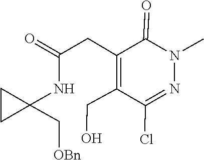 [SMILES: Cn1nc(Cl)c(CO)c(CC(=O)NC2(COCc3ccccc3)CC2)c1=O]

To a solution of 4-chloro-2-methyl-5,8-dihydro-1H-pyrano[3,4-d]pyridazine-1,7(2H)-dione (2.3 g, 10.7 mmol) in toluene (17 mL) was added 1-((benzyloxy)methyl)cyclopropanamine (1.7 g, 9.72 mmol) and Et₃N (1.2 g, 11.7 mmol) at room temperature. The mixture was stirred at 80° C. for 16 h. The reaction mixture was concentrated under reduced pressure and combined with crude material from a reaction performed under identical conditions on 3.1 mmol scale. The combined lots were purified by flash chromatography (eluting with 0-50% EA in PE) to afford the title compound (2.5 g, 51% yield).MS obsd. (ESI+): 392.5 (M+H)⁺

### Step C: 6-(1-((benzyloxy)methyl)cyclopropyl)-4-chloro-2-methylpyrido[3,4-d]pyridazine-1,7(2H,6H)-dione

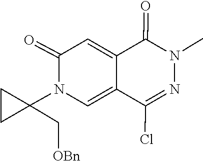 [SMILES: Cn1nc(Cl)c2cn(C3(COCc4ccccc4)CC3)c(=O)cc2c1=O]

To a solution of N-(1-((benzyloxy)methyl)cyclopropyl)-2-(6-chloro-5-(hydroxymethyl)-2-methyl-3-oxo-2,3-dihydropyridazin-4-yl)acetamide (2.3 g, 5.87 mmol) in MeCN (92 mL) was added IBX (3.3 g, 11.74 mmol). The mixture was stirred at 80° C. for 3 h. The reaction mixture was filtered, concentrated, and combined with crude material from a separate reaction run under identical conditions on 0.51 mmol scale. The combined lots were purified by flash chromatography (eluting with 0-30% EA in PE) followed by reverse phase HPLC (eluting with 0-60% MeCN in water) to afford the title compound (551 mg),MS obsd. (ESI+): 372.5 (M+H)⁺

### Step D: (R)-6-(1-((benzyloxy)methyl)cyclopropyl)-4-((1-(3-(difluoromethyl)-2-fluorophenyl)ethyl)amino)-2-methylpyrido[3,4-d]pyridazine-1,7(2H,6H)-dione

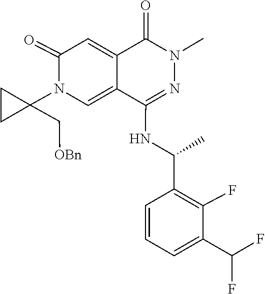 [SMILES: C[C@@H](Nc1nn(C)c(=O)c2cc(=O)n(C3(COCc4ccccc4)CC3)cc12)c1cccc(C(F)F)c1F]

To a solution of 6-(1-((benzyloxy)methyl)cyclopropyl)-4-chloro-2-methylpyrido[3,4-d]pyridazine-1,7 (2H,6H)-dione (138 mg, 0.37 mmol) in dioxane (1.6 mL) was added (R)-1-(3-(difluoromethyl)-2-fluorophenyl)ethanamine hydrochloride (100 mg, 0.45 mmol), Xantphos-Pd-G3 (70 mg, 0.07 mmol) and Cs₂CO₃ (363 mg, 1.11 mmol). The reaction mixture was heated under microwave irradiation at 130° C. for 3 hr. The reaction mixture was concentrated under reduced pressure and combined with crude material from a separate reaction run under identical conditions on 1.11 mmol scale. The combined lots were purified by flash chromatography (eluting with 0-5% MeOH in DCM) to afford the title compound (342 mg). MS obsd. (ESI+): 525.5 (M+H)⁺

### Step E: (R)-4-((1-(3-(difluoromethyl)-2-fluorophenyl)ethyl)amino)-6-(1-(hydroxymethyl)cyclopropyl)-2-methylpyrido[3,4-d]pyridazine-1,7(2H,6H)-dione (example 123)

BBr₃ (1 M in CH₂Cl₂, 0.02 mL) was added to a solution of (R)-6-(1-((benzyloxy)methyl)cyclopropyl)-4-((1-(3-(difluoromethyl)-2-fluorophenyl)ethyl)amino)-2-methylpyrido[3,4-d]pyridazine-1,7(2H,6H)-dione (10 mg, 0.02 mmol) in EA (1.0 mL) at 0° C. and stirred at room temperature for 16 hr. The reaction mixture was concentrated under reduced pressure and purified by flash chromatography (eluting with 0-5% MeOH in DCM) followed by HPLC (eluting with 0-40% MeCN in water) to afford the title compound (6 mg, 70% yield). MS obsd. (ESI+): 435.1 (M+H)⁺.

¹H NMR (400 MHz, CDCl₃) δ ppm: 8.15 (1H), 7.53-7.43 (2H), 7.23 (1H), 7.20-7.16 (1H), 6.91 (1H), 5.20 (1H), 3.99 (1H), 3.72 (1H), 3.41 (3H), 1.62 (3H), 1.31 (2H), 1.19 (2H).

### Example 124: (R)-4-((1-(3-(difluoromethyl)-2-fluorophenyl)ethyl)amino)-6-(1-((3-fluoroazetidin-1-yl)methyl)cyclopropyl)-2-methylpyrido[3,4-d]pyridazine-1,7(2H,6H)-dione

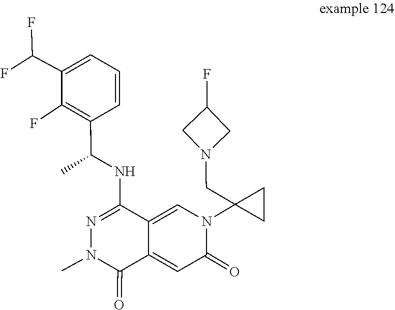 [SMILES: C[C@@H](Nc1nn(C)c(=O)c2cc(=O)n(C3(CN4CC(F)C4)CC3)cc12)c1cccc(C(F)F)c1F]

### Step A: (R)-(1-(4-((1-(3-(difluoromethyl)-2-fluorophenyl)ethyl)amino)-2-methyl-1,7-dioxo-1,2-dihydropyrido[3,4-d]pyridazin-6(7H)-yl)cyclopropyl)methyl-4-methylbenzenesulfonate

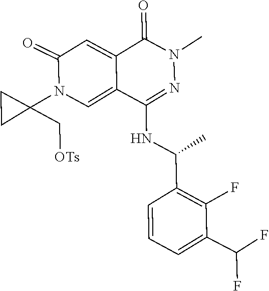 [SMILES: Cc1ccc(S(=O)(=O)OCC2(n3cc4c(N[C@H](C)c5cccc(C(F)F)c5F)nn(C)c(=O)c4cc3=O)CC2)cc1]

To a solution of (R)-4-((1-(3-(difluoromethyl)-2-fluorophenyl)ethyl)amino)-6-(1-(hydroxymethyl)cyclo propyl)-2-methylpyrido[3,4-d]pyridazine-1,7(2H,6H)-dione (20 mg, 0.05 mmol) in DCM (0.5 mL) was added 4-toluenesulfonyl chloride (22 mg, 0.12 mmol), Et₃N (14 mg, 0.14 mmol) and 4-dimethylaminopyridine (6 mg, 0.05 mmol) at 0° C. and the mixture was stirred at room temperature for 16 hr. The reaction mixture was concentrated under reduced pressure and purified by flash chromatography eluting with 0-5% MeOH in DCM to afford the title compound (24 mg, 71% yield). MS obsd. (ESI+): 589.2 (M+H)⁺.

### Step B: (R)-4-((1-(3-(difluoromethyl)-2-fluorophenyl)ethyl)amino)-6-(1-((3-fluoroazetidin-1-yl)methyl)cyclopropyl)-2-methylpyrido[3,4-d]pyridazine-1,7(2H,6H)-dione (example 124)

To a solution of (R)-(1-(4-((1-(3-(difluoromethyl)-2-fluorophenyl)ethyl)amino)-2-methyl-1,7-dioxo-1,2-dihydropyrido[3,4-d]pyridazin-6(7H)-yl)cyclopropyl)methyl-4-methylbenzenesulfonate (19 mg, 0.03 mmol) and 3-fluoroazetidine hydrochloride (36 mg, 0.32 mmol) in MeCN (0.4 mL) was added DIPEA (83 mg, 0.65 mmol) at 0° C. and the mixture was stirred at 60° C. for 16 hr. The reaction mixture was concentrated under reduced pressure and purified by flash chromatography (eluting with 0-5% MeOH in DCM) followed by preparative HPLC (eluting with 0-50% MeCN in water) to afford the title compound (9 mg, 67% yield). MS obsd. (ESI+): 492.6 (M+H)⁺. ¹H NMR (400 MHz, DMSO-d6) δ ppm: 8.73 (1H), 7.62 (1H), 7.48 (1H), 7.38-7.04 (3H), 6.79 (1H), 5.30-4.97 (2H), 3.58 (2H), 3.31-3.27 (2H), 3.25 (3H), 3.21-3.10 (2H), 1.54 (3H), 1.22-0.96 (4H).

### Example 125: (R)-4-((1-(3-(difluoromethyl)-2-fluorophenyl)ethyl)amino)-6-(1-(fluoromethyl)cyclopropyl)-2-methylpyrido[3,4-d]pyridazine-1,7(2H,6H)-dione

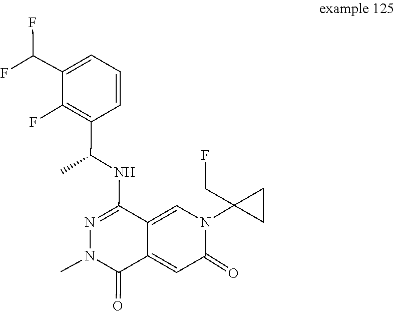 [SMILES: C[C@@H](Nc1nn(C)c(=O)c2cc(=O)n(C3(CF)CC3)cc12)c1cccc(C(F)F)c1F]

### Step A: 2-(6-chloro-5-(hydroxymethyl)-2-methyl-3-oxo-2,3-dihydropyridazin-4-yl)-N-(1-(fluoromethyl)cyclopropyl)acetamide

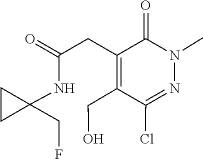 [SMILES: Cn1nc(Cl)c(CO)c(CC(=O)NC2(CF)CC2)c1=O]

To a solution of 4-chloro-2-methyl-5,8-dihydro-1H-pyrano[3,4-d]pyridazine-1,7(2H)-dione (500 mg, 2.33 mmol) in toluene (5.0 mL) was added 1-(fluoromethyl)cyclopropanamine hydrochloride (351 mg, 2.80 mmol) and Et₃N (283 mg, 2.80 mmol) at room temperature. The mixture was stirred at 80° C. for 24 h. The mixture was concentrated and combined with crude material from a separate reaction run under identical conditions on 0.23 mmol scale. The combined mixture was purified by flash chromatography eluting with 0-5% EA in PE to afford the title compound (612 mg). MS obsd. (ESI+): 304.4 (M+H)⁺.

### Step B: 4-chloro-6-(1-(fluoromethyl)cyclopropyl)-2-methylpyrido[3,4-d]pyridazine-1,7(2H,6H)-dione

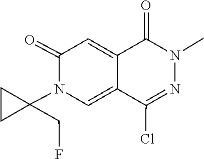 [SMILES: Cn1nc(Cl)c2cn(C3(CF)CC3)c(=O)cc2c1=O]

To a solution of 2-(6-chloro-5-(hydroxymethyl)-2-methyl-3-oxo-2,3-dihydropyridazin-4-yl)-N-(1-(fluo romethyl)cyclopropyl)acetamide (602 mg, 1.98 mmol) in MeCN (24.0 mL) was added IBX (1.1 g, 3.96 mmol) at room temperature. The mixture was stirred at 80° C. for 3 h. The reaction mixture was filtered, concentrated and purified by flash chromatography (eluting with 0-10% MeOH in DCM) to afford the title compound (354 mg, 62% yield). MS obsd. (ESI+): 284.2 (M+H)⁺.

### Step C: (R)-4-((1-(3-(difluoromethyl)-2-fluorophenyl)ethyl)amino)-6-(1-(fluoromethyl)cyclopropyl)-2-methylpyrido[3,4-d]pyridazine-1,7(2H,6H)-dione (example 125)

To solution of 4-chloro-6-(1-(fluoromethyl)cyclopropyl)-2-methylpyrido[3,4-d]pyridazine-1,7(2H,6H)-dione (50 mg, 0.18 mmol) in dioxane (0.6 mL) was added ((R)-1-(3-(difluoromethyl)-2-fluorophenyl)ethanamine hydrochloride (48 mg, 0.21 mmol), Xantphos-Pd-G3 (33 mg, 0.04 mmol) and Cs₂CO₃ (172 mg, 0.53 mmol). The reaction mixture was stirred at 130° C. for 3 h under microwave irradiation. The reaction mixture was concentrated under reduced pressure and purified by flash chromatography (eluting with 0-10% MeOH in DCM) followed by prep HPLC (0-40% MeCN in water) to afford the title compound (37 mg, 48% yield). MS obsd. (ESI+): 437.5 (M+H)⁺. ¹H NMR (400 MHz, DMSO-d6) δ ppm: 8.91 (1H), 7.62 (1H), 7.48 (1H), 7.38-7.06 (3H), 6.86 (1H), 5.19 (1H), 4.80-4.40 (2H), 3.24 (3H), 1.53 (3H), 1.40-1.27 (4H).

### Example 126: (R)-6-(1-(1,1-difluoroethyl)cyclopropyl)-4-((1-(3-(difluoromethyl)-2-fluorophenyl)ethyl)amino)-2-methyl-2,6-dihydropyrido[3,4-d]pyridazine-1,7-dione

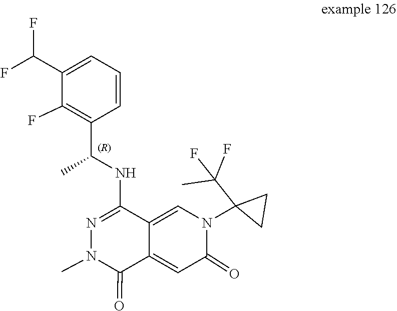

### Step A: benzyl 1-acetylcyclopropanecarboxylate

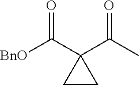 [SMILES: CC(=O)C1(C(=O)OCc2ccccc2)CC1]

To a solution of benzyl 3-oxobutanoate (5.0 g, 26.01 mmol) and K₂CO₃ (9.0 g, 65.03 mmol) in acetone (10.0 mL) was added 1,2-dibromoethane (9.8 g, 52.03 mmol). The mixture was stirred at 60° C. for 16 h. The mixture reaction was concentrated and the residue was purified by flash chromatography (eluting with 2% EA in PE) to afford the title compound (3.6 g, 63% yield).

¹H NMR (400 MHz, CDCl₃) δ ppm: 7.40-7.31 (5H), 5.17 (2H), 2.45 (3H), 1.53-1.47 (4H).

### Step B: benzyl 1-(1,1-difluoroethyl)cyclopropanecarboxylate

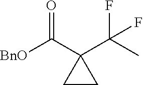 [SMILES: CC(F)(F)C1(C(=O)OCc2ccccc2)CC1]

To a solution of benzyl 1-acetylcyclopropanecarboxylate (2.0 g, 9.16 mmol) in bis(2-methoxyethyl)aminosulfur trifluoride (20.0 mL) was added EtOH (338 mg, 7.33 mmol) at 0° C. The mixture was stirred at 50° C. for 16 h. The mixture was quenched with aqueous Na2CO3 (aq.) and extracted with DCM. The organic layer was dried over anhydrous Na₂SO₄ and filtered and concentrated. The residue was purified by flash chromatography (eluting with 5% EA in PE) to afford the title compound (1.4 g, 65% yield). ¹H NMR (400 MHz, CDCl₃) δ ppm: 7.38-7.31 (5H), 5.14 (2H), 1.93 (3H), 1.34-1.32 (2H), 1.31-1.23 (2H).

### Step C: 1-(1,1-difluoroethyl)cyclopropane-1-carboxylic acid

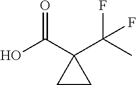

To a solution of benzyl 1-(1,1-difluoroethyl)cyclopropanecarboxylate (1.4 g, 5.99 mmol) in MeOH (20.0 mL) was added 10% Pd/C (509 mg) at room temperature, then the reaction mixture was stirred at 50° C. for 2 h. The mixture was filtered, and filtrate was concentrated to afford the title compound (828 mg, 92% yield).

¹H NMR (400 MHz, DMSO-d6) δ ppm: 12.77 (1H), 1.90 (3H), 1.19-1.17 (2H), 1.13-1.10 (2H).

### Step D: 1-(1,1-difluoroethyl)cyclopropanecarboxamide

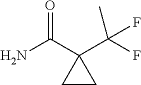

To a solution of 1-(1,1-difluoroethyl)cyclopropanecarboxylic acid (2.0 g, 13.3 mmol) in DMF (100 mL) was added NH₄Cl (10.7 g, 200 mmol), HOBT (1.8 g, 13.3 mmol), EDCI (5.1 g, 26.6 mmol) and TEA (9.3 mL, 67 mmol), then the reaction was stirred at room temperature for 16 h. The mixture was quenched with NH₄Cl (aq.) and extracted with EA. The organic layer was dried over anhydrous Na₂SO₄, filtered and concentrated and. The residue was purified by flash chromatography (eluting with 10% EA in PE) to afford the title compound (1.2 g, 60% yield). ¹H NMR (400 MHz, CDCl₃) δ ppm: 6.21 (1H), 5.62 (1H), 1.82 (3H), 1.30-1.24 (2H), 1.10-1.07 (2H).

### Step E: tert-butyl (1-(1,1-difluoroethyl)cyclopropyl)carbamate

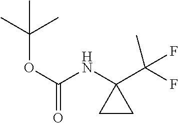

To a solution of [phenyl-(2,2,2-trifluoroacetyl)oxy-iodanyl] 2,2,2-trifluoroacetate (1.8 g, 4.18 mmol) in t-BuOH (12.0 mL) was added tert-butyl (1-(1,1-difluoroethyl)cyclopropyl)carbamate (623 mg, 4.18 mmol). The reaction mixture was stirred in the dark at room temperature for 12 h. The reaction was concentrated and the residue was purified by flash chromatography (eluting with 10% EA in PE) to afford an impure title compound (900 mg). This material was used without further purification.

¹H NMR (400 MHz, CDCl₃) δ ppm: 1.72 (3H), 1.49 (9H), 1.1 (2H), 0.94 (2H).

### Step F: 1-(1,1-difluoroethyl)cyclopropanamine hydrochloride

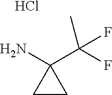

To a solution of tert-butyl N-[1-(1,1-difluoroethyl)cyclopropyl]carbamate (900 mg, crude) was added HCl/dioxane (4M, 20 mL). The reaction was stirred at room temperature for 1 h. The mixture was concentrated in vacuum to afford the title compound (322 mg, 49% yield (over 2 steps)). ¹H NMR (400 MHz, DMSO-d6) δ ppm: 9.09 (2H), 1.75 (3H), 1.23-1.12 (4H).

### Step G: 4-chloro-6-(1-(1,1-difluoroethyl)cyclopropyl)-2-methylpyrido[3,4-d]pyridazine-1,7(2H,6H)-dione

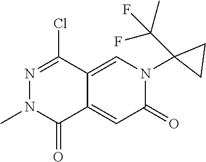 [SMILES: Cn1nc(Cl)c2cn(C3(C(C)(F)F)CC3)c(=O)cc2c1=O]

Prepared according to an analogous manner as Example 125 steps A and B, starting with 1-(1,1-difluoroethyl)cyclopropanamine hydrochloride. MS obsd. (ESI+): 316.2 [(M+H)⁺.

### Step E: (R)-6-(1-(1,1-difluoroethyl)cyclopropyl)-4-((1-(3-(difluoromethyl)-2-fluorophenyl)ethyl)amino)-2-methylpyrido[3,4-d]pyridazine-1,7(2H,6H)-dione (example 126)

To a solution of 4-chloro-6-(1-(1,1-difluoroethyl)cyclopropyl)-2-methylpyrido[3,4-d]pyridazine-1,7(2H,6H)-dione (60 mg, 190 μmol) in toluene (0.5 mL) was added (1R)-1-[3-(difluoromethyl)-2-fluoro-phenyl]ethanamine (43 mg, 228 μmol), cesium carbonate (185 mg, 0.57 mmol), Pd(OAc)₂ (9 mg, 38.01 μmol) and BINAP (24 mg, 38 μmol). The mixture was heated in a microwave reactor at 100° C. for 16 h. The reaction mixture was concentrated and the residue was purified by flash chromatography (eluting with 2% MeOH in DCM), followed by preparative HPLC (MeCN/H₂O) to afford the title compound (21 mg, 24% yield). MS obsd. (ESI+): 469.2 [(M+H)⁺].

### Example 127: (R)-2-methyl-4-((1-(2-methyl-3-(trifluoromethyl)phenyl)ethyl)amino)-6-(1-methyl-2-oxabicyclo[2.1.1]hexan-4-yl)-2,6-dihydropyrido[3,4-d]pyridazine-1,7-dione

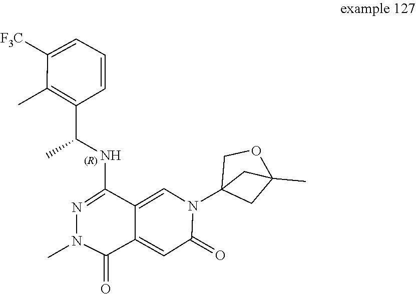

### Step A: 2-(6-chloro-5-(hydroxymethyl)-2-methyl-3-oxo-2,3-dihydropyridazin-4-yl)-N-(1-methyl-2-oxabicyclo[2.1.1]hexan-4-yl)acetamide

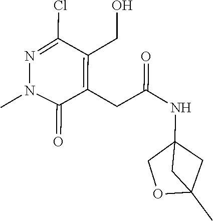

To a solution of 4-chloro-2-methyl-5,8-dihydropyrano[3,4-d]pyridazine-1,7-dione (180 mg, 836 μmol) in Toluene (5 mL) was added 1-methyl-2-oxabicyclo[2.1.1]hexan-4-amine hydrochloride (150 mg, 1.00 mmol) and triethylamine (140 μL, 1.00 mmol,) at 0° C. The reaction mixture was stirred for 16 hr at 80° C. To the reaction mixture was added aqueous NaCl and DCM. The organic layer was concentrated to dryness and the residue was purified by silica gel chromatography (eluting with 0%-5% MeOH in DCM) to afford the title compound (190 mg, 69% yield). MS obsd. (ESI+): 328.2 [(M+H)⁺].

### Step B: 4-chloro-2-methyl-6-(1-methyl-2-oxabicyclo[2.1.1]hexan-4-yl)-2,6-dihydropyrido[3,4-d]pyridazine-1,7-dione

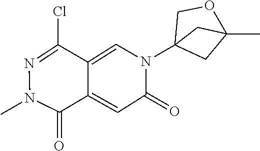

To a solution of 2-(6-chloro-5-(hydroxymethyl)-2-methyl-3-oxo-2,3-dihydropyridazin-4-yl)-N-(1-methyl-2-oxabicyclo[2.1.1]hexan-4-yl)acetamide(186 mg, 567 mol) in CH₃CN (5 mL) was added 2-Iodoxybenzoic acid (318 mg, 1.13 mmol). The reaction mixture was stirred at 80° C. for 3 hr. The reaction mixture was concentrated, diluted by DCM (20 mL), and filtered through a celite pad. The filtrate was concentrated and purified by silica gel chromatography column (eluting with 0%-5% MeOH in DCM) to afford the title compound (110 mg, 63% yield). MS obsd. (ESI+): 308.2 [(M+H)+].

### Step C: 2-methyl-4-(((R)-1-(2-methyl-3-(trifluoromethyl)phenyl)ethyl)amino)-6-((1R,4r)-1-methyl-2-oxabicyclo[2.1.1]hexan-4-yl)-2,6-dihydropyrido[3,4-d]pyridazine-1,7-dione (example 127)

To a solution of 4-chloro-2-methyl-6-(1-methyl-2-oxabicyclo[2.1.1]hexan-4-yl)-2,6-dihydropyrido[3,4-d]pyridazine-1,7-dione (40 mg, 130 μmol) in dioxane (1 mL) was added (1R)-1-[2-methyl-3-(trifluoromethyl)phenyl]ethanamine (40 mg, 195 μmol), cesium carbonate (127 mg, 390 μmol) and Xantphos Pd G3 (25 mg, 26.00 μmol). The reaction was stirred for 8 hr at 100° C. The solvent was removed in vacuo and the residue was purified by preparative TLC (DCM/MeOH=20/1) followed by preparative HPLC (ACN/water/0.1% NH₄HCO₃) to afford the title compound (8 mg, 13% yield). MS obsd. (ESI+): 475.0 [(M+H)+]. ¹H NMR (400 MHz, DMSO-d6) δ: 8.58 (1H), 7.73 (1H), 7.54 (1H), 7.36 (1H), 7.24 (1H), 6.81 (1H), 5.17 (1H), 3.91 (2H), 3.25 (3H), 2.54 (3H), 2.37 (2H), 2.20 (2H), 1.47 (6H).

### Examples 128 and 129: 4-(((R)-1-(3-(difluoromethyl)-2-fluorophenyl)ethyl)amino)-2-methyl-6-((R)-spiro[2.2]pentan-1-yl)-2,6-dihydropyrido[3,4-d]pyridazine-1,7-dione (example 128, stereochemistry at cyclopropyl is arbitrarily assigned) and 4-(((R)-1-(3-(difluoromethyl)-2-fluorophenyl)ethyl)amino)-2-methyl-6-((S)-spiro[2.2]pentan-1-yl)-2,6-dihydropyrido[3,4-d]pyridazine-1,7-dione (example 129, stereochemistry at cyclopropyl is arbitrarily assigned)

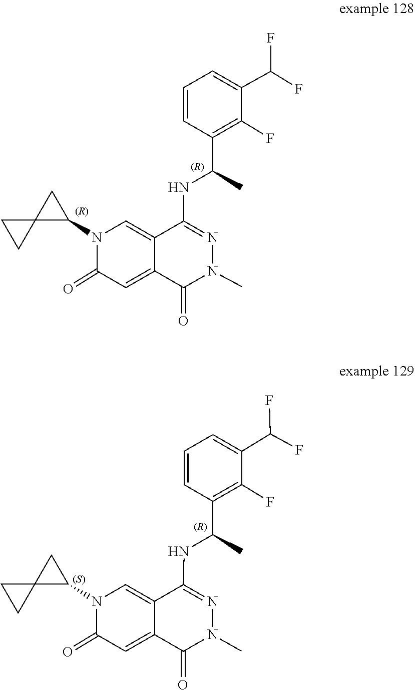

The diastereomeric mixture was prepared according to an analogous route to example 127. Separation of isomers afforded 4-(((R)-1-(3-(difluoromethyl)-2-fluorophenyl)ethyl)amino)-2-methyl-6-((R)-spiro[2.2]pentan-1-yl)-2,6-dihydropyrido[3,4-d]pyridazine-1,7-dione (example 128, stereochemistry at cyclopropyl is arbitrarily assigned) and 4-(((R)-1-(3-(difluoromethyl)-2-fluorophenyl)ethyl)amino)-2-methyl-6-((S)-spiro[2.2]pentan-1-yl)-2,6-dihydropyrido[3,4-d]pyridazine-1,7-dione (example 129, stereochemistry at cyclopropyl is arbitrarily assigned). Chiral HPLC: Column Name: AS-3 4.6*100 mm 3 um,MeOH[0.2% NH3(7M in MeOH)], Flow rate: 3.0 mL/min, temp: 40° C.

Example 128: Chiral HPLC Retention time=1.09 min. MS obsd. (ESI+): 431.4 (M+H)+.

Example 129: Chiral HPLC Retention time=1.62 min. MS obsd. (ESI+): 431.4 (M+H)+.

### Examples 130 and 131: 4-(((R)-1-(3-(difluoromethyl)-2-fluorophenyl)ethyl)amino)-6-((S)-6,6-difluorospiro[2.5]octan-1-yl)-2-methyl-2,6-dihydropyrido[3,4-d]pyridazine-1,7-dione (example 130, stereochemistry at cyclopropyl is arbitrarily assigned) and 4-(((R)-1-(3-(difluoromethyl)-2-fluorophenyl)ethyl)amino)-6-((R)-6,6-difluorospiro[2.5]octan-1-yl)-2-methyl-2,6-dihydropyrido[3,4-d]pyridazine-1,7-dione (example 131, stereochemistry at cyclopropyl is arbitrarily assigned)

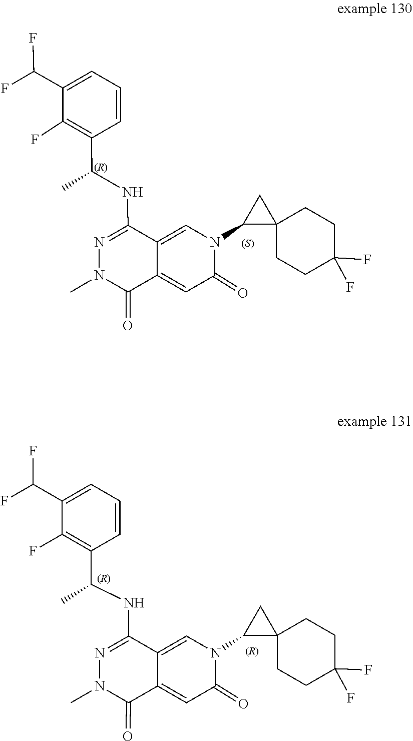

The diastereomeric mixture was prepared according to an essential analogous route to example 127. Separation of isomers via chiral SFC (Column: Regis(R,R)Whelk 01 (4.6*100 mm 3 um), CoSolvent: CO₂/MeOH[0.2% NH3(7M in MeOH)]=60:40) afforded 4-(((R)-1-(3-(difluoromethyl)-2-fluorophenyl)ethyl)amino)-6-((S)-6,6-difluorospiro[2.5]octan-1-yl)-2-methyl-2,6-dihydropyrido[3,4-d]pyridazine-1,7-dione (example 130, stereochemistry at cyclopropyl is arbitrarily assigned) and 4-(((R)-1-(3-(difluoromethyl)-2-fluorophenyl)ethyl)amino)-6-((R)-6,6-difluorospiro[2.5]octan-1-yl)-2-methyl-2,6-dihydropyrido[3,4-d]pyridazine-1,7-dione (example 131, stereochemistry at cyclopropyl is arbitrarily assigned). Chiral HPLC: Column Name: OJ-3 4.6*100 mm 3.0 um, MeOH[0.2% NH3(7M in MeOH)], Flow rate: 3.0 mL/min, temp: 40° C.

Example 130: Chiral HPLC Retention time=1.63 min. MS obsd. (ESI+): 509.5 (M+H)+.

Example 131: Chiral HPLC Retention time=1.80 min. MS obsd. (ESI+): 509.5 (M+H)+.

### Examples 132 and 133: 4-(((R)-1-(3-(difluoromethyl)-2-fluorophenyl)ethyl)amino)-6-((R)-2,2-dimethylcyclopropyl)-2-methyl-2,6-dihydropyrido[3,4-d]pyridazine-1,7-dione (example 132, stereochemistry at cyclopropyl is arbitrarily assigned) and 4-(((R)-1-(3-(difluoromethyl)-2-fluorophenyl)ethyl)amino)-6-((S)-2,2-dimethylcyclopropyl)-2-methyl-2,6-dihydropyrido[3,4-d]pyridazine-1,7-dione (example 133, stereochemistry at cyclopropyl is arbitrarily assigned)

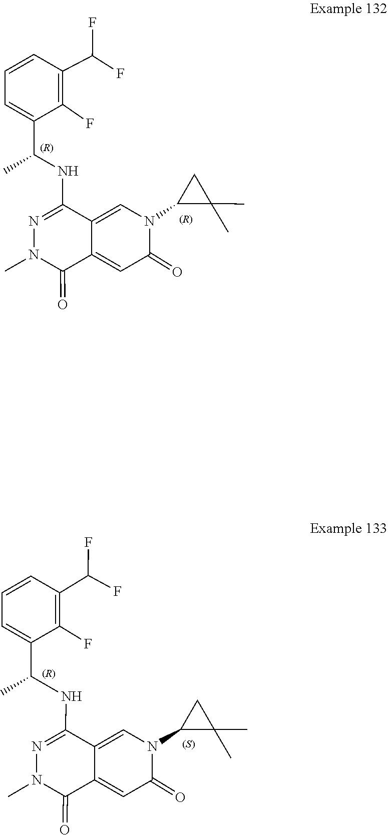

The diastereomeric mixture was prepared according to an essential analogous route to example 127. Separation of isomers via chiral SFC (Column Name: (R,R) Whelk-O1 4.6*100 mm 3.5 μm) afforded 4-(((R)-1-(3-(difluoromethyl)-2-fluorophenyl)-ethyl)amino)-6-((R)-2,2-dimethylcyclopropyl)-2-methyl-2,6-dihydropyrido[3,4-d]pyridazine-1,7-dione (example 132, stereochemistry at cyclopropyl is arbitrarily assigned) and 4-(((R)-1-(3-(difluoro-methyl)-2-fluorophenyl)ethyl)amino)-6-((S)-2,2-dimethylcyclopropyl)-2-methyl-2,6-dihydropyrido[3,4-d]pyridazine-1,7-dione (example 133, stereochemistry at cyclopropyl is arbitrarily assigned). Chiral HPLC: Column Name: (R,R)Whelk-O1 4.6*100 mm 3.0 um, MeOH[0.2% NH3(7M in MeOH)], Flow rate: 3.0 mL/min, temp: 40° C.

Example 132: Chiral HPLC Retention time=1.35 min. MS obsd. (ESI+): 433.4 (M+H)+.

Example 133: Chiral HPLC Retention time=1.71 min. MS obsd. (ESI+): 433.4 (M+H)+.

### Example 134: (R)-6-(3,3-difluoro-1-methylcyclobutyl)-4-((1-(3-(difluoromethyl)-2-fluorophenyl)ethyl)amino)-2-methyl-2,6-dihydropyrido[3,4-d]pyridazine-1,7-dione

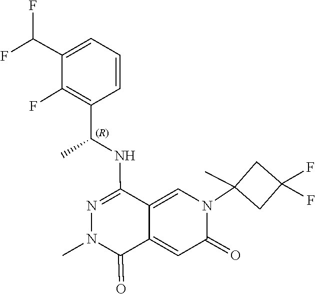

Prepared according to an essential analogous route to example 127. MS obsd. (ESI+): 469.1 (M+H)+. ¹H NMR (400 MHz, DMSO-d6) δ: 8.61 (1H), 7.66 (1H), 7.48 (1H), 7.37-7.09 (3H), 6.83 (1H), 5.23-5.16 (1H), 3.42-3.38 (2H), 3.25 (3H), 3.09-3.02 (2H), 1.68 (3H), 1.54 (3H).

### Examples 135 and 136: 6-((1R,2S)-[1,1′-bi(cyclopropan)]-2-yl)-4-(((R)-1-(3-(difluoromethyl)-2-fluorophenyl)ethyl)amino)-2-methyl-2,6-dihydropyrido[3,4-d]pyridazine-1,7-dione (example 135, stereochemistry at cyclopropyl is arbitrarily assigned) and 6-((1S,2R)-[1,1′-bi(cyclopropan)]-2-yl)-4-(((R)-1-(3-(difluoromethyl)-2-fluorophenyl)ethyl)amino)-2-methyl-2,6-dihydropyrido[3,4-d]pyridazine-1,7-dione (example 136, stereochemistry at cyclopropyl is arbitrarily assigned)

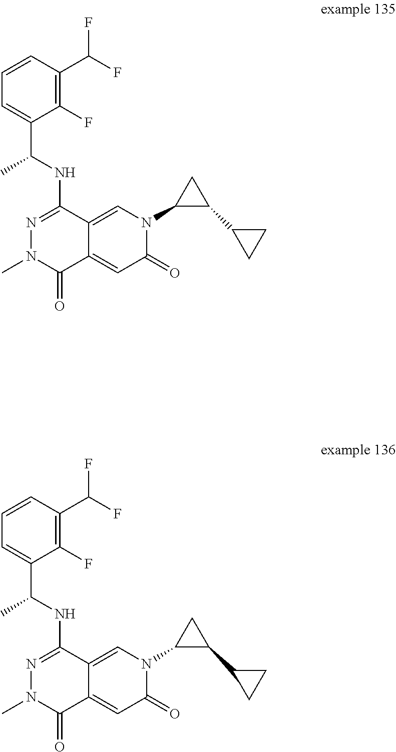 [SMILES: C[C@@H](Nc1nn(C)c(=O)c2cc(=O)n([C@H]3C[C@@H]3C3CC3)cc12)c1cccc(C(F)F)c1F ; C[C@@H](Nc1nn(C)c(=O)c2cc(=O)n([C@@H]3C[C@H]3C3CC3)cc12)c1cccc(C(F)F)c1F]

The diastereomeric mixture was prepared according to an essential analogous route to example 127 using trans-2-cyclopropylcyclopropanamine hydrochloride in Step A in place of 1-methyl-2-oxabicyclo[2.1.1]hexan-4-amine hydrochloride. Separation of isomers via chiral SFC (Column Name: Daicel AS 25*250 mm 10 m) afforded 6-((1R,2S)-[1,1′-bi(cyclopropan)]-2-yl)-4-(((R)-1-(3-(difluoromethyl)-2-fluorophenyl)ethyl)amino)-2-methyl-2,6-dihydropyrido[3,4-d]pyridazine-1,7-dione (example 135, stereochemistry at cyclopropyl is arbitrarily assigned) and 6-((1S,2R)-[1,1′-bi(cyclopropan)]-2-yl)-4-(((R)-1-(3-(difluoromethyl)-2-fluorophenyl)ethyl)amino)-2-methyl-2,6-dihydropyrido[3,4-d]pyridazine-1,7-dione (example 136, stereochemistry at cyclopropyl is arbitrarily assigned). Chiral HPLC: Column Name: AS-3 4.6*100 mm 3.0 um, MeOH[0.2% NH3(7M in MeOH)], Flow rate: 3.0 mL/min, temp: 40° C.

Example 135: Chiral HPLC Retention time=1.27 min. MS obsd. (ESI+): 445.0 (M+H)+.

Example 136: Chiral HPLC Retention time=1.69 min. MS obsd. (ESI+): 445.0 (M+H)+.

### Examples 137 and 138: 4-(((R)-1-(3-(difluoromethyl)-2-fluorophenyl)ethyl)amino)-2-methyl-6-((1S,2S)-2-(trifluoromethyl)cyclopropyl)-2,6-dihydropyrido[3,4-d]pyridazine-1,7-dione (example 137, stereochemistry at cyclopropyl is arbitrarily assigned) and 4-(((R)-1-(3-(difluoromethyl)-2-fluorophenyl)ethyl)amino)-2-methyl-6-((1R,2R)-2-(trifluoromethyl)cyclopropyl)-2,6-dihydropyrido[3,4-d]pyridazine-1,7-dione (example 138, stereochemistry at cyclopropyl is arbitrarily assigned)

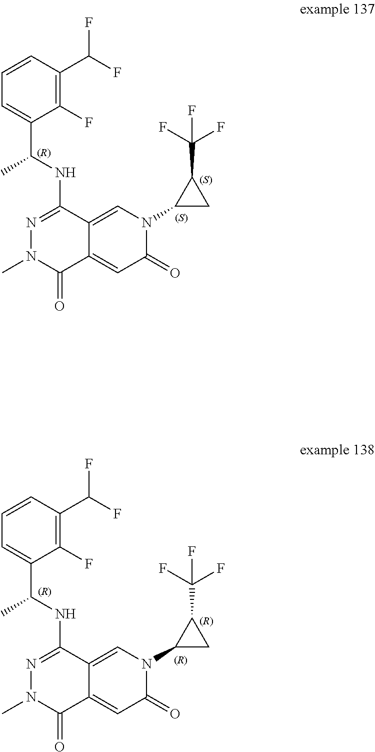

The diastereomeric mixture was prepared according to an essential analogous route to example 127 using trans-2-(trifluoromethyl)cyclopropanamine hydrochloride in Step A in place of 1-methyl-2-oxabicyclo[2.1.1]hexan-4-amine hydrochloride. Separation of isomers via chiral SFC (Column Name:Daicel OZ-3, 4.6*100 mm 3 μm, CO₂/MeOH(0.2% Methanol Ammonia)=70/30) afforded 4-(((R)-1-(3-(difluoromethyl)-2-fluorophenyl)ethyl)amino)-2-methyl-6-((1S,2S)-2-(trifluoromethyl)cyclopropyl)-2,6-dihydropyrido[3,4-d]pyridazine-1,7-dione (example 137, stereochemistry at cyclopropyl is arbitrarily assigned) and 4-(((R)-1-(3-(difluoromethyl)-2-fluorophenyl)ethyl)amino)-2-methyl-6-((1R,2R)-2-(trifluoromethyl)cyclopropyl)-2,6-dihydropyrido[3,4-d]pyridazine-1,7-dione (example 138, stereochemistry at cyclopropyl is arbitrarily assigned). Chiral HPLC: Column Name: OZ-3 4.6*100 mm 3.0 um, MeOH[0.2% NH3(7M in MeOH)], Flow rate: 3.0 mL/min, temp: 40° C.

Example 137: Chiral HPLC retention time=1.77 min. MS obsd. (ESI+): 473.4 (M+H)+. ¹H NMR (400 MHz, DMSO-d6) δ 8.68 (1H), 7.62 (1H), 7.48 (1H), 7.23 (3H), 6.86 (1H), 5.20 (1H), 3.96-3.81 (1H), 3.25 (3H), 2.51 (5H), 1.99-1.88 (1H), 1.65 (1H), 1.53 (3H).

Example 138: Chiral HPLC retention time=2.41 min. MS obsd. (ESI+): 473.4 (M+H)+. ¹H NMR (400 MHz, DMSO-d6) δ 8.68 (1H), 7.63 (1H), 7.49 (1H), 7.23 (3H), 6.85 (1H), 5.25-5.14 (1H), 3.94-3.81 (1H), 3.25 (3H), 1.99-1.88 (1H), 1.65 (1H), 1.53 (3H).

### Examples 139 and 140: 6-((1S,5R)-3-acetyl-3-azabicyclo[3.1.0]hexan-1-yl)-4-(((R)-1-(3-(difluoromethyl)-2-fluorophenyl)ethyl)amino)-2-methyl-2,6-dihydropyrido[3,4-d]pyridazine-1,7-dione (example 139, stereochemistry at cyclopropyl is arbitrarily assigned) and 6-((1R,5S)-3-acetyl-3-azabicyclo[3.1.0]hexan-1-yl)-4-(((R)-1-(3-(difluoromethyl)-2-fluorophenyl)ethyl)amino)-2-methyl-2,6-dihydropyrido[3,4-d]pyridazine-1,7-dione(example 140, stereochemistry at cyclopropyl is arbitrarily assigned)

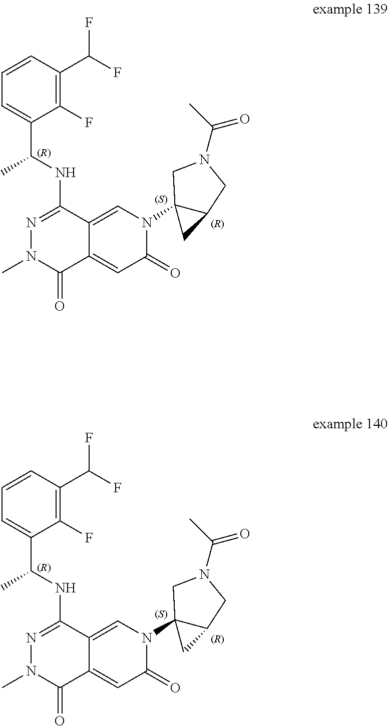

### Step A: Tert-butyl 1-(4-(((R)-1-(3-(difluoromethyl)-2-fluorophenyl)ethyl)amino)-2-methyl-1,7-di-oxo-1,7-dihydropyrido[3,4-d]pyridazin-6(2H)-yl)-3-azabicyclo[3.1.0]hexane-3-carboxylate

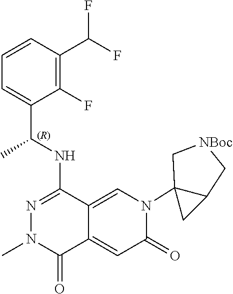

Prepared according to an essentially analogous route to example 127 using tert-butyl 1-amino-3-azabicyclo[3.1.0]hexane-3-carboxylate in Step A in place of 1-methyl-2-oxabicyclo[2.1.1]hexan-4-amine hydrochloride. MS obsd. (ESI+): 546.5 (M+H)+.

### Step B: 6-(3-azabicyclo[3.1.0]hexan-1-yl)-4-(((R)-1-(3-(difluoromethyl)-2-fluorophenyl)ethyl)-amino)-2-methyl-2,6-dihydropyrido[3,4-d]pyridazine-1,7-dione

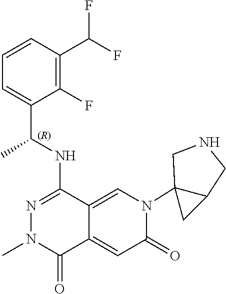

To a mixture of tert-butyl 1-(4-(((R)-1-(3-(difluoromethyl)-2-fluorophenyl)ethyl)amino)-2-methyl-1,7-dioxo-1,7-dihydropyrido[3,4-d]pyridazin-6(2H)-yl)-3-azabicyclo[3.1.0]hexane-3-carboxylate (80 mg, 147 μmol) in DCM (4 mL) was added TFA (2 mL). The mixture was stirred at rt for 2 hr and then concentrated. The mixture was basified to pH=8 with NH3-MeOH (7M) and concentrated to provide the crude title compound (65 mg, crude). MS obsd. (ESI⁺): 446.4 [(M+H)⁺].

### Step C: 6-(3-acetyl-3-azabicyclo[3.1.0]hexan-1-yl)-4-(((R)-1-(3-(difluoromethyl)-2-fluoro-phenyl)ethyl)amino)-2-methyl-2,6-dihydropyrido[3,4-d]pyridazine-1,7-dione

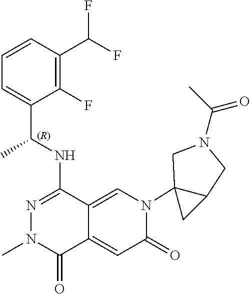

To a mixture of acetic acid (16.7 μL, 292 μmol) in DMF (2 mL) was added HATU (83 mg, 219 μmol), TEA (61.0 μL, 438 μmol),followed by 6-(3-azabicyclo-[3.1.0]hexan-1-yl)-4-(((R)-1-(3-(difluoromethyl)-2-fluorophenyl)ethyl)amino)-2-methyl-2,6-dihydropyrido[3,4-d]pyridazine-1,7-dione (65 mg, crude). The mixture was stirred at rt for 2 hr. The mixture was diluted with water and extracted into EA (4 mL*3). The combined organic layers were washed with H₂O (6 mL*3), brine (6 mL*3), and then dried over sodium sulfate, filtered and concentrated. The residue was purified by preparative TLC (DCM:MeOH=20:1) to provide the title compound (37 mg, 52% yield). MS obsd. (ESI⁺): 488.5 [(M+H)⁺].

### Step D: 6-((1S,5R)-3-acetyl-3-azabicyclo[3.1.0]hexan-1-yl)-4-(((R)-1-(3-(difluoromethyl)-2-fluorophenyl)ethyl)amino)-2-methyl-2,6-dihydropyrido[3,4-d]pyridazine-1,7-dione(example 139, stereochemistry at cyclopropyl is arbitrarily assigned) and 6-((1R,5S)-3-acetyl-3-azabicyclo[3.1.0]hexan-1-yl)-4-(((R)-1-(3-(difluoromethyl)-2-fluorophenyl)ethyl)amino)-2-methyl-2,6-dihydropyrido[3,4-d]pyridazine-1,7-dione(example 140, stereochemistry at cyclopropyl is arbitrarily assigned)

6-(3-acetyl-3-azabicyclo[3.1.0]hexan-1-yl)-4-(((R)-1-(3-(difluoromethyl)-2-fluorophenyl)ethyl)amino)-2-methyl-2,6-dihydropyrido[3,4-d]pyridazine-1,7-dione (37 mg, 75.9 mol) was separated by chiral SFC to provide the title compounds example 139 (9.0 mg, 19% yield) and example 140 (8.2 mg, 22% yield). Chiral HPLC: Column Name: (R,R)Whelk-O1 4.6*100 mm 3.5 um, EtOH[1% NH3(7M in MeOH)], flow rate: 3.0 mL/min, temp: 40° C.

Example 139: Chiral HPLC retention time=2.08 min. MS obsd. (ESI⁺): 488.5 [(M+H)⁺].

Example 140: Chiral HPLC retention time=2.47 min. MS obsd. (ESI⁺): 488.5 [(M+H)⁺].

### Examples 141 and 142: 6-((1S,5R)-3-(cyclopropanecarbonyl)-3-azabicyclo[3.1.0]hexan-1-yl)-4-(((R)-1-(3-(difluoromethyl)-2-fluorophenyl)ethyl)amino)-2-methyl-2,6-dihydropyrido[3,4-d]pyridazine-1,7-dione (example 141, stereochemistry at cyclopropane is arbitrarily assigned) and 6-((1R,5S)-3-(cyclopropanecarbonyl)-3-azabicyclo[3.1.0]hexan-1-yl)-4-(((R)-1-(3-(difluoromethyl)-2-fluorophenyl)ethyl)amino)-2-methyl-2,6-dihydropyrido[3,4-d]pyridazine-1,7-dione (example 142, stereochemistry at cyclopropane is arbitrarily assigned)

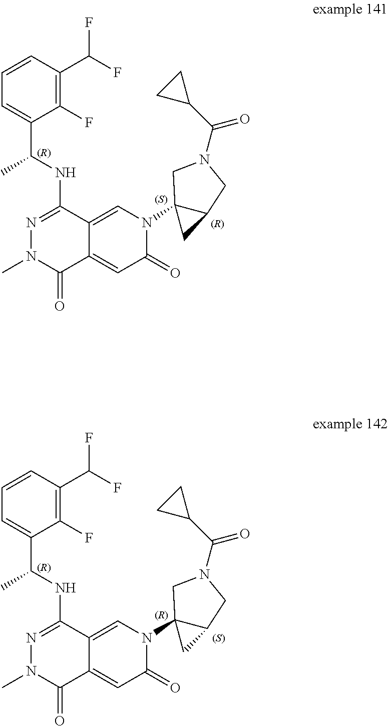

Prepared according to an essential analogous route to example 139/140 using cyclopropane carboxylic acid in Step C in place of acetic acid and chiral separation of the isomers via SFC Daicel OJ-3 (4.6*100 mm 3 um) EtOH[1% NH₃(7M in MeOH)] to provide the title compounds, example 141 (11 mg, 28% yield) and example 142 (12 mg, 29% yield). Chiral HPLC: Column Name: OJ-3 4.6*100 mm 3.0 um, EtOH[1% NH3(7M in MeOH)], flow rate: 3.0 mL/min, temp: 40° C.

Example 141: Chiral HPLC retention time=1.04 min. MS obsd. (ESI⁺): 514.5 [(M+H)⁺].

Example 142: Chiral HPLC retention time=1.41 min. MS obsd. (ESI⁺): 514.5 [(M+H)⁺].

### Examples 143 and 144: 6-((R)-1-acetyl-3-methylpyrrolidin-3-yl)-4-(((R)-1-(3-(difluoromethyl)-2-fluorophenyl)ethyl)amino)-2-methyl-2,6-dihydropyrido[3,4-d]pyridazine-1,7-dione (example 143, stereochemistry at pyrrolidine is arbitrarily assigned) and 6-((S)-1-acetyl-3-methylpyrrolidin-3-yl)-4-(((R)-1-(3-(difluoromethyl)-2-fluorophenyl)ethyl)amino)-2-methyl-2,6-dihydropyrido[3,4-d]pyridazine-1,7-dione (example 144, stereochemistry at pyrrolidine is arbitrarily assigned)

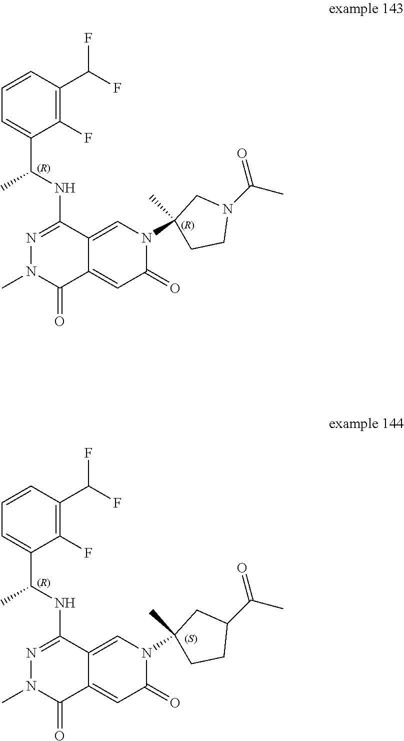

Prepared according to an essential analogous route to example 139/144 using tert-butyl 3-amino-3-methylpyrrolidine-1-carboxylate in Step A in place of using tert-butyl 1-amino-3-azabicyclo[3.1.0]hexane-3-carboxylate. Chiral separation of the isomers via SFC (Daicel OD (25*250 mm, 10 um), mobile phase: CO₂/MeOH[0.2% NH₃(7M in MeOH)]=85/15) to afford example 143 (42 mg, 37% yield) and example 144 (41 mg, 36% yield). Chiral HPLC: Column Name: OD-3 4.6*100 mm 3.0 um, MeOH[0.2% NH3(7M in MeOH)], flow rate: 3.0 mL/min, temp: 40° C.

Example 143: Chiral HPLC retention time=2.86 min. MS obsd. (ESI⁺): 490.6 [(M+H)⁺]. ¹H NMR (400 MHz, DMSO-d₆) δ: 8.57 (1H), 7.64 (1H), 7.48 (1H), 7.39-7.08 (3H), 6.84 (1H), 5.20 (1H), 4.42 (1H), 3.76-3.56 (2H), 3.37 (1H), 3.24 (3H), 2.69 (1H),2.46 (1H), 1.97 (3H), 1.56 (6H).

Example 144: Chiral HPLC retention time=3.90 min. MS obsd. (ESI⁺): 490.6 [(M+H)⁺]. ¹H NMR (400 MHz, DMSO-d₆) δ: 8.57 (1H), 7.63 (1H), 7.49 (1H), 7.38-7.08 (3H), 6.83 (1H), 5.19 (1H), 4.42 (1H), 3.74-3.56 (2H), 3.46-3.33 (1H), 3.25 (3H), 2.69 (1H), 2.49-2.40 (1H), 1.98 (3H), 1.56 (6H).

### Example 145: (R)-6-(2-azabicyclo[2.1.1]hexan-4-yl)-4-((1-(3-(difluoromethyl)-2-fluorophenyl)ethyl)amino)-2-methyl-2,6-dihydropyrido[3,4-d]pyridazine-1,7-dione

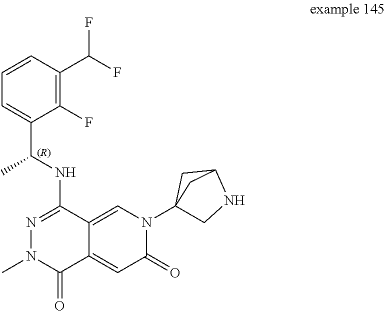

### Step A: tert-butyl 4-(benzyloxycarbonylamino)-2-azabicyclo[2.1.1]hexane-2-carboxylate

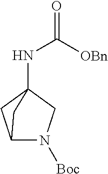 [SMILES: CC(C)(C)OC(=O)N1CC2(NC(=O)OCc3ccccc3)CC1C2]

To a solution of 2-tert-butoxycarbonyl-2-azabicyclo[2.1.1]hexane-4-carboxylic acid (800 mg, 3.52 mmol) in toluene (14.7 mL) was added triethylamine (1.47 mL, 10.6 mmol, 1.47 mL), followed by diphenyl azidophosphate (1.55 g, 5.63 mmol), and the reaction mixture was heated to reflux for 1 h. The reaction mixture was then cooled to room temperature and benzyl alcohol (762 mg, 7.04 mmol) was added. The resulting solution was heated to reflux overnight. After cooling to room temperature, the reaction mixture was concentrated and purified by flash chromatography (eluting with 0% to 50% EA in PE) to afford the title compound (917 mg, 78% yield). MS obsd. (ESI⁺): 277.3 [(M−tBu)⁺].

### Step B: Tert-butyl 4-amino-2-azabicyclo[2.1.1]hexane-2-carboxylate

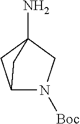 [SMILES: CC(C)(C)OC(=O)N1CC2(N)CC1C2]

To a solution of tert-butyl 4-(benzyloxycarbonylamino)-2-azabicyclo[2.1.1]hexane-2-carboxylate (917 mg, 3.17 mmol) in methanol (12 mL) was added Pd/C (439 mg, 10 wt %) and the mixture was stirred at rt for 16 h under hydrogen atmosphere. The mixture was filtered and the filtrate was evaporated to give the title compound (568 mg, 81% yield).

¹H NMR (400 MHz, DMSO-d₆) δ: 3.98 (1H), 2.96 (2H), 2.36 (2H), 1.72-1.55 (2H), 1.39 (9H).

### Step C: (R)-6-(2-azabicyclo[2.1.1]hexan-4-yl)-4-((1-(3-(difluoromethyl)-2-fluorophenyl)ethyl)amino)-2-methyl-2,6-dihydropyrido[3,4-d]pyridazine-1,7-dione (example 145)

Prepared according to an essentially analogous route to example 139/140 steps A and B using tert-butyl 4-(benzyloxycarbonylamino)-2-azabicyclo[2.1.1]hexane-2-carboxylate in place of tert-butyl 1-amino-3-azabicyclo[3.1.0]hexane-3-carboxylate. MS obsd. (ESI⁺): 446.5 [(M+H)⁺]. ¹H NMR (400 MHz, DMSO-d₆) δ: 8.57 (1H), 7.63 (1H), 7.48 (1H), 7.36 (1H), 7.29 (1H), 7.23 (1H), 7.17 (1H), 7.09 (1H), 6.79 (1H), 5.19 (1H), 3.61 (1H), 3.25 (3H), 3.04 (2H), 2.27-2.14 (2H), 2.04 (2H), 1.52 (3H).

### Example 146: (R)-6-(2-acetyl-2-azabicyclo[2.1.1]hexan-4-yl)-4-((1-(3-(difluoromethyl)-2-fluorophenyl)ethyl)amino)-2-methyl-2,6-dihydropyrido[3,4-d]pyridazine-1,7-dione

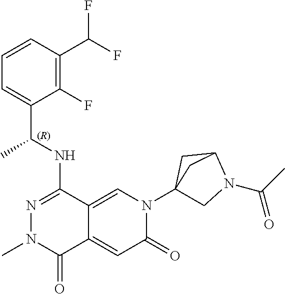

A solution of acetic acid (7 mg, 0.11 mmol), HATU (82 mg, 0.22 mmol) and triethylamine (44 mg, 0.43 mmol) in DMF (0.5 mL) was stirred for 15 min, then (R)-6-(2-azabicyclo[2.1.1]hexan-4-yl)-4-((1-(3-(difluoromethyl)-2-fluorophenyl)ethyl)amino)-2-methyl-2,6-dihydropyrido[3,4-d]pyridazine-1,7-dione (example 145, 48 mg, 0.11 mmol) was added and the mixture was stirred for 16 h. The mixture was diluted with DCM (60 mL) and washed with water (50 mL×2). The organic layer was dried over Na₂SO₄, filtered and concentrated, and the residue was purified by preparative HPLC (ACN/water/0.1% NH₄HCO₃) to give the title compound (21 mg, 40% yield). MS obsd. (ESI⁺): 488.5 [(M+H)⁺]. ¹H NMR (400 MHz, DMSO-d₆) δ: 8.55 (1H), 7.63 (1H), 7.49 (1H), 7.29 (1H), 7.23 (1H), 7.16 (1H), 6.84 (1H), 5.21 (1H), 4.64 (1H), 3.81-3.74 (1H), 3.63-3.55 (1H), 3.26 (3H), 2.43 (1H), 2.35 (2H), 2.28 (1H), 1.98 (3H), 1.53 (3H).

### Example 147: (R)-4-((1-(3-(difluoromethyl)-2-fluorophenyl)ethyl)amino)-6-(1-(difluoromethyl)cyclo-butyl)-2-methyl-2,6-dihydropyrido[3,4-d]pyridazine-1,7-dione

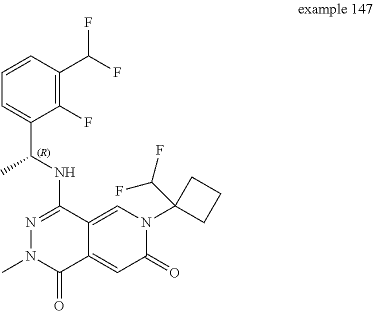

### Step A: 2-(6-chloro-5-(hydroxymethyl)-2-methyl-3-oxo-2,3-dihydropyridazin-4-yl)-N-(1-(difluoro-methyl)cyclobutyl)acetamide

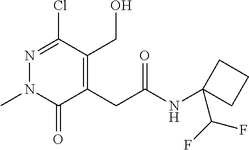 [SMILES: Cn1nc(Cl)c(CO)c(CC(=O)NC2(C(F)F)CCC2)c1=O]

To a mixture of 4-chloro-2-methyl-5,8-dihydro-2H-pyrano[3,4-d]pyridazine-1,7-dione (200 mg, 932 μmol) in toluene (4.40 mL) was added 1-(difluoromethyl)cyclobutan-1-amine hydrochloride (147 mg, 931.94 μmol), and trimethylaluminum (1 M in toluene, 1.40 mL). The mixture was stirred at rt for 2 hr then cooled down to 0° C. and quenched with saturated NH₄Cl. The mixture was extracted with EtOAc (4 mL*3) and the combined organic layers were subsequently washed with H₂O (6 mL*3) and brine (6 mL*3), dried over sodium sulfate and concentrated. The residue was purified by silica gel column chromatography (eluting with 3% MeOH in DCM) followed by preparative TLC (DCM:MeOH=20:1) to provide the title compound (175 mg, 56% yield).

MS obsd. (ESI⁺): 336.3 [(M+H)⁺].

### Step B: (R)-4-((1-(3-(difluoromethyl)-2-fluorophenyl)ethyl)amino)-6-(1-(difluoromethyl)cyclo-buty-1)-2-methyl-2,6-dihydropyrido[3,4-d]pyridazine-1,7-dione (example 147)

Prepared according to an essentially analogous route to example 127 steps B and C using 2-(6-chloro-5-(hydroxymethyl)-2-methyl-3-oxo-2,3-dihydropyridazin-4-yl)-N-(1-(difluoro-methyl)cyclobutyl)acetamide in place of 2-(6-chloro-5-(hydroxymethyl)-2-methyl-3-oxo-2,3-dihydropyridazin-4-yl)-N-(1-(fluoromethyl)cyclopropyl)acetamide in Step B. MS obsd. (ESI⁺): 469.5 [(M+H)⁺]. ¹H NMR (400 MHz, DMSO-d₆) δ: 8.55 (1H), 7.61 (1H), 7.48 (1H), 7.40-7.06 (3H), 6.84 (1H), 6.46 (1H), 5.26-5.15 (1H), 3.25 (3H), 2.75 (4H), 2.06-1.81 (2H), 1.53 (3H).

### Example 148: 4-[[(1R)-1-[3-(difluoromethyl)-2-fluoro-phenyl]ethyl]amino]-2-methyl-6-[(1S,5S)-3-oxabicyclo[3.1.0]hexan-1-yl]pyrido[3,4-d]pyridazine-1,7-dione

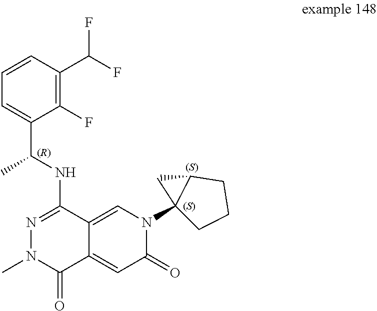

Prepared according to an essentially analogous route to example 125 using (1S,5S)-3-oxabicyclo[3.1.0]hexan-1-amine HCl in Step A in place of 1-(fluoromethyl)cyclopropanamine hydrochloride. MS obsd. (ESI⁺): 447.0 [(M+H)⁺]. ¹H NMR (400 MHz, DMSO-d₆) δ: 8.91 (1H), 7.64 (1H), 7.48 (1H), 7.39-7.07 (3H), 6.84 (1H), 5.23-5.16 (1H), 4.10-4.08 (1H), 3.96-3.94 (1H), 3.84-3.80 (2H), 3.24 (3H), 2.26-2.22 (1H), 1.54-1.49 (4H), 1.20 (1H).

### Example 149: (R)-6-(1-acetyl-3-methylazetidin-3-yl)-4-((1-(3-(difluoromethyl)-2-fluorophenyl)ethyl)amino)-2-methylpyrido[3,4-d]pyridazine-1,7(2H,6H)-dione

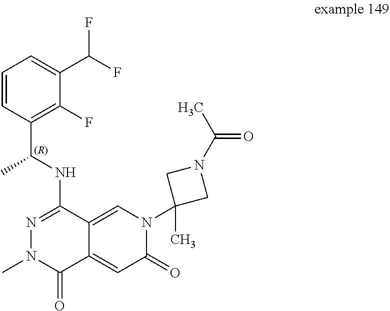

### Step A: tert-butyl 3-(4-chloro-2-methyl-1,7-dioxo-1,2-dihydropyrido[3,4-d]pyridazin-6(7H)-yl)-3-methylazetidine-1-carboxylate

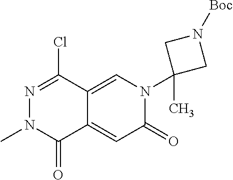 [SMILES: Cn1nc(Cl)c2cn(C3(C)CN(C(=O)OC(C)(C)C)C3)c(=O)cc2c1=O]

Prepared according to an essentially analogous route to example 125 steps A and B using tert-butyl 3-amino-3-methyl-azetidine-1-carboxylate in Step A in place of 1-(fluoromethyl)cyclopropanamine hydrochloride. MS obsd. (ESI⁺): 381.5 [(M+H)⁺].

### Step B: 4-chloro-2-methyl-6-(3-methylazetidin-3-yl)pyrido[3,4-d]pyridazine-1,7(2H,6H)-dione 2,2,2-trifluoroacetic acid

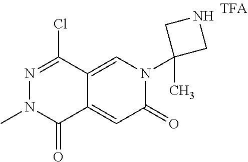 [SMILES: Cn1nc(Cl)c2cn(C3(C)CNC3)c(=O)cc2c1=O]

Tert-butyl 3-(4-chloro-2-methyl-1,7-dioxo-1,2-dihydropyrido[3,4-d]pyridazin-6(7H)-yl)-3-methylazetidine-1-carboxylate (125 mg, 328 μmol) was dissolved in TFA (1.5 mL) at room temperature. The mixture was stirred at room temperature for 0.5 h. The mixture was concentrated in vacuum to give 4-chloro-2-methyl-6-(3-methylazetidin-3-yl)pyrido[3,4-d]pyridazine-1,7(2H,6H)-dione 2,2,2-trifluoroacetic acid (92 mg, crude). The residue was directly used in the next step without further purification. MS obsd. (ESI⁺): 281.1 [(M+H)⁺].

### Step C: 6-(1-acetyl-3-methylazetidin-3-yl)-4-chloro-2-methylpyrido[3,4-d]pyridazine-1,7(2H,6H)-dione

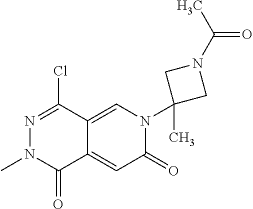 [SMILES: CC(=O)N1CC(C)(n2cc3c(Cl)nn(C)c(=O)c3cc2=O)C1]

To a solution of 4-chloro-2-methyl-6-(3-methylazetidin-3-yl)pyrido[3,4-d]pyridazine-1,7(2H,6H)-dione 2,2,2-trifluoroacetic acid (74 mg, 263 μmol) in DCM (4.0 mL) was added acetyl chloride (25 mg, 315 μmol) at room temperature. Then triethylamine (106 mg, 1.05 mmol) was added to the mixture. The mixture was stirred at room temperature for 1 hr. The reaction mixture was quenched with water and extracted with DCM. The combined organic layers were dried over sodium sulfate, filtered and concentrated. The residue was purified by flash column (eluted with 0-20% MeOH in DCM) to give the title compound (47 mg, 55% yield over two steps). MS obsd. (ESI⁺): 323.1 [(M+H)⁺].

### Step D: (R)-6-(1-acetyl-3-methylazetidin-3-yl)-4-((1-(3-(difluoromethyl)-2-fluorophenyl)ethyl)amino)-2-methylpyrido[3,4-d]pyridazine-1,7(2H,6H)-dione (example 149)

To a solution of (1R)-1-[3-(difluoromethyl)-2-fluoro-phenyl]ethanamine (41 mg, 218 μmol) in 1,4-dioxane (0.5 mL) was added 6-(1-acetyl-3-methylazetidin-3-yl)-4-chloro-2-methylpyrido[3,4-d]pyridazine-1,7(2H,6H)-dione (47 mg, 146 μmol), xantphos-Pd-G3 (28 mg, 29 mol) and Cs₂CO₃ (142 mg, 437 μmol) at room temperature. The reaction was purged stirred at 120° C. for 3 h. The mixture was concentrated. The residue was purified by flash column (eluted with 0-5% MeOH in DCM) followed by reverse phase column (C18, ACN/water) to give the title compound (example 149, 32 mg, 46% yield). MS obsd. (ESI⁺): 476.2 [(M+H)⁺]. ¹H NMR (400 MHz, DMSO-d₆) δ: 8.62 (1H), 7.64 (1H), 7.50 (1H), 7.36-7.06 (3H), 6.84 (1H), 5.21-5.18 (1H), 4.61-4.55 (1H), 4.49 (1H), 4.28-4.25 (1H), 4.02 (1H), 3.25 (3H), 1.83 (3H), 1.77 (3H), 1.55 (3H).

### Example 150: (R)-6-(1-(cyclopropanecarbonyl)-3-methylazetidin-3-yl)-4-((1-(3-(difluoromethyl)-2-fluorophenyl)ethyl)amino)-2-methyl-2,6-dihydropyrido[3,4-d]pyridazine-1,7-dione

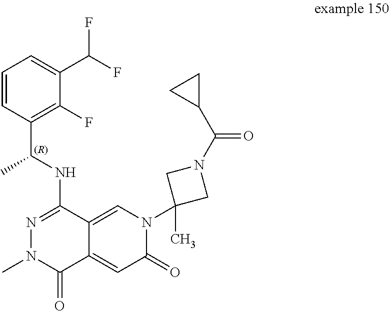

Prepared according to an essentially analogous route to example 149 steps C and D with cyclopropanecarbonyl chloride in Step C in place of acetyl chloride. MS obsd. (ESI⁺): 502.2 [(M+H)⁺]. ¹H NMR (400 MHz, DMSO-d₆) δ: 8.62 (1H), 7.65 (1H), 7.48 (1H), 7.36-7.06 (3H), 6.84 (1H), 5.23-5.16 (1H), 4.71-4.65 (1H), 4.52-4.41 (2H), 4.02 (1H), 3.25 (3H), 1.80 (3H), 1.63-1.58 (1H), 1.53 (3H), 0.78-0.67 (4H).

### Example 151: (R)-4-((1-(3-(difluoromethyl)-2-fluorophenyl)ethyl)amino)-6-(1-(1-methoxycyclopropane-1-carbonyl)-3-methylazetidin-3-yl)-2-methyl-2,6-dihydropyrido[3,4-d]pyridazine-1,7-dione

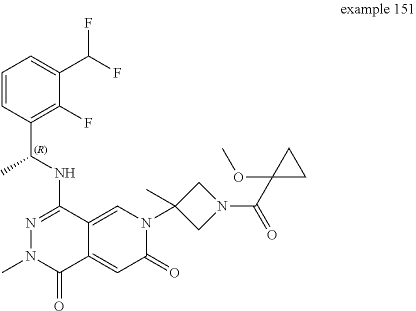

### Step A: 4-chloro-6-(1-(1-methoxycyclopropane-1-carbonyl)-3-methylazetidin-3-yl)-2-methyl-2,6-dihydropyrido[3,4-d]pyridazine-1,7-dione

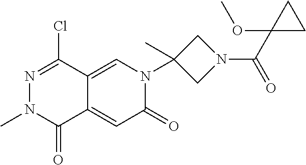 [SMILES: COC1(C(=O)N2CC(C)(n3cc4c(Cl)nn(C)c(=O)c4cc3=O)C2)CC1]

1-methoxycyclopropanecarboxylic acid (9 mg, 74 μmol) was dissolved in anhydrous DMF (0.5 mL), HATU (56 mg, 148 μmol), DIPEA (26 μL, 148 μmol) was added. The mixture was stirred for 20 min at rt. Then 4-chloro-2-methyl-6-(3-methylazetidin-3-yl)-2,6-dihydropyrido[3,4-d]pyridazine-1,7-dione “example 149/step B” (25 mg, 89 μmol) was added, the reaction mixture was stirred at rt for 16 hrs. The mixture was dissolved in DCM, washed with water, brine and dried over Na₂SO₄, the solvent was removed in vacuum, the residue was purified by pre-TLC (CH₃OH/DCM=1:6) to afford the title compound (20 mg, 71% yield). MS obsd. (ESI⁺): 379.3 [(M+H)⁺].

### Step B: (R)-4-((1-(3-(difluoromethyl)-2-fluorophenyl)ethyl)amino)-6-(1-(1-methoxycyclopropane-1-carbonyl)-3-methylazetidin-3-yl)-2-methyl-2,6-dihydropyrido[3,4-d]pyridazine-1,7-dione (example 151)

4-chloro-6-(1-(1-methoxycyclopropane-1-carbonyl)-3-methylazetidin-3-yl)-2-methyl-2,6-dihydropyrido[3,4-d]pyridazine-1,7-dione (20 mg, 52 μmol), (1R)-1-[3-(difluoromethyl)-2-fluoro-phenyl]ethanamine; hydrochloride (17.87 mg, 79 μmol), Xantphos-Pd-G3 (15 mg, 16 μmol), and cesium carbonate (51.6 mg, 158 μmol) were dissolved in dioxane (2 mL). The mixture was stirred at 100° C. for 2 hr. The mixture was filtered and washed with EA. The solution was concentrated under reduced pressure and the crude residue was purified by flash column (MeOH/DCM=1/30) to obtain the title compound (3 mg, 10% yield). MS obsd. (ESI+): 532.4 [(M+H)+]. 1H NMR (400 MHz, DMSO-d6) δ: 8.64 (1H), 7.65 (1H), 7.49 (1H), 7.37-7.09 (3H), 6.85 (1H), 5.26-5.11 (1H), 4.79 (1H), 4.52 (1H), 4.43 (1H), 4.07 (1H), 3.28 (3H), 3.25 (3H), 1.80 (3H), 1.54 (3H), 1.24 (2H), 0.99 (2H).

### Example 152: (R)-4-((1-(3-(difluoromethyl)-2-fluorophenyl)ethyl)amino)-2-methyl-6-(3-methylazetidin-3-yl)pyrido[3,4-d]pyridazine-1,7(2H,6H)-dione

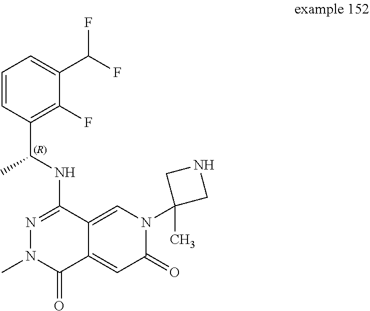

### Step A: (R)-tert-butyl-3-(4-((1-(3-(difluoromethyl)-2-fluorophenyl)ethyl)amino)-2-methyl-1,7-dioxo-1,2-dihydropyrido[3,4-d]pyridazin-6(7H)-yl)-3-methylazetidine-1-carboxylate

Prepared according to an essentially analogous route to example 125 using tert-butyl 3-amino-3-methylazetidine-1-carboxylate in place of 1-(fluoromethyl)cyclopropanamine hydrochloride. MS obsd. (ESI⁺): 534.3 [(M+H)⁺].

### Step B: (R)-4-((1-(3-(difluoromethyl)-2-fluorophenyl)ethyl)amino)-2-methyl-6-(3-methylazetidin-3-yl)pyrido[3,4-d]pyridazine-1,7(2H,6H)-dione (example 152)

To a solution of (R)-tert-butyl 3-(4-((1-(3-(difluoromethyl)-2-fluorophenyl)ethyl)amino)-2-methyl-1,7-dioxo-1,2-dihydropyrido[3,4-d]pyridazin-6(7H)-yl)-3-methylazetidine-1-carboxylate (105 mg, 0.20 mmol) in dioxane (2.5 mL) was added HCl/dioxane (4 M, 2.5 mL) and the mixture was stirred at room temperature for 2 h. The reaction mixture was concentrated and NaHCO₃ (aq.) was added to the residue until pH=8-9 was reached. The solution was extracted with ethyl acetate and the organic layers were dried over sodium sulfate, filtered and concentrated. The residue was purified by flash chromatography (eluting with 0-20% MeOH in DCM) followed by preparative HPLC (eluting with 0-60% MeCN in water) to afford the title compound (example 152, 48 mg, 51% yield). MS obsd. (ESI⁺): 434.7 [(M+H)⁺]. ¹H NMR (400 MHz, DMSO-d6) δ ppm: 8.36 (1H), 7.59 (1H), 7.51-7.44 (1H), 7.37-7.04 (2H), 6.71 (1H), 6.51 (1H), 5.41 (1H), 5.18-5.08 (1H), 3.81 (1H), 3.57-3.50 (2H), 3.47-3.42 (m, 1H), 3.15 (3H), 1.48 (3H), 1.44 (3H).

### Example 153: (R)-4-((1-(3-(difluoromethyl)-2-fluorophenyl)ethyl)amino)-6-(1-(1-fluorocyclopropane-1-carbonyl)-3-methylazetidin-3-yl)-2-methyl-2,6-dihydropyrido[3,4-d]pyridazine-1,7-dione

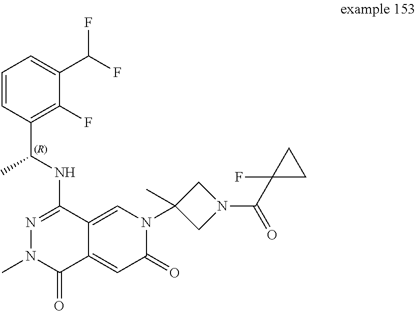

Prepared according to an essentially analogous route to example 146 with (R)-4-((1-(3-(difluoromethyl)-2-fluorophenyl)ethyl)amino)-2-methyl-6-(3-methylazetidin-3-yl)-2,6-dihydropyrido[3,4-d]pyridazine-1,7-dione in place of (R)-6-(2-azabicyclo[2.1.1]hexan-4-yl)-4-((1-(3-(difluoromethyl)-2-fluorophenyl)ethyl)amino)-2-methyl-2,6-dihydropyrido[3,4-d]pyridazine-1,7-dione and 1-fluorocyclopropanecarboxylic acid in place of acetic acid. MS obsd. (ESI⁺): 520.4 [(M+H)⁺]. ¹H NMR (400 MHz, DMSO-d₆) δ: 8.65 (1H), 7.65 (1H), 7.49 (1H), 7.38-7.05 (3H), 6.84 (1H), 5.27-5.14 (1H), 4.88 (1H), 4.57 (1H), 4.49 (1H), 4.12 (1H), 3.25 (3H), 1.81 (3H), 1.54 (3H), 1.32 (2H), 1.24 (2H).

### Example 154: (R)-6-(1-acetyl-3-(difluoromethyl)azetidin-3-yl)-4-((1-(3-(difluoromethyl)-2-fluorophenyl)ethyl)amino)-2-methylpyrido[3,4-d]pyridazine-1,7(2H,6H)-dione

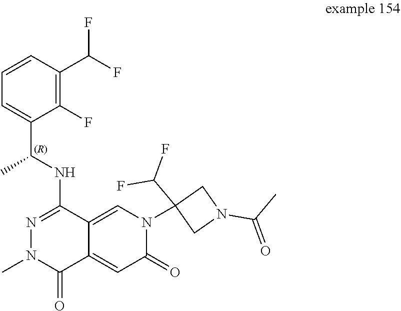

### Step A: 1-acetylazetidin-3-one

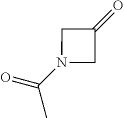

To a solution of azetidin-3-one hydrochloride (9.0 g, 83.69 mmol) in DCM (225 mL) was added Ac₂O (17.1 g, 167 mmol) at room temperature. Then triethylamine (21.2 g, 209 mmol) was added to the mixture. The mixture was stirred at room temperature for 1 h. The mixture was concentrated. The residue was purified by flash chromatography (eluted with 0-80% EA in PE) to give the title compound (8.0 g, 85% yield). ¹H NMR (400 MHz, CDCl₃) δ: 4.87 (2H), 4.77 (2H), 2.08 (3H).

### Step B: N-(1-acetylazetidin-3-ylidene)-2-methylpropane-2-sulfinamide

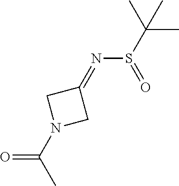

To a solution of 1-acetylazetidin-3-one (8.0 g, 70.72 mmol) in THF (160 mL) was added 2-methylpropane-2-sulfinamide (10.3 g, 84.9 mmol) and titanium tetraethoxide (48.4 g, 212 mmol) at room temperature. The mixture was stirred at 80° C. for 3 hr. The mixture was quenched with NH₄Cl (aq.). The reaction mixture was filtered and the filtrate was extrated with DCM. The organic layers were dried over sodium sulfate, filtered and concentrated. The residue was purified by flash chromatography (eluted with 0-5% MeOH in DCM) to give the title compound.

¹H NMR (400 MHz, CDCl₃) δ: 5.35 (1H), 5.20-5.15 (1H), 4.94-4.83 (2H), 1.99 (3H), 1.28 (9H).

### Step C: N-(1-acetyl-3-(difluoro(phenylsulfonyl)methyl)azetidin-3-yl)-2-methylpropane-2-sulfinamide

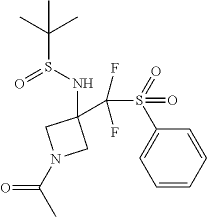 [SMILES: CC(=O)N1CC(NS(=O)C(C)(C)C)(C(F)(F)S(=O)(=O)c2ccccc2)C1]

To a solution of N-(1-acetylazetidin-3-ylidene)-2-methylpropane-2-sulfinamide (1.9 g, 8.78 mmol) in THE (40 mL) was added difluoromethylsulfonylbenzene (1.7 g, 8.78 mmol) at room temperature, then LiHDMS (1 M in THF, 10.5 mL) was added to the mixture at −78° C. The mixture was stirred at room temperature for 1 h. The mixture was quenched with NH₄Cl (aq.) and extracted with EA. The organic layers were dried over sodium sulfate, filtered and concentrated. The residue was purified by flash chromatography (eluted with 0-5% MeOH in DCM) to give the title compound (700 mg, 20% yield). MS obsd. (ESI⁺): 409.1 [(M+H)⁺].

### Step D: N-(1-acetyl-3-(difluoromethyl)azetidin-3-yl)-2-methylpropane-2-sulfinamide

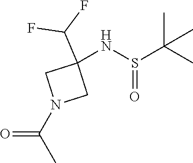 [SMILES: CC(=O)N1CC(NS(=O)C(C)(C)C)(C(F)F)C1]

To a mixture of N-(1-acetyl-3-(difluoro(phenylsulfonyl)methyl)azetidin-3-yl)-2-methylpropane-2-sulfinamide (700 mg, 1.71 mmol) in DMF (21 mL) and NaOAc/HOAc (8 M, 2.1 mL) was added Mg (208 mg, 8.57 mmol) one portion. The resulting mixture was stirred at room temperature for 1 h. The mixture was diluted with ethyl acetate and NH₄Cl (aq.). The organic phase was separated and the aqueous phase was extracted with ethyl acetate. The combined organic layers were dried over sodium sulfate, filtered and concentrated. The residue was purified by flash chromatography (eluted with 0-5% MeOH in DCM) to give the title compound (289 mg, 63% yield). MS obsd. (ESI⁺): 269.1 [(M+H)⁺].

### Step E: 1-(3-amino-3-(difluoromethyl)azetidin-1-yl)ethanone

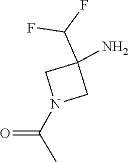

To a mixture of N-(1-acetyl-3-(difluoromethyl)azetidin-3-yl)-2-methylpropane-2-sulfinamide (289 mg, 1.08 mmol) in 1,4-dioxane (2.0 mL) was added HCl (4 M in 1,4-dioxane, 2.2 mL) at room temperature. The resulting mixture was stirred for 3 hr. The mixture was concentrated to afford the title compound HCl salt (216 mg, crude).

This residue was dissolved in in DCM (5.0 mL), to which was added NH₃ (7 M in MeOH, 308 μL) at room temperature. The mixture was stirred at room temperature for 1 h. The mixture was filtered and the filtrate was concentrated under vacuum to give the title compound (177 mg, crude). ¹H NMR (400 MHz, CDCl₃) δ: 5.95 (1H), 4.28 (1H), 4.15 (1H), 3.87 (1H), 3.75 (1H), 1.90 (3H).

### Step F: N-(1-acetyl-3-(difluoromethyl)azetidin-3-yl)-2-(6-chloro-5-(hydroxymethyl)-2-methyl-3-oxo-2,3-dihydropyridazin-4-yl)acetamide

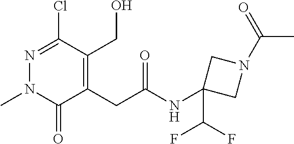 [SMILES: CC(=O)N1CC(NC(=O)Cc2c(CO)c(Cl)nn(C)c2=O)(C(F)F)C1]

To a solution of 4-chloro-2-methyl-5,8-dihydropyrano[3,4-d]pyridazine-1,7-dione (105 mg, 489.57 μmol, 1.0 eq.) in AcOH (35 mg, 587.48 μmol, 1.2 eq.) was added 1-(3-amino-3-(difluoromethyl)azetidin-1-yl)ethanone (88 mg, 538.52 μmol, 1.1 eq.) at room temperature. The mixture was purged with N₂, stirred at 110° C. for 2 h. The mixture was concentrated in vacuum. The residue was purified by flash chromatography (eluted with 0-5% MeOH in DCM) to give the title compound (124 mg, 327.39 μmol, 67% yield over 3 steps). MS obsd. (ESI⁺): 379.2 [(M+H)⁺].

### Step G: (R)-6-(1-acetyl-3-(difluoromethyl)azetidin-3-yl)-4-((1-(3-(difluoromethyl)-2-fluorophenyl)ethyl)amino)-2-methylpyrido[3,4-d]pyridazine-1,7(2H,6H)-dione (example 154)

Prepared according to an essentially analogous route to example 127 steps B and C using N-(1-acetyl-3-(difluoromethyl)azetidin-3-yl)-2-(6-chloro-5-(hydroxymethyl)-2-methyl-3-oxo-2,3-dihydropyridazin-4-yl)acetamide in Step B in place of 2-(6-chloro-5-(hydroxymethyl)-2-methyl-3-oxo-2,3-dihydropyridazin-4-yl)-N-(1-methyl-2-oxabicyclo[2.1.1]hexan-4-yl)acetamide. MS obsd. (ESI⁺): 512.2 [(M+H)⁺]. ¹H NMR (400 MHz, DMSO-d₆) δ: 8.63 (1H), 7.63 (1H), 7.51 (1H), 7.36-7.09 (3H), 6.92 (1H), 6.69 (1H), 5.24-5.17 (1H), 4.72-4.59 (3H), 4.32 (1H), 3.26 (3H), 1.85 (3H), 1.55 (3H).

### Example 155: (R)-4-((1-(3-(difluoromethyl)-2-fluorophenyl)ethyl)amino)-2-methyl-6-(3-(trifluoromethyl)bicyclo[1.1.1]pentan-1-yl)pyrido[3,4-d]pyridazine-1,7(2H,6H)-dione

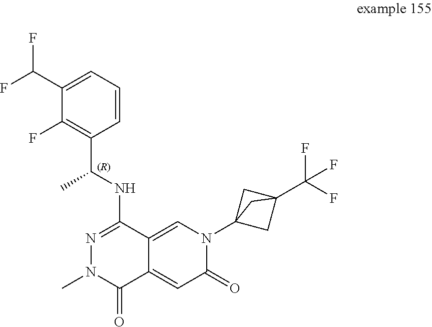

Prepared according to an essentially analogous route to example 127 using tert-butyl 1-amino-3-azabicyclo[3.1.0]hexane-3-carboxylate in Step A in place of 1-methyl-2-oxabicyclo[2.1.1]hexan-4-amine hydrochloride. MS obsd. (ESI+): 499.2 (M+H)+. ¹H NMR (400 MHz, DMSO-d₆) δ: 8.56 (1H), 7.65 (1H), 7.51 (1H), 7.36-7.09 (3H), 6.83 (1H), 5.24-5.18 (1H), 3.25 (3H), 3.71 (6H), 1.55 (3H).

### Example 156: (R)-4-((1-(3-(difluoromethyl)-2-fluorophenyl)ethyl)amino)-2-methyl-6-(3-(trifluoromethyl)oxetan-3-yl)-2,6-dihydropyrido[3,4-d]pyridazine-1,7-dione

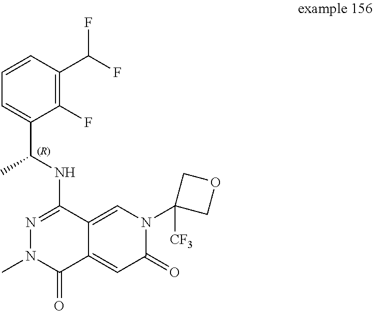

### Step A: 2-methyl-N-(oxetan-3-ylidene)propane-2-sulfinamide

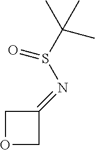

To a solution of oxetan-3-one (10 g, 138.77 mmol) in THE (30 mL) was added 2-methylpropane-2-sulfinamide (20.2 g, 167 mmol) and tetraethoxy titanium (63.3 g, 278 mmol). The reaction was stirred for 24 h at 60° C. To the reaction mixture was added ice water (aq., 500 mL) and the mixture was stirred for 20 min. The mixture was filtered and the filtrate was extracted with DCM (3*300 mL). The combined organic layers were dried over Na₂SO₄. The solvent was removed in vacuo and the residue was purified by chromatography (eluting with 0%-70% EA in PE) to afford the title compound (8.1 g, 33% yield). MS obsd. (ESI+): 176.1 (M+H)+.

### Step B: 2-methyl-N-(3-(trifluoromethyl)oxetan-3-yl)propane-2-sulfinamide

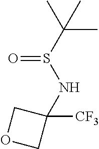

To a solution of 2-methyl-N-(oxetan-3-ylidene)propane-2-sulfinamide (8 g, 45.65 mmol) in THF (100 mL) was added trimethyl(trifluoromethyl)silane (10.4 g, 73.0 mmol) and TBAF (1 M, 41.1 mL) at −30° C. The reaction was stirred for 3 h at −30° C. The reaction was quenched by NH₄Cl (aq., 100 mL) and the mixture was extracted with DCM (2*100 mL). The combined organic layers were dried over Na₂SO₄ and the solvent was removed in vacuo. The residue was purified by flash column chromatography (eluting with 30%-70% EA in PE) to afford the title compound (1.1 g, 9.8% yield). MS obsd. (ESI+): 246.2 (M+H)+.

### Step C: 3-(trifluoromethyl)oxetan-3-amine hydrochloride

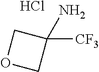

To a solution of 2-methyl-N-(3-(trifluoromethyl)oxetan-3-yl)propane-2-sulfinamide (1.1 g, 4.5 mmol) in dioxane (15 mL) was added HCl (4 M, 5.61 mL). The reaction was stirred for 2 h at rt. The solvent was removed in vacuo and the resulting solid was triturated with acetonitrile (2×10 mL) to afford the title compound (710 mg, 89.2% yield). ¹H NMR (400 MHz, DMSO-d6) δ ppm 9.57 (3H), 4.90 (2H), 4.75 (2H).

### Step D: 3-(trifluoromethyl)oxetan-3-amine

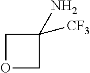

To a solution of 3-(trifluoromethyl)oxetan-3-amine hydrochloride (680 mg, 3.83 mmol) in H₂O (10 mL) was added NaOH (1 M, 7.66 mL). The reaction was stirred for 0.5 h at rt. The mixture was extracted with ethyl ether (3*20 mL) and the combined organic layer was dried over Na₂SO₄. The solvent was removed in vacuo to afford the title compound (310 mg, crude). ¹H NMR (400 MHz, DMSO-d6) δ ppm 4.61 (2H), 4.42 (2H), 2.73 (2H).

### Step E: 2-(6-chloro-5-(hydroxymethyl)-2-methyl-3-oxo-2,3-dihydropyridazin-4-yl)-N-(3-(trifluoromethyl)oxetan-3-yl)acetamide

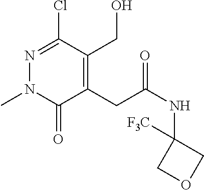 [SMILES: Cn1nc(Cl)c(CO)c(CC(=O)NC2(C(F)(F)F)COC2)c1=O]

To 3-(trifluoromethyl)oxetan-3-amine (200 mg, 1.42 mmol) was added acetic acid (79 mg, 1.31 mmol) and 4-chloro-2-methyl-5,8-dihydropyrano[3,4-d]pyridazine-1,7-dione (234 mg, 1.09 mmol). The reaction was stirred for 1 h at 110° C. The mixture was concentrated and purified by flash column chromatography (eluting with 0%-5% MeOH in DCM) to afford the title compound (174 mg, 45% yield). MS obsd. (ESI+): 356.2 (M+H)+.

### Step F: (R)-4-((1-(3-(difluoromethyl)-2-fluorophenyl)ethyl)amino)-2-methyl-6-(3-(trifluoromethyl)oxetan-3-yl)-2,6-dihydropyrido[3,4-d]pyridazine-1,7-dione (example 156)

The title compound was prepared in an analagous manner as example 127 steps B and C. MS obsd. (ESI⁺): 489.3 [(M+H)⁺]. ¹H NMR (400 MHz, DMSO-d6) δ ppm 8.54 (1H), 7.60 (1H), 7.49 (1H), 7.36-7.09 (3H), 6.92 (1H), 5.36-5.13 (3H), 4.97 (2H), 3.25 (3H), 1.54 (3H).

### Examples 157 and 158: 4-(((R)-1-(3-(difluoromethyl)-2-fluorophenyl)ethyl)amino)-8-methoxy-2-methyl-6-((S)-3-(trifluoromethyl)tetrahydrofuran-3-yl)-2,6-dihydropyrido[3,4-d]pyridazine-1,7-dione(example 157, stereochemistry at tetrahydropyran is arbitrarily assigned) and 4-(((R)-1-(3-(difluoromethyl)-2-fluorophenyl)ethyl)amino)-8-methoxy-2-methyl-6-((R)-3-(trifluoromethyl)tetrahydrofuran-3-yl)-2,6-dihydropyrido[3,4-d]pyridazine-1,7-dione (example 158, stereochemistry at tetrahydropyran is arbitrarily assigned)

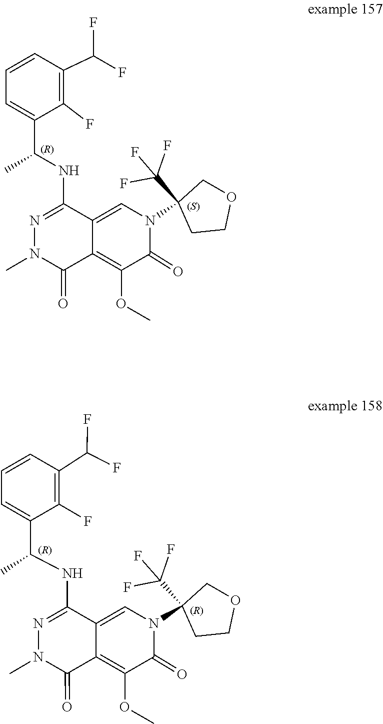

### Step A: 8-bromo-4-[[(1R)-1-[3-(difluoromethyl)-2-fluoro-phenyl]ethyl]amino]-2-methyl-6-[3-(trifluoromethyl)tetrahydrofuran-3-yl]pyrido[3,4-d]pyridazine-1,7-dione

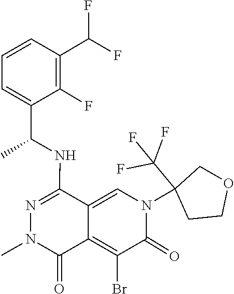 [SMILES: C[C@@H](Nc1nn(C)c(=O)c2c(Br)c(=O)n(C3(C(F)(F)F)CCOC3)cc12)c1cccc(C(F)F)c1F]

The title compound was prepared according to an analogous procedure as example 86 step A.

### Step B: 4-(((R)-1-(3-(difluoromethyl)-2-fluorophenyl)ethyl)amino)-8-methoxy-2-methyl-6-((S)-3-(trifluoromethyl)tetrahydrofuran-3-yl)-2,6-dihydropyrido[3,4-d]pyridazine-1,7-dione (example 157, stereochemistry at tetrahydropyran is arbitrarily assigned) and 4-(((R)-1-(3-(difluoromethyl)-2-fluorophenyl)ethyl)amino)-8-methoxy-2-methyl-6-((R)-3-(trifluoromethyl)tetrahydrofuran-3-yl)-2,6-dihydropyrido[3,4-d]pyridazine-1,7-dione (example 158, stereochemistry at tetrahydropyran is arbitrarily assigned)

To a solution of 8-bromo-4-[[(1R)-1-[3-(difluoromethyl)-2-fluoro-phenyl]ethyl]amino]-2-methyl-6-[3-(trifluoromethyl)tetrahydrofuran-3-yl]pyrido[3,4-d]pyridazine-1,7-dione (400 mg, 0.68 mmol) in MeOH (4 mL) was added sodium methanolate (222 mg, 4.13 mmol). The reaction was stirred for 1 h at 70° C. in a microwave reactor. The reaction was quenched by NH₄Cl (aq) and the mixture was extracted with DCM (3*10 mL). The combined organic layers were dried over Na₂SO₄. The solvent was removed in vacuo and the residue was purified by preparativeTLC (DCM/MeOH=20/1) and chiral-SFC (EtOH [1% NH₃ (7 M in MeOH)]) to afford 4-(((R)-1-(3-(difluoromethyl)-2-fluorophenyl)ethyl)amino)-8-methoxy-2-methyl-6-((S)-3-(trifluoromethyl)tetrahydrofuran-3-yl)-2,6-dihydropyrido[3,4-d]pyridazine-1,7-dione (example 157, stereochemistry at tetrahydropyran is arbitrarily assigned, 25 mg, 14% yield) and 4-(((R)-1-(3-(difluoromethyl)-2-fluorophenyl)ethyl)amino)-8-methoxy-2-methyl-6-((R)-3-(trifluoromethyl)tetrahydrofuran-3-yl)-2,6-dihydropyrido[3,4-d]pyridazine-1,7-dione (example 158, stereochemistry at tetrahydropyran is arbitrarily assigned, 21.4 mg, 11% yield). Chiral HPLC: Column Name: OD-3 4.6*100 mm 3.0 um, EtOH[1% NH3(7M in MeOH)], flow rate: 3 mL/min, temp: 40° C.

Example 157: Chiral HPLC retention time=1. 17 min. MS obsd. (ESI⁺): 533.3 [(M+H)⁺]. ¹H NMR (400 MHz, DMSO-d₆) δ: ppm 8.18 (s, 1H), 7.60 (t, J=7.4 Hz, 1H), 7.48 (t, J=6.8 Hz, 1H), 7.37-7.08 (m, 3H), 5.19 (p, J=6.8 Hz, 1H), 4.99 (d, J=10.8 Hz, 1H), 4.21-4.12 (m, 1H), 4.02-3.98 (m, 1H), 3.96-3.91 (m, 1H), 3.75 (s, 3H), 3.17 (s, 3H), 3.10-3.01 (m, 1H), 2.92-2.81 (m, 1H), 1.53 (d, J=7.0 Hz, 3H).

Example 158: Chiral HPLC retention time=2.60 min. MS obsd. (ESI⁺): 533.3 [(M+H)⁺]. ¹H NMR (400 MHz, DMSO-d₆) δ: ppm 8.16 (s, 1H), 7.57 (t, J=7.2 Hz, 1H), 7.48 (t, J=6.8 Hz, 1H), 7.36-7.09 (m, 3H), 5.17 (p, J=6.8 Hz, 1H), 4.98 (d, J=10.8 Hz, 1H), 4.26-4.13 (m, 1H), 4.03-3.97 (m, 1H), 3.93-3.90 (m, 1H), 3.75 (s, 3H), 3.18 (s, 3H), 3.10-2.99 (m, 1H), 2.91-2.79 (m, 1H), 1.52 (d, J=7.0 Hz, 3H).

### Example 159: 6-cyclopropyl-4-((1-(3-fluoro-5-(trifluoromethyl)phenyl)ethyl)amino)-2-methylpyrido[3,4-d]pyridazine-1,7(2H,6H)-dione

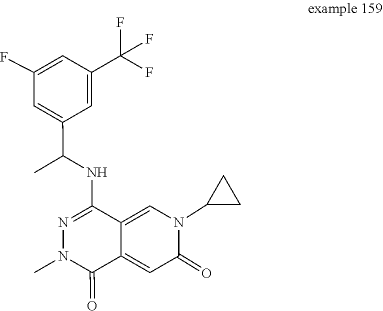 [SMILES: CC(Nc1nn(C)c(=O)c2cc(=O)n(C3CC3)cc12)c1cc(F)cc(C(F)(F)F)c1]

Prepared in an analogous manner as described for examples 96/97 step D. MS obsd. (ESI⁺): 423.1 [(M+H)⁺].

### Example 160: (R)-4-((1-(3,3-difluoro-2,3-dihydrobenzofuran-7-yl)ethyl)amino)-6-(1-(difluoromethyl)cyclopropyl)-2-methyl-2,6-dihydropyrido[3,4-d]pyridazine-1,7-dione

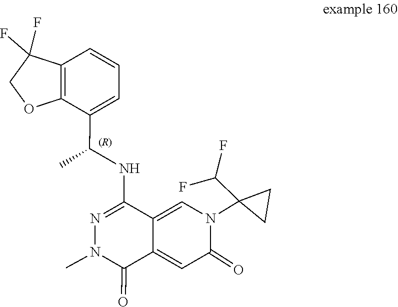

Prepared in an essentially analogous manner to example 70 step C, starting with (R)-1-(3,3-difluoro-2,3-dihydrobenzofuran-7-yl)ethan-1-amine hydrochloride (prepared according to the method of WO/2019/122129). MS obsd. (ESI⁺): 465.0 [(M+H)⁺]. ¹H NMR (400 MHz, DMSO-d₆) δ: 8.90 (1H), 7.53 (1H), 7.48 (1H), 7.12 (1H), 7.09 (1H), 6.88 (1H), 6.30 (1H), 5.13 (1H), 4.85 (2H), 3.26 (3H), 1.51 (3H), 1.49-1.41 (4H).

### Examples 161 and 162: 4-[[(1R)-1-[3-(1,1-difluoro-2-methoxy-ethyl)-2-fluoro-phenyl]ethyl]amino]-6-[1-(difluoromethyl)cyclopropyl]-2-methyl-pyrido[3,4-d]pyridazine-1,7-dione (example 161) and 4-[[(1S)-1-[3-(1,1-difluoro-2-methoxy-ethyl)-2-fluoro-phenyl]ethyl]amino]-6-[1-(difluoromethyl)cyclopropyl]-2-methyl-pyrido[3,4-d]pyridazine-1,7-dione (example 162)

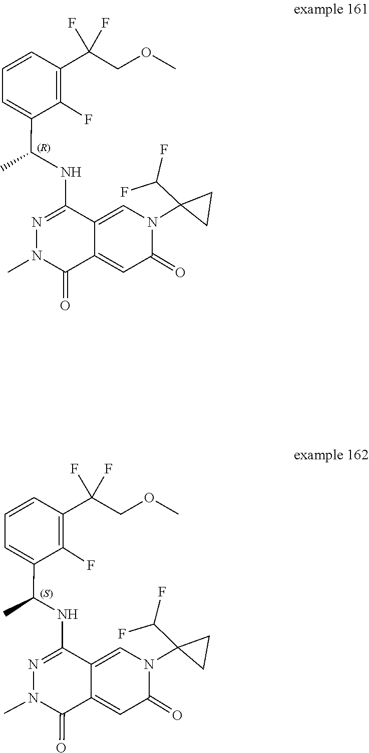

### Step A: 2-(3-bromo-2-fluorophenyl)-2,2-difluoroethan-1-ol

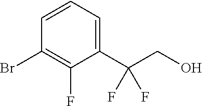 [SMILES: OCC(F)(F)c1cccc(Br)c1F]

To a solution of ethyl 2-(3-bromo-2-fluorophenyl)-2,2-difluoroacetate (1.25 g, 4.21 mmol) in THE (8 mL) was added lithium borohydride (2 M THF, 3.16 mL) at 0° C. The mixture was heated to 50° C. and stirred for 2 hr. Cold water was added and the mixture was extracted with EtOAc. The organic layer was dried over sodium sulfate, filtered and concentrated. The residue was purified by silica gel chromatography column (eluting with 20%-30% EA in PE) to afford the title compound (910 mg, 85% yield).

¹H NMR (400 MHz, CDCl₃) δ: 7.68 (1H), 7.53 (1H), 7.12 (1H), 4.16-4.05 (2H), 2.04 (1H).

### Step B: 1-bromo-3-(1,1-difluoro-2-methoxyethyl)-2-fluorobenzene

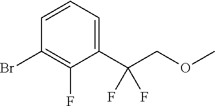

To a solution of 2-(3-bromo-2-fluorophenyl)-2,2-difluoroethan-1-ol (750 mg, 2.94 mmol) in DMF (5 mL) was added NaH (235 mg, 5.88 mmol, 60% in mineral oil) at 0° C. The reaction was stirred for 30 min at 0° C. Iodomethane (626 mg, 4.41 mmol, 1.5 eq.) was added and then the mixture was stirred for 30 min at rt. The mixture was then cooled back to 0° C. and water was added, followed by EA (10 ml). The ethyl acetate fraction was concentrated to dryness and the residue was purified by silica gel column chromatography (eluting with 0%-5% EA in PE) to afford the title compound. ¹H NMR (400 MHz, CDCl₃) δ: 7.66 (1H), 7.52 (1H), 7.11 (1H), 3.95 (2H), 3.43 (3H).

### Step C: 1-(3-(1,1-difluoro-2-methoxyethyl)-2-fluorophenyl)ethan-1-one

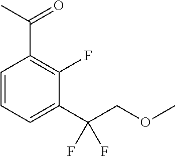 [SMILES: COCC(F)(F)c1cccc(C(C)=O)c1F]

To a solution of 1-bromo-3-(1,1-difluoro-2-methoxyethyl)-2-fluorobenzene (800 mg, 2.97 mmol) in dioxane (5 mL) was added tributyl(1-ethoxyvinyl)stannane (1.29 g, 3.57 mmol), TEA (752 mg, 7.43 mmol) and Bis(Triphenylphosphine)palladium (II) chloride (418 mg, 595 μmol). The reaction was stirred for 16 hr at 100° C. To the mixture was added KF (aq.) and the reaction was stirred for 1 hr at rt. The mixture was then treated with 4 mL HCl (1 M, dioxane). The reaction was stirred for 1 hr at rt. The mixture was neutralized by NaHCO₃ (aq.) and extracted with DCM (3*10 mL). The combined organic layers were dried over Na₂SO₄. The residue was purified by silica gel column chromatography (eluting with 5%-10% EA in PE) to afford the title compound (400 mg, crude). MS obsd. (ESI⁺): 233.2 [(M+H)⁺].

### Step D: N-(1-(3-(1,1-difluoro-2-methoxyethyl)-2-fluorophenyl)ethylidene)-2-methylpropane-2-sulfinamide

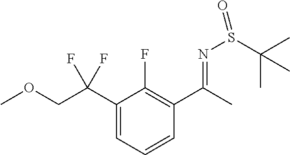 [SMILES: COCC(F)(F)c1cccc(/C(C)=N/S(=O)C(C)(C)C)c1F]

1-(3-(1,1-difluoro-2-methoxyethyl)-2-fluorophenyl)ethan-1-one (400 mg, 1.72 mmol), 2-methylpropane-2-sulfinamide (313 mg, 2.58 mmol) and titanium ethoxide (1.18 g, 5.17 mmol) were dissolved in THE (5 mL). The mixture was stirred at 80° C. for 16 hr under N₂ atmosphere. The mixture was poured into cold water (5 mL), filtered and extracted with EtOAc (20 mL). The organic phase was dried over by Na₂SO₄, concentrated under reduced pressure, and then purified by silica gel column chromatography (eluting with 10%-20% EA in PE) to afford the title compound (300 mg, 52% yield). MS obsd. (ESI⁺): 336.3 [(M+H)⁺].

### Step E: N-(1-(3-(1,1-difluoro-2-methoxyethyl)-2-fluorophenyl)ethyl)-2-methylpropane-2-sulfinamide

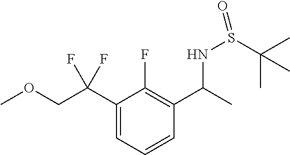 [SMILES: COCC(F)(F)c1cccc(C(C)NS(=O)C(C)(C)C)c1F]

To a solution of N-(1-(3-(1,1-difluoro-2-methoxyethyl)-2-fluorophenyl)ethylidene)-2-methylpropane-2-sulfinamide (300 mg, 894 μmol) in MeOH (2 mL) was added sodium borohydride (51 mg, 1.34 mmol) at 0° C. And the mixture was stirred for 30 min at 0° C. Cold water was poured into the mixture and itw as extracted with DCM. The organic phase was dried over sodium sulfate, filtered and concentrated. The residue was purified by silica gel column chromatography (eluting with 70%-80% EA in PE) to afford the title compound (200 mg, 66% yield). MS obsd. (ESI⁺): 338.3 [(M+H)⁺].

### Step F: 1-[3-(1,1-difluoro-2-methoxy-ethyl)-2-fluoro-phenyl]ethanamine hydrochloride

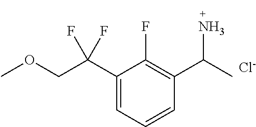 [SMILES: COCC(F)(F)c1cccc(C(C)N)c1F ; [Cl-]]

N-(1-(3-(1,1-difluoro-2-methoxyethyl)-2-fluorophenyl)ethyl)-2-methylpropane-2-sulfinamide (200 mg, 592 μmol) was dissolved in HCl-Dioxane (4M, 2 mL). The solution was stirred at rt for 1 hr. The solvent was removed and the crude residue was suspended in acetonitrile. The mixture was filtered to give the title compound (160 mg, crude). MS obsd. (ESI⁺): 235.3 [(M+H)⁺].

### Step G: 4-((1-(3-(1,1-difluoro-2-methoxyethyl)-2-fluorophenyl)ethyl)amino)-6-(1-(difluoromethyl)cyclopropyl)-2-methyl-2,6-dihydropyrido[3,4-d]pyridazine-1,7-dione

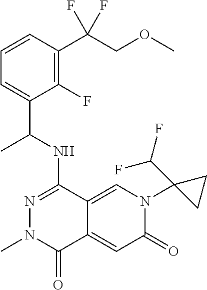 [SMILES: COCC(F)(F)c1cccc(C(C)Nc2nn(C)c(=O)c3cc(=O)n(C4(C(F)F)CC4)cc23)c1F]

Prepared in an essentially analogous manner to example 70 step C, starting with 1-[3-(1,1-difluoro-2-methoxy-ethyl)-2-fluoro-phenyl]ethanamine hydrochloride (step F). MS obsd. (ESI⁺): 499.0 [(M+H)⁺].

### Step H: 4-[[(1R)-1-[3-(1,1-difluoro-2-methoxy-ethyl)-2-fluoro-phenyl]ethyl]amino]-6-[1-(difluoromethyl)cyclopropyl]-2-methyl-pyrido[3,4-d]pyridazine-1,7-dione (example 161) and 4-[[(1S)-1-[3-(1,1-difluoro-2-methoxy-ethyl)-2-fluoro-phenyl]ethyl]amino]-6-[1-(difluoromethyl)cyclopropyl]-2-methyl-pyrido[3,4-d]pyridazine-1,7-dione (example 162)

The racemic mixture was separated by Chiral SFC (column: Daicel OD (25*250 mm, 10 um), CO₂/MeOH[0.2% NH₃(7M in MeOH)]=80/20) to afford 4-[[(1R)-1-[3-(1,1-difluoro-2-methoxy-ethyl)-2-fluoro-phenyl]ethyl]amino]-6-[1-(difluoromethyl)cyclopropyl]-2-methyl-pyrido[3,4-d]pyridazine-1,7-dione (example 161) and 4-[[(1S)-1-[3-(1,1-difluoro-2-methoxy-ethyl)-2-fluoro-phenyl]ethyl]amino]-6-[1-(difluoromethyl)cyclopropyl]-2-methyl-pyrido[3,4-d]pyridazine-1,7-dione (example 162). Analytical Chiral HPLC: Column name: OD-3 4.6*100 mm 3.0 um, MeOH [0.2% NH3(7M in MeOH)], flow rate: 3 mL/min, temp: 40° C.

Example 161: Retention time=1.05 min. MS obsd. (ESI⁺): 499.0 [(M+H)⁺].¹H NMR (400 MHz, DMSO-d6) δ: 8.88 (s, 1H), 7.59 (t, J=7.2 Hz, 1H), 7.41 (t, J=7.0 Hz, 1H), 7.25 (dd, J=14.6, 7.2 Hz, 2H), 6.86 (s, 1H), 6.30 (t, J=56.6 Hz, 1H), 5.18 (p, J=6.8 Hz, 1H), 3.98 (t, J=13.8 Hz, 2H), 3.35 (s, 3H), 3.23 (s, 3H), 1.52 (d, J=6.8 Hz, 5H), 1.41 (s, 2H).

Example 162: Retention time=1.43 min. MS obsd. (ESI⁺): 499.0 [(M+H)⁺]. ¹H NMR (400 MHz, DMSO-d6) δ: 8.88 (s, 1H), 7.59 (t, J=7.2 Hz, 1H), 7.41 (t, J=7.0 Hz, 1H), 7.26 (dd, J=14.6, 7.0 Hz, 2H), 6.86 (s, 1H), 6.30 (t, J=56.4 Hz, 1H), 5.24-5.11 (m, 1H), 3.98 (t, J=14.0 Hz, 2H), 3.36 (s, 3H), 3.23 (s, 3H), 1.52 (d, J=6.8 Hz, 5H), 1.41 (s, 2H).

### Example 163: (R)-6-(1-(difluoromethyl)cyclopropyl)-4-((1-(3-fluorobenzofuran-7-yl)ethyl)amino)-2-methyl-2,6-dihydropyrido[3,4-d]pyridazine-1,7-dione

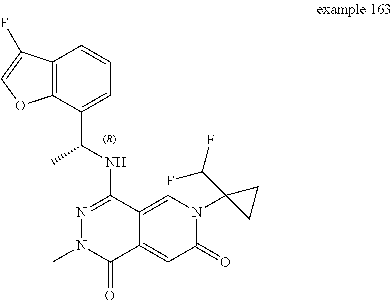

Prepared in an essentially analogous manner to example 70 step C, starting with (R)-1-(3-fluorobenzofuran-7-yl)ethan-1-amine hydrochloride (prepared according to the method of WO/2019/122129). MS obsd. (ESI⁺): 445.0 [(M+H)⁺]. ¹H NMR (400 MHz, DMSO-d₆) δ: 8.92 (1H), 8.29 (1H), 7.54 (1H), 7.42 (1H), 7.31 (1H), 7.24 (1H), 6.87 (1H), 6.30 (1H), 5.46 (1H), 3.19 (3H), 1.61 (3H), 1.51-1.41 (4H).

### Examples 164 and 165: 4-(((R)-1-(3-(difluoromethyl)-2-fluorophenyl)ethyl)amino)-2-methyl-6-((S)-3-(trifluoromethyl)tetrahydrofuran-3-yl)-2,6-dihydropyrido[3,4-d]pyridazine-1,7-dione (example 164, stereochemistry at tetrahydropyran is arbitrarily assigned) and 4-(((R)-1-(3-(difluoromethyl)-2-fluorophenyl)ethyl)amino)-2-methyl-6-((R)-3-(trifluoromethyl)tetrahydrofuran-3-yl)-2,6-dihydropyrido[3,4-d]pyridazine-1,7-dione (example 165, stereochemistry at tetrahydropyran is arbitrarily assigned)

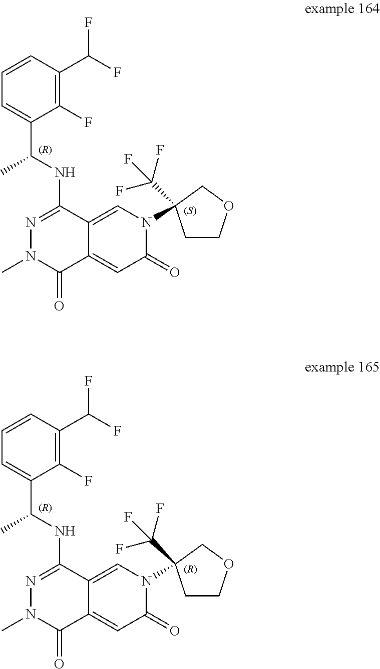

### Step A: 2-(6-chloro-5-(hydroxymethyl)-2-methyl-3-oxo-2,3-dihydropyridazin-4-yl)-N-(3-(trifluoromethyl)tetrahydrofuran-3-yl)acetamide

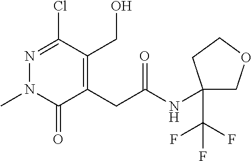 [SMILES: Cn1nc(Cl)c(CO)c(CC(=O)NC2(C(F)(F)F)CCOC2)c1=O]

To 3-(trifluoromethyl)tetrahydrofuran-3-amine (600 mg, 3.87 mmol) was added acetic acid (186 mg, 3.09 mmol) and 4-chloro-2-methyl-5,8-dihydropyrano[3,4-d]pyridazine-1,7-dione (553 mg, 2.58 mmol). The reaction was stirred for 1 h at 110° C. The mixture was then concentrated and purified by flash column chromatography (eluting with 0%-3% MeOH in DCM) to afford the title compound (280 mg, 29%). MS obsd. (ESI⁺): 370.3 [(M+H)⁺].

### Step B: 4-chloro-2-methyl-6-(3-(trifluoromethyl)tetrahydrofuran-3-yl)-2,6-dihydropyrido[3,4-d]pyridazine-1,7-dione

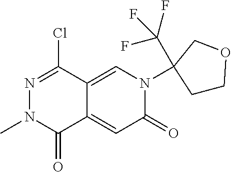 [SMILES: Cn1nc(Cl)c2cn(C3(C(F)(F)F)CCOC3)c(=O)cc2c1=O]

The title compound was prepared in an essentially analogous manner to example 125 step B. MS obsd. (ESI⁺): 350.2 [(M+H)⁺].

### Step C: 4-(((R)-1-(3-(difluoromethyl)-2-fluorophenyl)ethyl)amino)-2-methyl-6-((S)-3-(trifluoromethyl)tetrahydrofuran-3-yl)-2,6-dihydropyrido[3,4-d]pyridazine-1,7-dione (example 164, stereochemistry at tetrahydropyran is arbitrarily assigned) and 4-(((R)-1-(3-(difluoromethyl)-2-fluorophenyl)ethyl)amino)-2-methyl-6-((R)-3-(trifluoromethyl)tetrahydrofuran-3-yl)-2,6-dihydropyrido[3,4-d]pyridazine-1,7-dione (example 165, stereochemistry at tetrahydropyran is arbitrarily assigned)

The diastereomeric mixture was prepared in an essentially analogous manner to example 127 step C and diastereoisomers were separated via chiral SFC. Chiral SFC: (Column: Daicel OD-3 (4.6*100 mm, 3 um), Flow rate: 3.0 mL/min, Eluent:MeOH (0.2% 7M NH3 in MeOH), Temp 400C). Chiral HPLC conditions: Column Name: OD-3 4.6*100 mm 3.0 um, MeOH [0.2% NH3(7M in MeOH)], flow rate: 3 mL/min, temp: 40° C.

Example 164: Chiral HPLC Retention time=1.24 min. MS obsd. (ESI⁺): 503.5 [(M+H)⁺]. ¹H NMR (400 MHz, DMSO-d6) δ ppm 8.59 (1H), 7.62 (1H), 7.49 (1H), 7.36-7.09 (3H), 6.89 (1H), 5.21 (1H), 4.99 (1H), 4.17-4.14 (1H), 4.01 (1H), 3.96-3.90 (1H), 3.24 (3H), 3.09-3.03 (1H), 2.93-2.86 (1H), 1.54 (3H).

Example 165: Chiral HPLC Retention time=2.00 min. MS obsd. (ESI⁺): 503.5 [(M+H)⁺]. ¹H NMR (400 MHz, DMSO-d6) δ ppm 8.57 (1H), 7.60 (1H), 7.49 (1H), 7.36-7.09 (3H), 6.88 (1H), 5.19 (1H), 4.98 (1H), 4.19-4.16 (1H), 4.01 (1H), 3.96-3.90 (1H), 3.24 (3H), 3.11-3.01 (1H), 2.92-2.82 (1H), 1.54 (3H).

### Examples 166 and 167: 4-(((R)-1-(3-fluorobenzofuran-7-yl)ethyl)amino)-2-methyl-6-((S)-3-(trifluoromethyl)tetrahydrofuran-3-yl)-2,6-dihydropyrido[3,4-d]pyridazine-1,7-dione (example 166, stereochemistry at tetrahydropyran is arbitrarily assigned) and 4-(((R)-1-(3-fluorobenzofuran-7-yl)ethyl)amino)-2-methyl-6-((R)-3-(trifluoromethyl)tetrahydrofuran-3-yl)-2,6-dihydropyrido[3,4-d]pyridazine-1,7-dione (167, stereochemistry at tetrahydropyran is arbitrarily assigned)

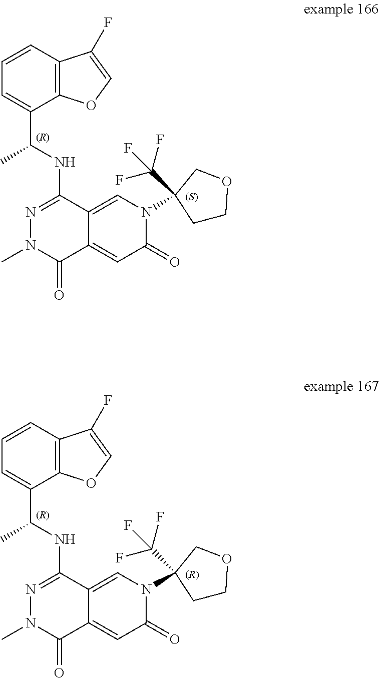

The diastereomeric mixture was prepared in an essentially analogous manner to examples 164/165 step C, starting with (R)-1-(3-fluorobenzofuran-7-yl)ethan-1-amine hydrochloride (prepared according to the method of WO/2019/122129). Diastereomers were separated via chiral SFC. Chiral SFC: (Column: Daicel AD-3 (4.6*100 mm, 3 um), Flow rate: 3.0 mL/min, Eluent: CO2/MeOH[0.2% NH3(7M in MeOH)]=85/15, Temp 40° C.). Chiral HPLC: Column Name: AD-3 4.6*100 mm 3.0 um, MeOH [0.2% NH3(7M in MeOH)], flow rate: 3 mL/min, temp: 40° C.

Example 166: Chiral HPLC retention time=1.44 min. MS obsd. (ESI⁺): 493.0 [(M+H)⁺]. ¹H NMR (400 MHz, DMSO-d₆) δ: 8.61 (1H), 8.30 (1H), 7.54 (1H), 7.43 (1H), 7.34 (1H), 7.30 (1H), 6.89 (1H), 5.49 (1H), 4.99 (1H), 4.16 (1H), 4.01 (1H), 3.96-3.91 (1H), 3.18 (3H), 3.10-3.01 (1H), 2.94-2.82 (1H), 1.62 (3H).

Example 167: Chiral HPLC retention time=2.19 min. MS obsd. (ESI⁺): 493.0 [(M+H)⁺]. ¹H NMR (400 MHz, DMSO-d₆) δ: 8.59 (1H), 8.29 (1H), 7.54 (1H), 7.40 (1H), 7.34 (1H), 7.32 (1H), 6.89 (1H), 5.47 (1H), 4.99 (1H), 4.17 (1H), 4.00 (1H), 4.03-3.90 (1H), 3.19 (3H), 3.13-2.99 (1H), 2.93-2.80 (1H), 1.62 (3H).

### Examples 168 and 169: 4-(((R)-1-(3,3-difluoro-2,3-dihydrobenzofuran-7-yl)ethyl)amino)-2-methyl-6-((S)-3-(trifluoromethyl)tetrahydrofuran-3-yl)-2,6-dihydropyrido[3,4-d]pyridazine-1,7-dione (example 168, stereochemistry at tetrahydropyran is arbitrarily assigned) and 4-(((R)-1-(3,3-difluoro-2,3-dihydrobenzofuran-7-yl)ethyl)amino)-2-methyl-6-((R)-3-(trifluoromethyl)tetrahydrofuran-3-yl)-2,6-dihydropyrido[3,4-d]pyridazine-1,7-dione (example 169, stereochemistry at tetrahydropyran is arbitrarily assigned)

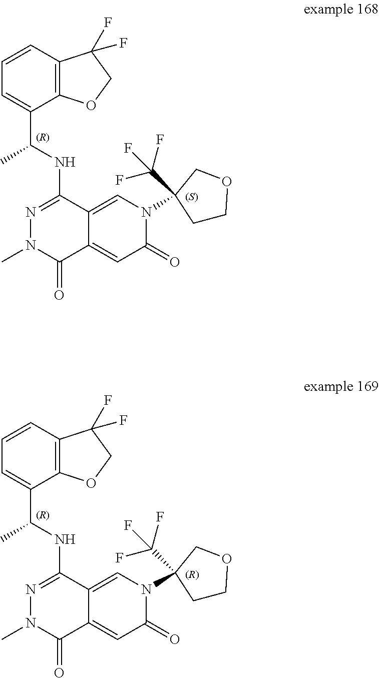

The diastereomeric mixture was prepared in an essentially analogous manner to examples 164/165 step C, starting with (R)-1-(3,3-difluoro-2,3-dihydrobenzofuran-7-yl)ethan-1-amine hydrochloride(prepared according to the method of WO/2019/122129). Chiral SFC: (Column: Daicel OD-3 (4.6*100 mm, 3 um), Flow rate: 3.0 mL/min, Eluent: (CO2/MeOH[0.2% NH3(7M in MeOH)]=75/25), Temp 30° C.). Chiral HPLC: Column Name: OD-3 4.6*100 mm 3.0 um, MeOH [0.2% NH3(7M in MeOH)], flow rate: 3.0 mL/min, temp: 40° C.

Example 168: Chiral HPLC retention time=0.989 min. MS obsd. (ESI⁺): 513.4 [(M+H)⁺]. ¹H NMR (400 MHz, DMSO-d₆) δ: 8.59 (1H), 7.54 (1H), 7.48 (1H), 7.22 (1H), 7.08 (1H), 6.90 (1H), 5.16 (1H), 4.99 (1H), 4.85 (2H), 4.16 (1H), 4.01 (1H), 3.97-3.89 (1H), 3.25 (3H), 3.09-3.03 (1H), 2.98-2.79 (1H), 1.52 (3H).

Example 169: Chiral HPLC retention time=2.13 min. MS obsd. (ESI⁺): 513.4 [(M+H)⁺]. ¹H NMR (400 MHz, DMSO-d₆) δ: 8.57 (1H), 7.50 (2H), 7.22 (1H), 7.10 (1H), 6.89 (1H), 5.14 (1H), 4.99 (1H), 4.85 (2H), 4.17 (1H), 4.00 (1H), 3.96-3.90 (1H), 3.26 (3H), 3.12-3.01 (1H), 2.91-2.80 (1H), 1.52 (3H).

### Examples 170 and 171: 6-((S)-1-acetyl-3-(trifluoromethyl)pyrrolidin-3-yl)-4-(((R)-1-(3,3-difluoro-2,3-dihydrobenzofuran-7-yl)ethyl)amino)-2-methyl-2,6-dihydropyrido[3,4-d]pyridazine-1,7-dione (example 170, stereochemistry at pyrrolidine is arbitrarily assigned) and 6-((R)-1-acetyl-3-(trifluoromethyl)pyrrolidin-3-yl)-4-(((R)-1-(3,3-difluoro-2,3-dihydrobenzofuran-7-yl)ethyl)amino)-2-methyl-2,6-dihydropyrido[3,4-d]pyridazine-1,7-dione (example 171, stereochemistry at pyrrolidine is arbitrarily assigned)

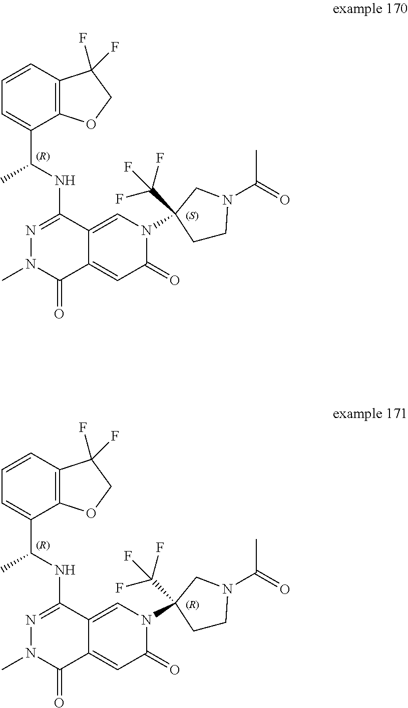

Title compounds were prepared according to an analogous manner as described for examples 100/101 step B, starting with (R)-1-(3,3-difluoro-2,3-dihydrobenzofuran-7-yl)ethan-1-amine hydrochloride (prepared according to the method of WO/2019/122129). Chiral HPLC: (Column: OD-3 (4.6*100 mm, 3 um), Flow rate: 3.0 mL/min, Eluent:MeOH (0.2% 7M NH3 in MeOH), Temp 400C).

Example 170: Chiral HPLC retention time=1.45 min. MS obsd. (ESI⁺): 554.4 [(M+H)⁺].

Example 171: Chiral HPLC retention time=2.33 min. MS obsd. (ESI⁺): 554.4 [(M+H)⁺].

### Examples 172 and 173: 6-((S)-1-acetyl-3-(trifluoromethyl)pyrrolidin-3-yl)-4-(((R)-1-(3-fluorobenzofuran-7-yl)ethyl)amino)-2-methyl-2,6-dihydropyrido[3,4-d]pyridazine-1,7-dione (example 172, stereochemistry at pyrrolidine is arbitrarily assigned) and 6-((R)-1-acetyl-3-(trifluoromethyl)pyrrolidin-3-yl)-4-(((R)-1-(3-fluorobenzofuran-7-yl)ethyl)amino)-2-methyl-2,6-dihydropyrido[3,4-d]pyridazine-1,7-dione (example 173, stereochemistry at pyrrolidine is arbitrarily assigned)

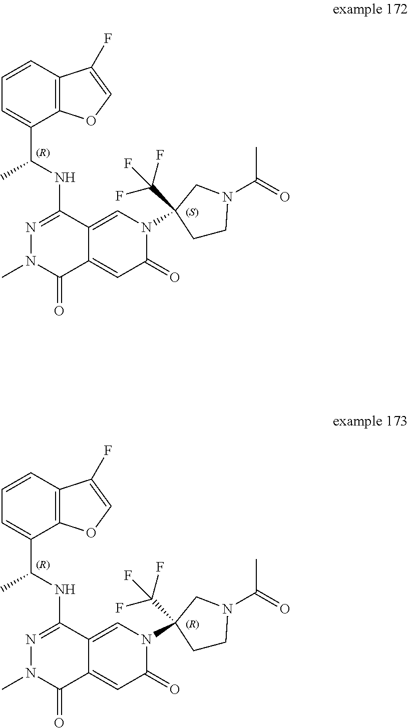

Prepared according to an analogous manner as described for examples 100/101 step B, starting with (R)-1-(3-fluorobenzofuran-7-yl)ethan-1-amine hydrochloride (prepared according to the method of WO/2019/122129). Chiral HPLC: (Column: OD-3 (4.6*100 mm, 3 um), Flow rate: 3.0 mL/min, Eluent:EtOH (1% 7M NH3 in MeOH), Temp 400C).

Example 172: Chiral HPLC retention time=1.47 min. MS obsd. (ESI⁺): 534.4 [(M+H)⁺].

Example 173: Chiral HPLC retention time=2.48 min. MS obsd. (ESI⁺): 534.4 [(M+H)⁺].

### Examples 174 and 175: (R)-4-((1-(3-(difluoro(pyridin-4-yl)methyl)-2-fluorophenyl)ethyl)amino)-6-(1-(difluoromethyl)cyclopropyl)-2-methyl-2,6-dihydropyrido[3,4-d]pyridazine-1,7-dione and (S)-4-((1-(3-(difluoro(pyridin-4-yl)methyl)-2-fluorophenyl)ethyl)amino)-6-(1-(difluoromethyl)cyclopropyl)-2-methyl-2,6-dihydropyrido[3,4-d]pyridazine-1,7-dione

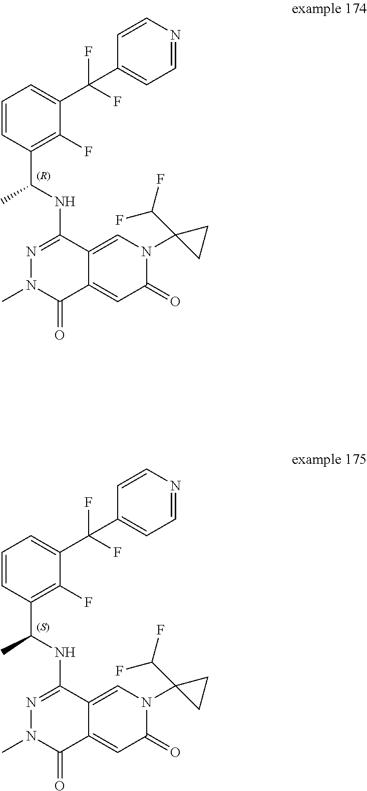

### Step A: N-methoxy-N-methylisonicotinamide

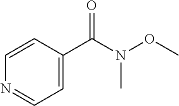

To a stirred solution of isonicotinic acid (30 g, 244 mmol) and N,O-dimethylhydroxylamine hydrochloride (26.2 g, 268 mmol) in DCM (500 mL) was added HATU (102 g, 268 mmol) and DIEA (94.48 g, 731.06 mmol) dropwise at 0° C. The resulting mixture was stirred at room temperature overnight. The reaction was quenched with water (500 mL). The resulting mixture was extracted with CHCl3/i-PrOH (3/1, 3×500 mL). The organic layers were combined, dried over anhydrous sodium sulfate, filtered and concentrated. The residue was purified by silica gel chromatography (0-100% EA/PE) to afford N-methoxy-N-methylisonicotinamide (30 g, 74% yield). MS obsd. (ESI⁺): 167.10 [(M+H)⁺].

### Step B: (3-bromo-2-fluorophenyl)(pyridin-4-yl)methanone

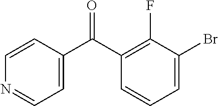 [SMILES: O=C(c1ccncc1)c1cccc(Br)c1F]

To a stirred solution of 1,3-dibromo-2-fluoro-benzene (38.2 g, 150 mmol) in THE (1000 mL) was added n-Butyllithium (60.2 mL, 150 mmol, 2.5 M in THF) at −78° C. under. The resulting solution was stirred for 15 min at −78° C., and then N-methoxy-N-methylisonicotinamide (25 g, 150 mmol) in minimal THE was added dropwise at −78° C. The resulting solution was stirred for 30 min at −78° C. The reaction was quenched with water (500 mL). The resulting mixture was extracted with ethyl acetate (3×1000 mL). The organic layers were combined, dried over anhydrous sodium sulfate, filtered and concentrated. The residue was purified by silica gel chromatography (0-30% EA/PE) to afford (3-bromo-2-fluorophenyl)(pyridin-4-yl)methanone (25 g, 59% yield). MS obsd. (ESI⁺): 279.85, 281.85 [(M+H)⁺].

### Step C: 4-((3-bromo-2-fluorophenyl)difluoromethyl)pyridine

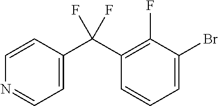 [SMILES: Fc1c(Br)cccc1C(F)(F)c1ccncc1]

To a solution of (3-bromo-2-fluoro-phenyl)-(4-pyridyl)methanone (25 g, 89.3 mmol) in DCM (100 mL) was added DAST (122 g, 757 mmol) at 0° C. under N2 atmosphere. The resulting mixture was stirred at r.t. for 48 h. The resulting mixture was poured into ice water (500 mL) slowly. The pH value of the solution was adjusted to pH 6˜7 with aqueous NaHCO3. The resulting mixture was extracted with ethyl acetate (3×1000 mL). The organic layers were combined, dried over anhydrous sodium sulfate, filtered and concentrated. The residue obtained was purified by silica gel chromatography (0-30% EA/PE) to afford 4-((3-bromo-2-fluorophenyl)difluoromethyl)pyridine (16 g, 59% yield). MS obsd. (ESI⁺): 301.85, 303.85 [(M+H)⁺].

### Step D. 1-(3-(difluoro(pyridin-4-yl)methyl)-2-fluorophenyl)ethan-1-one

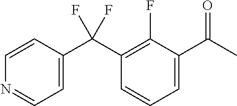 [SMILES: CC(=O)c1cccc(C(F)(F)c2ccncc2)c1F]

To a mixture of 4-((3-bromo-2-fluorophenyl)difluoromethyl)pyridine (4 g, 12.6 mmol) and tributyl(1-ethoxyvinyl)stannane (6.83 g, 18.9 mmol) in dioxane (50 mL) was added Pd(PPh3)2Cl2 (885 mg, 1.26 mmol) and TEA (3.83 g, 37.8 mmol) under N2 atmosphere. The mixture was stirred at 100° C. for 12 h. After cooling to room temperature, HCl (2 M) was added dropwise to pH˜2, and the mixture was stirred for 3 h. The mixture was filtered and the filtrate was extracted with EtOAc (3×200 mL). The combined organic layers were poured into aq. KF (50 mL) and stirred for 20 min. The mixture was filtered and the organic layer was washed with brine and dried over Na2SO4. The solvent was removed under reduced pressure and the residue was purified by silica gel chromatography (0-30% EA/PE) to give 1-(3-(difluoro(pyridin-4-yl)methyl)-2-fluorophenyl)ethan-1-one (2.5 g, 8.92 mmol, 70% yield). MS obsd. (ESI⁺): 266.05 [(M+H)⁺].

### Step E. N-(1-(3-(difluoro(pyridin-4-yl)methyl)-2-fluorophenyl)ethyl)-2-methylpropane-2-sulfinamide

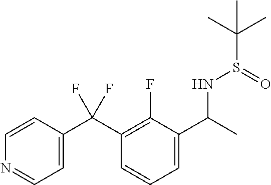 [SMILES: CC(NS(=O)C(C)(C)C)c1cccc(C(F)(F)c2ccncc2)c1F]

A solution of 1-(3-(difluoro(pyridin-4-yl)methyl)-2-fluorophenyl)ethan-1-one (6 g, 22.6 mmol), 2-methylpropane-2-sulfinamide (5.47 g, 45.2 mmol) and Ti(OEt)4 (20.6 g, 90.5 mmol) in THE (100 mL) was stirred for 2 h at 80° C. under N2 atmosphere. After cooling down to room temperature, NaBH4 (1.28 g, 33.9 mmol) and MeOH (2 mL) were added to the mixture and stirred at r.t. for 2 h. The residue was diluted with water and the solid was filtered out. The filtrate was concentrated under vacuum to afford the title compound (5 g, 13.50 mmol, 59% yield). MS obsd. (ESI⁺): 371.05 [(M+H)⁺].

### Step F. 1-(3-(difluoro(pyridin-4-yl)methyl)-2-fluorophenyl)ethan-1-amine hydrochloride

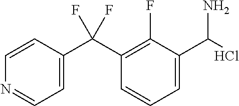 [SMILES: NC(Cl)c1cccc(C(F)(F)c2ccncc2)c1F]

To a solution of N-(1-(3-(difluoro(pyridin-4-yl)methyl)-2-fluorophenyl)ethyl)-2-methylpropane-2-sulfinamide (5 g, 13.5 mmol) in DCM (40 mL) was added HCl (5 mL, 20.0 mmol, 4M in dioxane). The resulting mixture was stirred at r.t. for overnight. The mixture was concentrated under reduced pressure. The residue was purified by Preparative HPLC with the following conditions (Column: Kinetex EVO C18, 21.2*250 mm, 5 mm; Mobile Phase A: Water (0.05% HCl), Mobile Phase B: ACN; Flow rate: 25 mL/min; Gradient: 3% B to 27% B in 9 min, 27% B; Wave Length: 254 nm; RT1(min): 8.72; Number Of Runs: 0) to afford 1-(3-(difluoro(pyridin-4-yl)methyl)-2-fluorophenyl)ethan-1-amine hydrochloride (382 mg, 1.22 mmol, 9.03% yield) as a light yellow solid. 1H NMR (400 MHz, DMSO-d6+D2O) δ 8.77-8.75 (2H), 7.83-7.77 (2H), 7.61-7.58 (2H), 7.51-7.47 (1H), 4.62-4.55 (1H), 2.61 (3H), 1.48 (3H). MS obsd. (ESI⁺): 267.15 [(M+H)⁺].

### Step G: (R)-4-((1-(3-(difluoro(pyridin-4-yl)methyl)-2-fluorophenyl)ethyl)amino)-6-(1-(difluoromethyl)cyclopropyl)-2-methyl-2,6-dihydropyrido[3,4-d]pyridazine-1,7-dione (example 174) (S)-4-((1-(3-(difluoro(pyridin-4-yl)methyl)-2-fluorophenyl)ethyl)amino)-6-(1-(difluoromethyl)cyclopropyl)-2-methyl-2,6-dihydropyrido[3,4-d]pyridazine-1,7-dione (example 175)

Prepared in an essentially analogous manner to example 70 step C, starting with 1-(3-(difluoro(pyridin-4-yl)methyl)-2-fluorophenyl)ethan-1-amine hydrochloride. Chiral HPLC: (Column: OD-3 (4.6*100 mm, 3 um), Flow rate: 3.0 mL/min, Eluent:MeOH (0.2% 7M NH3 in MeOH), Temp 40° C.).

Example 174: Chiral HPLC retention time=1.14 min. MS obsd. (ESI⁺): 532.3 [(M+H)⁺]. ¹H NMR (400 MHz, DMSO-d₆) δ: 8.84 (1H), 8.76 (2H), 7.63 (1H), 7.57 (3H), 7.34 (1H), 7.23 (1H), 6.84 (1H), 6.29 (1H), 5.06 (1H), 2.99 (3H), 1.51 (2H), 1.47 (3H), 1.40 (2H).

Example 175: Chiral HPLC retention time=1.52 min. MS obsd. (ESI⁺): 532.4 [(M+H)⁺]. ¹H NMR (400 MHz, DMSO-d₆) δ: 8.84 (1H), 8.76 (2H), 7.63 (1H), 7.57 (3H), 7.34 (1H), 7.23 (1H), 6.84 (1H), 6.29 (1H), 5.06 (1H), 2.99 (3H), 1.51 (2H), 1.47 (3H), 1.40 (2H).

### Examples 176 and 177: (R)-4-((1-(3-(difluoro(5-methylpyrazin-2-yl)methyl)-2-fluorophenyl)ethyl)amino)-6-(1-(difluoromethyl)cyclopropyl)-2-methyl-2,6-dihydropyrido[3,4-d]pyridazine-1,7-dione and (S)-4-((1-(3-(difluoro(5-methylpyrazin-2-yl)methyl)-2-fluorophenyl)ethyl)amino)-6-(1-(difluoromethyl)cyclopropyl)-2-methyl-2,6-dihydropyrido[3,4-d]pyridazine-1,7-dione

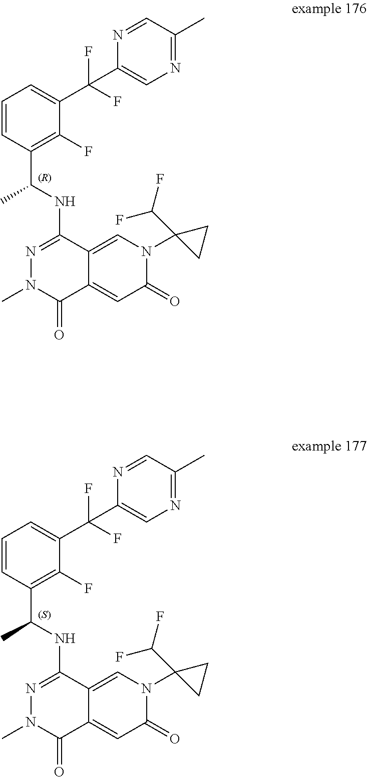

Prepared according to an analogous manner as described for examples 174/175 steps A-G, starting with 5-methylpyrazine-2-carboxylic acid. Chiral SFC: (Column: OD-3 (4.6*100 mm, 3 um), Flow rate: 3.0 mL/min, Eluent: CO2/MeOH[0.2% NH3(7M in MeOH)]=75/25, Temp 300C). Chiral HPLC: (Column: OD-3 (4.6*100 mm, 3 um), Flow rate: 3.0 mL/min, Eluent:MeOH (0.2% 7M NH3 in MeOH), Temp 40° C.).

Example 176: Chiral HPLC retention time=1.41 min. MS obsd. (ESI⁺): 547.2 [(M+H)⁺]. ¹H NMR (400 MHz, DMSO-d₆) δ: 9.00 (1H), 8.84 (1H), 8.59 (1H), 7.65-7.55 (2H), 7.32 (1H), 7.20 (1H), 6.85 (1H), 6.29 (1H), 5.05 (1H), 3.07 (3H), 2.58 (3H), 1.51 (2H), 1.46 (3H), 1.40 (2H).

Example 177: Chiral HPLC retention time=2.23 min. MS obsd. (ESI⁺): 547.2 [(M+H)⁺]. ¹H NMR (400 MHz, DMSO-d₆) δ: 9.01 (1H), 8.85 (1H), 8.60 (1H), 7.65-7.56 (2H), 7.33 (1H), 7.20 (1H), 6.86 (1H), 6.29 (1H), 5.05 (1H), 3.07 (3H), 2.59 (3H), 1.51 (2H), 1.46 (3H), 1.40 (2H).

### Example 178: (R)-4-((1-(3-(difluoromethyl)-2-fluorophenyl)ethyl)amino)-6-(1-(fluoromethyl)-2-oxabicyclo[2.1.1]hexan-4-yl)-2-methyl-2,6-dihydropyrido[3,4-d]pyridazine-1,7-dione

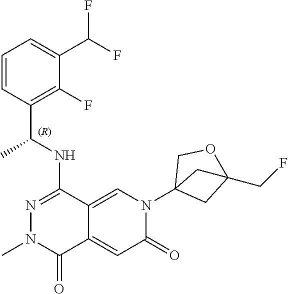

### Step A. 1-(fluoromethyl)-2-oxabicyclo[2.1.1]hexane-4-carbonitrile

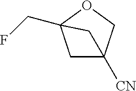

To a solution of 1-(iodomethyl)-2-oxabicyclo[2.1.1]hexane-4-carbonitrile (4.0 g, 16.1 mmol) in THE (30 mL) was added TBAF (32.1 ml, 32.1 mmol, 1M in THF). The resulting mixture was maintained under nitrogen and stirred at 80° C. for 3 h. After cooling down to rt, the reaction was quenched with water (50 mL). The resulting mixture was extracted with ethyl acetate (3×50 mL). The organic layers were combined, dried over anhydrous sodium sulfate, filtered and concentrated to afford the 1-(fluoromethyl)-2-oxabicyclo[2.1.1]hexane-4-carbonitrile (2.2 g, 97% yield). MS obsd. (ESI⁺): 142.1[M+H]⁺

### Step B. 1-(fluoromethyl)-2-oxabicyclo[2.1.1]hexane-4-carboxylic acid

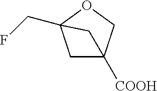 [SMILES: O=C(O)C12COC(CF)(C1)C2]

To a solution of 1-(fluoromethyl)-2-oxabicyclo[2.1.1]hexane-4-carbonitrile (2.2 g, 15.6 mmol) in THF (30 mL) and H2O (10 mL) was added LiOH (1.12 g, 46.8 mmol). The resulting mixture was maintained under nitrogen and stirred at 80° C. for 3 h. After cooling down to rt, the reaction was quenched with water (50 mL). The mixture was acidified with 5M HCl to pH=4. The resulting mixture was extracted with ethyl acetate (3×250 mL). The organic layers were combined, dried over anhydrous sodium sulfate, filtered and concentrated to afford the title compound (1.8 g, 72% yield). MS obsd. (ESI⁺): 160.1 [(M+H)⁺].

### Step C. benzyl (1-(fluoromethyl)-2-oxabicyclo[2.1.1]hexan-4-yl)carbamate

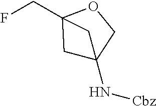 [SMILES: O=C(NC12COC(CF)(C1)C2)OCc1ccccc1]

To a solution of 1-(fluoromethyl)-2-oxabicyclo[2.1.1]hexane-4-carboxylic acid (1.8 g, 11.2 mmol) in toluene (20 mL) was added DPPA (4.10 g, 16.9 mmol), Et3N (2.27 g, 22.5 mmol), and benzyl alcohol (4.86 g, 45.0 mmol). The resulting mixture was maintained under nitrogen and stirred at 80° C. for overnight. After cooling down to rt, the reaction was quenched with water (50 mL), and then adjusted to pH 6˜7 with sodium bicarbonate. The solution was then extracted with ethyl acetate, washed with water, brine, dried over anhydrous Na2SO4 and concentrated under reduced pressure to give the crude product. The crude product was purified by reverse-phase flash (C18, CH3CN:H2O (0.05% TFA) to afford the title compound (2.4 g, 80% yield). 1H NMR (400 MHz, DMSO-d6) δ 8.20 (1H), 7.41-7.30 (5H), 5.03 (2H), 4.67-4.45 (2H), 3.68 (2H), 2.09-2.00 (2H), 1.79-1.73 (2H).

### Step D: 1-(fluoromethyl)-2-oxabicyclo[2.1.1]hexan-4-amine hydrogen chloride

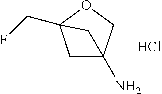

To a solution of benzyl N-[1-(fluoromethyl)-2-oxabicyclo[2.1.1]hexan-4-yl]carbamate (2400 mg, 9.05 mmol) in EA (50 mL) was added Pd/C (250 mg, 10 wt %). The flask was evacuated and flushed three times with nitrogen, followed by flushing with hydrogen. The mixture was stirred 6 h at 30° C. under an H2 atmosphere. After cooling down to r.t, the mixture was filtered through a Celite pad and concentrated under reduced pressure. The residue was dissolved in 4M HCl in dioxane and concentrated again to afford the title compound (1.40 g, 92% yield). MS obsd. (ESI⁺): 132.20 [(M−HCl+H)⁺]. 19 F NMR (400 MHz, DMSO-d6) δ-73.884.

### Step E: (R)-4-((1-(3-(difluoromethyl)-2-fluorophenyl)ethyl)amino)-6-(1-(fluoromethyl)-2-oxabicyclo[2.1.1]hexan-4-yl)-2-methyl-2,6-dihydropyrido[3,4-d]pyridazine-1,7-dione (example 178)

The title compound was prepared according to an essential analogous route to example 127, starting with 1-(fluoromethyl)-2-oxabicyclo[2.1.1]hexan-4-amine hydrochloride. MS obsd. (ESI⁺): 479.2 [(M+H)⁺]. ¹H NMR (400 MHz, DMSO-d₆) δ: 8.58 (1H), 7.63 (1H), 7.49 (1H), 7.36-7.09 (3H), 6.84 (1H), 5.26-5.15 (1H), 4.81 (1H), 4.69 (1H), 3.98 (2H), 3.26 (3H), 2.46 (2H), 2.36-2.34 (2H), 1.53 (3H).

### Example 179: (R)-4-((1-(3-(difluoromethyl)-2-fluorophenyl)ethyl)amino)-2-methyl-6-(1-methyl-2-oxabicyclo[2.1.1]hexan-4-yl)-2,6-dihydropyrido[3,4-d]pyridazine-1,7-dione

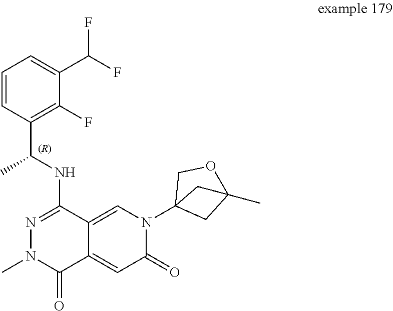

Prepared according to an essentially analogous route to example 127. MS obsd. (ESI⁺): 461.0 [(M+H)⁺].

### Examples 180 and 181: (R)-4-((1-(4,4-difluorochroman-8-yl)ethyl)amino)-6-(1-(difluoromethyl)cyclopropyl)-2-methyl-2,6-dihydropyrido[3,4-d]pyridazine-1,7-dione and (S)-4-((1-(4,4-difluorochroman-8-yl)ethyl)amino)-6-(1-(difluoromethyl)cyclopropyl)-2-methyl-2,6-dihydropyrido[3,4-d]pyridazine-1,7-dione

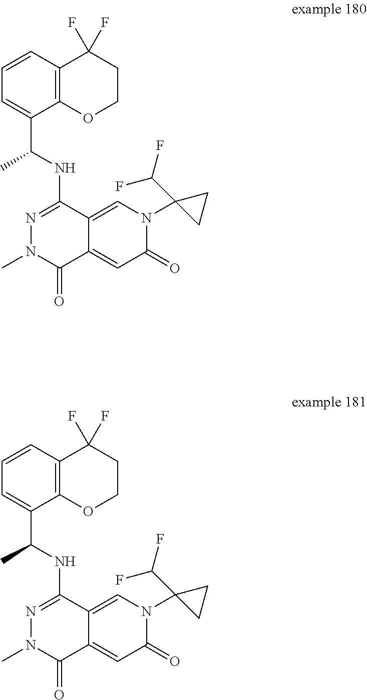 [SMILES: C[C@@H](Nc1nn(C)c(=O)c2cc(=O)n(C3(C(F)F)CC3)cc12)c1cccc2c1OCCC2(F)F ; C[C@H](Nc1nn(C)c(=O)c2cc(=O)n(C3(C(F)F)CC3)cc12)c1cccc2c1OCCC2(F)F]

Prepared in an essentially analogous manner to examples 95/96 step D, starting with 1-(4,4-difluorochroman-8-yl)ethan-1-amine hydrochloride (prepared according to the method of WO/2020/180770) and 4-chloro-6-[1-(difluoromethyl)cyclopropyl]-2-methyl-pyrido[3,4-d]pyridazine-1,7-dione. Chiral HPLC: Column: Cellulose-SC (4.6*100 mm, 3 um), Flow rate: 3.0 mL/min, Eluent:MeOH (0.2% 7M NH3 in MeOH), Temp 400C).

Example 180: Chiral HPLC retention time: 2.69 min. MS obsd. (ESI⁺): 479.4 [(M+H)⁺].

Example 181: Chiral HPLC retention time: 3.58 min. MS obsd. (ESI⁺): 479.5 [(M+H)⁺].

### Example 182: (R)-6-cyclopropyl-2-methyl-4-((1-(naphthalen-2-yl)ethyl)amino)-2,6-dihydropyrido[3,4-d]pyridazine-1,7-dione

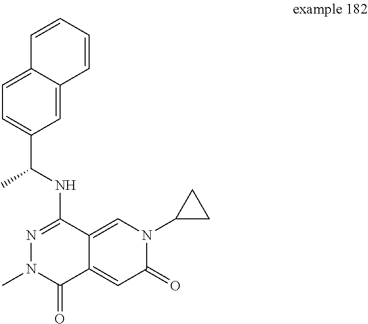 [SMILES: C[C@@H](Nc1nn(C)c(=O)c2cc(=O)n(C3CC3)cc12)c1ccc2ccccc2c1]

Prepared according to an analogous route as described for example 2 using (R)-1-(naphthalen-2-yl)ethan-1-amine. MS obsd. (ESI+): 387.3 (M+H)+

### Biological Assays

### SOS1-KRas(G12C) FRET Assay

Inhibition of the SOS1:KRAS interaction was measured using purified GST-tagged KRAS (res. 1-169, G12C, purified based on Hillig, et al., Proc Natl Acad Sci USA (2019); 116(7):2551-2560) and recombinant His10-SOS1 (res. 564-1049; purified based on Hillig, et al.). The final assay was performed at 20 uL with 0.5 nM SOS1 protein and 2.5 nM KRAS protein in a buffer of PBS, 0.1% BSA, 5 mM MgCl₂, 0.0025% Igepal, 100 mM KF, 5 mM DTT in a white 384 square well OptiPlate (PerkinElmer, Cat. 6007290). A 2× KRAS working solution was prepared in an assay buffer containing 5 nM GST-KRAS G12C and 2 nM anti-GST-Eu(K) (Cisbio, Cat. 61GSTKLA) and pre-incubated for 15 minutes at 25° C. Compounds were serially diluted in 100% DMSO from 2 mM (positive control, compound I-13, PCT Publ. No. WO2018/115380) or 20 mM and then diluted 1:20 in assay buffer before incubation with a solution of SOS1 protein mixed 1:5 with anti-6His-XL665 FRET donor (Cisbio, Cat. 61HISXL) for 15 minutes at 25° C. before addition of 2× KRAS working solution. The final DMSO concentration is 0.5%. Plates were incubated at RT for 2 hrs before the FRET signal was measured using Envision at emission 665 nm and 615 nm. FRET signal was converted to percentage of protein-protein interaction using the following equation:

$$\% Inhibition = 100 \% - ( C - N ) / ( P - N ) * 100 \%$$

C: signal with compound treatment
P: signal for positive control (DMSO)
N: signal for negative control (no SOS1 added)
IC₅₀ and Hill coefficients were obtained using Graph Pad Prism (Graph Pad software, Inc, USA) with non-linear regression analysis.

TABLE A
Inhibition of SOS1 and KRas(G12C) Interaction

| | SOS-KRas(G12C) |
|---|---|
| Example No. | FRET IC₅₀ [uM] |
| 1 | 1.03 |
| 2 | 0.328 |
| 3 | 0.345 |
| 4 | 0.0546 |
| 5 | 0.0964 |
| 6 | 0.161 |
| 7 | 1.05 |
| 8 | 1.31 |
| 9 | 0.286 |
| 10 | 2.25 |
| 11 | 0.592 |
| 12 | 1.33 |
| 13 | 0.0996 |
| 14 | 0.537 |
| 15 | 0.198 |
| 16 | 0.235 |
| 17 | 0.248 |
| 18 | 0.0538 |
| 19 | 0.806 |
| 20 | 0.284 |
| 21 | 0.3 |
| 22 | 0.35 |
| 23 | 1.95 |
| 24 | 0.239 |
| 25 | 3.86 |
| 26 | 100 |
| 27 | 1.53 |
| 28 | 76.17 |
| 29 | 0.0619 |
| 30 | 0.0207 |
| 31 | 0.146 |
| 32 | 1.05 |
| 33 | 0.0313 |
| 34 | 0.0439 |
| 35 | 0.0784 |
| 36 | 0.0333 |
| 37 | 0.0304 |
| 38 | 0.211 |
| 39 | 0.16 |
| 40 | 0.0211 |
| 41 | 0.0279 |
| 42 | 0.0549 |
| 43 | 0.0605 |
| 44 | 5.81 |
| 45 | 0.0234 |
| 46 | 0.0624 |
| 47 | 0.0824 |
| 48 | 0.192 |
| 49 | 0.0513 |
| 50 | 0.0544 |
| 51 | 0.0473 |
| 52 | 0.272 |
| 53 | 0.0303 |
| 54 | 0.285 |
| 55 | 0.0322 |
| 56 | 1.93 |
| 57 | 0.0219 |
| 58 | 0.0327 |
| 59 | 1.04 |
| 60 | 0.129 |
| 61 | 0.0259 |
| 62 | 0.0753 |
| 63 | >10 |
| 64 | 0.126 |
| 65 | >100 |
| 66 | 0.0725 |
| 67 | 0.0246 |
| 68 | 1.71 |
| 69 | 0.0348 |
| 70 | 0.0167 |
| 71 | 0.0698 |
| 72 | 0.0683 |
| 73 | 0.0608 |
| 74 | 0.091 |
| 75 | 0.127 |
| 76 | 0.0928 |
| 77 | 0.305 |
| 78 | 0.089 |
| 79 | 0.3 |
| 80 | 0.302 |
| 81 | 0.376 |
| 82 | 0.476 |
| 83 | 0.0487 |
| 84 | 0.229 |
| 85 | 0.192 |
| 86 | 0.0353 |
| 87 | 0.118 |
| 88 | 0.0835 |
| 89 | 0.122 |
| 90 | 0.0597 |
| 91 | 0.0974 |
| 92 | 0.567 |
| 93 | 0.0608 |
| 94 | 0.378 |
| 95 | 0.394 |
| 96 | 0.0292 |
| 97 | 0.0656 |
| 98 | 0.989 |
| 99 | 0.00903 |
| 100 | 1.92 |
| 101 | >100 |
| 102 | 3.79 |
| 103 | 7.8 |
| 104 | 0.037 |
| 105 | >10 |
| 106 | 0.366 |
| 107 | 0.271 |
| 108 | >10 |
| 109 | 0.12 |
| 110 | 0.0481 |
| 111 | 0.631 |
| 112 | >10 |
| 113 | >10 |
| 114 | 2.06 |
| 115 | >10 |
| 116 | >10 |
| 117 | 0.568 |
| 118 | >10 |
| 119 | 0.411 |
| 120 | 0.0199 |
| 121 | 0.125 |
| 122 | 0.315 |
| 123 | 0.366 |
| 124 | 0.886 |
| 125 | 0.0543 |
| 126 | 0.0678 |
| 127 | 0.090 |
| 128 | 0.287 |
| 129 | 0.112 |
| 130 | 0.0826 |
| 131 | 0.277 |
| 132 | 0.152 |
| 133 | 0.245 |
| 134 | 0.282 |
| 135 | 0.0705 |
| 136 | 0.185 |
| 137 | 0.0912 |
| 138 | 0.434 |
| 139 | 0.12 |
| 140 | 0.0313 |
| 141 | 0.025 |
| 142 | 0.0772 |
| 143 | 0.0078 |
| 144 | 0.209 |
| 145 | 0.083 |
| 146 | 0.0706 |
| 147 | 0.0324 |
| 148 | 0.0405 |
| 149 | 0.0343 |
| 150 | 0.0055 |
| 151 | 0.564 |
| 152 | 0.248 |
| 153 | 0.0869 |
| 154 | 0.0185 |
| 155 | 0.159 |
| 156 | 0.0666 |
| 157 | 0.0242 |
| 158 | 0.382 |
| 159 | 3.48 |
| 160 | 0.0367 |
| 161 | 0.0249 |
| 162 | >10 |
| 163 | 0.0756 |
| 164 | 0.0193 |
| 165 | 0.424 |
| 166 | 0.0348 |
| 167 | 0.607 |
| 168 | 0.0135 |
| 169 | 0.262 |
| 170 | 0.0065 |
| 171 | 1.74 |
| 172 | 0.0138 |
| 173 | 2.79 |
| 174 | 0.0044 |
| 175 | 4.49 |
| 176 | 0.0201 |
| 177 | >10 |
| 178 | 0.076 |
| 179 | 0.0684 |
| 180 | 0.179 |
| 181 | >10 |
| 182 | 2.44 |

### Surface Plasmon Resonance (SPR) SOS1 Binding Assay

Binding to SOS1 was measured using a SPR assay with purified recombinant human SOS1 substrate (res. 564-1049 with N-terminal Avi tag; purified and biotinylated based on Hillig, et al., Proc Natl Acad Sci USA (2019); 116(7):2551-2560). SPR measurements were performed on a Biacore 8K SPR instrument (GE Healthcare, Sweden). Assays were performed at 25° C. using Series S SA sensor chips pre-coated with streptavidin (GE Healthcare, Cat. BR100531). Biotinylated SOS1 diluted in sample buffer (20 mM Tris HCl, 150 mM NaCl, 1 mM DTT, 0.05% TWEEN 20, 1 mM MgCl₂, pH 8.0) was captured to one flow cell of the chip to about 3,000 resonance units (RU) using sample buffer supplemented with 5% DMSO as a running buffer. Serial dilutions of the assayed compounds in the running buffer at 100, 50 or 0.5 μM were injected for 60 s at a flow rate of 30 μL/min and association phases were recorded. Dissociation of the samples was monitored for 600 s. Data processing was performed using Biacore Insight Software (Biacore, GE Healthcare). Sensorgrams recorded on a SA flow cell without captured protein were subtracted from sensorgrams recorded on the SOS1 surface. Blank injections of running buffer were used for double referencing and solvent correction was applied to all sample sensorgrams to correct for buffer mismatches. K_{D}s were estimated using a kinetic or steady state, where applicable, fitting model describing a reversible equilibrium with 1:1 binding between SOS1 and the compound.

TABLE B
Binding Affinity of Selected Compounds for SOS1

| | KD for |
|---|---|
| Example No. | hSOS1 (nM) |
| 2 | 57 |
| 3 | 63 |
| 4 | 22 |
| 5 | 22 |
| 6 | 44 |
| 7 | 259 |
| 9 | 64 |
| 10 | 136 |
| 11 | 95 |
| 12 | 290 |
| 13 | 25 |
| 14 | 87 |
| 15 | 40 |
| 16 | 84 |
| 17 | 31 |
| 18 | 16 |
| 19 | 161 |
| 20 | 63 |
| 21 | 70 |
| 22 | 74 |
| 24 | 34 |
| 25 | 225 |
| 26 | >50000 |
| 27 | 134 |
| 28 | 3020 |
| 29 | 42 |
| 30 | 9 |
| 31 | 45 |
| 32 | 229 |
| 33 | 8 |
| 34 | 10 |
| 35 | 17 |
| 36 | 16 |
| 38 | 43 |
| 39 | 43 |
| 40 | 9 |
| 41 | 14 |
| 42 | 34 |
| 43 | 24 |
| 44 | >5000 |
| 45 | 10 |
| 46 | 21 |
| 47 | 6 |
| 48 | 75 |
| 49 | 13 |
| 50 | 29 |
| 51 | 17 |
| 52 | 76 |
| 53 | 7 |
| 54 | 125 |
| 55 | 14 |
| 56 | 530 |
| 57 | 8 |
| 58 | 15 |
| 59 | 441 |
| 60 | 43 |
| 61 | 10 |
| 62 | 13 |
| 64 | 31 |
| 66 | 26 |
| 67 | 10 |
| 68 | 1490 |
| 69 | 10 |
| 70 | 12 |
| 71 | 31 |
| 72 | 60 |
| 73 | 27 |
| 74 | 69 |
| 75 | 73 |
| 76 | 37 |
| 77 | 61 |
| 78 | 55 |
| 79 | 124 |
| 80 | 303 |
| 81 | 134 |
| 82 | 51 |
| 83 | 11 |
| 84 | 28 |

What is claimed is:

1. A compound of Formula (I): 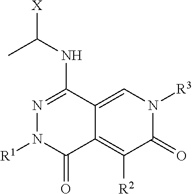
or a pharmaceutically acceptable salt thereof, wherein:
X is phenyl, naphthyl, 3-10 membered cycloalkyl, 3-10 membered heterocyclyl, or 5-10 membered heteroaryl, each of which is optionally substituted by 1-3 independently selected R^{A};
R¹ is C₁-C₆ alkyl, —(C₁-C₆ alkyl)-NR^{B}R^{C}, C₁-C₆ hydroxyalkyl, or 5-6 membered heteroaryl;
R² is hydrogen, halogen, cyano, hydroxyl, —NR^{D}R^{E}, C₁-C₆ alkyl, C₁-C₆ alkoxy optionally substituted with 3-10 membered cycloalkyl or heterocyclyl, C(O)C₁-C₆alkyl, C₁-C₆ hydroxycycloalkyl optionally substituted with 1-3 fluoro, C₁-C₆ hydroxyheterocyclyl optionally substituted with 1-3 fluoro, or 3-10 membered heterocyclyl optionally substituted with amino;
R³ is
(A) C₃-C₈ cycloalkyl optionally substituted with hydroxyl, cyano, C₁-C₆ alkyl or 1-3 fluoro, C₃-C₆ cycloalkyl, 5-10 membered heteroaryl optionally substituted with 1-3 fluoro, 3-10 membered —CH₂-heteroyclyl substituted with 1-3 fluoro, C₁-C₆ haloalkyl, C₁-C₆ hydroxyalkyl —C(O)NR^{F}R^{G} or C₁-C₆ alkyl, or
(B) 3-10 membered heterocyclyl optionally independently substituted with one or more halogen, C₁-C₆ alkyl, C₁-C₆ haloalkyl, —C(O)C₁-C₆alkyl, —C(O)C₁-C₆ cycloalkyl optionally substituted by halogen, C₁-C₆ alkyl or C₁-C₆ alkoxy, or —C(O)NR^{F}R^{G};
each R^{A} is independently selected from one or more halogen, cyano, nitro, —(C₁-C₆ alkyl)ₚ-NR^{H}R^{I}; C₁-C₆ alkyl, C₁-C₆ alkoxy, C₁-C₆ haloalkoxy, C₁-C₆ hydroxyalkyl optionally substituted with 1-3 fluoro, C₁-C₆ alkoxyalkyl optionally independently substituted with one or more 1-3 fluoro or C₁-C₆ alkyl, C₁-C₆ haloalkyl optionally substituted with 3-10 membered heteroyclyl, —CH₂C(O)NR^{F}R^{G} optionally substituted with 1-3 fluoro, 5-6 membered —CH₂-heteroaryl substituted with 1-3 fluoro, and 3-10 membered cycloalkyl optionally substituted with —(C₁-C₆ alkyl)ₚ-NR^{J}R^{K};
each R^{B}, R^{C}, R^{D}, R^{E}, R^{F}, R^{G}, R^{H}, R^{I}, R^{J}, R^{K} are independently selected from hydrogen and C₁-C₆ alkyl, or R^{B} and R^{C} together with the atom to which they are bonded can form a 5-10 membered heterocyclyl optionally substituted with one or more of halogen, C₁-C₆ alkyl, and C₁-C₆ alkoxy; and
p is 0 or 1.

2. The compound of claim 1, wherein R¹ is C₁-C₆ alkyl.

3. The compound of claim 1, wherein R¹ is methyl.

4. The compound of claim 1, wherein R² is hydrogen.

5. The compound of claim 1, wherein R² is C₁-C₆ alkoxy.

6. The compound of claim 5, wherein R² is methoxy

7. The compound of claim 1, wherein R³ is C₃-C₆ cycloalkyl optionally substituted with hydroxyl, cyano, 5-10 membered heteroaryl, C₁-C₆ haloalkyl, —C(O)NR^{F}R^{G}, or C₁-C₆ alkyl.

8. The compound of claim 7, wherein R³ is cyclopropyl optionally substituted with hydroxyl, cyano, 5-10 membered heteroaryl, C₁-C₆ haloalkyl, —C(O)NR^{F}R^{G}, or C₁-C₆ alkyl.

9. The compound of claim 1, wherein R³ is a 3-10 membered heterocyclyl optionally substituted with halogen, C₁-C₆ alkyl, C₁-C₆ haloalkyl, —C(O)C₁-C₆ alkyl, or —C(O)NR^{F}R^{G}.

10. The compound of claim 9, wherein R³ is tetrahydropyran optionally substituted with halogen, C₁-C₆ alkyl, C₁-C₆ haloalkyl, —C(O)C₁-C₆ alkyl, or —C(O)NR^{F}R^{G}.

11. The compound of claim 1, wherein X is phenyl, naphthyl, 3-10 membered cycloalkyl, 3-10 membered heterocyclyl or 5-10 membered heteroaryl, each of which is optionally substituted by 1-3 independently selected R^{A}.

12. The compound of claim 1, wherein X is selected from:

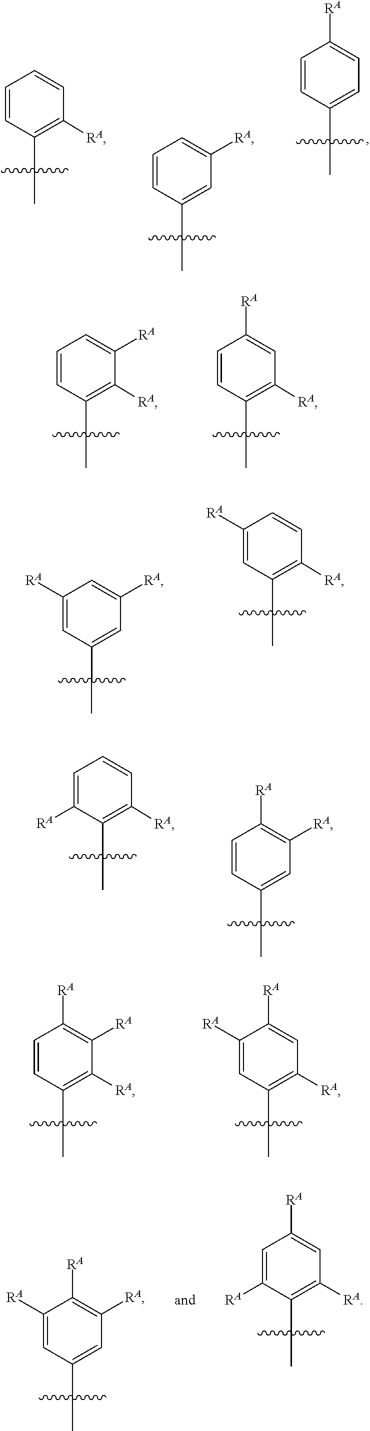

13. The compound of claim 1, wherein each R^{A} is independently selected from cyano, halogen, C₁-C₃ alkyl, and C₁-C₃ haloalkyl optionally substituted with 3-10 membered heteroyclyl.

14. The compound of claim 13, wherein each R^{A} is independently selected from cyano, methyl, fluoro, methoxy, difluoromethyl, trifluoromethyl, trifluoromethoxy, and hydroxyethyl.

15. The compound of claim 14, wherein at least one R^{A} is fluoro.

16. The compound of claim 14, wherein at least one R^{A} is difluoromethyl.

17. A compound selected from the group consisting of the compounds in Examples 1-182, or a pharmaceutically acceptable salt thereof.

18. A pharmaceutical composition comprising a compound of any one of claims 1-17, or a pharmaceutically acceptable salt thereof, and at least one pharmaceutically acceptable excipient.

19. A method for treating cancer in a subject in need thereof, comprising administering to the subject an effective amount of a compound of any one of claims 1-17.

20. The method according to claim 19, wherein the cancer is a Ras pathway-associated cancer.
